(19) United States
(12) Patent Application Publication
(10) Pub. No.: US 2024/0277872 A1
(43) Pub. Date: Aug. 22, 2024

(54) MODIFIED MRNA, MODIFIED NON-CODING RNA, AND USES THEREOF

(71) Applicants: The Broad Institute, Inc., Cambridge, MA (US); Massachusetts Institute of Technology, Cambridge, MA (US)

(72) Inventors: Xiao Wang, Cambridge, MA (US); Hailing Shi, Cambridge, MA (US); Abhishek Aditham, Cambridge, MA (US)

(73) Assignees: The Broad Institute, Inc., Cambridge, MA (US); Massachusetts Institute of Technology, Cambridge, MA (US)

(21) Appl. No.: 18/560,348

(22) PCT Filed: May 11, 2022
(86) PCT No.: PCT/US2022/028849

§ 371 Date: Nov. 10, 2023

Related U.S. Application Data
(60) Provisional application No. 63/288,522, filed on Dec. 10, 2021.
(60) Provisional application No. 63/187,752, filed on May 12, 2021.

(51) Int. Cl. A61K 48/00; A61K 38/53; C12N 15/10; C12N 15/67

(52) U.S. Cl. CPC A61K 48/005; A61K 38/53; C12N 15/1013; C12N 15/67; C12Y 605/01003; C12N 2310/313; C12N 2310/321; C12N 2310/322; C12N 2310/336

(57) ABSTRACT

Disclosed herein are modified niRNAs and modified non-coding RNAs with poly(A) tails containing modified nucleotides and/or secondary structures, which may be made by ligation of a tailing nucleic acid onto the 3′ terminal end of an RNA. Also provided are compositions comprising one or more modified mRNAs or modified non-coding RNAs provided herein, and methods of using said compositions for therapeutic or agricultural applications.

### CROSS REFERENCE TO RELATED APPLICATIONS

This application claims the benefit under 35 U.S.C. § 119(e) of U.S. Provisional Application No. 63/187,752, filed May 12, 2021, entitled “MODIFIED MRNA AND USES THEREOF,” and U.S. Provisional Application No. 63/288,522, filed Dec. 10, 2021, entitled “MODIFIED MRNA AND USES THEREOF,” the entire disclosures of each of which are hereby incorporated by reference in their entireties.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on May 10, 2022, is named B119570130WO00-SEQ-JQM, and is 9,854 bytes in size.

### BACKGROUND

Messenger RNA (mRNA) technology is an emerging alternative to conventional small molecule therapeutics and vaccine approaches because it is potent, programmable, and capable of rapid production of mRNAs with desired sequences. mRNA therapeutics is a rapidly developing field and has been used for the expression of therapeutic proteins, ranging from vascular regeneration factors to vaccines for COVID-19, influenza, and Zika virus. Despite recent clinical successes, mRNA therapeutics still faces challenges of instability, toxicity, short-term efficacy, and potential allergic responses. Increasing the stability of mRNAs to enhance their efficacy in vivo remains an important problem that must be solved to increase the feasibility of mRNA therapeutics for clinical applications.

### SUMMARY

Provided herein are modified mRNAs with modified nucleotides and/or structural features to improve stability in cells and thereby enhance protein production, as well as methods of making and using such modified mRNAs. Conventional mRNAs comprise poly-A tails with multiple adenosine nucleotides at the 3′ end, which can be degraded by cellular exonucleases, which remove 3′ nucleotides. Once exonucleases remove the poly-A tail and begin removing nucleotides of the open reading frame, the mRNA is unable to be translated into an encoded protein. mRNAs that are more resistant to 3′ exonuclease activity are degraded more slowly and are thus more stable, having increased half-lives in cells, and more protein can be produced from a given mRNA molecule. Modified nucleotides containing one or more structural modifications to the nucleobase, sugar, or phosphate linkage of the mRNA can interfere with 3′ exonuclease activity, rendering the mRNA more stable. However, the same structural modifications that inhibit 3′ exonucleases can also hinder the ability of polyadenylating enzymes to incorporate them into a poly-A tail, making it difficult to incorporate modified nucleotides into a poly-A tail. Surprisingly, ligating an oligonucleotide containing as few as three modified nucleotides onto the 3′ end of an mRNA containing a pre-existing poly-A tail results in a marked improvement in mRNA stability, compared to ligation of an oligonucleotide with no modified nucleotides other than a blocking 3′ terminal nucleotide to prevent oligonucleotide self-ligation (FIG. 5). Similar improvements in stability were observed by ligation of an oligonucleotide containing structural sequences capable of forming a secondary structure, such as a G-quadruplex or aptamer. Such structural sequences are thought to prevent exonucleases from accessing 3′ terminal nucleotides. Multiple types of modified nucleotides and structural sequences, both alone and in combination with each other, imparted improved stability to mRNAs when added to the 3′ terminus, rendering the modified mRNAs more resistant to RNase-mediated degradation, which resulted in increased protein production from these modified mRNAs relative to control mRNAs. These results indicate that this approach of modifying the poly-A tail of mRNAs to hinder exonuclease activity provides broad utility in the production of modified mRNAs. Additionally, modified mRNAs produced by the methods provided herein may be circularized by ligating the terminal ends of a linear mRNA to produce a circular mRNA. The techniques described herein for improving the stability of a mRNA may also be suitable for improving the stability of a non-coding RNA, for the reason that non-coding RNA is also vulnerable to 3′ exonuclease activity.

Accordingly, the present disclosure provides, in some aspects, a modified mRNA comprising:
(i) an open reading frame (ORF) encoding a protein; and
(ii) a poly-A region,
wherein the poly-A region is 3′ to the open reading frame and comprises 10 or more nucleotides, wherein 1% to 90% of the nucleotides of the poly-A region are modified nucleotides, and wherein 3 or more of the 10 last nucleotides of the poly-A region are modified nucleotides.

In some embodiments, the poly-A region comprises 25 or more adenosine nucleotides, wherein 1% to 90% of the nucleotides of the poly-A region are modified nucleotides, and 3 or more of the 25 last nucleotides of the poly-A region are modified nucleotides.

In some embodiments, 4 or more of the 25 last nucleotides of the poly-A region are modified nucleotides.

In some embodiments, 2 or more consecutive nucleotides of the 25 last nucleotides of the poly-A region are linked by a modified internucleotide linkage.

In some embodiments, 3 or more consecutive nucleotides of the 25 last nucleotides of the poly-A region are modified nucleotides independently selected from a deoxyribonucleotide, a 2′-modified nucleotide, and a phosphorothioate-linked nucleotide.

In some embodiments, the 3 or more modified nucleotides are consecutive nucleotides located at the 3′ terminus of the poly-A region.

In some embodiments, 6 or more consecutive nucleotides of the 25 last nucleotides of the poly-A region comprise the same type of nucleotide or internucleoside modification.

In some embodiments, 3 or more of the 10 last nucleotides of the poly-A region are modified nucleotides.

In some embodiments, at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the poly-A region are modified nucleotides.

In some embodiments, at least 4, 5, 6, 7, 8, 9, 10, 15, 20, or 25 of the 25 last nucleotides of the poly-A region are modified nucleotides.

In some embodiments, the modified mRNA comprises a 5′ untranslated region (5′ UTR) and a 3′ untranslated region (3′ UTR), wherein the ORF is between the 5′ UTR and the 3′ UTR, wherein the 3′ UTR is between the ORF and the poly-A region.

In some embodiments, the modified mRNA is a circular mRNA, wherein the poly-A region is between the 3′ UTR and the 5′ UTR.

In some aspects, the present disclosure provides a modified mRNA comprising:
(i) an open reading frame (ORF) encoding a protein;
(ii) a poly-A region;
(iii) one or more copies of a structural sequence comprising at least two nucleotides that are capable of forming a secondary structure,
   wherein the poly-A region is 3′ to the open reading frame and comprises 10 or more nucleotides,
   wherein the one or more copies of the structural sequence are 3′ to the poly-A region, and
   wherein the modified mRNA comprises a secondary structure, wherein the secondary structure comprises one or more copies of the structural sequence.

In some embodiments, the poly-A region is 3′ to the open reading frame and comprises 25 or more nucleotides, wherein the one or more copies of the structural sequence are 3′ to the poly-A region, and wherein the modified mRNA comprises a secondary structure, wherein the secondary structure comprises one or more copies of the structural sequence.

In some embodiments, the modified mRNA comprises a 5′ untranslated region (5′ UTR) and a 3′ untranslated region (3′ UTR), wherein the ORF is between the 5′ UTR and the 3′ UTR, wherein the 3′ UTR is between the ORF and the poly-A region.

In some embodiments, the modified mRNA is a circular mRNA, wherein the one or more copies of the structural sequence are between the poly-A region and the 5′ UTR.

In some embodiments, the structural sequence is a G-quadruplex sequence.

In some embodiments, the G-quadruplex is an RNA G-quadruplex sequence.

In some embodiments, the RNA G-quadruplex sequence comprises the nucleic acid sequence of SEQ ID NO: 2.

In some embodiments, the modified mRNA comprises at least 3 copies of the nucleic acid sequence of SEQ ID NO: 2.

In some embodiments, the G-quadruplex is a DNA G-quadruplex sequence.

In some embodiments, the DNA G-quadruplex sequence comprises the nucleic acid sequence of SEQ ID NO: 3.

In some embodiments, the modified mRNA comprises at least 3 copies of the nucleic acid sequence of SEQ ID NO: 3.

In some embodiments, the structural sequence is a telomeric repeat sequence.

In some embodiments, the telomeric repeat sequence comprises the nucleic acid sequence of SEQ ID NO: 4.

In some embodiments, the modified mRNA comprises at least 3 copies of the nucleic acid sequence of SEQ ID NO: 4.

In some embodiments, the secondary structure of the mRNA is an aptamer that is capable of binding to a target molecule.

In some embodiments, the poly-A region of the modified mRNA comprises at least one modified nucleotide.

In some embodiments, at least one modified nucleotide comprises a modified nucleobase.

In some embodiments, the modified nucleobase is selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

In some embodiments, at least one modified nucleotide comprises a modified sugar.

In some embodiments, the modified sugar is selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose.

In some embodiments, at least one modified nucleotide comprises a 2′ modification.

In some embodiments, the 2′ modification is selected from the group consisting of a locked-nucleic acid (LNA) modification (i.e., a nucleotide comprising an additional carbon atom bound to the 2′ oxygen and 4′ carbon of ribose), 2′-fluoro (2′-F), 2′-O-methoxy-ethyl (2′-MOE), and 2′-O-methylation (2′-OMe). In some embodiments, at least one modified nucleotide comprises a modified phosphate.

In some embodiments, the modified phosphate is selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

In some embodiments, the poly-A region comprises at least 3, at least 4, at least 5, or at least 6 phosphorothioates.

In some embodiments, the poly-A region comprises at least 6 phosphorothioates.

In some embodiments, the poly-A region comprises at least 3 guanine nucleotides and least 3 phosphorothioates.

In some embodiments, the poly-A region comprises at least 6 nucleotides comprising a 2′ modification.

In some embodiments, the poly-A region comprises at least 3 deoxyribose sugars.

In some embodiments, the poly-A region comprises at least 5, at least 10, at least 15, at least 20, or at least 23 deoxyribose sugars.

In some embodiments, the poly-A region comprises at least 23 deoxyribose sugars.

In some embodiments, the 3′ terminal nucleotide of the mRNA does not comprise hydroxy at the 3′ position of the 3′ terminal nucleotide.

In some embodiments, the 3′ terminal nucleotide of the mRNA comprises an inverted nucleotide.

In some embodiments, the 3′ terminal nucleotide of the mRNA comprises a dideoxyadenosine, dideoxycytidine, dideoxyguanosine, dideoxythymidine, dideoxyuridine, or inverted-deoxythymidine.

In some embodiments, the 3′ terminal nucleotide of the mRNA comprises a dideoxycytidine.

In some embodiments, the mRNA comprises a peptide-binding sequence. In some embodiments, the peptide-binding sequence is a poly-A binding protein (PABP)-binding sequence

In some embodiments, the modified mRNA comprises a first modified nucleotide and a second modified nucleotide, wherein the first and second modified nucleosides comprise different structures.

In some embodiments, the poly-A region comprises at least 25-500 nucleotides.

In some embodiments, the poly-A region comprises at least 50, at least 100, at least 150, or at least 200 nucleotides.

In some embodiments, at least 25%, at least 30%, at least 40%, at least 50%, at least 60%, at least 70%, at least 80%, at least 90%, at least 95%, at least 96%, at least 97%, at least 98%, or at least 99% of nucleotides of the poly-A region are adenosine nucleotides.

In some embodiments, the modified mRNA is a linear mRNA, wherein the linear mRNA comprises a 5′ cap.

In some embodiments, the 5′ cap comprises a 7-methylguanosine.

In some embodiments, the 5′ cap further comprises one or more phosphates connecting the 7-methylguanosine to an adjacent nucleotide of the modified mRNA.

In some embodiments, the 5′ cap comprises a 3′-O-Me-m7G(5′)ppp(5′)G.

In some embodiments, one or more phosphates of the 5′ cap is a modified phosphate selected from the group consisting of phosphorothioate, triazole ring, dihalogenmethylenebisphosphonate, imidodiphosphate, and methylenebis(phosphonate).

In some embodiments, the modified mRNA comprises a 5′ UTR comprising 1 or more modified nucleotides. In some embodiments, the modified mRNA comprises an ORF comprising 1 or more modified nucleotides.

In some aspects, the present disclosure provides a modified non-coding RNA comprising:
(i) a non-coding RNA sequence; and
(ii) a poly-A region,
wherein the poly-A region is 3′ to the non-coding RNA sequence and comprises 10 or more nucleotides, wherein 1% to 90% of the nucleotides of the poly-A region are modified nucleotides, and wherein 3 or more of the 10 last nucleotides of the poly-A region are modified nucleotides.

In some embodiments, the poly-A region is 3′ to the open reading frame and comprises 25 or more adenosine nucleotides, wherein 1% to 90% of the nucleotides of the poly-A region are modified nucleotides, and wherein 3 or more of the 25 last nucleotides of the poly-A region are modified nucleotides.

In some embodiments, 4 or more of the 25 last nucleotides of the poly-A region are modified nucleotides.

In some embodiments, 2 or more consecutive nucleotides of the 25 last nucleotides of the poly-A region are linked by a modified internucleotide linkage.

In some embodiments, 3 or more consecutive nucleotides of the 25 last nucleotides of the poly-A region are modified nucleotides independently selected from a deoxyribonucleotide, a 2′-modified nucleotide, and a phosphorothioate-linked nucleotide.

In some embodiments, the 3 or more modified nucleotides are consecutive nucleotides located at the 3′ terminus of the poly-A region.

In some embodiments, 6 or more consecutive nucleotides of the 25 last nucleotides of the poly-A region comprise the same type of nucleotide or internucleoside modification.

In some embodiments, at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the poly-A region are modified nucleotides.

In some embodiments, at least 4, 5, 6, 7, 8, 9, 10, 15, 20, or 25 of the 25 last nucleotides of the poly-A region are modified nucleotides.

In some embodiments, the modified non-coding RNA is a circular non-coding RNA, wherein the poly-A region is 5′ to the non-coding RNA sequence.

In some embodiments, the modified non-coding RNA further comprises one or more copies of a structural sequence comprising at least two nucleotides that are capable of forming a secondary structure, wherein the one or more copies of the structural sequence are 3′ to the poly-A region, and wherein the modified non-coding RNA comprises a secondary structure, and wherein the secondary structure comprises one or more copies of the structural sequence.

In some embodiments, the modified non-coding RNA is a circular mRNA, wherein the one or more copies of the structural sequence are between the poly-A region and the non-coding RNA sequence.

In some embodiments, the structural sequence is a G-quadruplex sequence.

In some embodiments, the G-quadruplex is an RNA G-quadruplex sequence.

In some embodiments, the RNA G-quadruplex sequence comprises the nucleic acid sequence of SEQ ID NO: 2.

In some embodiments, the modified non-coding RNA comprises at least 3 copies of the nucleic acid sequence of SEQ ID NO: 2.

In some embodiments, the G-quadruplex is a DNA G-quadruplex sequence.

In some embodiments, the DNA G-quadruplex sequence comprises the nucleic acid sequence of SEQ ID NO: 3.

In some embodiments, the modified non-coding RNA comprises at least 3 copies of the nucleic acid sequence of SEQ ID NO: 3.

In some embodiments, the structural sequence is a telomeric repeat sequence.

In some embodiments, the telomeric repeat sequence comprises the nucleic acid sequence of SEQ ID NO: 4.

In some embodiments, the modified non-coding RNA comprises at least 3 copies of the nucleic acid sequence of SEQ ID NO: 4.

In some embodiments, the secondary structure of the non-coding RNA is an aptamer that is capable of binding to a target molecule.

In some embodiments, at least one modified nucleotide comprises a modified nucleobase.

In some embodiments, the modified nucleobase is selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

In some embodiments, at least one modified nucleotide comprises a modified sugar.

In some embodiments, the modified sugar is selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose.

In some embodiments, at least one modified nucleotide comprises a 2′ modification.

In some embodiments, the 2′ modification is selected from the group consisting of a locked-nucleic acid (LNA) modification (i.e., a nucleotide comprising an additional carbon atom bound to the 2′ oxygen and 4′ carbon of ribose), 2′-fluoro (2′-F), 2′-O-methoxy-ethyl (2′-MOE), and 2′-O-methylation (2′-OMe).

In some embodiments, at least one modified nucleotide comprises a modified phosphate.

In some embodiments, the modified phosphate is selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

In some embodiments, the poly-A region comprises at least 3, at least 4, at least 5, or at least 6 phosphorothioates.

In some embodiments, the poly-A region comprises at least 6 phosphorothioates.

In some embodiments, the poly-A region comprises at least 3 guanine nucleotides and least 3 phosphorothioates.

In some embodiments, the poly-A region comprises at least 6 nucleotides comprising a 2′ modification.

In some embodiments, the poly-A region comprises at least 3 deoxyribose sugars.

In some embodiments, the poly-A region comprises at least 5, at least 10, at least 15, at least 20, or at least 23 deoxyribose sugars.

In some embodiments, the poly-A region comprises at least 23 deoxyribose sugars.

In some embodiments, the 3′ terminal nucleotide of the non-coding RNA does not comprise hydroxy at the 3′ position of the 3′ terminal nucleotide.

In some embodiments, the 3′ terminal nucleotide of the non-coding RNA comprises an inverted nucleotide.

In some embodiments, the 3′ terminal nucleotide of the mRNA comprises a dideoxyadenosine, dideoxycytidine, dideoxyguanosine, dideoxythymidine, dideoxyuridine, or inverted-deoxythymidine.

In some embodiments, the 3′ terminal nucleotide of the mRNA comprises a dideoxycytidine.

In some embodiments, the modified non-coding RNA comprises a first modified nucleotide and a second modified nucleotide, wherein the first and second modified nucleosides comprise different structures.

In some embodiments, the poly-A region comprises at least 25-500 nucleotides.

In some embodiments, the poly-A region comprises at least 50, at least 100, at least 150, or at least 200 nucleotides.

In some embodiments, at least 25%, at least 30%, at least 40%, at least 50%, at least 60%, at least 70%, at least 80%, at least 90%, at least 95%, at least 96%, at least 97%, at least 98%, or at least 99% of nucleotides of the poly-A region are adenosine nucleotides.

In some aspects, the present disclosure provides a method of producing a modified mRNA, the method comprising ligating a first RNA comprising an open reading frame encoding a protein to a tailing nucleic acid comprising one or more modified nucleotides, in the presence of an RNA ligase, whereby the RNA ligase forms a covalent bond between the 3′ nucleotide of the RNA and the 5′ nucleotide of the tailing nucleic acid to produce the modified mRNA.

In some embodiments, the modified mRNA comprises a 5′ untranslated region (5′ UTR) and a 3′ untranslated region (3′ UTR), wherein the ORF is between the 5′ UTR and the 3′ UTR, wherein the 3′ UTR is between the ORF and the poly-A region.

In some embodiments, the method further comprises circularizing the modified mRNA in the presence of a ribozyme, wherein the modified mRNA comprises a 3′ intron and a 5′ intron, wherein the 3′ intron is 5′ to the 5′ UTR, wherein the 5′ intron is 3′ to the poly-A region, whereby the ribozyme forms a covalent bond between a nucleotide that is 3′ to the 3′ intron and a nucleotide that is 5′ to the 5′ intron to produce a circular mRNA that does not comprise the 5′ intron or the 3′ intron, wherein the poly-A region is between the 3′ UTR and the 5′ UTR of the circular mRNA.

In some embodiments, the method further comprises the steps of:
(i) introducing a 5′ terminal phosphate group onto the first nucleotide of the modified mRNA;
(ii) cleaving one or more 3′ terminal nucleotides of the modified mRNA to produce a modified mRNA with a 3′ terminal hydroxyl group; and
(iii) circularizing the modified mRNA produced in step (ii) in the presence of a circularizing ligase;
whereby the circularizing ligase forms a covalent bond between the 3′ nucleotide of the modified mRNA and the 5′ nucleotide of the modified mRNA to produce a circular modified mRNA, wherein the poly-A region is between the 3′ UTR and the 5′ UTR.

In some aspects, the present disclosure provides a method of producing a modified mRNA, the method comprising ligating an RNA comprising an open reading frame encoding a protein to a tailing nucleic acid comprising one or more copies of a structural sequence in the presence of an RNA ligase, whereby the ligase forms a covalent bond between the 3′ nucleotide of the RNA and the 5′ nucleotide of the tailing nucleic acid to produce the modified mRNA.

In some embodiments, the modified mRNA comprises a 5′ untranslated region (5′ UTR) and a 3′ untranslated region (3′ UTR), wherein the ORF is between the 5′ UTR and the 3′ UTR, wherein the 3′ UTR is between the ORF and the poly-A region, wherein the poly-A region is between the 3′ UTR and the one or more copies of the structural sequence.

In some embodiments, the method further comprises circularizing the modified mRNA in the presence of a ribozyme, wherein the modified mRNA comprises a 3′ intron and a 5′ intron, wherein the 3′ intron is 5′ to the 5′ UTR, wherein the 5′ intron is 3′ to the one or more copies of the structural sequence, whereby the ribozyme forms a covalent bond between a nucleotide that is 3′ to the 3′ intron and a nucleotide that is 5′ to the 5′ intron to produce a circular mRNA that does not comprise the 5′ intron or the 3′ intron, wherein the one or more copies of the structural sequence are between the poly-A region and the 5′ UTR of the circular mRNA.

In some embodiments, the method further comprises the steps of:
(i) introducing a 5′ terminal phosphate group onto the first nucleotide of the modified mRNA;
(ii) cleaving one or more 3′ terminal nucleotides of the modified mRNA to produce a modified mRNA with a 3′ terminal hydroxyl group; and
(iii) circularizing the modified mRNA produced in step (ii) in the presence of a circularizing ligase;
whereby the circularizing ligase forms a covalent bond between the 3′ nucleotide of the modified mRNA and the 5′ nucleotide of the modified mRNA to produce a circular modified mRNA, wherein the one or more copies of the structural sequence are between the 3′ UTR and the 5′ UTR.

In some embodiments, the modified mRNA is circularized in the presence of a scaffold nucleic acid, wherein the scaffold nucleic acid is a nucleic acid that is capable of hybridizing with the modified mRNA, wherein the modified mRNA forms a circular secondary structure when bound to the scaffold nucleic acid.

In some embodiments, the scaffold nucleic acid comprises:
(a) a first hybridization sequence comprising 5 or more nucleotides, wherein the first hybridization sequence is complementary to at least the first five (5) nucleotides of the modified mRNA; and
(b) a second hybridization sequence comprising 5 or more nucleotides, wherein the second hybridization sequence is complementary to at least the last five (5) nucleotides of the modified mRNA;
wherein at least the first five (5) nucleotides of the modified mRNA hybridize with the first hybridization sequence, and at least the last five (5) nucleotides of the modified mRNA hybridize with the second hybridization sequence.

In some embodiments, a last nucleotide of the first hybridization sequence and a first nucleotide of the second hybridization sequence are adjacent in the scaffold nucleic acid and not separated by any other nucleotides.

In some embodiments, the modified mRNA comprises:
(i) a first self-hybridization sequence that is 5′ to the open reading frame;
(ii) a second self-hybridization sequence that is 3′ to the open reading frame;
(iii) a first non-hybridization sequence that is 5′ to the first self-hybridization sequence; and
(iv) a second non-hybridization sequence that is 3′ to the second self-hybridization sequence,
wherein the first and second self-hybridization sequences are capable of hybridizing with each other,
wherein the first and second self-hybridization sequences are not capable of hybridizing with each other.

In some embodiments, hybridization of the first and second self-hybridization sequences forms a secondary structure in which the 5′ terminal nucleotide and the 3′ terminal nucleotide of the modified mRNA are separated by a distance of less than 100 Å.

In some embodiments, the 5′ terminal nucleotide and the 3′ terminal nucleotide are separated by a distance of less than 90 Å, less than 80 Å, less than 70 Å, less than 60 Å, less than 50 Å, less than 40 Å, less than 30 Å, less than 20 Å, or less than 10 Å.

In some embodiments, the circularizing ligase is T4 RNA ligase.

In some embodiments, the structural sequence is a G-quadruplex sequence.

In some embodiments, the G-quadruplex is an RNA G-quadruplex sequence.

In some embodiments, the RNA G-quadruplex sequence comprises the nucleic acid sequence of SEQ ID NO: 2.

In some embodiments, the tailing nucleic acid comprises at least 3 copies of the nucleic acid sequence of SEQ ID NO: 2.

In some embodiments, the G-quadruplex is a DNA G-quadruplex sequence.

In some embodiments, the DNA G-quadruplex sequence comprises the nucleic acid sequence of SEQ ID NO: 3.

In some embodiments, the tailing nucleic acid comprises at least 3 copies of the nucleic acid sequence of SEQ ID NO: 3.

In some embodiments, the structural sequence is a telomeric repeat sequence.

In some embodiments, the telomeric repeat sequence comprises the nucleic acid sequence of SEQ ID NO: 4.

In some embodiments, the tailing nucleic acid comprises at least 3 copies of the nucleic acid sequence of SEQ ID NO: 4.

In some embodiments, the structural sequence is an aptamer sequence comprising at least two nucleotides that are capable of interacting to form an aptamer, wherein the aptamer is a secondary structure that is capable of binding to a target molecule.

In some embodiments, the tailing nucleic acid comprises at least one modified nucleotide.

In some embodiments, the 5′ nucleotide of the RNA does not comprise a 5′ terminal phosphate group;
wherein the 3′ nucleotide of the RNA comprises a 3′ terminal hydroxyl group;
wherein the 5′ nucleotide of the tailing nucleic acid comprises a 5′ terminal phosphate group; and
wherein the 3′ nucleotide of the tailing nucleic acid does not comprise a 3′ terminal hydroxyl group.

In some embodiments, the 5′ nucleotide of the RNA does not comprise a 5′ terminal hydroxyl group;
wherein the 3′ nucleotide of the RNA comprises a 3′ terminal phosphate group;
wherein the 5′ nucleotide of the tailing nucleic acid comprises a 5′ terminal hydroxyl group;
wherein the 3′ nucleotide of the tailing nucleic acid does not comprise a 3′ terminal phosphate group; and
wherein the RNA ligase is an RtcB ligase.

In some embodiments, at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the tailing nucleic acid are modified nucleotides.

In some embodiments, at least 4, 5, 6, 7, 8, 9, 10, 15, 20, or 25 of the 25 last nucleotides of the tailing nucleic acid are modified nucleotides.

In some embodiments, at least one modified nucleotide comprises a modified nucleobase.

In some embodiments, the modified nucleobase is selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (i6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

In some embodiments, at least one modified nucleotide comprises a modified sugar.

In some embodiments, the modified sugar is selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose.

In some embodiments, at least one modified nucleotide comprises a 2′ modification.

In some embodiments, the 2′ modification is selected from the group consisting of a locked-nucleic acid (LNA) modification (i.e., a nucleotide comprising an additional carbon atom bound to the 2′ oxygen and 4′ carbon of ribose), 2′-fluoro (2′-F), 2′-O-methoxy-ethyl (2′-MOE), and 2′-O-methylation (2′-OMe).

In some embodiments, at least one modified nucleotide comprises a modified phosphate.

In some embodiments, the modified phosphate is selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

In some embodiments, the tailing nucleic acid comprises at least 3, at least 4, at least 5, or at least 6 phosphorothioates.

In some embodiments, the tailing nucleic acid comprises at least 6 phosphorothioates.

In some embodiments, the tailing nucleic acid comprises at least 3 guanine nucleotides and least 3 phosphorothioates.

In some embodiments, the tailing nucleic acid comprises at least 6 nucleotides comprising a 2′ modification.

In some embodiments, the tailing nucleic acid comprises at least 3 deoxyribose sugars.

In some embodiments, the tailing nucleic acid comprises at least 5, at least 10, at least 15, at least 20, or at least 23 deoxyribose sugars.

In some embodiments, the tailing nucleic acid comprises at least 23 deoxyribose sugars.

In some embodiments, the 3′ terminal nucleotide of the tailing nucleic acid comprises a dideoxyadenosine, dideoxycytidine, dideoxyguanosine, dideoxythymidine, dideoxyuridine, or inverted-deoxythymidine.

In some embodiments, the tailing nucleic acid comprises a first modified nucleotide and a second modified nucleotide, wherein the first and second modified nucleotides comprise different structures.

In some embodiments, at least 25%, at least 30%, at least 40%, at least 50%, at least 60%, at least 70%, at least 80%, at least 90%, at least 95%, at least 96%, at least 97%, at least 98%, or at least 99% of the poly-A region of the modified mRNA are adenosine nucleotides.

In some embodiments, the poly-A region of the modified mRNA comprises at least 25-500 nucleotides.

In some embodiments, the poly-A region of the modified mRNA comprises at least 50, at least 100, at least 150, or at least 200 nucleotides.

In some embodiments, the modified mRNA is a linear mRNA, wherein the linear mRNA comprises a 5′ cap.

In some embodiments, the 5′ cap comprises a 7-methylguanosine.

In some embodiments, the 5′ cap further comprises one or more phosphates connecting the 7-methylguanosine to an adjacent nucleotide of the modified mRNA.

In some embodiments, the 5′ cap comprises a 3′-O-Me-m7G(5′)ppp(5′)G.

In some embodiments, one or more phosphates of the 5′ cap is a modified phosphate selected from the group consisting of phosphorothioate, triazole ring, dihalogenmethylenebisphosphonate, imidodiphosphate, and methylenebis(phosphonate).

In some embodiments, the RNA ligase is T4 RNA ligase.

In some aspects, the present disclosure provides a method of producing a modified non-coding RNA, the method comprising ligating a first RNA comprising a non-coding RNA sequence to a tailing nucleic acid comprising one or more modified nucleotides, in the presence of an RNA ligase, whereby the RNA ligase forms a covalent bond between the 3′ nucleotide of the RNA and the 5′ nucleotide of the tailing nucleic acid to produce the modified non-coding RNA.

In some embodiments, the modified non-coding RNA comprises a poly-A region that is 3′ to the non-coding RNA sequence.

In some embodiments, the method further comprises circularizing the modified non-coding RNA in the presence of a ribozyme, wherein the modified non-coding RNA comprises a 3′ intron and a 5′ intron, wherein the 3′ intron is 5′ to the non-coding RNA sequence, wherein the 5′ intron is 3′ to the poly-A region, whereby the ribozyme forms a covalent bond between a nucleotide that is 3′ to the 3′ intron and a nucleotide that is 5′ to the 5′ intron to produce a circular non-coding RNA that does not comprise the 5′ intron or the 3′ intron, wherein the poly-A region is between the 3′ and 5′ nucleotides of the non-coding RNA.

In some embodiments, the method further comprises steps of:
(i) introducing a 5′ terminal phosphate group onto the first nucleotide of the modified non-coding RNA;
(ii) cleaving one or more 3′ terminal nucleotides of the modified non-coding RNA to produce a modified non-coding RNA with a 3′ terminal hydroxyl group; and
(iii) circularizing the modified non-coding RNA produced in step (ii) in the presence of a circularizing ligase;
whereby the circularizing ligase forms a covalent bond between the 3′ nucleotide of the modified non-coding RNA and the 5′ nucleotide of the modified non-coding RNA to produce a circular modified non-coding RNA, wherein the poly-A region is between the 3′ and 5′ nucleotides of the non-coding RNA.

In some embodiments, the tailing nucleic acid further comprises one or more copies of a structural sequence.

In some embodiments, the modified non-coding RNA comprises a poly-A region is between the non-coding RNA sequence and the one or more copies of the structural sequence.

In some embodiments, the method further comprises circularizing the modified non-coding RNA in the presence of a ribozyme, wherein the modified non-coding RNA comprises a 3′ intron and a 5′ intron, wherein the 3′ intron is 5′ to the non-coding RNA sequence, wherein the 5′ intron is 3′ to the one or more copies of the structural sequence, whereby the ribozyme forms a covalent bond between a nucleotide that is 3′ to the 3′ intron and a nucleotide that is 5′ to the 5′ intron to produce a circular non-coding RNA that does not comprise the 5′ intron or the 3′ intron, wherein the one or more copies of the structural sequence are between the poly-A region and the non-coding RNA sequence of the circular non-coding RNA.

In some embodiments, the method further comprises the steps of:
(i) introducing a 5′ terminal phosphate group onto the first nucleotide of the modified non-coding RNA;
(ii) cleaving one or more 3′ terminal nucleotides of the modified non-coding RNA to produce a modified non-coding RNA with a 3′ terminal hydroxyl group; and
(iii) circularizing the modified non-coding RNA produced in step (ii) in the presence of a circularizing ligase;
whereby the circularizing ligase forms a covalent bond between the 3′ nucleotide of the modified non-coding RNA and the 5′ nucleotide of the modified non-coding RNA to produce a circular modified non-coding RNA, wherein the one or more copies of the structural sequence are between the poly-A region and the non-coding RNA sequence.

In some embodiments, the modified non-coding RNA is circularized in the presence of a scaffold nucleic acid, wherein the scaffold nucleic acid is a nucleic acid that is capable of hybridizing with the modified non-coding RNA, wherein the modified non-coding RNA forms a circular secondary structure when bound to the scaffold nucleic acid.

In some embodiments, the scaffold nucleic acid comprises:
(a) a first hybridization sequence comprising 5 or more nucleotides, wherein the first hybridization sequence is complementary to at least the first five (5) nucleotides of the modified non-coding RNA; and
(b) a second hybridization sequence comprising 5 or more nucleotides, wherein the second hybridization sequence is complementary to at least the last five (5) nucleotides of the modified non-coding RNA;
wherein at least the first five (5) nucleotides of the modified non-coding RNA hybridize with the first hybridization sequence, and at least the last five (5) nucleotides of the modified non-coding RNA hybridize with the second hybridization sequence.

In some embodiments, a last nucleotide of the first hybridization sequence and a first nucleotide of the second hybridization sequence are adjacent in the scaffold nucleic acid and not separated by any other nucleotides.

In some embodiments, the modified non-coding RNA comprises:
(i) a first self-hybridization sequence that is 5′ to the open reading frame;
(ii) a second self-hybridization sequence that is 3′ to the open reading frame;
(iii) a first non-hybridization sequence that is 5′ to the first self-hybridization sequence; and
(iv) a second non-hybridization sequence that is 3′ to the second self-hybridization sequence,
wherein the first and second self-hybridization sequences are capable of hybridizing with each other, and wherein the first and second self-hybridization sequences are not capable of hybridizing with each other.

In some embodiments, hybridization of the first and second self-hybridization sequences forms a secondary structure in which the 5′ terminal nucleotide and the 3′ terminal nucleotide of the modified non-coding RNA are separated by a distance of less than 100 Å.

In some embodiments, the 5′ terminal nucleotide and the 3′ terminal nucleotide are separated by a distance of less than 90 Å, less than 80 Å, less than 70 Å, less than 60 Å, less than 50 Å, less than 40 Å, less than 30 Å, less than 20 Å, or less than 10 Å.

In some embodiments, the circularizing ligase is T4 RNA ligase.

In some embodiments, the structural sequence is a G-quadruplex sequence.

In some embodiments, the G-quadruplex is an RNA G-quadruplex sequence.

In some embodiments, the RNA G-quadruplex sequence comprises the nucleic acid sequence of SEQ ID NO: 2.

In some embodiments, the tailing nucleic acid comprises at least 3 copies of the nucleic acid sequence of SEQ ID NO: 2.

In some embodiments, the G-quadruplex is a DNA G-quadruplex sequence.

In some embodiments, the DNA G-quadruplex sequence comprises the nucleic acid sequence of SEQ ID NO: 3.

In some embodiments, the tailing nucleic acid comprises at least 3 copies of the nucleic acid sequence of SEQ ID NO: 3.

In some embodiments, the structural sequence is a telomeric repeat sequence.

In some embodiments, the telomeric repeat sequence comprises the nucleic acid sequence of SEQ ID NO: 4.

In some embodiments, the tailing nucleic acid comprises at least 3 copies of the nucleic acid sequence of SEQ ID NO: 4.

In some embodiments, the structural sequence is an aptamer sequence comprising at least two nucleotides that are capable of interacting to form an aptamer, wherein the aptamer is a secondary structure that is capable of binding to a target molecule.

In some embodiments, the 5′ nucleotide of the RNA does not comprise a 5′ terminal phosphate group;
wherein the 3′ nucleotide of the RNA comprises a 3′ terminal hydroxyl group;
wherein the 5′ nucleotide of the tailing nucleic acid comprises a 5′ terminal phosphate group; and
wherein the 3′ nucleotide of the tailing nucleic acid does not comprise a 3′ terminal hydroxyl group.

In some embodiments, the 5′ nucleotide of the RNA does not comprise a 5′ terminal hydroxyl group;
wherein the 3′ nucleotide of the RNA comprises a 3′ terminal phosphate group;
wherein the 5′ nucleotide of the tailing nucleic acid comprises a 5′ terminal hydroxyl group;
wherein the 3′ nucleotide of the tailing nucleic acid does not comprise a 3′ terminal phosphate group; and
wherein the RNA ligase is an RtcB ligase.

In some embodiments, at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the tailing nucleic acid are modified nucleotides.

In some embodiments, at least 4, 5, 6, 7, 8, 9, 10, 15, 20, or 25 of the 25 last nucleotides of the tailing nucleic acid are modified nucleotides.

In some embodiments, at least one modified nucleotide comprises a modified nucleobase.

In some embodiments, the modified nucleobase is selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

In some embodiments, at least one modified nucleotide comprises a modified sugar.

In some embodiments, the modified sugar is selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, S′-biotin-ribose, 2′-O,4′-C-methylene-linked, 2′-O,4′-C-amino-linked ribose, and 2′-O,4′-C-thio-linked ribose.

In some embodiments, at least one modified nucleotide comprises a 2′ modification.

In some embodiments, the 2′ modification is selected from the group consisting of a locked-nucleic acid (LNA) modification (i.e., a nucleotide comprising an additional carbon atom bound to the 2′ oxygen and 4′ carbon of ribose), 2′-fluoro (2′-F), 2′-O-methoxy-ethyl (2′-MOE), and 2′-O-methylation (2′-OMe).

In some embodiments, at least one modified nucleotide comprises a modified phosphate.

In some embodiments, the modified phosphate is selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

In some embodiments, the tailing nucleic acid comprises at least 3, at least 4, at least 5, or at least 6 phosphorothioates.

In some embodiments, the tailing nucleic acid comprises at least 6 phosphorothioates.

In some embodiments, the tailing nucleic acid comprises at least 3 guanine nucleotides and least 3 phosphorothioates.

In some embodiments, the tailing nucleic acid comprises at least 6 nucleotides comprising a 2′ modification.

In some embodiments, the tailing nucleic acid comprises at least 3 deoxyribose sugars.

In some embodiments, the tailing nucleic acid comprises at least 5, at least 10, at least 15, at least 20, or at least 23 deoxyribose sugars.

In some embodiments, the tailing nucleic acid comprises at least 23 deoxyribose sugars.

In some embodiments, the 3′ terminal nucleotide of the tailing nucleic acid comprises a dideoxyadenosine, dideoxycytidine, dideoxyguanosine, dideoxythymidine, dideoxyuridine, or inverted-deoxythymidine.

In some embodiments, the tailing nucleic acid comprises a first modified nucleotide and a second modified nucleotide, wherein the first and second modified nucleotides comprise different structures.

In some embodiments, at least 25%, at least 30%, at least 40%, at least 50%, at least 60%, at least 70%, at least 80%, at least 90%, at least 95%, at least 96%, at least 97%, at least 98%, or at least 99% of the poly-A region of the modified non-coding RNA are adenosine nucleotides.

In some embodiments, the poly-A region of the modified non-coding RNA comprises at least 25-500 nucleotides.

In some embodiments, the poly-A region of the modified non-coding RNA comprises at least 50, at least 100, at least 150, or at least 200 nucleotides.

In some embodiments, the RNA ligase is T4 RNA ligase.

In some aspects, the present disclosure provides a modified mRNA produced by any one of the methods provided herein.

In some embodiments, the mRNA encodes an antigen or a therapeutic protein.

In some embodiments, the antigen is a viral antigen, bacterial antigen, protozoal antigen, or fungal antigen.

In some embodiments, the therapeutic protein is an enzyme, transcription factor, cell surface receptor, growth factor, or clotting factor.

In some embodiments, the open reading frame is codon-optimized for expression in a cell.

In some embodiments, the modified mRNA is codon-optimized for expression in a mammalian cell.

In some embodiments, the modified mRNA is codon-optimized for expression in a human cell.

In some aspects, the present disclosure provides a modified non-coding RNA produced by any one of the methods provided herein.

In some embodiments, the modified non-coding RNA is a guide RNA (gRNA), a prime editing guide RNA (pegRNA), or a long non-coding RNA (lncRNA).

In some aspects, the present disclosure provides a lipid nanoparticle comprising any one of the modified mRNAs or modified non-coding RNAs provided herein.

In some aspects, the present disclosure provides a cell comprising any one of the modified mRNAs or modified non-coding RNAs provided herein.

In some embodiments, the cell is a mammalian cell.

In some embodiments, the cell is a human cell.

In some aspects, the present disclosure provides a composition comprising any of the modified mRNAs, modified non-coding RNAs, lipid nanoparticles, or cells provided herein.

In some aspects, the present disclosure provides a pharmaceutical composition comprising any of the modified mRNAs, modified non-coding RNAs, lipid nanoparticles, or cells provided herein, and a pharmaceutically acceptable excipient.

In some aspects, the present disclosure provides a method comprising introducing any of the modified mRNAs, modified non-coding RNAs, or lipid nanoparticles provided herein into a cell.

In some aspects, the present disclosure provides a method comprising intruding any of the modified mRNAs, modified non-coding RNAs, lipid nanoparticles, cells, or compositions provided herein, into a subject.

In some aspects, the present disclosure provides a method of vaccinating a subject, the method comprising intruding any of the modified mRNAs, lipid nanoparticles, cells, or compositions provided herein, into a subject, wherein the open reading frame of the mRNA encodes an antigen.

In some aspects, the present disclosure provides a method of replacing an enzyme in a subject, the method comprising intruding any of the modified mRNAs, lipid nanoparticles, cells, or compositions provided herein, into a subject, wherein the open reading frame of the mRNA encodes an enzyme.

In some aspects, the present disclosure provides a method of modifying the genome of a subject, the method comprising introducing any of the modified non-coding RNAs or compositions provided herein into a subject.

In some embodiments, the subject is a mammal.

In some embodiments, the subject is a human.

In some aspects, the present disclosure provides any of the modified mRNAs, modified non-coding RNAs, lipid nanoparticles, cells, or compositions provided herein, for use as a medicament.

In some aspects, the present disclosure provides a kit comprising an RNA and a tailing nucleic acid of any of the methods provided herein.

In some embodiments, the kit further comprises an RNA ligase.

In some aspects, the present disclosure provides a kit comprising any of the pharmaceutical compositions provided herein and a delivery device.

In some aspects, the present disclosure provides a method for purifying a modified mRNA or a modified non-coding RNA, comprising contacting a mixture comprising a modified mRNA or a modified non-coding RNA with a purification medium, wherein the modified mRNA or modified non-coding RNA interacts with the purification medium to form a modified RNA-purification medium conjugate, separating the modified RNA-purification medium conjugate from the mixture, and eluting the modified mRNA or modified non-coding RNA from the modified RNA-purification medium conjugate with a solvent.

In some embodiments, the purification medium comprises a paramagnetic bead.

### BRIEF DESCRIPTION OF THE DRAWINGS

FIG. 1 shows the structures of naturally occurring modified nucleosides, including m⁶Am, m¹A, pseudouridine, m⁶A, m⁷G, ac⁴C, Nm, and m⁵C, which can be used in the modified mRNAs or methods of making modified mRNAs provided herein.

FIG. 2A shows the design of modified linear mRNAs (Design A) and modified circular mRNAs (Design B). Filled circles represent modified nucleotides in the open reading frame that improve protein production. Open circles represent modified nucleotides in the poly(A) region that improve RNA stability. FIG. 2B shows the arrangement of elements in a typical mRNA, which contains, in 5′-to-3′ order, a 5′ UTR, an open reading frame, a 3′ UTR, and a poly-A tail.

FIG. 3 shows data relating to the relative efficiency of protein production from modified mRNAs relative to unmodified mRNAs. Modified mRNAs encoding green fluorescent protein (GFP) were synthesized and polyadenylated to add poly(A) tails, with the polyadenylation reactions including limited amounts (5% or 25%) of modified adenosine triphosphates, as indicated. Unmodified mRNAs encoding mCherry were synthesized and polyadenylated using canonical nucleotides. Mixtures of modified and unmodified mRNAs were transfected into cells, and the ratio of GFP/mCherry was measured at days 1-3 post-transfection.

FIG. 4A shows an overview of the experimental scheme used for specific poly(A) tail modifications that leave the coding sequence unaltered. Cellular exonucleases deadenylate the poly(A) tail, but random incorporation of modified nucleoside triphosphates (NTPs) by poly(A) polymerase may slow degradation of the 3′ end of the mRNA. (SEQ ID NO: 1) FIG. 4B shows an overview of the experimental scheme used for installation of chemically defined structures at the 3′ end of the mRNA. Chemically synthesized oligonucleotides with defined compositions were ligated to the 3′ end of GFP-encoding mRNAs containing a template-encoded poly(A) sequence. Ligation of chemically synthesized oligonucleotides allowed for the production of unnatural internucleotide linkages and incorporation of defined quantities of modified nucleotides to the end of each mRNA.

FIG. 5 shows barplots of the abundance of GFP, which was encoded by modified mRNAs, normalized to the abundance of mCherry, which was encoded by unmodified mRNA, at 24, 48, and 72 hours post-transfection of both mRNAs into HeLa cells. Mean+/−SD. P values were calculated with unpaired t-test without assuming consistent SD by Graphpad Prism 7.01. *P<0.01, **P<0.001, ***P<0.0001, ****P<0.00001.

FIG. 6A shows a representative RNase H assay showing RNase H activity on mRNAs ligated to some RNA or DNA nucleotides. Ligations were performed on in vitro transcribed mRNA, which was then purified by AMPure bead cleanup as described in the methods section. All samples were characterized for integrity on a separate gel. Samples that are shown in the gel were all treated using the RNase H assay protocol described in the methods section. Ladder shown is 400 ng of Century-Plus RNA Markers. FIG. 6B shows an E. coli RNase R digestion assay performed on select RNA/DNA oligos used as substrates in ligations. Chain-terminating nucleotides do not prevent RNase R digestion, but an mRNA containing 23 deoxyadenosine nucleotides and a terminal dideoxycytidine exhibited robust stability against RNase R degradation. Ladder contains ssDNA primers with lengths listed to the left.

FIG. 7A shows a schematic of messenger-oligonucleotide conjugated RNA (mocRNA) synthesis, with an overview of chemical modifications and structures of synthetic oligos used for ligations. Chemically synthesized oligos with defined composition were ligated to the 3′ end of humanized Monster Green Fluorescent Protein (GFP) mRNAs containing a template-encoded 60 nt poly(A) sequence (GFP-60A), to produce translatable mocRNAs. FIG. 7B shows schematics of the RNase H assay used to quantify ligation reaction efficiency of mocRNAs. Oligonucleotides used for ligations were 30 nt. DNA probes target the 3′ UTR of mRNA such that the 5′ end of the probe is 106 nt upstream of the poly(A) tail. This generates a 5′ mRNA fragment (824 nt) and a 3′ mRNA fragment (166 nt including the 60 nt poly(A) tail for unligated mRNA; ˜ 200 nt for ligated mRNA). The 3′ cleavage product displays a band shift on a denaturing gel upon ligation M, Marker, Century-Plus RNA Markers.

FIG. 8A shows barplots of GFP fluorescence signal normalized to mCherry fluorescence signal and the mock ligation control at 24 hours, 48 hours, and 72 hours post-transfection. Gray dash lines, y=1. mean±s.d, n fields of view (FOV) indicated under respective bars. Each condition had at least 3 biological replicates, of which 4 FOV were imaged from each. P values were calculated by ordinary two-way ANOVA (Dunnett's multiple comparisons test, comparison of means across timepoints), with multiple comparisons to the sample 29rA_ddC. ***P<0.001, ****P<0.0001. FIG. 8B shows representative separate and overlay images of mCherry fluorescence, GFP fluorescence, and Hoechst nuclei staining in HeLa cells 48 hours after transfection of the indicated RNA construct under the same confocal imaging setting. Scale bar, 25 μm. FIG. 8C shows correlation of the means of bulk GFP/mCherry RNA ratios (RT-qPCR, mean±s.e.m., also see Table 7) and bulk GFP/mCherry fluorescence ratios (mean±s.d.) 48 hours after transfection. FIG. 8D shows representative images of STARmap amplicons representing GFP RNA and mCherry RNA in situ in Hela cells fixed 48 hours after transfection with indicated mRNA vectors, acquired under the same confocal imaging setting Nuclei are indicated with DAPI staining. Colocalized GFP and mCherry amplicons (shown in insets; right column) were potentially lipid transfection vesicles (white arrows), and thus excluded from downstream STARmap quantification of RNA species.

FIG. 9A shows kinetic characterization of Firefly luciferase-degron compared to an untagged luciferase. mRNAs encoding each protein were transfected into HeLa cells, which were treated with cycloheximide (CHX) at time=0. Resulting relative luminescent units (RLU) were measured in cells at 2 hr intervals following CHX treatment, to estimate a decay half-life for proteins. FIG. 9B shows Firefly luciferase-degron RLU normalized to mock ligation signal (8 hr post-transfection). Corresponding normalized Firefly RLU values at each timepoint were tested for significance using an ordinary one-way ANOVA test, compared to mock ligation for each timepoint. *P<0.05, **P<0.01, ***P<0.001, ****P<0.0001. FIG. 9C shows representative STARmap images (channel overlay) taken at 24, 48, and 72 hr timepoints from mocRNA-transfected Hela cells. Images were taken as single slices from Z-stacks obtained from each field of view. White arrows in mock ligation, 24 hr sample, show representative transfection vesicles (regions of large size and overlapping GFP/mCherry signal). Gray puncta indicate GFP mRNA or mCherry mRNA. Nuclei are indicated by DAPI staining. Image contrast was adjusted equally among images in ImageJ. FIG. 9D shows a time course of STARmap mRNA counts and quantification in mocRNA-transfected Hela cells GFP and mCherry mRNA species are counted, with the exclusion of large aggregates (i.e., transfection vesicles). Three biological replicates for each experimental condition, with 4 FOVs taken from each sample. Violin plot elements: lines, lower/upper adjacent values; bars, interquartile ranges; white dot, median. Single cell numbers are listed above corresponding distributions. Statistical testing is performed using Welch's t test with comparisons to 29rA_ddC at each respective timepoint. *P<0.05, **P<0.01, ***P<0.001, ****P<0.0001.

FIG. 10A shows schematics of general chemical strategies to increase mRNA exo- and endonuclease resistance through the incorporation of modified nucleotide triphosphates (NTPs). X, modified nucleoside. FIG. 10B shows chemical structure of adenosine-5′-0(1-thiotriphosphate) (S-ATP) used in E-PAP and IVT spike-in reactions. Sulfur modification of alpha phosphate, when incorporated into RNA, is identical to a phosphorothioate (PS) linkage (shown in FIG. 7B). FIG. 10C shows schematics depicting the different strategies of incorporation of phosphorothioate (PS) linkages into mRNA. RNA polymerase (i.e., co-transcriptional) and poly(A) polymerase incorporation of adenosine-5′-O-(1-thiotriphosphate) (S-ATP) was used to install nuclease-resistant PS linkages into mRNA. Insets: denaturing gel showing the effects of each modification strategy on the length distribution of mRNAs. Gray A's: chemically modified adenosines, black A's: unmodified adenosines. M, Marker, Century-Plus RNA Markers. FIG. 10D shows barplots of GFP protein abundance from modified GFP mRNA generated various strategies, normalized to mCherry and the average of the untreated mRNA control at each time point (24 hours, 48 hours, and 72 hours) after transfection into HeLa cells. Mean±s.d.; n, number of FOVs indicated under respective bars. Each condition consisted of at least 3 biological replicates, of which 4 FOVs were imaged from each. Dashed line: y=1. P values are calculated by ordinary two-way ANOVA (Dunnett's multiple comparisons test, comparison of means across timepoints), with multiple comparisons to untreated mRNA unless specified in the figure. **P<0.01, ****P<0.0001.

FIG. 11A shows barplots of GFP protein abundance normalized to mCherry and the “untreated” control in neurons 24 hours and 48 hours after transfection. mean±s.d., n (FOV)=18. Each condition consisted of at least 3 biological replicates, of which 6 FOV/stacks were imaged from each. Gray dash line: y=1. P values were calculated with ordinary two-way ANOVA (Dunnett's multiple comparisons test) compared to the untreated sample for each separate time point. ****P<0.0001. FIG. 11B shows representative images of GFP and mCherry fluorescence in neurons 24 hours after transfection imaged under the same confocal microscopy setting. Nuclei are indicated by Hoechst staining. Scale bar, 25 μm.

FIG. 12 shows representative RNase H assays showing mocRNA vectors prepared by the ligation of IVT GFP-60A mRNAs and synthetic oligos. DNA probe targets the 3′ UTR of mRNA such that the 5′ end of the probe is 106 nt upstream of the poly(A) tail. This generates a 5′ mRNA fragment (824 nt) and a 3′ mRNA fragment (166 nt including 60 nt poly(A), Lanes 1 & 2). The 3′ cleavage product displays a band shift on a denaturing gel upon ligation. M, Marker which is Century-Plus RNA Markers. Ligated and unligated tails are labeled accordingly.

FIG. 13A shows violin plots of single-cell quantification of GFP and mCherry fluorescence ratios (In[1+ratio]) in HeLa cells 24 hours, 48 hours, and 72 hours after transfected with indicated mRNA vectors. Violin plot elements, lines, lower/upper adjacent values; bars, interquartile ranges; white dot, median. n indicated in parentheses. P values are calculated by Welch's t test (unpaired, two-tailed), with comparisons to the sample 29rA_ddC as a control. **P<0.01, ***P<0.001, ****P<0.0001. FIG. 13B shows representative image stack maximum projection of STARmap characterization of GFP and mCherry RNA in Hela cells 48 hours after lipofectamine-mediated transfection. GFP and mCherry mRNA species trapped in lipofectamine-mediated vesicles appeared overlapped and formed large, merged foci. mRNA species released from the vesicle appeared as individual dots in the cytosol, each representing a single mRNA molecule. Scale bar, 20 μm. FIG. 13C shows single-cell analysis of GFP/mCherry mRNA copy numbers (amplicons) quantified by STARmap. Violin plot elements: lines, lower/upper adjacent values; bars, interquartile ranges; white dot, median. Number of cells in parentheses. Gray dash line, median of the sample 29rA_ddC. P values are calculated by Welch's t test (unpaired, two-tailed), with comparisons to the sample 29rA_ddC as a control. *P<0.05, **P<0.01, ***P<0.001, ****P<0.0001. FIG. 13D shows correlation of the medians of single-cell GFP/mCherry RNA ratios and single-cell GFP/mCherry fluorescence ratios 48 hours after transfection.

FIG. 14A shows GFP-60A mocRNAs ligated to length-adjusted PS+G4 oligos (26rA_G4_C9orf72_RNA_6xSrG, 26rA_G4_C9orf72_DNA_6xSG, and 26rA_G4_telo_DNA_6xSG). Fluorescence time-course measurements were performed following transfection of GFP mocRNAs into HeLa cells, along with an mCherry mRNA internal control. Resulting GFP/mCherry fluorescence values for each sample were further normalized to the average value for 6xSr(AG) at each time point. Statistical testing was performed using ordinary two-way ANOVA (Dunnett's multiple comparisons test, comparison of means across timepoints), with comparisons performed to 6xSr(AG). ****P<0.0001. FIG. 14B shows in vitro translation of Firefly-PEST mocRNA constructs. Rabbit reticulocyte lysates were used as in vitro translation systems for Firefly-PEST mocRNA constructs, along with an unmodified internal Renilla luciferase control. Firefly RLU/Renilla RLU were measured from each reaction to compare possible modes of translational enhancement afforded by different mocRNAs. Statistical testing was performed using one-way ANOVA (nonparametric, Kruskal-Wallis, Dunn's multiple comparisons test), with comparisons made to the “mock ligation” sample. *P<0.05. FIG. 14C shows kinetic characterization of Firefly-degron encoding mocRNA constructs. Renilla (internal control) RLU normalized to mock ligation value at 8 hours post-transfection. Corresponding mocRNA values at each timepoint were tested for significance using a one-way ANOVA (Kruskal-Wallis test, Dunn's multiple comparisons test), compared to mock ligation. The internal control signal appeared to be consistent between different samples.

FIG. 15 shows GFP mRNAs subjected to poly(A) tailing by E. coli poly(A) polymerase (E-PAP), with varying amounts of chemically modified ATP derivatives spiked in. Tail-modified GFP mRNAs were transfected into HeLa cells, along with tail-unmodified mCherry transfection control (E-PAP tailed, 100% ATP) Bars represent GFP/mCherry fluorescence normalized by the average of the 100% ATP, E-PAP tailed GFP mRNA sample at each corresponding time point. The percentages indicate the relative molar ratio used between modified and unmodified ATP in each reaction. Chemically modified GFP mRNAs were co-transfected with unmodified mCherry mRNA, and the resulting GFP/mCherry fluorescence ratios were measured at 24, 48, and 72 hours post transfection in HeLa cell culture. ATP: adenosine 5′ triphosphate; m6ATP: N6-methyladenosine 5′ triphosphate; 2′-O-me ATP: 2′ O-methyladenosine-5′-triphosphate; 5-ATP: adenosine-5′-O-(1-thiotriphosphate); dATP: 2′-deoxyadenosine 5′-triphosphate; amino-dATP: 2′-amino-2′-deoxyadenosine-5′-triphosphate. mean±s.d. n=4. Gray dash line: y=1. P values are calculated by ordinary two-way ANOVA (Dunnett's multiple comparisons test, comparison of means across timepoints), with comparisons performed to E-PAP tailing (100% ATP). ****P<0.0001.

FIG. 16A shows quantification of HeLa cell numbers from confocal microscopy images in FIG. 8. Hoechst-stained nuclei were segmented in CellProfiler, and cell numbers in each field of view (FOV) were calculated for each mocRNA condition and time point. Cell numbers were normalized to average cell number for the mock ligation condition at every time point. Comparisons were performed to the “no ligation” sample using an ordinary two-way ANOVA (Dunnett's multiple comparisons test, comparison of means across timepoints). *P<0.05, **P<0.01. FIG. 16B shows RT-qPCR quantification of innate immune response in transfected HeLa cells. RT-qPCR of IFNB1 mRNA in samples transfected with each GFP-60A ligation construct, normalized to human ACTB mRNA and normalized again to the mock ligation sample. Values were further log 10 transformed prior to significance testing and graphing. Each condition consists of at least 3 biological replicates, with 3 technical replicates per biological sample. Averages of 3 technical replicates (for each biological condition) are shown as individual points, such that each data point corresponds to a specific biological replicate (mean+s.e.m of biological replicates). Unmodified GFP mRNA refers to IVT hMGFP mRNA (E-PAP poly(A) tailed) without N1-methylpseudouridine substitution (i.e., contains 100% uridine). Log10-normalized samples were analyzed for significance using Welch's t test (unpaired, two-tailed, parametric). Samples were referenced to 29rA_ddC mocRNA for pairwise comparisons. Number of biological replicates used for each condition (n) indicated in parentheses above the corresponding sample. *P<0.05, **P<0.01, ***P<0.001, ****P<0.0001. FIG. 16C shows fraction of dead rat cortical neurons determined from mocRNA transfections. Primary rat cortical neuron cultures were transfected with 250 ng GFP-60A mocRNA with a 250 ng mCherry mRNA internal control. Cells were then imaged at 24- or 48-hours post-transfection, using Hoechst to stain live and dead nuclei, and NucRed Dead (647) to stain dead nuclei. The relative numbers of dead to total nuclei were calculated to provide percentage dead cells in each transfection condition. Poly(I:C) at 50 ng was used as a positive control for toxicity. Comparisons were performed using ordinary two-way ANOVA (Dunnett's multiple comparisons test, comparison of means across timepoints), with comparisons to the transfection only sample. **P<0.01.

FIG. 17 shows 72-hour Firefly RLU/Renilla RLU, normalized to the average of “mock ligation” sample values. For each condition, n=9, except for 29rA_ddC with n=18. This corresponds to 3 biological replicates×3 technical replicates (per biological replicate), or 6 biological replicates×3 technical replicates for 29rA_ddC. MocRNA constructs were prepared using Firefly luciferase-encoding mRNA. Firefly luciferase mRNA (250 ng) and unligated Renilla luciferase mRNA (250 ng) were co-transfected into HeLa cells using Lipofectamine MessengerMax (LMRNA001), according to the manufacturer's protocol. HeLa cells were reseeded after 6 hour incubation, and luminescence was measured at 72 hours post-transfection using the Promega Dual-Glo Luciferase Assay System (E2920).

FIG. 18A shows experimental procedure of in vivo bioluminescence imaging. Untreated or 6xSr(AG)_invdT conjugated Firefly luciferase mRNA (2 μg) was intramuscularly injected into either the left thigh or right thigh using in vivo-jetRNA (Polyplus: 101000013), according to the manufacturer's protocol. Luciferin (150 mg/kg, VivoGlo™) was injected intraperitoneally 6 hours after mRNA injection. 15 min later, in-vivo bioluminescence imaging was performed. “ug” refers to μg. FIG. 18B shows in vivo bioluminescence was measured under the 3 min of exposure time. The injection sides of untreated and 6xSr(AG)_invdT conjugated Firefly luciferase mRNA are indicated at the bottom of the image. FIG. 18C shows statistical results of in vivo bioluminescence produced by untreated or 6xSr(AG)_invdT conjugated Firefly luciferase mRNA. * p<0.05. Paired T-test.

### DETAILED DESCRIPTION

Provided herein are modified mRNAs with modified nucleotides and/or structural features in or downstream of the poly-A tail of the mRNA to improve stability in cells and thereby enhance protein production. Also provided are methods of making modified mRNAs by ligating a tailing nucleic acid onto the 3′ terminus of an mRNA to introduce a defined number of modified nucleic acids or structural sequences at the 3′ of the modified mRNA produced by the ligation. Additionally, the present disclosure provides pharmaceutical compositions comprising one or more of the modified mRNAs provided herein, and kits containing reagents to produce the modified mRNAs described herein. Conventional mRNAs comprise poly-A tails with multiple adenosine nucleotides at the 3′ end, which can be degraded by cellular exonucleases, which remove 3′ nucleotides. Once exonucleases remove the poly-A tail and begin removing nucleotides of the open reading frame, the mRNA is unable to be translated into an encoded protein. As one of the primary determinants of mRNA stability in a cell is the time required to degrade the poly-A tail, mRNAs that are more resistant to 3′ exonuclease activity are degraded more slowly. Modified mRNAs of the present disclosure have longer half-lives, and are thus more stable, in cells. The more stable an mRNA is in a cell, the longer it will take to be degraded, and thus more protein can be translated from a given RNA molecule with a longer half-life. Modified nucleotides containing one or more structural changes to the nucleobase, sugar, and/or phosphate linkage of the mRNA can interfere with 3′ exonuclease activity, rendering the mRNA more stable. However, the same structural modifications that inhibit 3′ exonucleases can also interfere with the ability of polyadenylating enzymes to incorporate them into a poly-A tail, hindering the addition of modified nucleotides to a poly-A tail through conventional polyadenylation methods. Surprisingly, ligating an oligonucleotide containing as few as three modified nucleotides onto the 3′ end of an mRNA containing a pre-existing poly-A tail resulted in a marked improvement in mRNA stability. The ligation of an oligonucleotide containing structural sequences capable of forming a secondary structure, such as a G-quadruplex or aptamer, which prevent exonucleases from accessing 3′ terminal nucleotides, also markedly improved mRNA stability relative to RNAs without such secondary structures. Multiple classes of modified nucleotides and structural sequences, both alone and in combination with each other, increased the stability of mRNAs when added to the 3′ terminus, suggesting that modifying the poly-A tail of an mRNA to hinder exonuclease activity provides broad utility in the production of modified mRNAs. Modified mRNAs with increased stability in cells, and thus the ability to produce more of an encoded protein from a given RNA molecule, are useful for use in vaccines and other RNA-based therapies, such as the delivery of mRNAs encoding essential enzymes, clotting factors, transcription factors, or cell surface receptors.

### Definitions

A “messenger RNA” (“mRNA”), as used herein, refers to a nucleic acid comprising an open reading frame encoding a protein, and a poly-A region. An mRNA may also comprise a 5′ untranslated region (5′ UTR) that is 5′ to (upstream of) the open reading frame, and a 3′ untranslated region that is 3′ to (downstream of) the open reading frame.

An “open reading frame encoding a protein,” as used herein, refers to a nucleic acid sequence comprising a coding sequence, that leads to the production of the protein when the open reading frame is translated. The nucleic acid sequence may be an RNA sequence, in which case translation of the RNA sequence produces a polypeptide with the amino acid sequence of the protein. The nucleic acid sequence may be a DNA sequence, in which case the protein is produced when an RNA polymerase uses the DNA sequence to transcribe an RNA molecule comprising an RNA sequence that is complementary to the DNA sequence, and translation of the RNA sequence produces a polypeptide with the amino acid sequence of the protein. An open reading frame typically begins with a START codon, such as AUG in the RNA sequence (ATG in the DNA sequence), and ends with a STOP codon, such as UAG, UAA, or UGA in the RNA sequence (TAG, TAA, or TGA in the DNA sequence), with the number of bases between the G of the START codon and the T or U of the STOP codon being a multiple of 3 (e.g., 3, 6, 9).

An RNA molecule that can be translated is referred to as a messenger RNA, or mRNA. An DNA or RNA sequence encodes a gene through codons. A codon refers to a group of three nucleotides within a nucleic acid, such as DNA or RNA, sequence. An anticodon refers to a group of three nucleotides within a nucleic acid, such as a transfer RNA (tRNA), that are complementary to a codon, such that the codon of a first nucleic acid associates with the anticodon of a second nucleic acid through hydrogen bonding between the bases of the codon and anticodon. For example, the codon S′-AUG-3′ on an mRNA has the corresponding anticodon 3′-UAC-5′ on a tRNA. During translation, a tRNA with an anticodon complementary to the codon to be translated associates with the codon on the mRNA, generally to deliver an amino acid that corresponds to the codon to be translated, or to facilitate termination of translation and release of a translated polypeptide from a ribosome.

Translation is the process in which the RNA coding sequence is used to direct the production of a polypeptide. The first step in translation is initiation, in which a ribosome associates with an mRNA, and a first transfer RNA (tRNA) carrying a first amino acid associates with the first codon, or START codon. The next phase of translation, elongation, involves three steps. First, a second tRNA with an anticodon that is complementary to codon following the START codon, or second codon, and carrying a second amino acid, associates with the mRNA Second, the carbon atom of terminal, non-side chain carboxylic acid moiety of the first amino acid reacts with the nitrogen of the terminal, non-side chain amino moiety of the second amino acid carried, forming a peptide bond between the two amino acids, with the second amino acid being bound to the second tRNA, and the first amino acid bound to the second amino acid, but not the first tRNA. Third, the first tRNA dissociates from the mRNA, and the ribosome advances along the mRNA, such that the position at which the first tRNA associated with the ribosome is now occupied by the second tRNA, and the position previously occupied by the second tRNA is now free for an additional tRNA carrying an additional amino acid to associate with the mRNA. These three steps of 1) association of a tRNA carrying amino acid, 2) formation of a peptide bond, which adds an additional amino acid to a growing polypeptide, and 3) advancement of the ribosome along the mRNA, continue until the ribosome reaches a STOP codon, which results in termination of translation. Generally, tRNAs that associate with STOP codons do not carry an amino acid, so the association of a tRNA that does not carry an amino acid during the elongation step results in cleavage of the bond between the polypeptide and the tRNA carrying the final amino acid in the polypeptide, such that the polypeptide is released from the ribosome. Alternatively, ribosomes may dissociate from the mRNA and release the polypeptide if no tRNA associates with the STOP codon.

A “nucleic acid,” or “polynucleotide,” as used herein, refers to an organic molecule comprising two or more covalently bonded nucleotides. A “nucleotide,” as used herein, refers to an organic molecule comprising a 1) a nucleoside comprising a sugar covalently bonded to a nitrogenous base (nucleobase); and 2) a phosphate group that is covalently bonded to the sugar of the nucleoside. Nucleotides in a polynucleotide are typically joined by a phosphodiester bond, in which the 3′ carbon of the sugar of a first nucleotide is linked to the 5′ carbon of the sugar of a second nucleic acid by a bridging phosphate group. Typically, the bridging phosphate comprises two non-bridging oxygen atoms, which are bonded only to a phosphorus atom of the phosphate, and two bridging oxygen atoms, each of which connects the phosphorus atom to either the 3′ carbon of the first nucleotide or the 5′ carbon of the second nucleotide. In a nucleic acid sequence describing the order of nucleotides in a nucleic acid, a first nucleotide is said to be 5′ to (upstream of) a second nucleotide if the 3′ carbon of first nucleotide is connected to the 5′ carbon of the second nucleotide. Similarly, a second nucleotide is said to be 3′ to (downstream of) a first nucleotide if the 5′ carbon of the second nucleotide is connected to the 3′ carbon of the first nucleotide. Nucleic acid sequences are typically read in 5′->3′ order, starting with the 5′ nucleotide and ending with the 3′ nucleotide.

A “modified nucleotide,” as used herein, refers to a nucleotide with a structure that is not the canonical structure of an adenosine nucleotide, cytidine nucleotide, guanine nucleotide, or uracil nucleotide. A canonical structure of a molecule refers to a structure that is generally known in the art to be the structure referred to by the name of the molecule. As used herein, a “modified nucleotide” may also refer to a nucleotide which comprises a nucleobase or sugar (ribose or deoxyribose) that is not canonical. A “modified nucleotide” may also refer to a nucleotide that is covalently linked to a second nucleotide through an internucleoside linkage that is not a canonical internucleoside linkage (i.e., not a phosphodiester internucleoside linkage, e.g., a phosphorothioate internucleoside linkage). A canonical structure of an adenosine ribonucleotide, which comprises an adenine base, ribose sugar, and one or more phosphate groups, is shown below, in the form of adenosine monophosphate:

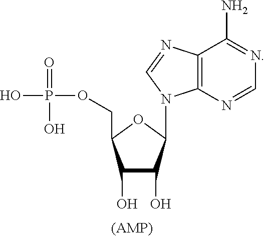

The canonical structure of AMP also refers to structures in which one or more hydroxyl groups of the phosphate and/or one or more hydroxyl groups of the sugar are deprotonated, and structures in which an oxygen atom of the phosphate and/or the 3′ oxygen atom of the sugar are bound to an adjacent nucleotide in a nucleic acid sequence.

The canonical structure of a cytosine nucleotide which comprises a cytosine base, ribose sugar, and one or more phosphate groups, is shown below, in the form of cytidine monophosphate:

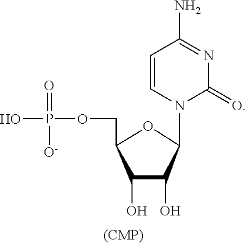

The canonical structure of CMP also refers to structures in which one or more hydroxyl groups of the phosphate and/or one or more hydroxyl groups of the sugar are deprotonated, and structures in which an oxygen atom of the phosphate and/or the 3′ oxygen atom of the sugar are bound to an adjacent nucleotide in a nucleic acid sequence.

The canonical structure of a guanine nucleotide which comprises a guanine base, ribose sugar, and one or more phosphate groups, is shown below, in the form of guanosine monophosphate:

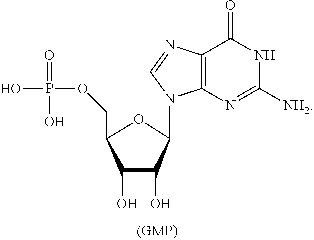

The canonical structure of GMP also refers to structures in which one or more hydroxyl groups of the phosphate and/or one or more hydroxyl groups of the sugar are deprotonated, and structures in which an oxygen atom of the phosphate and/or the 3′ oxygen atom of the sugar are bound to an adjacent nucleotide in a nucleic acid sequence.

The canonical structure of a uracil nucleotide which comprises a uracil base, ribose sugar, and one or more phosphate groups, is shown below, in the form of uridine monophosphate:

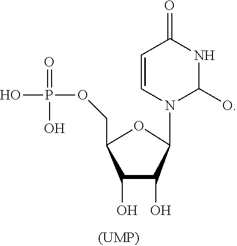

The canonical structure of UMP also refers to structures in which one or more hydroxyl groups of the phosphate and/or one or more hydroxyl groups of the sugar are deprotonated, and structures in which an oxygen atom of the phosphate and/or the 3′ oxygen atom of the sugar are bound to an adjacent nucleotide in a nucleic acid sequence.

The structure of a modified nucleotide may differ from the structure of a canonical nucleotide due to one or more modifications in the sugar, nitrogenous base, or phosphate of the nucleotide. In some embodiments, the modified nucleotide comprises a modified nucleoside that is not the canonical structure of an adenine nucleoside, cytosine nucleoside, guanine nucleoside, or uracil nucleoside. As used herein

An example of a canonical structure of adenosine, an adenine nucleoside, is reproduced below:

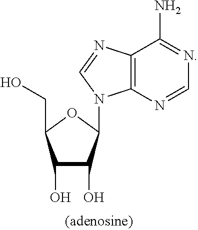

The canonical structure of adenosine also refers to structures in which one or more hydroxyl groups of the phosphate and/or one or more hydroxyl groups of the sugar are deprotonated, structures in which the 5′ carbon is bound to a 5′ phosphate in a nucleic acid sequence, and structures in which a 3′ oxygen atom is bound to a 5′ phosphate group of an adjacent nucleotide in a nucleic acid sequence.

An example of a canonical structure of cytidine, a cytosine nucleoside, is reproduced below:

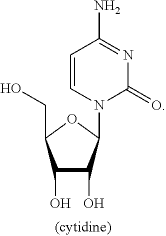

The canonical structure of cytidine also refers to structures in which one or more hydroxyl groups of the phosphate and/or one or more hydroxyl groups of the sugar are deprotonated, structures in which the 5′ carbon is bound to a 5′ phosphate in a nucleic acid sequence, and structures in which a 3′ oxygen atom is bound to a 5′ phosphate group of an adjacent nucleotide in a nucleic acid sequence.

An example of a canonical structure of guanosine, a guanine nucleoside, is reproduced below:

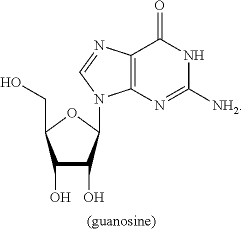

The canonical structure of guanosine also refers to structures in which one or more hydroxyl groups of the phosphate and/or one or more hydroxyl groups of the sugar are deprotonated, structures in which the 5′ carbon is bound to a 5′ phosphate in a nucleic acid sequence, and structures in which a 3′ oxygen atom is bound to a 5′ phosphate group of an adjacent nucleotide in a nucleic acid sequence.

An example of a canonical structure of uridine, a uracil nucleoside, is reproduced below:

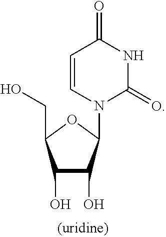

The canonical structure of uridine also refers to structures in which one or more hydroxyl groups of the phosphate and/or one or more hydroxyl groups of the sugar are deprotonated, structures in which the 5′ carbon is bound to a 5′ phosphate in a nucleic acid sequence, and structures in which a 3′ oxygen atom is bound to a 5′ phosphate group of an adjacent nucleotide in a nucleic acid sequence.

A “structural sequence,” as used herein, refers to a nucleic acid sequence comprising at least two nucleotides that are capable of interacting with each other to form a secondary structure in a nucleic acid comprising the structural sequence.

An “aptamer,” as used herein, refers to a nucleic acid comprising a secondary structure that is capable of binding to a target molecule.

A “ligase,” as used herein, refers to an enzyme that is capable of forming a covalent bond between two nucleotides, and the process of “ligation” refers to the formation of the covalent bond between the two nucleotides.

A “tailing nucleic acid,” as used herein, refers to a nucleic acid that is ligated onto the 3′ end of another nucleic acid.

Modified mRNAs

In some aspects, the present disclosure provides modified mRNAs comprising i) one or more modified nucleotides; and/or ii) one or more copies (repeating units) of a structural sequence, with the modified nucleotides and/or structural sequence being part of or 3′ to the poly-A region of the mRNA. The poly-A region, also called the poly(A) region or poly(A) tail, of an mRNA is a region of an mRNA that is 3′ to (downstream of) the open reading frame, comprising multiple, consecutive adenosine nucleotides, typically 50-300 consecutive adenosine nucleotides, and may encompass multiple non-adenosine nucleotides downstream of the consecutive adenosine nucleotides. In cells, after transcription of a DNA sequence, which produces a precursor messenger RNA (pre-mRNA), the poly-A tail is added by a polyadenylating enzyme, such as a poly-A polymerase (PAP), resulting in a long sequence of multiple, consecutive adenosine nucleotides, at the 3′ end of the RNA. The poly-A region plays multiple roles that are important in the production of proteins encoded by mRNAs. First, the poly-A region provides an attachment site for poly-A binding proteins (PABPs), which associate with the mRNA in the nucleus and promote export into the cytoplasm (see, e.g., Tudek et al. Philos Trans R Soc Lond B Biol Sci. 2018. 373(1762): 20180169). Additionally, the presence of a poly-A tail in an mRNA facilitates the initiation of translation (see, e.g., Gallie. Genes & Dev. 1991. 5:2108-2116, and Munroe et al. Mol Cell Biol. 1990. 10(7):3441-3455). Finally, the poly-A tail stabilizes the mRNA by protecting the open reading frame from the activity of exonucleases, such as polynucleotide phosphorylase (PNPase), which remove 3′ nucleotides from an mRNA. As an exonuclease removes nucleotides, the mRNA becomes progressively shorter, and once all of the nucleotides downstream of the open reading frame are removed, the nucleotides removed by the exonuclease will be nucleotides of the open reading frame. Removal of nucleotides from the open reading frame prevents translation of the encoded protein. Additionally, the association of an exonuclease with the mRNA near the open reading frame can inhibit translation by sterically hindering ribosomes and tRNAs from associating with the mRNA. Removal of the poly-A tail is often cited as a rate-limiting step in mRNA degradation, with the life span of an mRNA in a cell being determined by the time required to remove its poly-A tail (see, e.g., Dreyfus et al., Cell. 2002. 111(5):611-613). The composition of a poly-A tail of an mRNA varies, but contains approximately 75 adenosine nucleotides in yeast cells and 250 adenosine nucleotides in mammalian cells.

In some embodiments of the modified mRNAs provided herein, the modified mRNA comprises one or more modified nucleotides in the poly-A region or 3′ to (downstream of) the poly-A region of the mRNA. In some embodiments, the poly-A region includes one or more nucleotides that are not canonical adenosine nucleotides. In some embodiments, the poly-A region includes one or more nucleotides that are not adenosine nucleotides. In some embodiments, the poly-A region comprises one or more nucleotides that are 3′ to (downstream of) a nucleic acid sequence comprising multiple, consecutive adenosine nucleotides. In some embodiments, the poly-A region comprises at least 25 consecutive adenosine nucleotides, which may be canonical adenosine nucleotides or modified adenosine nucleotides. In some embodiments, the poly-A region comprises 25-500 consecutive adenosine nucleotides, which may be canonical adenosine nucleotides or modified adenosine nucleotides. In some embodiments, the poly-A region comprises 25-300 consecutive adenosine nucleotides. In some embodiments, the poly-A region comprises at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 consecutive adenosine nucleotides.

In some embodiments, one or more of the modified nucleotides of the modified mRNA comprise a modified phosphate group. A modified phosphate group is a phosphate group that differs from the canonical structure of phosphate. An example of a canonical structure of a phosphate is shown below:

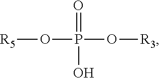

where R₅ and R₃ are atoms or molecules to which the canonical phosphate is bonded. For example, for a phosphate in a nucleic acid sequence, R₅ may refer to the upstream nucleotide of the nucleic acid, and R₃ may refer to the downstream nucleotide of the nucleic acid. The canonical structure of phosphate also refers to structures in which one or more hydroxyl groups of the phosphate are deprotonated, or in which an oxygen atom of the phosphate is bonded to an adjacent nucleotide in a nucleic acid sequence. Non-limiting examples of modified phosphate groups that can be substituted for a canonical phosphate in a nucleic acid include phosphorothioate (PS), phosphorodithioate, thiophosphate, 5′-O-methylphosphonate, 3′-0)-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

In some embodiments of the modified mRNAs comprising modified nucleotides provided herein, at least one modified nucleotide comprises a modified nucleobase. In some embodiments, at least one modified nucleotide comprises a modified sugar. In some embodiments, at least one modified nucleotide comprises a modified phosphate. In some embodiments, at least one modified nucleotide comprises a modified nucleobase selected from the group consisting of: xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, NJ-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (i6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, at least one modified nucleotide comprises a modified sugar selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose. In certain embodiments, at least one modified nucleobase is a 2′-O-(unsubstituted C₁₋₆ alkoxy)-(unsubstituted C₁₋₆ alkyl) nucleobase (e.g., 2′-O-(unsubstituted C₁₋₆ alkoxy)-(unsubstituted C₁₋₆ alkyl) RNA nucleobase). In certain embodiments, at least one modified nucleobase is a 2′-O-methoxy-ethyl nucleobase (e.g., 2′-O-methoxy-ethyl RNA nucleobase). In some embodiments, at least one modified nucleotide comprises a 2′ modification. In some embodiments, the 2′ modification is selected from the group consisting of a locked-nucleic acid (LNA) modification (i.e., a nucleotide comprising an additional carbon atom bound to the 2′ oxygen and 4′ carbon of ribose), 2′-fluoro (2′-F), 2′-O-methoxy-ethyl (2′-MOE), and 2′-O-methylation (2′-OMe).

In some embodiments, at least one modified nucleotide comprises a modified phosphate selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

In some embodiments, the modified mRNA comprises more than one type of modified nucleotide. In some embodiments, the modified mRNA comprises at least a first modified nucleotide, and a second modified nucleotide that has a different structure from the first modified nucleotide. Nucleotides may differ in structure due to differences in the nucleobase, sugar, and/or phosphate group. In some embodiments, the modified mRNA comprises at least a first modified phosphate, and a second modified phosphate that has a different structure from the first modified phosphate. In some embodiments, the modified mRNA comprises a first modified nucleoside and a second modified nucleoside.

Aspects of the present disclosure relate to modified mRNAs comprising poly-A regions with 25 or more adenine nucleotides. In certain embodiments, the poly-A region is 3′ to the open reading frame and comprises 10 or more, 15 or more, 20 or more, 30 or more, 40 or more, or 50 or more adenosine nucleotides. In certain embodiments, the poly-A region is 3′ to the open reading frame and comprises between 10 and 15, between 15 and 20, between 20 and 25, between 25 and 35, between 35 and 50, between 50 and 70, or between 70 and 100 adenosine nucleotides, inclusive. An adenine nucleotide is a nucleotide comprising an adenine nucleoside and a phosphate group. An adenine nucleoside comprises a sugar and an adenine base. In some embodiments, the poly-A region comprises 25 or more canonical adenine nucleotides. A canonical adenosine nucleotide comprises an adenine base, ribose sugar, and phosphate group, as

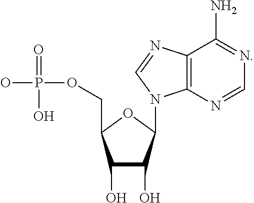

arranged in the structure of adenosine monophosphate (AMP) below: In some embodiments, the one or more of the hydroxyl groups of the phosphate and/or the 3′ hydroxyl group of the ribose are deprotonated, comprising an oxygen ion instead of an —OH group, as shown by the structure:

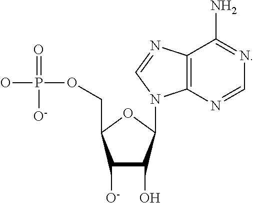

When present in a nucleic acid sequence of an mRNA, a canonical adenosine comprises the following structure and is connected to adjacent nucleotides in the following manner:

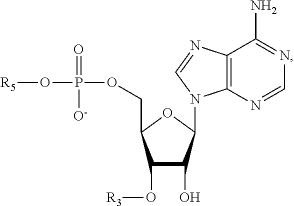

where R₅ is an adjacent nucleotide that is 5′ to (upstream of) the adenosine nucleotide in the mRNA, and R₃ is an adjacent nucleotide that is 3′ to (downstream of) the adenosine nucleotide in the mRNA. In some embodiments, the canonical adenosine nucleotide is the 3′ terminal nucleotide (last nucleotide) of a linear mRNA, R₃ is a hydrogen, and the 3′ terminal nucleotide comprises a 3′ terminal hydroxyl (—OH) group. In some embodiments, the canonical adenosine nucleotide is the 3′ terminal nucleotide (last nucleotide) of a linear mRNA, and R₃ is an electron.

In some embodiments of the modified mRNAs provided herein, the mRNA comprises a 5′ untranslated region (5′ UTR) and a 3′ untranslated region (3′ UTR). 5′ and 3′ UTRs are sequences within an mRNA that do not encode amino acids of the protein encoded by the mRNA, and are thus not part of the open reading frame. The 5′ UTR is 5′ to (upstream of) the open reading frame. The 3′ UTR is 3′ to (downstream of) the open reading frame. In some embodiments, the 3′ UTR comprises one or more nucleotides that are 3′ to the open reading frame and 5′ to (upstream of) the poly-A region of the mRNA.

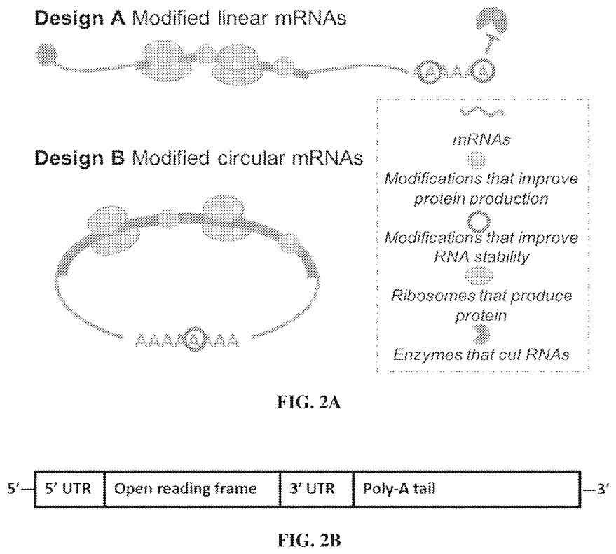

In some embodiments of the mRNAs provided herein, the mRNA comprises, in 5′-to-3′ order: 1) a 5′ UTR; 2) an open reading frame; 3) a 3′ UTR; and 4) a poly-A region (FIG. 2B). In some embodiments, the last nucleotide of the 5′ UTR is 5′ to (upstream of) the first nucleotide of the open reading frame. In some embodiments, the first nucleotide of the open reading frame is 3′ to (downstream of) the last nucleotide of the 5′ UTR, and the last nucleotide of the open reading frame is 5′ to (upstream of) the first base of the 3′ UTR. In some embodiments, the open reading frame is between the last nucleotide of the 5′ UTR and the first nucleotide of the 3′ UTR. In some embodiments, the first nucleotide of the 3′ UTR is 3′ to (downstream of) the last nucleotide of the open reading frame, and the last nucleotide of the 3′ UTR is 5′ to (upstream of) the first base of the poly-A region. In some embodiments, the 3′ UTR is between the last nucleotide of the open reading frame and the first nucleotide of the poly-A region. In some embodiments, the first nucleotide of the poly-A region is 3′ to (downstream of) the last nucleotide of the 3′ UTR.

In some embodiments, the mRNA is a linear mRNA. A linear mRNA is an mRNA with a 5′ terminal nucleotide and a 3′ terminal nucleotide. The 5′ terminal nucleotide of a linear mRNA is covalently bonded to only one adjacent nucleotide of the mRNA, with the adjacent nucleotide occurring 3′ to the 5′ terminal nucleotide in the nucleic acid sequence of the mRNA. The 3′ terminal nucleotide of a linear mRNA is covalently bonded to only one adjacent nucleotide of the mRNA, with the adjacent nucleotide occurring 5′ to the 3′ terminal nucleotide in the nucleic acid sequence of the mRNA. In a nucleic acid sequence comprising every nucleotide of a linear mRNA in 5′-to-3′ order, the 5′ terminal nucleotide is the first nucleotide in the sequence, and the 3′ terminal nucleotide is the last nucleotide in the sequence.

In some embodiments of the linear mRNAs provided herein, the mRNA comprises a 5′ cap. Most mRNAs produced in eukaryotic cells include a 5′ cap that is added during processing of the pre-mRNA into a mature mRNA. The 5′ cap plays multiple roles in the process of mRNA production, export, and translation. First, assembly of the spliceosome, which mediates removal of introns from the pre-mRNA requires binding of the nuclear cap-binding complex (CBC) to the 5′ cap. Furthermore, interactions between the CBC and nuclear pores mediate the export of mRNA from into the cytoplasm, beginning with the 5′ end. Finally, CBC bound to the 5′ cap mediates the recruitment of multiple factors, such as CBP80, CTIF, eIF3g, eIF4III, Met-tRNAi, and ribosomal subunits, which are required for the initiation of translation (see, e.g., Ramanathan et al. Nucleic Acids Res. 2016. 44(16): 7511-7526). In some embodiments, the 5′ cap comprises a 7-methylguanosine. In some embodiments, the 7-methylguanosine comprises the structure:

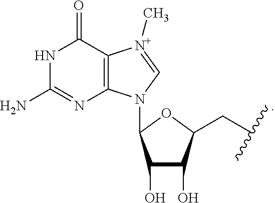

In some embodiments, the 5′ cap comprises one or more phosphates connecting the 7-methylguanosine to an adjacent nucleotide of the modified mRNA. In some embodiments, one or more phosphates of the 5′ cap is a modified phosphate selected from the group consisting of phosphorothioate, triazole ring, dihalogenmethylenebisphosphonate, imidodiphosphate, and methylenebis(phosphonate). In some embodiments, the 7-methylguanosine is connected to an adjacent nucleotide of the mRNA by a 5′-to-5′ triphosphate bridge. In some embodiments, the 5′ cap comprises the structure:

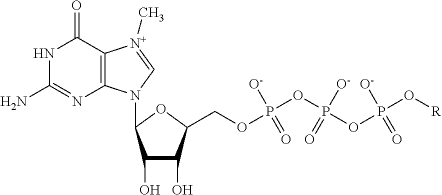

with R being the 5′ carbon of the first transcribed nucleotide of the mRNA. In some embodiments, the 5′ cap comprises a 3′-O-Me-m7G(5′)ppp(5′)G.

In some embodiments, the mRNA is a circular mRNA. A circular mRNA is an mRNA with no 5′ terminal nucleotide or 3′ terminal nucleotide. Every nucleotide in a circular mRNA is covalently bonded to both 1) a 5′ adjacent nucleotide; and 2) a 3′ adjacent nucleotide. In a circular mRNA with a nucleic acid sequence comprising every nucleotide of the circular mRNA in 5′-to-3′ order, the last nucleotide of the nucleic acid sequence is covalently bonded to the first nucleotide of the nucleic acid sequence. In some embodiments of circular mRNAs with a 5′ UTR, a 3′ UTR, and a poly-A region, the poly-A region is 3′ to (downstream from) the 3′ UTR and 5′ to (upstream of) the 5′ UTR.

In some embodiments of the modified mRNAs provided herein, the modified mRNA comprises one or more copies of a structural sequence that are 3′ to the poly-A region of the mRNA. In some embodiments, nucleotides of the secondary structure interact by hydrogen bonding. In some embodiments, the secondary structure is a G-quadruplex. A G-quadruplex, or G-quadruplex, is a secondary structure formed by guanine-rich nucleic acid sequences. A guanine-rich nucleic acid sequence comprises multiple guanine nucleotides. Typically, at least 50% of the nucleotides in a guanine-rich nucleic acid sequence are guanine nucleotides. A G-quadruplex comprises at least one plane containing four guanines (G-tetrad), with each guanine binding to two other guanines by Hoogsteen hydrogen bonding. Hoogsteen hydrogen bonding refers to hydrogen bonding between nitrogenous bases of nucleotides or nucleosides other than canonical base pairing (A:T, A:U, and G:C). The guanines of the G-tetrad surround an empty space, which may comprise a positive cation, such as a potassium ion, to stabilize the G-tetrad. A G-quadruplex comprises at least two G-tetrads arranged in a parallel orientation.

In some embodiments of modified mRNAs comprising one or more structural sequences, the structural sequence is a G-quadruplex sequence A nucleic acid comprising a G-quadruplex sequence is capable of forming a G-quadruplex comprising one or more nucleotides of the G-quadruplex sequence. In some embodiments, the G-quadruplex sequence comprises one or more spacer nucleotides that are not guanine nucleotides. In some embodiments, the G-quadruplex sequence is an RNA G-quadruplex sequence. In some embodiments, the RNA G-quadruplex sequence comprises the nucleic acid sequence GGGGCC (SEQ ID NO. 2). In some embodiments, the modified mRNA comprises at least 3 copies of the nucleotide sequence of SEQ ID NO: 2. In some embodiments, the G-quadruplex sequence is a DNA G-quadruplex sequence. In some embodiments, the DNA G-quadruplex sequence comprises the nucleic acid sequence GGGGCC (SEQ ID NO: 3). In some embodiments, the modified mRNA comprises at least 3 copies of the nucleotide sequence of SEQ ID NO: 3. In some embodiments, the structural sequence comprises a telomeric repeat sequence. In some embodiments, the telomeric repeat sequence comprises the nucleic acid sequence set forth as one of SEQ ID NOs: 4 or 5. In some embodiments, the telomeric repeat sequence comprises the nucleic acid sequence set forth as SEQ ID NO: 4. In some embodiments, the modified mRNA comprises at least 3 copies of the nucleotide sequence of SEQ ID NO: 4.

In some embodiments, the structural sequence is an aptamer sequence comprising at least two nucleotides that are capable of interacting to form an aptamer. Non-limiting examples of target molecules that can be bound by aptamers include cytokines, cell surface receptors, and transcription factors. In some embodiments, the secondary structure formed by the one or more copies of the structural sequence is an aptamer that is capable of binding to a target molecule. Exemplary aptamers are known in the art and include multiple RNA structures capable of binding cell surface receptors such as CD4, CTLA-4, TGF-β receptors, and receptor tyrosine kinases. See., e.g., Germer et al. Int J Biochem Mol Biol., 2013. 4(1):27-40.

In some embodiments, the modified mRNA comprises 1-20 copies of the structural sequence. In some embodiments, the modified mRNA comprises at least 1, at least 2, at least 3, at least 4, at least 5, at least 6, at least 7, at least 8, or at least 9 copies of the structural sequence. In some embodiments, the modified mRNA comprises about 4 copies of the structural sequence. In some embodiments, the modified mRNA comprises multiple different structural sequences. In some embodiments, the modified mRNA comprises at least a first structural sequence, and a second structural sequence comprising a different nucleic acid sequence from the first structural sequence. In some embodiments, the modified mRNA comprises at least one G-quadruplex sequence and at least one telomeric repeat sequence.

In some embodiments of the modified mRNAs comprising one or more copies of a structural sequence provided herein, the poly-A region of the modified mRNA comprises at least one modified nucleotide. In some embodiments, at least one modified nucleotide comprises a modified nucleobase. In some embodiments, at least one modified nucleotide comprises a modified sugar. In some embodiments, at least one modified nucleotide comprises a modified phosphate. In some embodiments, at least one modified nucleotide comprises a modified nucleobase selected from the group consisting of: xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, at least one modified nucleotide comprises a modified sugar selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose. In some embodiments, at least one modified nucleotide comprises a 2′ modification. In some embodiments, the 2′ modification is selected from the group consisting of a locked-nucleic acid (LNA) modification (i.e., a nucleotide comprising an additional carbon atom bound to the 2′ oxygen and 4′ carbon of ribose), 2′-fluoro (2′-F), 2′-O-methoxy-ethyl (2′-MOE), and 2′-O-methylation (2′-OMe).

In some embodiments, at least one modified nucleotide comprises a modified phosphate selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, the poly-A region of the mRNA comprises at least 3, at least 4, or at least 5 phosphorothioates, and does not comprise a 3′ terminal hydroxyl. In some embodiments, the poly-A region of the mRNA comprises at least 3 phosphorothioates, and does not comprise a 3′ terminal hydroxyl. In some embodiments, the poly-A region of the mRNA comprises at least 3 guanine nucleotides and at least 3 phosphorothioates, and does not comprise a 3′ terminal hydroxyl. In some embodiments, the poly-A region of the mRNA comprises at least 3 deoxyribose sugars, and does not comprise a 3′ terminal hydroxyl. In some embodiments, the poly-A region of the mRNA comprises at least 20 deoxyribose sugars, and does not comprise a 3′ terminal hydroxyl. In some embodiments, the poly-A region of the mRNA comprises at least 3 copies of a G-quadruplex sequence, and does not comprise a 3′ terminal hydroxyl. In some embodiments, the poly-A region of the mRNA comprises at least 6 phosphorothioates, and does not comprise a 3′ terminal hydroxyl. In some embodiments, the poly-A region of the mRNA comprises at least 6 sequential phosphorothioates, and does not comprise a 3′ terminal hydroxyl. In some embodiments, the poly-A region of the mRNA comprises at least 6 phosphorothioates and 3 guanine nucleosides, and does not comprise a 3′ terminal hydroxyl. In some embodiments, the poly-A region of the mRNA comprises at least 3 copies of a G-quadruplex sequence and at least 6 phosphorothioates, and does not comprise a 3′ terminal hydroxyl. In some embodiments, the poly-A region of the mRNA comprises at least 3 copies of a telomeric repeat sequence, and at least 6 phosphorothioates, and does not comprise a 3′ terminal hydroxyl. In some embodiments, the 3′ terminal nucleotide that does not comprise a 3′ terminal hydroxyl is a dideoxycytidine or an inverted-deoxythymidine.

In some embodiments, the modified mRNA comprises more than one type of modified nucleotide. In some embodiments, the modified mRNA comprises at least a first modified nucleoside, and a second modified nucleoside that has a different structure from the first modified nucleoside. In some embodiments, the modified mRNA comprises at least a first modified phosphate, and a second modified phosphate that has a different structure from the first modified phosphate. In some embodiments, the modified mRNA comprises a modified nucleoside and a modified nucleoside.

In some embodiments of the modified mRNAs comprising a secondary structure provided herein, the mRNA comprises a 5′ UTR and a 3′ UTR. In some embodiments, the 5′ UTR is 5′ to (upstream of) the open reading frame. In some embodiments, the mRNA comprises, in 5′-to-3′ order, 1) a 5′ UTR; 2) an open reading frame; 3) a 3′ UTR; 4) a poly-A region; and 5) one or more copies of a structural sequence. In some embodiments, the 3′ UTR is 3′ to (downstream of) the open reading frame. In some embodiments, the poly-A region is 3′ to (downstream of) the 3′ UTR. In some embodiments, the one or more copies of the structural sequence, and the secondary structure formed by the structural sequences, are 3′ to (downstream of) the poly-A region. In some embodiments, the mRNA is a linear mRNA. In some embodiments, the linear mRNA comprises a 5′ cap. In some embodiments, the 5′ cap comprises a 7-methylguanosine. In some embodiments, the 5′ cap comprises one or more phosphates connecting the 7-methylguanosine to an adjacent nucleotide of the modified mRNA. In some embodiments, the 7-methylguanosine is connected to an adjacent nucleotide of the mRNA by a 5′-to-5′ triphosphate bridge. In some embodiments, one or more phosphates of the 5′ cap is a modified phosphate selected from the group consisting of phosphorothioate, triazole ring, dihalogenmethylenebisphosphonate, imidodiphosphate, and methylenebis(phosphonate). In some embodiments, the 5′ cap comprises a 3′-O-Me-m7G(5′)ppp(5′)G. In some embodiments, the poly-A region of the mRNA comprises at least 3, at least 4, or at least 5 phosphorothioates, and does not comprise a 3′ terminal hydroxyl. In some embodiments, the poly-A region of the mRNA comprises at least 3 phosphorothioates, and does not comprise a 3′ terminal hydroxyl. In some embodiments, the poly-A region of the mRNA comprises at least 3 guanine nucleotides and at least 3 phosphorothioates, and does not comprise a 3′ terminal hydroxyl. In some embodiments, the poly-A region of the mRNA comprises at least 3 deoxyribose sugars, and does not comprise a 3′ terminal hydroxyl. In some embodiments, the poly-A region of the mRNA comprises at least 20 deoxyribose sugars, and does not comprise a 3′ terminal hydroxyl. In some embodiments, the poly-A region of the mRNA comprises at least 3 copies of a G-quadruplex sequence, and does not comprise a 3′ terminal hydroxyl. In some embodiments, the poly-A region of the mRNA comprises at least 6 phosphorothioates, and does not comprise a 3′ terminal hydroxyl. In some embodiments, the poly-A region of the mRNA comprises at least 6 sequential nucleotides comprising a 2′ modification, and does not comprise a 3′ terminal hydroxyl. In some embodiments, the poly-A region of the mRNA comprises at least 6 sequential phosphorothioates, and does not comprise a 3′ terminal hydroxyl. In some embodiments, the poly-A region of the mRNA comprises at least 6 phosphorothioates and 3 guanine nucleosides, and does not comprise a 3′ terminal hydroxyl. In some embodiments, the poly-A region of the mRNA comprises at least 3 copies of a G-quadruplex sequence and at least 6 phosphorothioates, and does not comprise a 3′ terminal hydroxyl. In some embodiments, the poly-A region of the mRNA comprises at least 3 copies of a telomeric repeat sequence, and at least 6 phosphorothioates, and does not comprise a 3′ terminal hydroxyl. In some embodiments, the 3′ terminal nucleotide that does not comprise a 3′ terminal hydroxyl is a dideoxycytidine or an inverted-deoxythymidine.

In some embodiments of the modified mRNAs comprising a secondary structure provided herein, the modified mRNA comprises, in 5′-to-3′ order, 1) a 5′ UTR; 2) an open reading frame; 3) a 3′ UTR; 4) a poly-A region; and 5) one or more copies of a structural sequence. In some embodiments, the modified mRNA is a circular mRNA. In some embodiments of the circular mRNA, the one or more copies of the structural sequence are between the poly-A region and the 5′ UTR. In some embodiments, the secondary structure is between the poly-A region and the 5′ UTR.

In some embodiments of the modified mRNAs provided herein, 1% to 90% of the nucleotides of the poly-A region are modified nucleotides. In some embodiments, at least 1%, at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the poly-A region are modified nucleotides.

In some embodiments of the modified mRNAs provided herein, 3 or more of the last 25 nucleotides of the poly-A region are modified nucleotides. In some embodiments, at least 4, at least 5, at least 6, at least 7, at least 8, at least 9, at least 10, at least 11, at least 12, at least 13, at least 14, at least 15, at least 20, or 25 of the last 25 nucleotides of the poly-A region are modified nucleotides.

In some embodiments of the modified mRNAs provided herein, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the nucleotides of the poly-A region are adenosine nucleotides. One or more adenosine nucleotides of the poly-A region may be canonical adenosine nucleotides or modified adenosine nucleotides comprising a different structure from the canonical adenosine nucleotide. Non-limiting examples of modified adenosine nucleotides include N6-isopentenyladenosine (i6A), 2-methyl-thio-N6-isopentenyladenosine (ms2i6A), 2-methylthio-N6-methyladenosine (ms2m6A), N6-(cis-hydroxyisopentenyl)adenosine (io6A), 2-methylthio-N6-(cis-hydroxyisopentenyl)adenosine (ms2io6A), N6-glycinylcarbamoyladenosine (g6A), N6-threonylcarbamoyladenosine (t6A), 2-methylthio-N6-threonyl carbamoyladenosine (ms2t6A), N6-methyl-N6-threonylcarbamoyladenosine (m6t6A), N6-hydroxynorvalylcarbamoyladenosine (hn6A), 2-methylthio-N6-hydroxynorvalyl carbamoyladenosine (ms2hn6A), 2′-O-ribosyladenosine (phosphate) (Ar(p)), N6,N6-dimethyladenosine (m62A), N6,2′-O-dimethyladenosine (m6Am), N6,N6,O-2′-trimethyladenosine (m62Am), 1,2′-O-dimethyladenosine (m1Am), N6-acetyladenosine (ac6A), 2′-thioadenosine (2′SA), 5′-thioadenosine (5′SA), 2′-O-(2-azidoethyl)-adenosine, 2′-azido-adenosine, deoxyadenosine (dA), dideoxyadenosine (ddA), and amino-deoxyadenosine (amino-dA).

In some embodiments of the modified mRNAs provided herein, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the nucleotides of the poly-A region are canonical adenosine nucleotides. In some embodiments, the poly-A region further comprises 1 or more nucleotides that are not adenosine nucleotides (e.g., canonical or non-canonical adenosine nucleotides). In some embodiments, at least 1%, at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, at least 50%, at least 55%, at least 60%, at least 65%, at least 70%, at least 80%, or at least 90% of the nucleotides of the poly-A region are nucleotides that are not adenosine nucleotides.

In some embodiments of the modified mRNAs provided herein, the poly-A region comprises at least 25-500 nucleotides. In some embodiments, the poly-A region comprises at least 25, at least 30, at least 50, at least 100, at least 150, or at least 200 nucleotides. In some embodiments, the poly-A region comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 nucleotides. In some embodiments, the poly-A region comprises about 200 to about 300 nucleotides. In some embodiments, the poly-A region comprises about 250 nucleotides.

In some embodiments, the poly-A region comprises at least 3, at least 4, or at least 5 phosphorothioates, and does not comprise a 3′ terminal hydroxyl. In some embodiments, the poly-A region of the mRNA comprises at least 3 phosphorothioates, and does not comprise a 3′ terminal hydroxyl. In some embodiments, the poly-A region of the mRNA comprises at least 3 guanine nucleotides and at least 3 phosphorothioates, and does not comprise a 3′ terminal hydroxyl. In some embodiments, the poly-A region of the mRNA comprises at least 3 deoxyribose sugars, and does not comprise a 3′ terminal hydroxyl. In some embodiments, the poly-A region of the mRNA comprises at least 20 deoxyribose sugars, and does not comprise a 3′ terminal hydroxyl. In some embodiments, the poly-A region of the mRNA comprises at least 3 copies of a G-quadruplex sequence, and does not comprise a 3′ terminal hydroxyl. In some embodiments, the poly-A region of the mRNA comprises at least 6 nucleotides comprising a 2′ modification, and does not comprise a 3′ terminal hydroxyl. In some embodiments, the poly-A region of the mRNA comprises at least 6 phosphorothioates, and does not comprise a 3′ terminal hydroxyl. In some embodiments, the poly-A region of the mRNA comprises at least 6 sequential nucleotides comprising a 2′ modification, and does not comprise a 3′ terminal hydroxyl. In some embodiments, the poly-A region of the mRNA comprises at least 6 sequential phosphorothioates, and does not comprise a 3′ terminal hydroxyl. In some embodiments, the poly-A region of the mRNA comprises at least 6 phosphorothioates and 3 guanine nucleosides, and does not comprise a 3′ terminal hydroxyl. In some embodiments, the poly-A region of the mRNA comprises at least 3 copies of a G-quadruplex sequence and at least 6 phosphorothioates, and does not comprise a 3′ terminal hydroxyl. In some embodiments, the poly-A region of the mRNA comprises at least 3 copies of a telomeric repeat sequence, and at least 6 phosphorothioates, and does not comprise a 3′ terminal hydroxyl. In some embodiments, the 3′ terminal nucleotide that does not comprise a 3′ terminal hydroxyl is a dideoxycytidine or an inverted-deoxythymidine.

### Modified Non-Coding RNAs

Those of ordinary skill in the relevant art will readily recognize that any of the techniques disclosed herein for improving the stability of a mRNA in a cell (e.g., by improving resistance of the mRNA toward 3′ exonuclease activity) may also be suitable for improving the stability of an RNA that does not encode protein (a “non-coding” RNA) in a cell. Accordingly, in some aspects, the present disclosure provides modified non-coding RNAs comprising i) one or more modified nucleotides; and/or ii) one or more copies (repeating units) of a structural sequence, with the modified nucleotides and/or structural sequence being part of or 3′ to the RNA. A non-coding RNA described herein does not comprise an open reading frame (ORF). A non-coding RNA may or may not comprise a 3′ poly-A region. A non-coding RNA that does not comprise a 3′ poly-A region may be modified to comprises a 3′ poly-A region (e.g., by ligating the non-coding RNA to an oligonucleotide comprising a poly-A region by a method disclosed herein or otherwise known in the art). A non-coding RNA may be an RNA comprising a region of complementarity with part of a mRNA transcript or genomic sequence of a cell. A non-coding RNA may be a non-coding RNA that is suitable for genome editing. Examples of non-coding RNA include, but are not limited to, small interfering RNA (siRNA), short hairpin RNA (shRNA), long non-coding RNA (lncRNA), guide RNA (gRNA) for Clustered Regularly Interspaced Short Palindromic Repeats (CRISPR)/Cas9 genome editing, non-CRISPR/Cas9 gRNA (e.g., adenosine deaminases acting on RNA (ADAR)-recruiting gRNA), or prime editing guide RNA (pegRNA). See, e.g., Chen, et al., Acta Pharm Sin B. 2021; 11(2):340-354; Chen, et al., Adv Drug Deliv Rev. 2021; 168:246-258; Hendel, et al., Nat Biotechnol. 2015; 33:985-989; Qu, et al., Nat Biotechnol. 2019; 37(9): 1059-1069; Yi, et al., Nat Biotechnol. 2022. Epub ahead of print; and Nelson, et al., Nat Biotechnol. 2022; 40(3): 402-410. Any technique described herein for generating a modified mRNA may also be used to generate a modified non-coding RNA, unless specifically noted otherwise.

In some embodiments, a modified non-coding RNA provided herein comprises a non-coding RNA that comprises a 3′ poly-A region. In some embodiments, a modified non-coding RNA provided herein comprises a non-coding RNA that does not typically comprise a 3′ poly-A region (e.g., a gRNA). In some embodiments, a modified non-coding RNA provided herein comprises a non-coding RNA that is ligated at its 3′ end to the 5′ end of an oligonucleotide comprising a poly-A region, thereby producing a modified non-coding RNA comprising a poly-A region described herein. A non-coding RNA may be ligated to an oligonucleotide comprising a poly-A region by any method disclosed herein or otherwise known in the art.

In some embodiments of the modified non-coding RNAs provided herein, the modified non-coding RNA comprises one or more modified nucleotides in the poly-A region or 3′ to (downstream of) a poly-A region that is present in the non-coding RNA. In some embodiments, the poly-A region includes one or more nucleotides that are not canonical adenosine nucleotides. In some embodiments, the poly-A region includes one or more nucleotides that are not adenosine nucleotides. In some embodiments, the poly-A region comprises one or more nucleotides that are 3′ to (downstream of) a nucleic acid sequence comprising multiple, consecutive adenosine nucleotides. In some embodiments, the poly-A region comprises at least 25 consecutive adenosine nucleotides, which may be canonical adenosine nucleotides or modified adenosine nucleotides. In some embodiments, the poly-A region comprises 25-500 consecutive adenosine nucleotides, which may be canonical adenosine nucleotides or modified adenosine nucleotides. In some embodiments, the poly-A region comprises 25-300 consecutive adenosine nucleotides. In some embodiments, the poly-A region comprises at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 consecutive adenosine nucleotides.

In some embodiments, one or more of the modified nucleotides of the modified non-coding RNA comprise a modified phosphate group. A modified phosphate group is a phosphate group that differs from the canonical structure of phosphate. An example of a canonical structure of a phosphate is shown below:

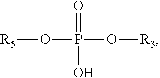

where R₅ and R₃ are atoms or molecules to which the canonical phosphate is bonded. For example, for a phosphate in a nucleic acid sequence, R₅ may refer to the upstream nucleotide of the nucleic acid, and R₃ may refer to the downstream nucleotide of the nucleic acid. The canonical structure of phosphate also refers to structures in which one or more hydroxyl groups of the phosphate are deprotonated, or in which an oxygen atom of the phosphate is bonded to an adjacent nucleotide in a nucleic acid sequence. Non-limiting examples of modified phosphate groups that can be substituted for a canonical phosphate in a nucleic acid include phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

In some embodiments, of the modified non-coding RNAs comprising modified nucleotides provided herein, at least one modified nucleotide comprises a modified nucleobase. In some embodiments, at least one modified nucleotide comprises a modified sugar. In some embodiments, at least one modified nucleotide comprises a modified phosphate. In some embodiments, at least one modified nucleotide comprises a modified nucleobase selected from the group consisting of: xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, NJ-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (i6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, at least one modified nucleotide comprises a modified sugar selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose. In certain embodiments, at least one modified nucleobase is a 2′-O-(unsubstituted C₁₋₆ alkoxy)-(unsubstituted C₁₋₆ alkyl) nucleobase (e.g., 2′-O-(unsubstituted C₁₋₆ alkoxy)-(unsubstituted C₁₋₆ alkyl) RNA nucleobase). In certain embodiments, at least one modified nucleobase is a 2′-O-methoxy-ethyl nucleobase (e.g., 2′-O-methoxy-ethyl RNA nucleobase). In some embodiments, at least one modified nucleotide comprises a 2′ modification. In some embodiments, the 2′ modification is selected from the group consisting of a locked-nucleic acid (LNA) modification (i.e., a nucleotide comprising an additional carbon atom bound to the 2′ oxygen and 4′ carbon of ribose), 2′-fluoro (2′-F), 2′-O-methoxy-ethyl (2′-MOE), and 2′-O-methylation (2′-OMe).

In some embodiments, at least one modified nucleotide comprises a modified phosphate selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

In some embodiments, the modified non-coding RNA comprises more than one type of modified nucleotide. In some embodiments, the modified non-coding RNA comprises at least a first modified nucleotide, and a second modified nucleotide that has a different structure from the first modified nucleotide. Nucleotides may differ in structure due to differences in the nucleobase, sugar, and/or phosphate group. In some embodiments, the modified non-coding RNA comprises at least a first modified phosphate, and a second modified phosphate that has a different structure from the first modified phosphate. In some embodiments, the modified non-coding RNA comprises a first modified nucleoside and a second modified nucleoside.

Aspects of the present disclosure relate to modified non-coding RNAs comprising poly-A regions with 25 or more adenine nucleotides. In certain embodiments, the poly-A region is at the 3′ end of the non-coding RNA and comprises 10 or more, 15 or more, 20 or more, 30 or more, 40 or more, or 50 or more adenosine nucleotides. In certain embodiments, the poly-A region is at the 3′ end of the non-coding RNA and comprises between 10 and 15, between 15 and 20, between 20 and 25, between 25 and 35, between 35 and 50, between 50 and 70, or between 70 and 100 adenosine nucleotides, inclusive. An adenine nucleotide is a nucleotide comprising an adenine nucleoside and a phosphate group. An adenine nucleoside comprises a sugar and an adenine base. In some embodiments, the poly-A region comprises 25 or more canonical adenine nucleotides. A canonical adenosine nucleotide comprises an adenine base, ribose sugar, and phosphate group, as arranged in the structure of adenosine monophosphate (AMP) below:

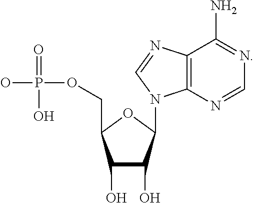

In some embodiments, the one or more of the hydroxyl groups of the phosphate and/or the 3′ hydroxyl group of the ribose are deprotonated, comprising an oxygen ion instead of an —OH group, as shown by the structure:

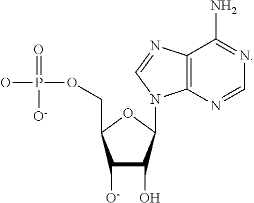

When present in a nucleic acid sequence of a non-coding RNA, a canonical adenosine comprises the following structure and is connected to adjacent nucleotides in the following manner:

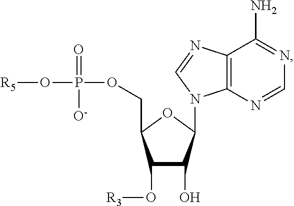

where R₅ is an adjacent nucleotide that is 5′ to (upstream of) the adenosine nucleotide in the non-coding RNA, and R₃ is an adjacent nucleotide that is 3′ to (downstream of) the adenosine nucleotide in the non-coding RNA. In some embodiments, the canonical adenosine nucleotide is the 3′ terminal nucleotide (last nucleotide) of a linear non-coding RNA, R₃ is a hydrogen, and the 3′ terminal nucleotide comprises a 3′ terminal hydroxyl (—OH) group. In some embodiments, the canonical adenosine nucleotide is the 3′ terminal nucleotide (last nucleotide) of a linear non-coding RNA, and R₃ is an electron.

In some embodiments of the non-coding RNAs provided herein, the non-coding RNA comprises, in 5′-to-3′ order: 1) the non-coding RNA; and 2) a poly-A region present within or ligated to the 3′ end of the non-coding RNA 1. In some embodiments, the first nucleotide of the poly-A region that is ligated to the non-coding RNA is 3′ to (downstream of) the last nucleotide of the non-coding RNA.

In some embodiments, the non-coding RNA is a linear non-coding RNA. A linear non-coding RNA is a non-coding RNA with a 5′ terminal nucleotide and a 3′ terminal nucleotide. The 5′ terminal nucleotide of a linear non-coding RNA is covalently bonded to only one adjacent nucleotide of the non-coding RNA, with the adjacent nucleotide occurring 3′ to the 5′ terminal nucleotide in the nucleic acid sequence of the non-coding RNA. The 3′ terminal nucleotide of a linear non-coding RNA is covalently bonded to only one adjacent nucleotide of the non-coding RNA, with the adjacent nucleotide occurring 5′ to the 3′ terminal nucleotide in the nucleic acid sequence of the non-coding RNA. In a nucleic acid sequence comprising every nucleotide of a linear non-coding RNA in 5′-to-3′ order, the 5′ terminal nucleotide is the first nucleotide in the sequence, and the 3′ terminal nucleotide is the last nucleotide in the sequence.

In some embodiments of the linear non-coding RNA provided herein, the non-coding RNA comprises a 5′ cap. In some embodiments, the 5′ cap comprises one or more phosphates connecting the 7-methylguanosine to an adjacent nucleotide of the modified non-coding RNA. In some embodiments, one or more phosphates of the 5′ cap is a modified phosphate selected from the group consisting of phosphorothioate, triazole ring, dihalogenmethylenebisphosphonate, imidodiphosphate, and methylenebis(phosphonate). In some embodiments, the 7-methylguanosine is connected to an adjacent nucleotide of the non-coding RNA by a 5′-to-5′ triphosphate bridge. In some embodiments, the 5′ cap comprises the structure:

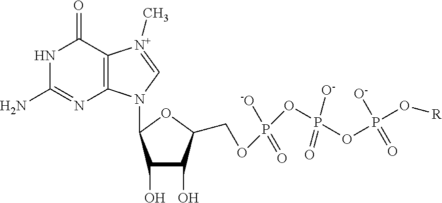

with R being the 5′ carbon of the first transcribed nucleotide of the non-coding RNA. In some embodiments, the 5′ cap comprises a 3′-O-Me-m7G(5′)ppp(5′)G.

In some embodiments, the linear non-coding RNA does not comprise a 5′ cap.

In some embodiments, the non-coding RNA is a circular non-coding RNA. A circular non-coding RNA is an non-coding RNA with no 5′ terminal nucleotide or 3′ terminal nucleotide. Every nucleotide in a circular non-coding RNA is covalently bonded to both 1) a 5′ adjacent nucleotide; and 2) a 3′ adjacent nucleotide. In a circular non-coding RNA with a nucleic acid sequence comprising every nucleotide of the circular non-coding RNA in 5′-to-3′ order, the last nucleotide of the nucleic acid sequence is covalently bonded to the first nucleotide of the nucleic acid sequence. In some embodiments of circular non-coding RNAs, the last nucleotide of a poly-A region within or ligated to the 3′ end of a non-coding RNA is 5′ to the first nucleotide of the non-coding RNA.

In some embodiments of the modified non-coding RNAs provided herein, the modified non-coding RNA comprises one or more copies of a structural sequence that are 3′ to a poly-A region within or ligated to the non-coding RNA. In some embodiments, nucleotides of the secondary structure interact by hydrogen bonding. In some embodiments, the secondary structure is a G-quadruplex. A G-quadruplex, or G-quadruplex, is a secondary structure formed by guanine-rich nucleic acid sequences.

In some embodiments of modified non-coding RNAs comprising one or more structural sequences, the structural sequence is a G-quadruplex sequence. A nucleic acid comprising a G-quadruplex sequence is capable of forming a G-quadruplex comprising one or more nucleotides of the G-quadruplex sequence. In some embodiments, the G-quadruplex sequence comprises one or more spacer nucleotides that are not guanine nucleotides. In some embodiments, the G-quadruplex sequence is an RNA G-quadruplex sequence. In some embodiments, the RNA G-quadruplex sequence comprises the nucleic acid sequence GGGGCC (SEQ ID NO: 2). In some embodiments, the modified non-coding RNA comprises at least 3 copies of the nucleotide sequence of SEQ ID NO: 2. In some embodiments, the G-quadruplex sequence is a DNA G-quadruplex sequence. In some embodiments, the DNA G-quadruplex sequence comprises the nucleic acid sequence GGGGCC (SEQ ID NO: 3). In some embodiments, the modified non-coding RNA comprises at least 3 copies of the nucleotide sequence of SEQ ID NO. 3. In some embodiments, the structural sequence comprises a telomeric repeat sequence. In some embodiments, the telomeric repeat sequence comprises the nucleic acid sequence set forth as one of SEQ ID NOs: 4 or 5. In some embodiments, the telomeric repeat sequence comprises the nucleic acid sequence set forth as SEQ ID NO: 4. In some embodiments, the modified non-coding RNA comprises at least 3 copies of the nucleotide sequence of SEQ ID NO: 4.

In some embodiments, the structural sequence is an aptamer sequence comprising at least two nucleotides that are capable of interacting to form an aptamer. Non-limiting examples of target molecules that can be bound by aptamers include cytokines, cell surface receptors, and transcription factors. In some embodiments, the secondary structure formed by the one or more copies of the structural sequence is an aptamer that is capable of binding to a target molecule. Exemplary aptamers are known in the art and include multiple RNA structures capable of binding cell surface receptors such as CD4, CTLA-4, TGF-ß receptors, and receptor tyrosine kinases. See., e.g., Germer et al. Int J Biochem Mol Biol., 2013. 4(1):27-40.

In some embodiments, the modified non-coding RNA comprises 1-20 copies of the structural sequence. In some embodiments, the modified non-coding RNA comprises at least 1, at least 2, at least 3, at least 4, at least 5, at least 6, at least 7, at least 8, or at least 9 copies of the structural sequence. In some embodiments, the modified non-coding RNA comprises about 4 copies of the structural sequence. In some embodiments, the modified non-coding RNA comprises multiple different structural sequences. In some embodiments, the modified non-coding RNA comprises at least a first structural sequence, and a second structural sequence comprising a different nucleic acid sequence from the first structural sequence. In some embodiments, the modified non-coding RNA comprises at least one G-quadruplex sequence and at least one telomeric repeat sequence.

In some embodiments of the modified non-coding RNAs comprising one or more copies of a structural sequence provided herein, the poly-A region of the modified non-coding RNA comprises at least one modified nucleotide. In some embodiments, at least one modified nucleotide comprises a modified nucleobase. In some embodiments, at least one modified nucleotide comprises a modified sugar. In some embodiments, at least one modified nucleotide comprises a modified phosphate. In some embodiments, at least one modified nucleotide comprises a modified nucleobase selected from the group consisting of: xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, at least one modified nucleotide comprises a modified sugar selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose. In some embodiments, at least one modified nucleotide comprises a 2′ modification. In some embodiments, the 2′ modification is selected from the group consisting of a locked-nucleic acid (LNA) modification (i.e., a nucleotide comprising an additional carbon atom bound to the 2′ oxygen and 4′ carbon of ribose), 2′-fluoro (2′-F), 2′-O-methoxy-ethyl (2′-MOE), and 2′-O-methylation (2′-OMe). In some embodiments, at least one modified nucleotide comprises a modified phosphate selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, the poly-A region of the non-coding RNA comprises at least 3, at least 4, or at least 5 phosphorothioates, and does not comprise a 3′ terminal hydroxyl. In some embodiments, the poly-A region of the non-coding RNA comprises at least 3 phosphorothioates, and does not comprise a 3′ terminal hydroxyl. In some embodiments, the poly-A region of the non-coding RNA comprises at least 3 guanine nucleotides and at least 3 phosphorothioates, and does not comprise a 3′ terminal hydroxyl. In some embodiments, the poly-A region of the non-coding RNA comprises at least 3 deoxyribose sugars, and does not comprise a 3′ terminal hydroxyl. In some embodiments, the poly-A region of the non-coding RNA comprises at least 20 deoxyribose sugars, and does not comprise a 3′ terminal hydroxyl. In some embodiments, the poly-A region of the non-coding RNA comprises at least 3 copies of a G-quadruplex sequence, and does not comprise a 3′ terminal hydroxyl. In some embodiments, the poly-A region of the non-coding RNA comprises at least 6 nucleotides comprising a 2′ modification, and does not comprise a 3′ terminal hydroxyl. In some embodiments, the poly-A region of the non-coding RNA comprises at least 6 phosphorothioates, and does not comprise a 3′ terminal hydroxyl. In some embodiments, the poly-A region of the non-coding RNA comprises at least 6 sequential nucleotides comprising a 2′ modification, and does not comprise a 3′ terminal hydroxyl. In some embodiments, the poly-A region of the non-coding RNA comprises at least 6 sequential phosphorothioates, and does not comprise a 3′ terminal hydroxyl. In some embodiments, the poly-A region of the non-coding RNA comprises at least 6 phosphorothioates and 3 guanine nucleosides, and does not comprise a 3′ terminal hydroxyl. In some embodiments, the poly-A region of the non-coding RNA comprises at least 3 copies of a G-quadruplex sequence and at least 6 phosphorothioates, and does not comprise a 3′ terminal hydroxyl. In some embodiments, the poly-A region of the non-coding RNA comprises at least 3 copies of a telomeric repeat sequence, and at least 6 phosphorothioates, and does not comprise a 3′ terminal hydroxyl. In some embodiments, the 3′ terminal nucleotide that does not comprise a 3′ terminal hydroxyl is a dideoxycytidine or an inverted-deoxythymidine.

In some embodiments, the modified non-coding RNA comprises more than one type of modified nucleotide. In some embodiments, the modified non-coding RNA comprises at least a first modified nucleoside, and a second modified nucleoside that has a different structure from the first modified nucleoside. In some embodiments, the modified non-coding RNA comprises at least a first modified phosphate, and a second modified phosphate that has a different structure from the first modified phosphate. In some embodiments, the modified non-coding RNA comprises a modified nucleoside and a modified nucleoside.

In some embodiments of the modified non-coding RNAs comprising a secondary structure provided herein, the modified non-coding RNA comprises, in 5′-to-3′ order, 1) the 5′ non-coding RNA; 2) a poly-A region within or ligated to the 3′ end of the non-coding RNA; and 3) one or more copies of a structural sequence. In some embodiments, the one or more copies of the structural sequence, and the secondary structure formed by the structural sequences, are 3′ to (downstream of) the poly-A region. In some embodiments, the non-coding RNA is a linear non-coding RNA. In some embodiments, the linear non-coding RNA comprises a 5′ cap. In some embodiments, the 5′ cap comprises a 7-methylguanosine. In some embodiments, the 5′ cap comprises one or more phosphates connecting the 7-methylguanosine to an adjacent nucleotide of the modified non-coding RNA. In some embodiments, the 7-methylguanosine is connected to an adjacent nucleotide of the non-coding RNA by a 5′-to-5′ triphosphate bridge. In some embodiments, one or more phosphates of the 5′ cap is a modified phosphate selected from the group consisting of phosphorothioate, triazole ring, dihalogenmethylenebisphosphonate, imidodiphosphate, and methylenebis(phosphonate). In some embodiments, the 5′ cap comprises a 3′-O-Me-m7G(5′)ppp(5′)G. In some embodiments, the linear non-coding RNA does not comprise a 5′ cap. In some embodiments, the poly-A region of the non-coding RNA comprises at least 3, at least 4, or at least 5 phosphorothioates, and does not comprise a 3′ terminal hydroxyl. In some embodiments, the poly-A region of the non-coding RNA comprises at least 3 phosphorothioates, and does not comprise a 3′ terminal hydroxyl. In some embodiments, the poly-A region of the non-coding RNA comprises at least 3 guanine nucleotides and at least 3 phosphorothioates, and does not comprise a 3′ terminal hydroxyl. In some embodiments, the poly-A region of the non-coding RNA comprises at least 3 deoxyribose sugars, and does not comprise a 3′ terminal hydroxyl. In some embodiments, the poly-A region of the non-coding RNA comprises at least 20 deoxyribose sugars, and does not comprise a 3′ terminal hydroxyl. In some embodiments, the poly-A region of the non-coding RNA comprises at least 3 copies of a G-quadruplex sequence, and does not comprise a 3′ terminal hydroxyl. In some embodiments, the poly-A region of the non-coding RNA comprises at least 6 nucleotides comprising a 2′ modification, and does not comprise a 3′ terminal hydroxyl. In some embodiments, the poly-A region of the non-coding RNA comprises at least 6 phosphorothioates, and does not comprise a 3′ terminal hydroxyl. In some embodiments, the poly-A region of the non-coding RNA comprises at least 6 sequential nucleotides comprising a 2′ modification, and does not comprise a 3′ terminal hydroxyl. In some embodiments, the poly-A region of the non-coding RNA comprises at least 6 sequential phosphorothioates, and does not comprise a 3′ terminal hydroxyl. In some embodiments, the poly-A region of the non-coding RNA comprises at least 6 phosphorothioates and 3 guanine nucleosides, and does not comprise a 3′ terminal hydroxyl. In some embodiments, the poly-A region of the non-coding RNA comprises at least 3 copies of a G-quadruplex sequence and at least 6 phosphorothioates, and does not comprise a 3′ terminal hydroxyl. In some embodiments, the poly-A region of the non-coding RNA comprises at least 3 copies of a telomeric repeat sequence, and at least 6 phosphorothioates, and does not comprise a 3′ terminal hydroxyl. In some embodiments, the 3′ terminal nucleotide that does not comprise a 3′ terminal hydroxyl is a dideoxycytidine or an inverted-deoxythymidine.

In some embodiments of the modified non-coding RNAs comprising a secondary structure provided herein, the modified non-coding RNA comprises, in 5′-to-3′ order, 1) the non-coding RNA; 2) a poly-A region within or ligated to the non-coding RNA; and 3) one or more copies of a structural sequence. In some embodiments, the modified non-coding RNA is a circular non-coding RNA. In some embodiments of the circular non-coding RNA, the one or more copies of the structural sequence are between the poly-A region within or ligated to the non-coding RNA and the 5′ nucleotide of the non-coding RNA.

In some embodiments of the modified non-coding RNAs provided herein, 1% to 90% of the nucleotides of the poly-A region are modified nucleotides. In some embodiments, at least 1%, at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the poly-A region are modified nucleotides.

In some embodiments of the modified non-coding RNAs provided herein, 3 or more of the last 25 nucleotides of the poly-A region are modified nucleotides. In some embodiments, at least 4, at least 5, at least 6, at least 7, at least 8, at least 9, at least 10, at least 11, at least 12, at least 13, at least 14, at least 15, at least 20, or 25 of the last 25 nucleotides of the poly-A region are modified nucleotides.

In some embodiments of the modified non-coding RNAs provided herein, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the nucleotides of the poly-A region are adenosine nucleotides. One or more adenosine nucleotides of the poly-A region may be canonical adenosine nucleotides or modified adenosine nucleotides comprising a different structure from the canonical adenosine nucleotide. Non-limiting examples of modified adenosine nucleotides include N6-isopentenyladenosine (i6A), 2-methyl-thio-N6-isopentenyladenosine (ms2i6A), 2-methylthio-N6-methyladenosine (ms2m6A), N6-(cis-hydroxyisopentenyl)adenosine (io6A), 2-methylthio-N6-(cis-hydroxyisopentenyl)adenosine (ms2io6A), N6-glycinylcarbamoyladenosine (g6A), N6-threonylcarbamoyladenosine (i6A), 2-methylthio-N6-threonyl carbamoyladenosine (ms2t6A), N6-methyl-N6-threonylcarbamoyladenosine (m6t6A), N6-hydroxynorvalylcarbamoyladenosine (hn6A), 2-methylthio-N6-hydroxynorvalyl carbamoyladenosine (ms2hn6A), 2′-O-ribosyladenosine (phosphate) (Ar(p)), N6,N6-dimethyladenosine (m62A), N6,2′-O-dimethyladenosine (m6Am), N6,N6,O-2′-trimethyladenosine (m62Am), 1,2′-O-dimethyladenosine (m1Am), N6-acetyladenosine (ac6A), 2′-thioadenosine (2′SA), 5′-thioadenosine (5′SA), 2′-O-(2-azidoethyl)-adenosine, 2′-azido-adenosine, deoxyadenosine (dA), dideoxyadenosine (ddA), and amino-deoxyadenosine (amino-dA).

In some embodiments of the modified non-coding RNAs provided herein, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the nucleotides of the poly-A region are canonical adenosine nucleotides. In some embodiments, the poly-A region further comprises 1 or more nucleotides that are not adenosine nucleotides (e.g., canonical or non-canonical adenosine nucleotides). In some embodiments, at least 1%, at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, at least 50%, at least 55%, at least 60%, at least 65%, at least 70%, at least 80%, or at least 90% of the nucleotides of the poly-A region are nucleotides that are not adenosine nucleotides.

In some embodiments of the modified non-coding RNAs provided herein, the poly-A region comprises at least 25-500 nucleotides. In some embodiments, the poly-A region comprises at least 25, at least 30, at least 50, at least 100, at least 150, or at least 200 nucleotides. In some embodiments, the poly-A region comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 nucleotides. In some embodiments, the poly-A region comprises about 200 to about 300 nucleotides. In some embodiments, the poly-A region comprises about 250 nucleotides.

In some embodiments, the poly-A region comprises at least 3, at least 4, or at least 5 phosphorothioates, and does not comprise a 3′ terminal hydroxyl. In some embodiments, the poly-A region of the non-coding RNA comprises at least 3 phosphorothioates, and does not comprise a 3′ terminal hydroxyl. In some embodiments, the poly-A region of the non-coding RNA comprises at least 3 guanine nucleotides and at least 3 phosphorothioates, and does not comprise a 3′ terminal hydroxyl. In some embodiments, the poly-A region of the non-coding RNA comprises at least 3 deoxyribose sugars, and does not comprise a 3′ terminal hydroxyl. In some embodiments, the poly-A region of the non-coding RNA comprises at least 20 deoxyribose sugars, and does not comprise a 3′ terminal hydroxyl. In some embodiments, the poly-A region of the non-coding RNA comprises at least 3 copies of a G-quadruplex sequence, and does not comprise a 3′ terminal hydroxyl. In some embodiments, the poly-A region of the non-coding RNA comprises at least 6 nucleotides comprising a 2′ modification, and does not comprise a 3′ terminal hydroxyl. In some embodiments, the poly-A region of the non-coding RNA comprises at least 6 phosphorothioates, and does not comprise a 3′ terminal hydroxyl. In some embodiments, the poly-A region of the non-coding RNA comprises at least 6 sequential nucleotides comprising a 2′ modification, and does not comprise a 3′ terminal hydroxyl. In some embodiments, the poly-A region of the non-coding RNA comprises at least 6 sequential phosphorothioates, and does not comprise a 3′ terminal hydroxyl. In some embodiments, the poly-A region of the non-coding RNA comprises at least 6 phosphorothioates and 3 guanine nucleosides, and does not comprise a 3′ terminal hydroxyl. In some embodiments, the poly-A region of the non-coding RNA comprises at least 3 copies of a G-quadruplex sequence and at least 6 phosphorothioates, and does not comprise a 3′ terminal hydroxyl. In some embodiments, the poly-A region of the non-coding RNA comprises at least 3 copies of a telomeric repeat sequence, and at least 6 phosphorothioates, and does not comprise a 3′ terminal hydroxyl. In some embodiments, the 3′ terminal nucleotide that does not comprise a 3′ terminal hydroxyl is a dideoxycytidine or an inverted-deoxythymidine.

Methods of Producing Modified mRNAs and Modified Non-Coding RNAs

In some aspects, the present disclosure provides methods of producing modified mRNAs, comprising ligating an RNA, such as an RNA comprising an open reading frame encoding a protein or a non-coding RNA, to a tailing nucleic acid comprising one or more modified nucleotides in the presence of a ligase, whereby the ligase forms a covalent bond between the 3′ nucleotide of the RNA and the 5′ nucleotide of the tailing nucleic acid to produce a modified RNA (e.g., a modified mRNA or a modified non-coding RNA). When a ligase forms a covalent bond between two linear nucleic acids, a new nucleic acid is produced, with the produced nucleic acid comprising the nucleic acid sequences of both nucleic acids. Ligation of the 3′ terminal nucleotide of a first nucleic acid to the 5′ terminal nucleotide of a second nucleic acid produces a third nucleic acid, with the third nucleic acid comprising the sequence of the first nucleic acid and the second nucleic acid, and the second nucleic acid sequence being 3′ to (downstream of) the first nucleic acid sequence. Ligation by an RNA ligase occurs in several steps. First, an amino (—NH₂) group of an amino acid (e.g., a lysine) of the ligase bonds to a phosphate group of adenosine triphosphate (ATP), such that an adenosine monophosphate (AMP) group is bound to the RNA ligase. Second, a 5′ terminal phosphate of the second nucleic acid displaces the phosphate of the RNA ligase-bound AMP. Finally, an oxygen of the 3′ terminal hydroxyl group of the first nucleic acid binds to the phosphorus atom of the 5′ terminal phosphate of the second nucleic acid. This final step forms a phosphodiester bond between terminal nucleotides of the nucleic acids, thereby forming a single nucleic acid with a continuous sugar-phosphate backbone. In some embodiments, the ligase is an RNA ligase. In some embodiments, the RNA ligase is a T4 RNA ligase.

In some embodiments of the methods of producing modified mRNAs or modified non-coding RNA provided herein, the RNA to which a tailing nucleic acid is ligated is synthesized by in vitro transcription (IVT). IVT is a process in which an RNA, such as a precursor mRNA (pre-mRNA), mRNA, or non-coding RNA, is generated through transcription of a DNA template by an RNA polymerase. Generally, the DNA template comprises a promoter, such as a bacteriophage promoter, that is upstream of the DNA sequence to be transcribed. The RNA polymerase binds to the promoter, and begins transcription of the DNA sequence, producing an RNA transcript with a nucleic acid sequence that is present in the template, with the exception that thymidine (T) nucleotides in the DNA sequence are replaced with uracil (U) nucleotides in the RNA sequence. The RNA transcript produced by IVT may be modified prior to ligation of a tailing nucleic acid, such as by the addition of a 5′ cap, cleavage of one or more nucleotides from the RNA, or polyadenylation to extend the poly-A region. In some embodiments, the DNA template comprises a poly-A region, such that IVT produces an mRNA or non-coding RNA with a poly-A region. See, e.g., Becker et al. Methods Mol Biol., 2011. 703:29-41.

In some embodiments of the methods of producing modified mRNAs or modified non-coding RNAs provided herein, the 3′ nucleotide of the RNA comprises a 3′ terminal hydroxyl group, and the 5′ nucleotide of the tailing nucleic acid comprises a 5′ terminal phosphate group. The combination of a 3′ terminal hydroxyl group on the RNA and a 5′ terminal phosphate group on the tailing nucleic acid allows for efficient ligation of the two nucleic acids. In some embodiments, the RNA does not comprise a 5′ terminal phosphate group. An RNA may lack a 5′ terminal phosphate group due to the addition of a 5′ cap or another chemical modification. A 5′ terminal phosphate may also be removed from an RNA by a phosphatase enzyme to produce an RNA that lacks a 5′ terminal phosphate. Lack of a 5′ terminal phosphate group on the RNA prevents an RNA ligase from ligating multiple copies of an mRNA or non-coding RNA together. In some embodiments, the tailing nucleic acid does not comprise a 3′ terminal hydroxyl group. An RNA may lack a 3′ terminal hydroxyl group if the last nucleotide of the tailing nucleic acid comprises a modified nucleotide that does not contain a 3′ hydroxyl group, such as a dideoxyadenosine, dideoxycytidine, dideoxyguanosine, dideoxythymidine, or inverted-deoxythymidine. Lack of a 3′ terminal hydroxyl group on the tailing nucleic acid prevents an RNA ligase from ligating multiple tailing nucleic acids together. In some embodiments, the 5′ nucleotide of the RNA does not comprise a 5′ terminal phosphate group; the 3′ nucleotide of the RNA comprises a 3′ terminal hydroxyl group; the 5′ nucleotide of the tailing nucleic acid comprises a 5′ terminal phosphate group; and the 3′ nucleotide of the tailing nucleic acid does not comprise a 3′ terminal hydroxyl group. In some embodiments, the tailing nucleic acid comprises at least 3, at least 4, or at least 5 phosphorothioates, and does not comprise a 3′ terminal hydroxyl. In some embodiments, the tailing nucleic acid comprises at least 3 phosphorothioates, and does not comprise a 3′ terminal hydroxyl. In some embodiments, the tailing nucleic acid comprises at least 3 guanine nucleotides and at least 3 phosphorothioates, and does not comprise a 3′ terminal hydroxyl. In some embodiments, the tailing nucleic acid comprises at least 3 deoxyribose sugars, and does not comprise a 3′ terminal hydroxyl. In some embodiments, the tailing nucleic acid comprises at least 20 deoxyribose sugars, and does not comprise a 3′ terminal hydroxyl. In some embodiments, the tailing nucleic acid comprises at least 3 copies of a G-quadruplex sequence, and does not comprise a 3′ terminal hydroxyl. In some embodiments, the tailing nucleic acid comprises at least 6 nucleotides comprising a 2′ modification, and does not comprise a 3′ terminal hydroxyl. In some embodiments, the tailing nucleic acid comprises at least 6 phosphorothioates, and does not comprise a 3′ terminal hydroxyl. In some embodiments, the tailing nucleic acid comprises at least 6 sequential nucleotides comprising a 2′ modification, and does not comprise a 3′ terminal hydroxyl. In some embodiments, the tailing nucleic acid comprises at least 6 sequential phosphorothioates, and does not comprise a 3′ terminal hydroxyl. In some embodiments, the tailing nucleic acid comprises at least 6 phosphorothioates and 3 guanine nucleosides, and does not comprise a 3′ terminal hydroxyl. In some embodiments, the tailing nucleic acid comprises at least 3 copies of a G-quadruplex sequence and at least 6 phosphorothioates, and does not comprise a 3′ terminal hydroxyl. In some embodiments, the tailing nucleic acid comprises at least 3 copies of a telomeric repeat sequence, and at least 6 phosphorothioates, and does not comprise a 3′ terminal hydroxyl. In some embodiments, the 3′ terminal nucleotide that does not comprise a 3′ terminal hydroxyl is a dideoxycytidine or an inverted-deoxythymidine. In some embodiments, the ligase used to ligate the tailing nucleic acid to the RNA is an RNA ligase. In some embodiments, the RNA ligase is a T4 RNA ligase. In some embodiments, the T4 RNA ligase is a T4 RNA ligase 1. In some embodiments, the T4 RNA ligase is a T4 RNA ligase 2.

In some embodiments of the methods of producing modified mRNAs or modified non-coding RNAs provided herein, the 5′ nucleotide of the RNA does not comprise a 5′ terminal hydroxyl group, the 3′ nucleotide of the RNA comprises a 3′ terminal phosphate group, the 5′ nucleotide of the tailing nucleic acid comprises a 5′ terminal hydroxyl group, the 3′ nucleotide of the tailing nucleic acid does not comprise a 3′ terminal phosphate group, and the RNA ligase is an RtcB ligase, which ligates a first nucleotide comprising a 3′ terminal phosphate group to a second nucleotide comprising a 5′ terminal hydroxyl group.

Some embodiments of the methods of making modified mRNAs or modified non-coding RNA provided herein further comprise producing a circular mRNA or circular non-coding RNA. After a linear modified mRNA or modified non-coding RNA is produced by ligating an RNA and a tailing nucleic acid, circularization of the modified mRNA or modified non-coding RNA comprises several additional steps. First, a 5′ terminal phosphate is introduced onto the first nucleotide of the modified mRNA or modified non-coding RNA, a process known as phosphorylation. In some embodiments, the 5′ terminal phosphate is introduced by a kinase. A “kinase” refers to an enzyme that introduces a phosphate group to a molecule, forming a covalent bond between the phosphate group and the molecule, in a process referred to as “phosphorylation.” Second, the modified mRNA or modified non-coding RNA is manipulated to produce a modified mRNA or modified non-coding RNA with a 3′ terminal hydroxyl group. In some embodiments, the modified mRNA or modified non-coding RNA is manipulated by cleaving one or more of the last nucleotides of the modified RNA, to produce a modified mRNA or modified non-coding RNA with a 3′ terminal hydroxyl group. In some embodiments, the modified mRNA or modified non-coding RNA is cleaved by a restriction enzyme, ribozyme, or endoribonuclease. In some embodiments, cleavage of one or more last nucleotides of the modified mRNA or modified non-coding RNA occurs before phosphorylation of the first nucleotide of the modified RNA. In some embodiments, cleavage occurs after phosphorylation. A modified mRNA or modified non-coding RNA comprising a terminal phosphate group at one end and a terminal hydroxyl group at the other end can be circularized by ligation of both terminal nucleotides. An RNA ligase that ligates terminal nucleotides of a linear nucleic acid to produce a circular nucleic acid may be called a “circularizing ligase” In some embodiments, the circularizing ligase is an RNA ligase. In some embodiments, the circularizing ligase is a SplintR ligase. In some embodiments, the circularizing ligase is a T4 RNA ligase. In some embodiments, the circularizing ligase is a T4 RNA ligase 1. In some embodiments, the circularizing ligase is a T4 RNA ligase 2. In some embodiments, the modified mRNA or modified non-coding RNA comprises a 5′ terminal hydroxyl group and a 3′ terminal phosphate group, and the circularizing ligase is RtcB ligase, which is capable of ligating nucleotides with a 3′ terminal phosphate and 5′ terminal hydroxyl group. For ligation to occur, the 5′ and 3′ terminal nucleotides of the modified mRNA or modified non-coding RNA must be close enough for the RNA ligase to form a bond between both nucleotides. Methods of placing both nucleotides of a linear nucleic acid close enough for ligation to occur, and of circularizing an RNA, are generally known in the art (see, e.g., Petkovic et al., Nucleic Acids Res., 2015. 43(4):2454-2465). In some embodiments, the modified mRNA or modified non-coding RNA is incubated with a scaffold nucleic acid, which is capable of hybridizing (hydrogen bonding) to the modified RNA so that the modified mRNA or modified non-coding RNA forms a circular secondary structure when hybridized (bound) to the scaffold nucleic acid.

When an RNA forms a circular secondary structure, the 5′ and 3′ terminal nucleotides are in close physical proximity, which is required for an RNA ligase to form a covalent bond between them. In some embodiments of methods of circularizing an mRNA or non-coding RNA, one or more of the last nucleotides of the RNA are bound to a first hybridization sequence in the scaffold nucleic acid, and one or more of the first nucleotides of the mRNA or non-coding RNA are bound to a second hybridization sequence in the scaffold nucleic acid that is 3′ to (downstream of) the first hybridization sequence. In some embodiments, the first hybridization sequence comprises 5 or more nucleotides, and the first hybridization sequence is complementary to at least the first five (5) nucleotides of the modified mRNA or modified non-coding RNA. In some embodiments, the first hybridization sequence comprises 10 or more, 15 or more, 20 or more, 25 or more, 30 or more, 35 or more, 40 or more, 45 or more, or 50 or more nucleotides, and at least 90%, at least 95%, at least 96%, at least 97%, at least 98%, at least 99%, or up to 100% of the nucleotides of the first hybridization sequence are complementary are complementary to the last N nucleotides of the modified mRNA or modified non-coding RNA, where N is the length of the first hybridization sequence. In some embodiments, the second hybridization sequence comprises 5 or more nucleotides, and the second hybridization sequence is complementary to at least the last five (5) nucleotides of the modified mRNA or modified non-coding RNA. In some embodiments, the second hybridization sequence comprises 10 or more, 15 or more, 20 or more, 25 or more, 30 or more, 35 or more, 40 or more, 45 or more, or 50 or more nucleotides, and at least 90%, at least 95%, at least 96%, at least 97%, at least 98%, at least 99%, or up to 100% of the nucleotides of the second hybridization sequence are complementary are complementary to the last N nucleotides of the modified mRNA or modified non-coding RNA, where N is the length of the second hybridization sequence. In some embodiments, at least the first five (5) nucleotides of the modified mRNA or modified non-coding RNA hybridize with the first hybridization sequence. In some embodiments, at least the last five (5) nucleotides of the modified mRNA or modified non-coding RNA hybridize with the second hybridization sequence. In some embodiments, at least the first five (5) nucleotides of the modified mRNA or modified non-coding RNA hybridize with the first hybridization sequence, and at least the last five (5) nucleotides of the modified mRNA or modified non-coding RNA hybridize with the second hybridization sequence. In some embodiments, the last nucleotide of the first hybridization sequence and the first nucleotide of the second hybridization sequence are adjacent in the scaffold nucleic acid, and are not separated by any other nucleotides.

In some embodiments of the methods of producing circular RNAs provided herein, a scaffold nucleic acid is not used to promote the formation of a circular secondary structure by the modified mRNA or modified non-coding RNA. Instead, the modified mRNA or modified non-coding RNA comprises a first hybridization sequence at the 5′ end that is complementary to a second hybridization sequence at the 3′ end. In some embodiments, each hybridization sequence comprises at least five (5) nucleotides. In some embodiments, each hybridization sequence comprises at least 10, at least 15, at least 20, at least 25, at least 30, at least 35, at least 40, at least 45, or at least 50 nucleotides.

In some embodiments of the methods of producing circular RNAs provided herein, the modified mRNA or modified non-coding RNA is not circularized through the use of a scaffold nucleic acid and circularizing ligase, but rather is circularized by a ribozyme, a nucleic acid that catalyzes a reaction, such as the formation of a covalent bond between two nucleotides. In some embodiments, prior to circularization, the modified mRNA or modified non-coding RNA comprises a 3′ intron that is 5′ to (upstream of) the 5′ UTR of the mRNA or the first nucleotide of the non-coding mRNA, and a 5′ intron that is 3′ to (downstream of) the poly-A region and/or one or more structural sequences of the mRNA or non-coding RNA. Ribozymes and other enzymes that catalyze splicing of pre-mRNA to remove introns can catalyze the formation of a covalent bond between the nucleotide that is 5′ to the 5′ intron and the nucleotide that is 3′ to 3′ intron, resulting in the formation of a circular mRNA or non-coding RNA. See, e.g., Wesselhoeft et al., Nat Commun. 2018. 9:2629.

In some embodiments of the methods of producing circular RNAs provided herein, the modified mRNA or modified non-coding RNA is not circularized through the use of a scaffold nucleic, but rather is circularized through the use of complementary sequences that promote the formation of a secondary structure by the mRNA of non-coding RNA that places the 5′ and 3′ terminal nucleotides of the mRNA or non-coding RNA in close proximity. In some embodiments, prior to circularization the modified mRNA comprises (i) a first self-hybridization sequence that is 5′ to the open reading frame, or 5′ to the non-coding RNA; (ii) a second self-hybridization sequence that is 3′ to the open reading frame, or 3′ to the non-coding RNA; (iii) a first non-hybridization sequence that is 5′ to the first self-hybridization sequence; and (iv) a second non-hybridization sequence that is 3′ to the second self-hybridization sequence. The first and second self-hybridization sequences are capable of hybridizing with each other, but the first and second self-hybridization sequences are not capable of hybridizing with each other. In some embodiments, hybridization of the first and second self-hybridization sequences forms a secondary structure in which the 5′ terminal nucleotide and the 3′ terminal nucleotide of the modified mRNA or modified non-coding RNA are separated by a distance of less than 100 Å. In some embodiments, the 5′ terminal nucleotide and the 3′ terminal nucleotide are separated by a distance of less than 90 Å, less than 80 Å, less than 70 Å, less than 60 Å, less than 50 Å, less than 40 Å, less than 30 Å, less than 20 Å, or less than 10 Å See, e.g., Carmona, Ellese Marie. 2019. Circular RNA: Design Criteria for Optimal Therapeutical Utility. Doctoral dissertation, Harvard University, Graduate School of Arts & Sciences; Petkovic et al. Nucleic Acids Res., 2015. 43(4):2454-2465; and WO 2020/237227.

In some embodiments of the methods of producing modified mRNAs or modified non-coding RNAs provided herein, the modified mRNA or modified non-coding RNA produced by the method comprises one or more copies of a structural sequence that are 3′ to the poly-A region of the mRNA or non-coding RNA. In some embodiments, the tailing nucleic acid comprises the one or more copies of the structural sequence. In some embodiments, nucleotides of the structural sequences interact by hydrogen bonding. In some embodiments, the secondary structure is a G-quadruplex. In some embodiments, the structural sequence is a G-quadruplex sequence. In some embodiments, the G-quadruplex sequence comprises one or more spacer nucleotides that are not guanine nucleotides. In some embodiments, the G-quadruplex sequence is an RNA G-quadruplex sequence. In some embodiments, the RNA G-quadruplex sequence comprises the nucleic acid sequence GGGGCC (SEQ ID NO: 2). In some embodiments, the tailing nucleic acid comprises at least 3 copies of the nucleic acid sequence of SEQ ID NO: 2. In some embodiments, the G-quadruplex sequence is an DNA G-quadruplex sequence. In some embodiments, the DNA G-quadruplex sequence comprises the nucleic acid sequence GGGGCC (SEQ ID NO: 3). In some embodiments, the tailing nucleic acid comprises at least 3 copies of the G-quadruplex sequence of SEQ ID NO: 3. In some embodiments, the structural sequence comprises a telomeric repeat sequence. In some embodiments, the telomeric repeat sequence comprises the nucleic acid sequence set forth as one of SEQ ID NOs: 4 or 5 (TAGGGT or TACCCT, respectively). In some embodiments, the telomeric repeat sequence comprises the nucleic acid sequence set forth as SEQ ID NO: 4. In some embodiments, the tailing nucleic acid comprises at least 3 copies of the nucleic acid sequence of SEQ ID NO: 4. In some embodiments, the structural sequence is an aptamer sequence comprising at least two nucleotides that are capable of interacting to form an aptamer. In some embodiments, the secondary structure formed by the one or more copies of the structural sequence is an aptamer that is capable of binding to a target molecule. Formation of an aptamer by an mRNA or non-coding RNA allows for the mRNA or non-coding RNA to be localized to a given region of a cell containing a target molecule, such as a receptor.

In some embodiments of the modified mRNAs or modified non-coding RNAs produced by the methods provided herein, the modified mRNA or modified non-coding RNA comprises 1-20 copies of the structural sequence. In some embodiments, the modified mRNA or modified non-coding RNA comprises at least 1, at least 2, at least 3, at least 4, at least 5, at least 6, at least 7, at least 8, or at least 9 copies of the structural sequence. In some embodiments, the modified mRNA or modified non-coding RNA comprises about 4 copies of the structural sequence. In some embodiments, the modified mRNA or modified non-coding RNA comprises multiple different structural sequences. In some embodiments, the modified mRNA or modified non-coding RNA comprises at least a first structural sequence, and a second structural sequence comprising a different nucleic acid sequence from the first structural sequence.

In some embodiments of the modified mRNAs or modified non-coding RNAs produced by the methods provided herein, the poly-A region of the modified mRNA or modified non-coding RNA comprises at least one modified nucleotide. In some embodiments, the tailing nucleic acid comprises at least one modified nucleotide. In some embodiments, at least one modified nucleotide comprises a modified nucleobase. In some embodiments, at least one modified nucleotide comprises a modified sugar. In some embodiments, at least one modified nucleotide comprises a modified phosphate. In some embodiments, at least one modified nucleotide comprises a modified nucleobase selected from the group consisting of: xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, at least one modified nucleotide comprises a modified sugar selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose. In some embodiments, at least one modified nucleotide comprises a 2′ modification. In some embodiments, the 2′ modification is selected from the group consisting of a locked-nucleic acid (LNA) modification (i.e., a nucleotide comprising an additional carbon atom bound to the 2′ oxygen and 4′ carbon of ribose), 2′-fluoro (2′-F), 2′-O-methoxy-ethyl (2′-MOE), and 2′-O-methylation (2′-OMe). In some embodiments, at least one modified nucleotide comprises a modified phosphate selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate. In some embodiments, the poly-A region of the mRNA or non-coding RNA comprises at least 3, at least 4, or at least 5 phosphorothioates, and does not comprise a 3′ terminal hydroxyl. In some embodiments, the poly-A region of the mRNA or non-coding RNA comprises at least 3 phosphorothioates, and does not comprise a 3′ terminal hydroxyl. In some embodiments, the poly-A region of the mRNA or non-coding RNA comprises at least 3 guanine nucleotides and at least 3 phosphorothioates, and does not comprise a 3′ terminal hydroxyl. In some embodiments, the poly-A region of the mRNA or non-coding RNA comprises at least 3 deoxyribose sugars, and does not comprise a 3′ terminal hydroxyl. In some embodiments, the poly-A region of the mRNA or non-coding RNA comprises at least 20 deoxyribose sugars, and does not comprise a 3′ terminal hydroxyl. In some embodiments, the poly-A region of the mRNA or non-coding RNA comprises at least 3 copies of a G-quadruplex sequence, and does not comprise a 3′ terminal hydroxyl. In some embodiments, the poly-A region of the mRNA or non-coding RNA comprises at least 6 nucleotides comprising a 2′ modification, and does not comprise a 3′ terminal hydroxyl. In some embodiments, the poly-A region of the mRNA or non-coding RNA comprises at least 6 phosphorothioates, and does not comprise a 3′ terminal hydroxyl. In some embodiments, the poly-A region of the mRNA or non-coding RNA comprises at least 6 sequential nucleotides comprising a 2′ modification, and does not comprise a 3′ terminal hydroxyl. In some embodiments, the poly-A region of the mRNA or non-coding RNA comprises at least 6 sequential phosphorothioates, and does not comprise a 3′ terminal hydroxyl. In some embodiments, the poly-A region of the mRNA or non-coding RNA comprises at least 6 phosphorothioates and 3 guanine nucleosides, and does not comprise a 3′ terminal hydroxyl. In some embodiments, the poly-A region of the mRNA or non-coding RNA comprises at least 3 copies of a G-quadruplex sequence and at least 6 phosphorothioates, and does not comprise a 3′ terminal hydroxyl. In some embodiments, the poly-A region of the mRNA or non-coding RNA comprises at least 3 copies of a telomeric repeat sequence, and at least 6 phosphorothioates, and does not comprise a 3′ terminal hydroxyl. In some embodiments, the 3′ terminal nucleotide that does not comprise a 3′ terminal hydroxyl is a dideoxycytidine or an inverted-deoxythymidine.

In some embodiments of the modified mRNAs or modified non-coding RNAs produced by the methods provided herein, the modified mRNA or modified non-coding RNA comprises more than one type of modified nucleotide. In some embodiments, the modified mRNA or modified non-coding RNA comprises at least a first modified nucleoside, and a second modified nucleoside that has a different structure from the first modified nucleoside. In some embodiments, the modified mRNA or modified non-coding RNA comprises at least a first modified phosphate, and a second modified phosphate that has a different structure from the first modified phosphate. In some embodiments, the modified mRNA or modified non-coding RNA comprises a modified nucleoside and a modified nucleoside.

In some embodiments of the modified mRNAs or modified non-coding RNAs produced by the methods provided herein, 1% to 90% of the nucleotides of the poly-A region are modified nucleotides. In some embodiments, at least 1%, at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the poly-A region are modified nucleotides.

In some embodiments of the modified mRNAs or modified non-coding RNAs produced by the methods provided herein, 3 or more of the last 25 nucleotides of the poly-A region are modified nucleotides. In some embodiments, at least 4, at least 5, at least 6, at least 7, at least 8, at least 9, at least 10, at least 11, at least 12, at least 13, at least 14, at least 15, at least 20, or 25 of the last 25 nucleotides of the poly-A region are modified nucleotides.

In some embodiments of the modified mRNAs or modified non-coding RNAs produced by the methods provided herein, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the nucleotides of the poly-A region are adenosine nucleotides. One or more adenosine nucleotides of the poly-A region may be canonical adenosine nucleotides or modified adenosine nucleotides comprising a different structure from the canonical adenosine nucleotide.

In some embodiments of the modified mRNAs or modified non-coding RNAs produced by the methods provided herein, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the nucleotides of the poly-A region are canonical adenosine nucleotides.

In some embodiments of the modified mRNAs or modified non-coding RNAs produced by the methods provided herein, the poly-A region comprises at least 25-500 nucleotides. In some embodiments, the poly-A region comprises at least 25, at least 30, at least 50, at least 100, at least 150, or at least 200 nucleotides. In some embodiments, the poly-A region comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 nucleotides. In some embodiments, the poly-A region comprises about 200 to about 300 nucleotides. In some embodiments, the poly-A region comprises about 250 nucleotides.

In some embodiments of the methods of producing modified mRNAs provided herein, prior to the ligation of a tailing nucleic acid, the RNA comprises an open reading frame and a poly-A region prior to ligation of a tailing nucleic acid. In some embodiments of the methods of producing modified non-coding RNAs provided herein, prior to the ligation of a tailing nucleic acid, the RNA comprises a non-coding RNA and may or may not comprise a poly-A region prior to ligation of a tailing nucleic acid. In some embodiments, prior to ligation of a tailing nucleic acid, the poly-A region of the RNA comprises at least 25-500 nucleotides. In some embodiments, the poly-A region comprises at least 25, at least 30, at least 50, at least 100, at least 150, or at least 200 nucleotides. In some embodiments, the poly-A region comprises at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 nucleotides. In some embodiments, the poly-A region comprises about 200 to about 300 nucleotides. In some embodiments, the poly-A region comprises about 250 nucleotides.

In some embodiments, prior to ligation of a tailing nucleic acid, the tailing nucleic acid comprises at least 10-500 nucleotides. In some embodiments, the tailing nucleic acid comprises at least 10, at least 15, at least 20, at least 25, at least 30, at least 50, at least 100, at least 150, or at least 200 nucleotides. In some embodiments, the tailing nucleic acid comprises at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 nucleotides. In some embodiments, the poly-A region comprises about 10 to about 50 nucleotides.

In some embodiments of the methods of producing modified mRNAs provided herein, prior to ligation of a tailing nucleic acid, the RNA comprises, in 5′-to-3′ order, a 5′ UTR, an open reading frame, a 3′ UTR, and a poly-A region. In some embodiments, the open reading frame is between the 5′ UTR and the 3′ UTR. In some embodiments, the 3′ UTR is between the open reading frame and the poly-A region.

In some embodiments of the methods of producing modified non-coding RNAs provided herein, prior to ligation of a tailing nucleic acid, the RNA comprises, in 5′-to-3′ order, a non-coding RNA, and optionally a poly-A region. In some embodiments, the first nucleotide of the poly-A region is 3′ to the last nucleotide of the non-coding RNA. In some embodiments, prior to ligation of a tailing nucleic acid, a non-coding RNA does not comprise a poly-A tail. Accordingly, in some embodiments, the tailing nucleic acid comprises a poly-A region described herein that is added to the 3′ end of the non-coding RNA by ligating the tailing nucleic acid to the 3′ end of the non-coding RNA, thereby producing a modified non-coding RNA comprising a poly-A region.

In some embodiments of the methods of producing modified mRNAs or modified non-coding RNAs provided herein, prior to ligation of a tailing nucleic acid, the RNA comprises a 5′ cap. In some embodiments, the 5′ cap comprises a 7-methylguanosine. In some embodiments, the 5′ cap comprises one or more phosphates that connect the 7-methylguanosine to an adjacent nucleotide of the RNA. In some embodiments, a 5′ cap is added after ligation of the tailing nucleic acid. In some embodiments, prior to ligation of a tailing nucleic acid, the RNA does not comprise a 5′ cap (e.g., the RNA is a mRNA or non-coding RNA that does not comprise a 5′ cap).

In some aspects of the methods of producing modified mRNAs or modified non-coding RNAs provided herein comprising ligating a tailing nucleic acid to an mRNA or non-coding RNA, the tailing nucleic acid comprises one or more modified nucleotides. In some embodiments, the tailing nucleic acid comprises at least one modified nucleotide comprising a modified nucleoside. In some embodiments, at least one modified nucleotide comprises a modified nucleoside comprising a modified nucleobase and/or a modified sugar. In some embodiments, at least one modified nucleotide comprises a modified nucleoside comprising a modified nucleobase and a modified sugar. In some embodiments, at least one modified nucleotide comprises a modified nucleobase. In some embodiments, at least one modified nucleotide comprises a modified sugar. In some embodiments, at least one modified nucleotide comprises a modified phosphate. In some embodiments, at least one modified nucleotide comprises a modified nucleobase selected from the group consisting of: xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A). In some embodiments, at least one modified nucleotide comprises a modified sugar selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-0)-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose. In some embodiments, at least one modified nucleotide comprises a 2′ modification. In some embodiments, the 2′ modification is selected from the group consisting of a locked-nucleic acid (LNA) modification (i.e., a nucleotide comprising an additional carbon atom bound to the 2′ oxygen and 4′ carbon of ribose), 2′-fluoro (2′-F), 2′-O-methoxy-ethyl (2′-MOE), and 2′-O-methylation (2′-OMe). In some embodiments, at least one modified nucleotide comprises a modified phosphate selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

In some embodiments of the methods of producing modified mRNAs or modified non-coding RNAs provided herein, the tailing nucleic acid comprises more than one type of modified nucleotide. In some embodiments, the tailing nucleic acid comprises at least a first modified nucleoside, and a second modified nucleoside that has a different structure from the first modified nucleoside. In some embodiments, the tailing nucleic acid comprises at least a first modified phosphate, and a second modified phosphate that has a different structure from the first modified phosphate. In some embodiments, the tailing nucleic acid comprises a modified nucleoside and a modified nucleoside.

In some embodiments, 1% to 90% of the nucleotides of the tailing nucleic acid are modified nucleotides. In some embodiments, at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the tailing nucleic acid are modified nucleotides. In some embodiments, 3 or more of the 25 last nucleotides of the tailing nucleic acid are modified nucleotides. In some embodiments, at least 4, 5, 6, 7, 8, 9, 10, 15, 20, or 25 of the 25 last nucleotides of the tailing nucleic acid are modified nucleotides.

In some embodiments of the methods of producing modified mRNAs or modified non-coding RNAs provided herein, the tailing nucleic acid comprises one or more structural sequences. In some embodiments, the tailing nucleic acid comprises one or more copies of a G-quadruplex sequence. In some embodiments, the G-quadruplex sequence is an RNA G-quadruplex sequence. In some embodiments, the RNA G-quadruplex sequence comprises the nucleic acid sequence GGGGCC (SEQ ID NO: 2). In some embodiments, the G-quadruplex sequence is an DNA G-quadruplex sequence. In some embodiments, the DNA G-quadruplex sequence comprises the nucleic acid sequence GGGGCC (SEQ ID NO: 3). In some embodiments, the tailing nucleic acid comprises one or more copies of a telomeric repeat sequence. In some embodiments, the telomeric repeat sequence comprises the nucleic acid sequence set forth as one of SEQ ID NOs: 4 or 5 (TAGGGT or TACCCT, respectively). In some embodiments, the telomeric repeat sequence comprises the nucleic acid sequence set forth as SEQ ID NO: 4. In some embodiments, the structural sequence is an aptamer sequence comprising at least two nucleotides that are capable of interacting to form an aptamer. In some embodiments, the secondary structure formed by the one or more copies of the structural sequence is an aptamer that is capable of binding to a target molecule.

In some embodiments of the methods of producing modified mRNAs or modified non-coding RNAs provided herein, the tailing nucleic acid comprises 1-20 copies of a structural sequence. In some embodiments, the tailing nucleic acid comprises at least 1, at least 2, at least 3, at least 4, at least 5, at least 6, at least 7, at least 8, or at least 9 copies of the structural sequence. In some embodiments, the tailing nucleic acid comprises about 4 copies of the structural sequence. In some embodiments, the tailing nucleic acid comprises multiple different structural sequences. In some embodiments, the tailing nucleic acid comprises at least a first structural sequence, and a second structural sequence comprising a different nucleic acid sequence from the first structural sequence. Each of the different first and second structural sequences may be any of the structural sequences provided herein, or different sequences. In further embodiments, the methods of producing modified mRNAs or modified non-coding RNAs also relate to methods for isolating (e.g., purifying, enriching) the modified mRNAs or modified non-coding RNAs provided herein. In some embodiments, a method of isolating (e.g., purifying, enriching) a modified mRNA or modified non-coding RNA comprises contacting a mixture comprising the modified mRNA or modified non-coding RNA (e.g., a ligation mixture) with a purification medium, wherein the modified mRNA or modified non-coding RNA interacts with the purification medium to form a modified RNA-purification medium conjugate. In some embodiments, a purification medium that has formed a modified RNA-purification medium conjugate is separated from the mixture by means of one or more physical or chemical properties, such as, but not limited to, size (mass) or charge. In some embodiments, the modified mRNA or modified non-coding RNA is eluted from the purification medium (i.e., separated from the purification medium) by treating the modified RNA-purification medium conjugate with a solvent. In some embodiments, the solvent is an aqueous solvent (e.g., water). In certain embodiments, the solvent is a mixture of two or more (e.g., three) solvents. In certain embodiments, the solvent is a mixture of water and an organic solvent (e.g., acetonitrile, methanol, ethanol, tetrahydrofuran). In certain embodiments, the solvent further comprises a mobile phase modifying substance. In certain embodiments, the mobile phase modifying substance is an acid (e.g., trifluoroacetic acid, acetic acid, formic acid, phosphoric acid), base (ammonia, ammonium hydroxide, ammonium bicarbonate), or salt (a phosphate, an acetate, a citrate, ammonium formate, or a borate). In some embodiments, the purification medium is a solid purification medium. In some embodiments, the purification medium comprises a bead. In some embodiments, the purification medium comprises a resin. In some embodiments, the purification medium comprises a paramagnetic bead. Examples of purification media suitable for the purification of RNA are well known to those skilled in the art and include, for example, various commercially available purification media (see, e.g., Beckman Coulter Life Sciences #A63987). In certain embodiments, a step described in this paragraph is performed at a temperature between 0 and 20, between 20 and 25, between 25 and 36, between 36 and 38° C., inclusive. In certain embodiments, a step described in this paragraph is performed at a pressure between 0.9 and 1.1 atm, inclusive.

Compositions Comprising Modified mRNAs or Modified Non-Coding RNAs and Methods of Use

In some aspects, the present disclosure provides compositions comprising any one of the modified mRNAs or modified non-coding RNAs provided herein. In some embodiments, the modified mRNA or modified non-coding RNA is made by any of the methods provided herein comprising ligating a tailing nucleic acid onto an RNA. Compositions comprising a modified mRNA are useful for delivering the modified mRNA to a cell in order to vaccinate the subject against a foreign antigen, or express a therapeutic protein to treat a condition or disorder. Compositions comprising a modified non-coding RNA are useful for modulating the expression of genes in a cell or subject, or for editing the genome of a cell or subject, and may be used to treat a condition or disorder. Compositions comprising modified mRNAs or modified non-coding RNAs are also useful for exerting a desired effect in a subject in the absence of disease, such as for agricultural uses. For example, an mRNA encoding a biological pesticide or growth-augmenting factor or a non-coding RNA for genome editing may be used to increase the tolerance of a plant to pests, or modulate growth in a manner that increases crop yield, respectively. Any of the modified mRNAs or modified non-coding RNA described herein or a composition thereof may be used to enhance the delivery and/or stability of mRNAs or modified non-coding RNA to plants or plant cells, and may be used to augment techniques for plant genome engineering that are well established in the art. See, e.g., Stoddard, et al. PLOS One. 2016; 11 (5): e0154634.

In some embodiments, the open reading frame of the mRNA is codon-optimized for expression in a cell of a subject. As used herein, “codon-optimized” refers to the preferential use of codons that are more efficiently translated in a cell. Multiple codons can encode the same amino acid, with the translation rate and efficiency of each codon being determined by multiple factors, such as the intracellular concentration of aminoacyl-tRNAs comprising a complementary anticodon. Codon optimization of a nucleic acid sequence may include replacing one or more codons with codons that encode the same amino acid as, but are more efficiently translated than, the replaced codons. For example, the amino acid threonine (Thr) may be encoded by ACA, ACC, ACG, or ACT (ACU in RNA), but in mammalian host cells ACC is the most commonly used codon; in other species, different Thr codons may be preferred for codon-optimized. An mRNA with a codon-optimized open reading frame is thus expected to be translated more efficiently, and produce more polypeptides in a given amount of time, than an mRNA with an open reading frame that is not codon-optimized. In some embodiments, the open reading frame is codon-optimized for expression in a human cell.

In some embodiments of the modified mRNAs provided herein, the open reading frame encodes an antigen or a therapeutic protein. As used herein, a “therapeutic protein” refers to a protein that prevents, reduces, or alleviates one or more signs or symptoms of a disease when expressed in a subject, such as a human subject that has, or is at risk of developing, a disease or disorder. A therapeutic protein may be an essential enzyme or transcription factor encoded by a gene that is mutated in a subject. For example, IPEX syndrome in humans is caused by a mutation in the FOXP3 gene, which hinders development of FOXP3+ regulatory T cells and results in increased susceptibility to autoimmune and inflammatory disorders. Expression of an essential enzyme or transcription factor from an mRNA may therefore compensate for a mutation in the gene encoding the enzyme or transcription factor in a subject. As used herein, “antigen” refers to a molecule (e.g., a protein) that, when expressed in a subject, elicits the generation of antibodies in the subject that bind to the antigen. In some embodiments, the antigen is a protein derived from a virus (viral antigen) or a fragment thereof. In some embodiments, the antigen is a protein derived from a bacterium (bacterial antigen) or a fragment thereof. In some embodiments, the antigen is a protein derived from a protozoan (protozoal antigen) or a fragment thereof. In some embodiments, the antigen is a protein derived from a fungus (fungal antigen) or a fragment thereof. A fragment of a full-length protein refers to a protein with an amino acid sequence that is present in, but shorter than, the amino acid sequence of the full-length protein.

In some aspects, the present disclosure provides lipid nanoparticles comprising any of the modified mRNAs or modified non-coding RNAs provided herein. A lipid nanoparticle refers to a composition comprising one or more lipids that form an aggregate of lipids, or an enclosed structure with an interior surface and an exterior surface. Lipids used in the formulation of lipid nanoparticles for delivering mRNA or non-coding RNA are generally known in the art, and include ionizable amino lipids, non-cationic lipids, sterols, and polyethylene glycol-modified lipids. See, e.g., Buschmann et al. Vaccines. 2021. 9(1):65. In some embodiments, the modified mRNA or modified non-coding RNA is surrounded by the lipids of the lipid nanoparticle and present in the interior of the lipid nanoparticle. In some embodiments, the mRNA or non-coding RNA is dispersed throughout the lipids of the lipid nanoparticle. In some embodiments, the lipid nanoparticle comprises an ionizable amino lipid, a non-cationic lipid, a sterol, and/or a polyethylene glycol (PEG)-modified lipid.

In some aspects, the present disclosure provides cells comprising any of the modified mRNAs or modified non-coding RNAs provided herein. In some embodiments, the cell is a human cell comprising any one of the modified mRNAs or modified non-coding RNAs provided herein. A “cell” is the basic structural and functional unit of all known independently living organisms. It is the smallest unit of life that is classified as a living thing. Some organisms, such as most bacteria, are unicellular (consist of a single cell). Other organisms, such as plants, fungi, and animals, including cattle, horses, chickens, turkeys, sheep, swine, dogs, cats, and humans, are multicellular. In some embodiments, the half-life of the modified mRNA or modified non-coding RNA in the cell is 15-900 minutes. In some embodiments, the half-life of the modified mRNA or modified non-coding RNA in the cell is 30-600 minutes. In some embodiments, the half-life of the modified mRNA or modified non-coding RNA in the cell is 60-300 minutes. In some embodiments, the half-life of the modified mRNA or modified non-coding RNA is at least 15, at least 20, at least 25, at least 30, at least 35, at least 40, at least 45, at least 50, at least 55, at least 60 minutes. In some embodiments, the half-life of the modified mRNA or modified non-coding RNA in the cell is at least 30, at least 60, at least 90, at least 120, at least 150, at least 180, at least 210, at least 240, at least 270, at least 300, at least 330, at least 360, at least 390, at least 420, at least 450, at least 480, at least 510, at least 540, at least 570, at least 600, at least 630, at least 660, at least 690, at least 720, at least 750, at least 780, at least 810, at least 840, or at least 870 minutes.

In some aspects, the present disclosure provides compositions comprising any of the modified mRNAs, modified non-coding RNAs, lipid nanoparticles, or cells provided herein. In some embodiments, the composition is a pharmaceutical composition comprising any one of the modified mRNAs, modified non-coding RNAs, lipid nanoparticles, or cells provided herein, and a pharmaceutically acceptable excipient. Pharmaceutically acceptable excipients, carriers, buffers, stabilisers, isotonicising agents, preservatives or antioxidants, or other materials well known to those skilled in the art. Such materials should be non-toxic and should not interfere with the efficacy of the active ingredient. The precise nature of the carrier or other material may depend on the route of administration, e.g., parenteral, intramuscular, intradermal, sublingual, buccal, ocular, intranasal, subcutaneous, intrathecal, intratumoral, oral, vaginal, or rectal.

In some aspects, the present disclosure provides a method of administering to a subject any of the modified mRNAs, modified non-coding RNAs, lipid nanoparticles, cells, compositions, or pharmaceutical compositions provided herein. In some embodiments, the any of the modified mRNAs or modified non-coding RNAs described herein can be used in conjunction with a variety of reagents or materials (e.g., one or more lipid nanoparticles, cells, compositions, or pharmaceutical compositions) or with certain production, purification, formulation, and delivery processes and techniques known in the art, such as those exemplified in, but not limited to, U.S. Pat. Nos. 9,950,065, 10,576,146, 11,045,418, 8,754,062, 10,808,242, 9,957,499, 10,155,785, 11,059,841, 10,876,104, 10,975,369, 9,580,711, 9,670,152, 9,850,202, 9,896,413, 10,399,937, 10,052,284, 10,959,953, and 10,961,184, each of which are incorporated by reference herein.

In some embodiments, the subject is a human. In some embodiments, the administration is parenteral, intramuscular, intradermal, sublingual, buccal, ocular, intranasal, subcutaneous, intrathecal, intratumoral, oral, vaginal, or rectal.

In some embodiments, the composition is to be stored below 50° C., below 40° C., below 30° C., below 20° C., below 10° C., below 0° C., below −10° C., below −20° C., below −30° C., below −40° C., below −50° C., below −60° C., below −70° C., or below −80° C., such that the nucleic acids are relatively stable over time.

In some embodiments, the modified mRNA or modified non-coding RNA is introduced into a cell in a subject by in vivo electroporation. In vivo electroporation is the process of introducing nucleic acids or other molecules into a cell of a subject using a pulse of electricity, which promote passage of the nucleic acids or other molecules through the cell membrane and/or cell wall. See, e.g., Somiari et al. Molecular Therapy., 2000. 2(3): 178-187. The nucleic acid or molecule to be delivered is administered to the subject, such as by injection, and a pulse of electricity is applied to the injection site, whereby the electricity promotes entry of the nucleic acid into cells at the site of administration. In some embodiments, the nucleic acid is administered with other elements, such as buffers and/or excipients, that increase the efficiency of electroporation.

In some aspects, the present disclosure provides a kit comprising any of the RNAs and any of the tailing nucleic acids provided herein. The RNA and tailing nucleic acid can be combined in the presence of an RNA ligase to produce a modified mRNA or modified non-coding RNA, such as one of the modified mRNAs or modified non-coding RNAs provided herein. In some embodiments, the kit comprises a ligase. In some embodiments, the kit comprises an RNA ligase. In some embodiments, the kit comprises a T4 RNA ligase. In some embodiments, a kit comprises a T4 RNA ligase 1. In some embodiments, a kit comprises a T4 RNA ligase 2. In some embodiments, the kit comprises an RtcB RNA ligase. In some embodiments, the kit further comprises a buffer for carrying out the ligation. In some embodiments, the kit further comprises a nucleotide triphosphate, such as ATP, to provide energy required by the ligase. In some embodiments, the kit is to be stored below 50° C., below 40° C., below 30° C., below 20° C., below 10° C., below 0° C., below −10° C., below −20° C., below −30° C., below −40° C., below −50° C., below −60° C., below −70° C., or below −80° C., such that the nucleic acids are relatively stable over time.

In some aspects, the present disclosure provides a kit comprising any of the pharmaceutical compositions provided herein and a delivery device. A delivery device refers to machine or apparatus suitable for administering a composition to a subject, such as a syringe or needle. In some embodiments, the kit is to be stored below 50° C., below 40° C., below 30° C., below 20° C., below 10° C., below 0° C., below −10° C., below −20° C., below −30° C., below −40° C., below −50° C., below −60° C., below −70° C., or below −80° C., such that the nucleic acids of the pharmaceutical composition are relatively stable over time.

### EXAMPLES

In order that the disclosure may be more fully understood, the following examples are set forth. The examples are offered to illustrate the modified mRNAs, pharmaceutical compositions, kits, and methods provided herein and are not to be construed in any way as limiting their scope.

### Example 1: Production of Modified mRNAs

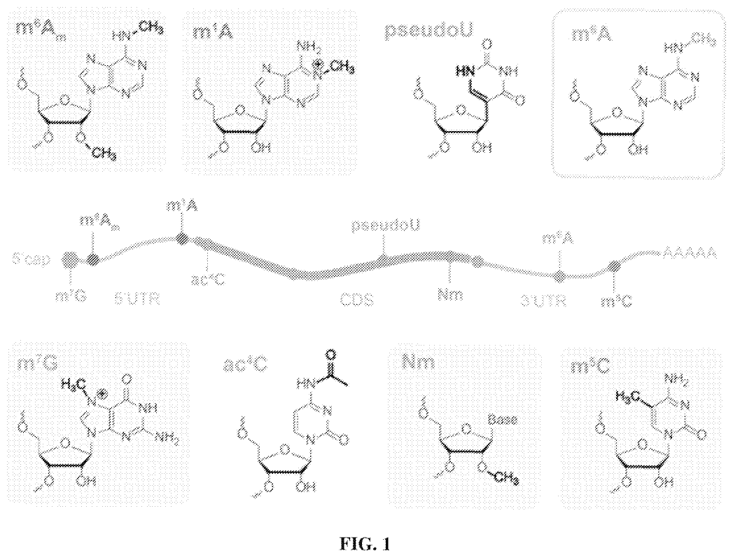

Modified mRNAs are produced by in vitro transcription (IVT) of a DNA template encoding a 5′ untranslated region (UTR), open reading frame encoding a desired protein, and 3′ UTR. A DNA template may also contain a nucleic acid sequence containing repeated thymidine bases (poly(T) sequence) downstream of the template encoding the 3′ UTR. When transcribing RNA from a poly(T) DNA sequence, RNA polymerases stutter, adding multiple adenosine bases to a transcribed RNA without always progressing along the DNA template. This results in the addition of a long RNA sequence containing only adenosine bases, known as a poly(A) tail, being added to the 3′ end of the RNA (FIG. 1).

Alternatively, RNA transcripts without poly(A) tails may be produced by in vitro transcription of a DNA template that does not contain a poly(T) sequence, and poly(A) tails can be added to these transcripts separately in a tailing reaction. RNA molecules are incubated with adenosine triphosphate (ATP) or modified ATPs in the presence of enzyme that is capable of adding nucleotides to the 3′ end of an RNA molecule, such as poly(A) polymerase (PAP). Incubation of RNA and a polyadenylating enzyme with a mixture of ATP and one or more modified ATPs results in the addition of a poly(A) tail Modified mRNAs produced by either of these methods described above are linear mRNAs, which have 5′ and 3′ terminal nucleotides.

Modified mRNAs may be circular mRNAs, which are a single-stranded mRNA molecule without a 5′ or 3′ end (FIG. 2A). Circular mRNAs are produced by incubating a linear mRNA to be circularized with another single-stranded nucleic acid, such as a DNA oligonucleotide, comprising i) a nucleotide sequence that is complementary to a sequence at the 3′ end of the mRNA (3′ DNA complement), and ii) a nucleotide sequence that is complementary to a sequence at the 5′ end of the mRNA, (5′ DNA complement), wherein the 3′ DNA complement is immediately downstream (3′) of the 5′ DNA complement on the DNA oligonucleotide. mRNA hybridizes with the complementary oligonucleotide, such that the 3′ terminal nucleotide of the mRNA is 5′ to the 5′ terminal nucleotide of the mRNA. A ligase, such as SplintR ligase, forms a phosphodiester bond between the two terminal bases of the mRNA, resulting in the formation of a circular mRNA molecule with no terminal nucleotides.

### Example 2: Effect of Modified Bases on Protein Production Efficiency from mRNA

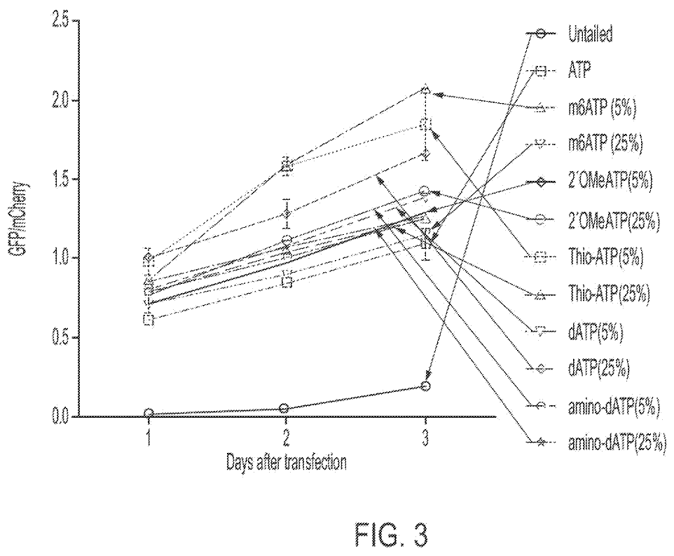

RNAs encoding either GFP or mCherry and lacking poly(A) tails were produced by in vitro transcription. RNAs were polyadenylated as described in Example 1 using different compositions of nucleotides to produce mRNAs with different poly(A) tails. RNAs encoding GFP were polyadenylated with a) ATP, b) mixtures of 95% ATP and 5% modified ATP, c) mixtures of 75% ATP and 25% modified ATP, or d) no ATP (untailed) as negative control. Modified ATPs tested included m6ATP, 2′OMeATP, Thio-ATP, dATP, and amino-dATP. RNAs encoding mCherry were polyadenylated with ATP to produce control mRNAs with canonical poly(A) tails. Mixtures of GFP-encoding mRNA and control mCherry-encoding mRNA were transfected into HeLa cells. At 1, 2, and 3 days post-transfection, the amounts of GFP and mCherry proteins produced in each cell population were quantified by fluorescence microscopy, and the ratios of GFP/mCherry produced were calculated. Each of the GFP-encoding mRNAs containing modified ATPs in the poly(A) tail resulted in a greater GFP/mCherry ratio, relative to GFP-encoding mRNA produced by polyadenylation with only ATP (FIG. 3). Generally, the use of 25% modified ATP in the polyadenylation reaction resulted in a more pronounced increase in the GFP/mCherry ratio than the use of only 5% modified ATP, indicating that more frequent inclusion of modified adenosines into the poly(A) tail further improved protein production efficiency from modified mRNAs.

### Example 3: Biochemical and Functional Characterization of Modified mRNAs

Modified mRNAs are characterized according to multiple biochemical parameters, including purity and the proportion of bases in a given region of the mRNA, such as the poly(A) tail, that are modified bases. NMR spectroscopy is used to evaluate the identity of an mRNA in a composition. Gel electrophoresis is used to evaluate the purity of a composition containing mRNA, with a pure composition containing a single mRNA species producing a single band on a gel, and an impure composition containing multiple mRNA molecules of different sizes producing multiple bands, or a smeared band, on a gel. Liquid column mass spectrometry (LC/MS) is used to evaluate the incorporation of modified nucleotides. Modified nucleotides have different, generally larger, molecular weights than canonical nucleotides, and so the incorporation of more modified nucleotides into an mRNA will result in a greater shift, usually an increase, in the mass of the mRNA molecule.

Cell-based screens are used to evaluate the effects of modified bases on protein translation. Modified mRNAs, in parallel with unmodified mRNAs comprising canonical bases, are transfected into separate populations of human cells. Following transfection, the rates of protein production are evaluated by one of multiple methods known in the art, including flow cytometry and ELISA. The stability of modified or unmodified mRNAs within transfected cells is evaluated by lysing transfected cells at desired timepoints post-transfection, isolating nucleic acids, preparing cDNA from mRNA in lysates by reverse transcription, and quantifying the amount of cDNA corresponding to transfected mRNAs using quantitative PCR. The induction of an innate immune response by transfected mRNAs is quantified using one of multiple methods known in the art, such as ELISA for phosphorylated signaling domains of Toll-like receptors or adaptor proteins, or qRT-PCR-based quantification of genes that are activated by the detection of foreign RNA, such as OAS1.

In therapeutic approaches, the modified mRNAs are administered to human or animal subjects, so that cellular ribosomes of the subject produce the protein or proteins encoded by the mRNA. The mRNA may encode a bioluminescent protein, such as luciferase, so that the efficiency of protein production in the subject may be measured using a luciferase imaging system. The mRNA may encode an antigen, so that production of the antigen in cells of the subject results in the subject producing antibodies and/or T cells specific to the antigen. The immune response generated by the subject towards the antigen is evaluated by methods known in the art, including ELISA to quantify antibodies specific to the antigen, neutralization assays to quantify neutralizing antibodies, and flow cytometry to quantify multiple types of immune cells, including T cells or antigen-specific T cells.

### Example 4: Production of Modified mRNAs by Ligation

### Introduction

Messenger RNA (mRNA) therapeutics and vaccines are quickly becoming established as a new class of drugs, as evidenced by recent clinical trials and approvals of mRNA vaccines for SARS-COV-2.^{1,2} mRNA vectors are viewed as a promising alternative to conventional protein-based drugs due to their programmability, rapid production of protein in vivo, relatively low cost manufacturing, and potential scalability of targeting multiple proteins simultaneously.³⁻⁵ While mRNAs have been shown to robustly generate transgenic proteins in vivo, the relatively short half-life of mRNA may limit the clinical applications of this therapeutic platform.^{3,6} This issue has previously been circumvented during animal studies with multiple injections of RNA (e.g. “booster” doses), as in the case of some vaccine studies,⁷⁻⁹ but this strategy could potentially limit therapeutic applications and widespread distribution.

Chemical modifications are effective strategies to boost the translational potential and reduce the toxicity of mRNAs for in vivo applications. Incorporation of modified UTP derivatives (e.g. pseudouridine & N1-methylpseudouridine) has been widely used to decrease innate immune toxicity upon RNA transfection.¹⁰⁻¹² Circular mRNAs have been reported to have enhanced half-lives over their linear counterparts, presumably due to their lack of degradable 5′ and 3′ RNA ends.¹³⁻¹⁵ However, circular mRNAs have suffered from overall lower expression levels due to their reliance on IRES elements that do not robustly tolerate the incorporation of modified nucleotides.¹⁵ Additionally, exonuclease-resistant nucleotides have been incorporated into the mRNA body and mRNA poly(A) tail, with variable increases in RNA half-life being reported.^{16,17} While the random incorporation of modified nucleoside triphosphates (NTPs) by RNA polymerases into the mRNA body shows promise, this strategy dramatically reduces the chemical space of NTPs that can be tested, since many modified NTPs are not well-tolerated by ribosomal machinery and thus reduce overall translational efficiency.¹⁸⁻²⁰ An alternative strategy is to selectively incorporate modified NTPs during enzymatic poly(A) tailing.^{16,17} While promising, this strategy relies on poly(A) polymerase enzymes, which face limitations of small chemical repertoires tolerated by three enzymes and inability to incorporate modified nucleotides in a site-specific manner.

An alternative strategy to create mRNA vectors with enhanced protein production capacity through 3′ end ligation of synthetic modified RNA oligonucleotides, is presented herein. Canonical mRNA degradation pathways in eukaryotes are thought to typically begin with 3′ deadenylation, followed by the recruitment of a decapping complex and exposure of the mRNA to 5′ and 3′ cellular exonucleases²¹. mRNAs bearing exonuclease-resistant poly(A) tails were tested for their ability to resist deadenylation and produce more protein, relative to mRNAs with unmodified poly(A) tails, in cells.

### Results

### Preliminary Modified ATP Incorporation During Poly(A) Tailing

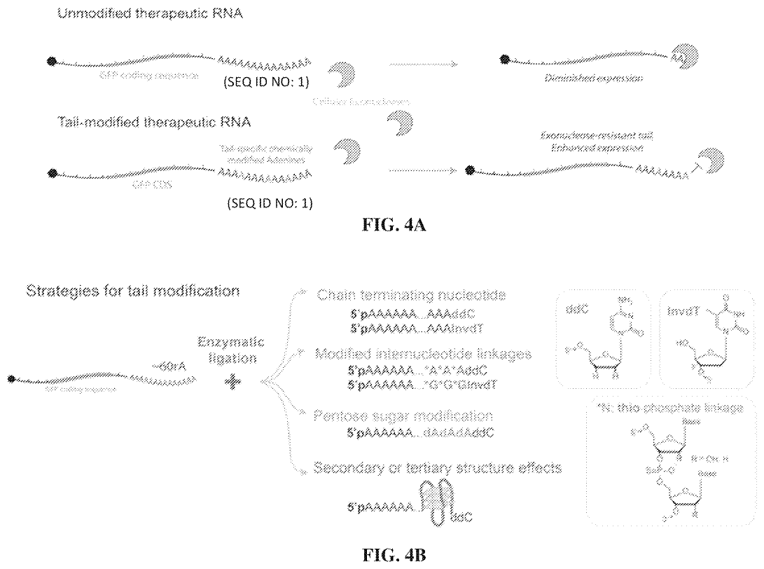

Multiple chemically modified ATP derivatives were screened for their poly(A) stabilization activity. Specifically, modified ATPs were spiked into poly(A) tailing reactions using GFP mRNA templates, using similar tailing protocols described previously (FIG. 4A).¹⁷ GFP-encoding mRNAs with modified poly(A) tails and mCherry-encoding mRNAs with unmodified poly(A) tails were co-transfected into Hela cells. Each transfection contained only one type of modified GFP-encoding mRNA, and the control mCherry-encoding mRNA. By measuring the relative GFP/mCherry fluorescence ratio over a three-day time course, minor differences in mRNA translational half-life as a result of modified NTP incorporation into the poly(A) tail were observed.

Monitoring fluorescence in HeLa cells over three days revealed increases in fluorescent protein production as a result of poly(A) tailing reactions with modified ATP spike-ins, particularly for dATP (2′-deoxyadenosine) and alpha-thiol ATP (Adenosine-5′-O-(1-Thiotriphosphate)) (FIG. 1). E. coli poly(A) polymerase likely incorporated modified ATP sporadically and at substoichiometric levels. It is also possible that E. coli poly(A) polymerase excluded some modified nucleotides entirely, producing unmodified poly(A) tails despite the presence of modified ATPs in the polyadenylation reaction.

### Chemically Modified Oligonucleotide Ligations Enhance Translational Lifetime

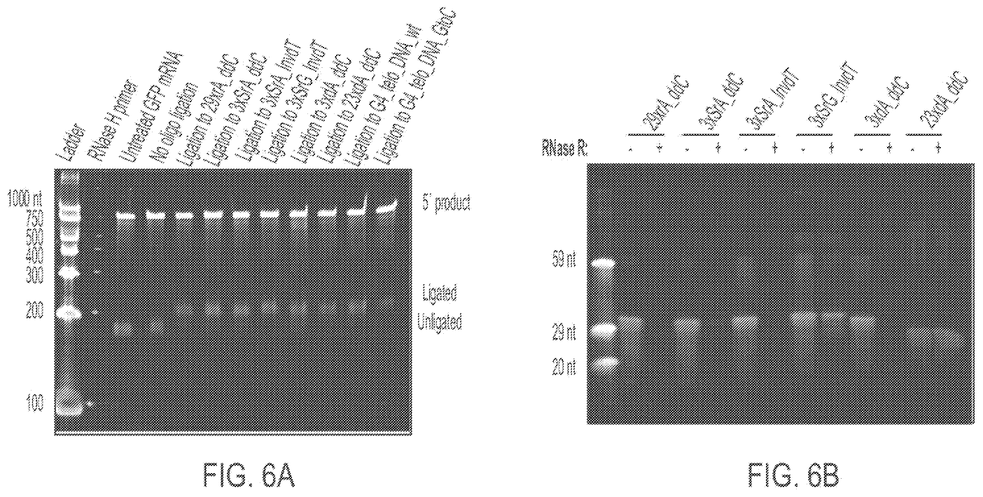

To test different designs of site-specific chemical modifications and incorporate alternative internucleotide linkages, an alternative modification strategy was pursued, in which synthetic oligonucleotides were ligated onto the 3′ ends of mRNAs containing a pre-existing poly(A) tail (FIG. 4B). In vitro transcription from DNA templates containing a GFP coding sequence and a poly(A) tail-encoding sequence was used to create population of GFP-encoding mRNAs with homogeneous lengths. Efficiencies of 3′ oligonucleotide ligation were determined using RNase H reactions targeting the 3′ UTR, which resulted in clear separation of ligated and unligated mRNA 3′ ends on a gel (FIG. 6A). Ligations using T4 RNA Ligase I (Promega) was observed to work with nearly 100% efficiency, as evidenced by RNase H reactions (FIG. 6A).

To compare the efficacy of different chemical modifications, all oligonucleotides were designed to be 29 nucleotides long. Each oligonucleotide contained a 5′ phosphate, to facilitate ligation to the 3′ end of the mRNA, and a 3′ blocking group (dideoxyC [ddC] or inverted-dT [InvdT]) that lacked a 3′ hydroxyl group, to prevent self-ligation of oligonucleotides. This ensured that ligation would attach one, and only one, copy of the oligonucleotide to the mRNA. Furthermore, at least 6-8 nucleotides at the 5′ end of the oligonucleotides were unmodified rA nucleotides, to provide an unstructured handle for the T4 RNA Ligase I reaction. The modified RNA and DNA oligonucleotide sequences can be found in Table 1.

Oligonucleotides were ligated onto the 3′ end of GFP-encoding mRNAs described in the preceding paragraph, containing a ˜60 nucleotide template-encoded poly(A) tail for ease of characterization using a previously described RNase H protocol.

TABLE 1
Sequences of tailing oligonucleotides.

| Modified | | | Anhydrous |
|---|---|---|---|
| oligonucleotide | | | Molecular |
| sequence name | Sequence (IDT format) | Bases | Weight |
| 29xrA_ddC | /5Phos/tArArA rArArA rArArA | 29 | 9838.2 |
| | rArArA rArArA rArArA rArArA | | |
| | rArArA rArArA rArA/3ddC/ | | |
| | (SEQ ID NO: 6) | | |
| 3xSrA_ddC | /5Phos/rArArA rArArA rArArA | 29 | 9886.4 |
| | rArArA rArArA rArArA rArArA | | |
| | rArArA rArA*rA* rA*rA/3ddC/ | | |
| | (SEQ ID NO: 7) | | |
| 3xSrA_InvdT | /5Phos/rArArA rArArA rArArA | 29 | 9917.4 |
| | rArArA rArArA rArArA rArArA | | |
| | rArArA rArArA* rA*A*/3InvdT/ | | |
| | (SEQ ID NO: 8) | | |
| 3xSrG_InvdT | /5Phos/rArArA rArArA rArArA | 29 | 9965.4 |
| | rArArA rArArA rArArA rArArA | | |
| | [ArArA rArArG* rG*rG*/3InvdT/ | | |
| | (SEQ ID NO: 9) | | |
| 3xdA_ddC | /5Phos/rArArA rArArA rArArA | 29 | 9790.2 |
| | rArArA rArArA rArArA rArArA | | |
| | rArArA rArAA AA/3ddC/ (SEQ ID | | |
| | NO: 10) | | |
| 23xdA_ddC | /5Phos/rArArA rArArA AAA AAA | 29 | 9470.3 |
| | AAA AAA AAA AAA AAA | | |
| | AA/3ddC/ (SEQ ID NO: 11) | | |
| G4_telo_DNA_GtoC | /5Phos/r ArArA rArArA TAC CCT | 29 | 9118.9 |
| | TAC CCT TAC CCT TAC CC/3ddC/ | | |
| | (SEQ ID NO: 12) | | |
| G4_telo_DNA_WT | /5Phos/rArArA rArArA TAG GGT | 29 | 9599.2 |
| | TAG GGT TAG GGT TAG | | |
| | GG/3ddC/ (SEQ ID NO: 13) | | |
| 6xSr(AG) | /5Phos/rArArA rArArA rArArA | 29 | 10013.6 |
| | rArArA rArArA rArArA ArArA | | |
| | rArArA* rA*rA*rG* rG*G*/3InvdT/ | | |
| | (SEQ ID NO: 14) | | |
| G4_C9orf72_RNA_ | /5Phos/rArArA rArArA rArGrG | 29 | 10077.4 |
| 6xSrG | rGrGrC rCrGrG rGrGrC rCrGrG | | |
| | rGrGrC* rC*rG*rG* rG*rG*/3InvdT/ | | |
| | (SEQ ID NO: 15) | | |
| G4_C9orf72_DNA_ | /5Phos/rArArA rArArA rAGG GGC | 29 | 9725.5 |
| 6xSG | CGG GGC CGG GGC* C*G*G* | | |
| | G*G*/3InvdT/ (SEQ ID NO: 16) | | |
| G4_telo_DNA_6xSG | /5Phos/rArArA rArÅrA TAG GGT | 29 | 9726.5 |
| | TAG GGT TAG GGT* T*A*G* | | |
| | G*G*/3InvdT/ (SEQ ID NO: 17) | | |

Ligated, modified GFP-encoding mRNAs were transfected into HeLa cells along with unligated mCherry mRNA (E-PAP poly(A)-tailed), which served as an internal transfection control. Cell samples were imaged to quantify relative GFP/mCherry fluorescence intensity ratios at 24 hr, 48 hr, and 72 hr post-transfection, to estimate the effects of particular 3′ end modifications on translational lifetime.

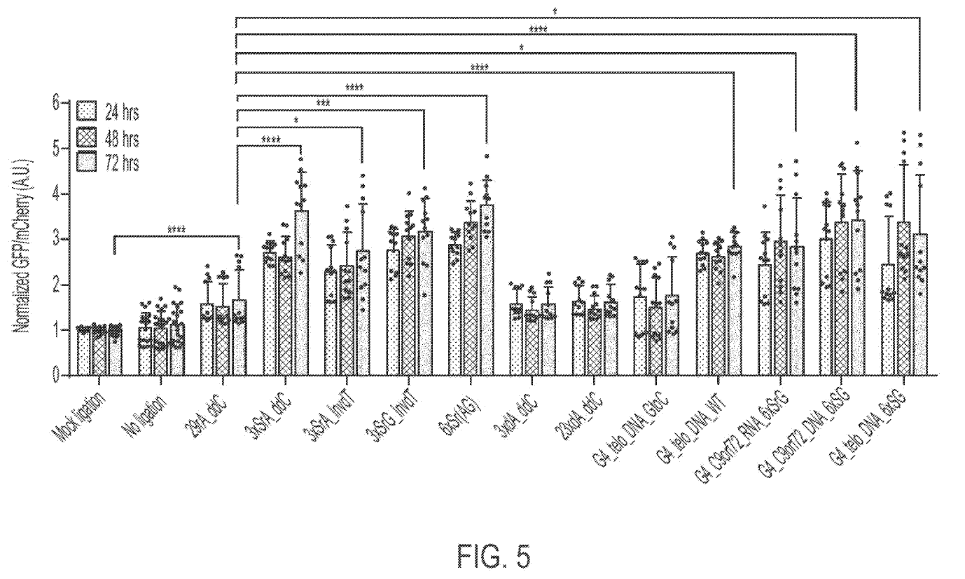

Ligation of the control oligonucleotide, containing 29 unmodified rA linkages and a 3′ ddC (29xrA_ddC), slightly increased GFP fluorescence (between 50-55%, Table 2) in HeLa cell culture, compared to the unligated and mock ligation controls. This was likely due to the extension of the poly(A) tail by 28 nucleotides, and possibly partially due to the presence of the chain-terminating ddC nucleotide. Additionally, ligation products of oligonucleotides containing 3 sequential phosphorothioate (PS) linkages (3xSrA_ddC, 3xSrA_InvdT, and 3xSrG_InvdT) showed 140%-210% increased GFP production compared with that of the 29 nt poly(rA) control oligo at each timepoint (FIG. 5). This observation is generally consistent with phosphorothioate linkages bearing nuclease-resistant activity, as used in antisense oligonucleotide therapy.²²

TABLE 2
Statistics of GFP/mCherry fluorescence ratio shown in FIG. 5.

| | | No | 29rA_{—} | 3 × SrA_{—} | 3 × SrA_{—} | 3 × SrG_{—} | | 3 × dA_{—} | 23 × dA_{—} |
|---|---|---|---|---|---|---|---|---|---|
| 24 hour | Mock | ligation | ddC | ddC | InvdT | InvdT | 6 × Sr(AG) | ddC | ddC |
| Mean | 1 | 1.05 | 1.59 | 2.73 | 2.31 | 2.78 | 2.90 | 1.60 | 1.66 |
| Std. | 0.032 | 0.33 | 0.47 | 0.23 | 0.57 | 0.44 | 0.26 | 0.30 | 0.30 |
| Deviation | | | | | | | | | |
| Std. | 0.0057 | 0.074 | 0.13 | 0.067 | 0.16 | 0.127 | 0.074 | 0.091 | 0.088 |
| Error of | | | | | | | | | |
| Mean | | | | | | | | | |
| Lower | 0.99 | 0.90 | 1.30 | 2.58 | 1.95 | 2.5 | 2.73 | 1.40 | 1.47 |
| 95% | | | | | | | | | |
| CI of | | | | | | | | | |
| mean | | | | | | | | | |
| Upper | 1.01 | 1.21 | 1.89 | 2.87 | 2.68 | 3.06 | 3.06 | 1.8 | 1.85 |
| 95% | | | | | | | | | |
| CI of | | | | | | | | | |
| mean | | | | | | | | | |
| | Mock | No | 29rA_{—} | 3 × SrA_{—} | 3 × SrA_{—} | 3 × SrG_{—} | | 3 × dA_{—} | 23 × dA_{—} |
| | ligation | ligation | ddC | ddC | InvdT | InvdT | 6 × Sr(AG) | ddC | ddC |
| 48 hr | | | | | | | | | |
| Mean | 1 | 1.05 | 1.54 | 2.63 | 2.43 | 3.07 | 3.39 | 1.45 | 1.49 |
| Std. | 0.052 | 0.36 | 0.48 | 0.41 | 0.71 | 0.55 | 0.45 | 0.28 | 0.28 |
| Deviation | | | | | | | | | |
| Std. | 0.0092 | 0.081 | 0.14 | 0.12 | 0.21 | 0.16 | 0.13 | 0.081 | 0.080 |
| Error of | | | | | | | | | |
| Mean | | | | | | | | | |
| Lower | 0.98 | 0.88 | 1.24 | 2.37 | 1.98 | 2.72 | 3.11 | 1.27 | 1.32 |
| 95% | | | | | | | | | |
| CI of | | | | | | | | | |
| mean | | | | | | | | | |
| Upper | 1.02 | 1.21 | 1.85 | 2.90 | 2.88 | 3.42 | 3.68 | 1.63 | 1.67 |
| 95% | | | | | | | | | |
| CI of | | | | | | | | | |
| mean | | | | | | | | | |
| 72 hr | | | | | | | | | |
| Mean | 1 | 1.15 | 1.69 | 3.64 | 2.76 | 3.20 | 3.77 | 1.58 | 1.64 |
| Std. | 0.089 | 0.42 | 0.64 | 0.84 | 1.01 | 0.70 | 0.55 | 0.36 | 0.35 |
| Deviation | | | | | | | | | |
| Std. | 0.016 | 0.093 | 0.19 | 0.24 | 0.29 | 0.20 | 0.16 | 0.11 | 0.10 |
| Error of | | | | | | | | | |
| Mean | | | | | | | | | |
| Lower | 0.97 | 0.96 | 1.28 | 3.10 | 2.12 | 2.76 | 3.42 | 1.35 | 1.42 |
| 95% | | | | | | | | | |
| CI of | | | | | | | | | |
| mean | | | | | | | | | |
| Upper | 1.03 | 1.35 | 2.09 | 4.17 | 3.40 | 3.64 | 4.11 | 1.81 | 1.86 |
| 95% | | | | | | | | | |
| CI of | | | | | | | | | |
| mean | | | | | | | | | |

| | | | G4_telo_{—} | G4_telo_{—} | G4_C9orf72_{—} | G4_C9orf72_{—} | G4_telo_{—} |
|---|---|---|---|---|---|---|---|
| | Mock | No | DNA | DNA | RNA | DNA | DNA |
| | ligation | ligation | GtoC | WT | 6 × SrG | 6 × SG | 6 × SG |
| 24 hour | | | | | | | |
| Mean | 1 | 1.05 | 1.76 | 2.71 | 2.46 | 3.02 | 2.47 |
| Std. | 0.032 | 0.33 | 0.69 | 0.27 | 0.70 | 0.80 | 1.04 |
| Deviation | | | | | | | |
| Std. | 0.0057 | 0.074 | 0.20 | 0.078 | 0.201 | 0.23 | 0.30 |
| Error of | | | | | | | |
| Mean | | | | | | | |
| Lower | 0.99 | 0.90 | 1.32 | 2.54 | 2.02 | 2.51 | 1.81 |
| 95% | | | | | | | |
| CI of | | | | | | | |
| mean | | | | | | | |
| Upper | 1.01 | 1.21 | 2.20 | 2.88 | 2.91 | 3.52 | 3.12 |
| 95% | | | | | | | |
| CI of | | | | | | | |
| mean | | | | | | | |
| 48 hr | | | | | | | |
| Mean | 1 | 1.05 | 1.51 | 2.63 | 2.95 | 3.41 | 3.37 |
| Std. | 0.052 | 0.36 | 0.66 | 0.31 | 1.02 | 1.03 | 1.25 |
| Deviation | | | | | | | |
| Std. | 0.0092 | 0.081 | 0.19 | 0.089 | 0.29 | 0.30 | 0.36 |
| Error of | | | | | | | |
| Mean | | | | | | | |
| Lower | 0.98 | 0.88 | 1.09 | 2.44 | 2.31 | 2.76 | 2.57 |
| 95% | | | | | | | |
| CI of | | | | | | | |
| mean | | | | | | | |
| Upper | 1.02 | 1.21 | 1.93 | 2.83 | 3.60 | 4.07 | 4.17 |
| 95% | | | | | | | |
| CI of | | | | | | | |
| mean | | | | | | | |
| 72 hr | | | | | | | |
| Mean | 1 | 1.15 | 1.78 | 2.86 | 2.87 | 3.43 | 3.14 |
| Std. | 0.089 | 0.42 | 0.83 | 0.30 | 1.04 | 1.08 | 1.28 |
| Deviation | | | | | | | |
| Std. | 0.016 | 0.093 | 0.24 | 0.086 | 0.30 | 0.31 | 0.37 |
| Error of | | | | | | | |
| Mean | | | | | | | |
| Lower | 0.97 | 0.96 | 1.25 | 2.67 | 2.21 | 2.75 | 2.32 |
| 95% | | | | | | | |
| CI of | | | | | | | |
| mean | | | | | | | |
| Upper | 1.03 | 1.35 | 2.31 | 3.05 | 3.53 | 4.11 | 3.95 |
| 95% | | | | | | | |
| CI of | | | | | | | |
| mean | | | | | | | |

Detailed P values are listed in the format of Sample 1 v.s. Sample 2: 72 hr comparison. Mock ligation v.s. 29rA_ddC: 4e-7; 29rA_ddC v.s. 3XSrA_ddC: 2e-6; 29rA_ddC v.s. 3XSrA_InvdT: 0.005; 29rA_ddC v.s. 3XSrG_InvdT: 1e-5; 29rA_ddC v.s. 6XSr(AG): <1e-15; 29rA_ddC v.s. G4_telo_DNA_WT: 9e-6. 29rA_ddC v.s. G4_C9orf72_RNA_6 xSrG: 0.003; 29rA_ddC v.s. G4_C9orf72_DNA_6 xSrG: 8e-5; 29rA_ddC v.s. G4_telo_DNA_6 xSG: 0.002.

Surprisingly, a 3xthio-rG_invdT linkage demonstrated slightly greater GFP fluorescence than the 3xthio-rA_invdT (170%-200% vs. 140%-180% normalized GFP/mCherry) at every time point, although this difference was relatively small (Table 2; FIG. 5). This result may be related to the specificity of mRNA deadenylation enzymes for adenine over guanosine.^{23,24} However, these short, unstructured sequence differences played a relatively minor role in altering mRNA translational lifetime. Furthermore, 3xSrA_ddC and 3xSrA_InvdT demonstrated 170%-210% and 140%-180% normalized GFP/mCherry production, respectively (accounting for all timepoints; Table 2). This suggests that changing the identities of small chain-terminating nucleotides used in ligations (3′ dideoxy-C & 3′ inverted dT) may result in minor enhancements to mRNA stability.

Given the success of RNase-resistant phosphorothioate linkages, RNA nucleotides in oligonucleotides were replaced by RNase-resistant DNA nucleotides to determine their effects on protein translation yield. Unexpectedly, the oligonucleotide containing 23 deoxyadenosines (23xdA_ddC) did not substantially enhance translational half-life (FIG. 5), despite the oligonucleotide's resistance to in vitro RNase R digestion (FIG. 6B). However, DNA quadruplex (telomere-derived) ssDNA sequences displayed stabilizing effects that were consistently greater than the unstructured 23 deoxyadenosine and “G to C” ssDNA oligo control ligations (FIG. 5). It was hypothesized that mRNAs possessing unstructured 3′ ssDNA ends may be susceptible to cellular ssDNA exonucleases, or alternatively trigger RNase H activity if they possess homology to the mRNA.²⁵⁻²⁷

Finally, ligation with oligonucleotides with an increased number of phosphorothioate modifications, as well as combination with quadruplex (G4) secondary structures, was explored to determine whether these modifications could act synergistically to stabilize modified mRNAs. 6 sequential phosphorothioate linkages in an unstructured ssRNA oligo (6xSr(AG)) provided the most consistent level of stabilization, with standard deviation of 0.26-0.6 over all timepoints (Table 2; FIG. 5). The ssDNA and ssRNA G4 oligos containing 6 sequential phosphorothioate linkages (G4_C9orf72_RNA_6 xSrG, G4_C9orf72_DNA 6xSrG and G4_telo_DNA 6xSG) also resulted in enhanced translation over the control oligos, but the performances of these constructs were more variable among different replicates, demonstrating S.D. ranges of 0.7-1; 0.8-1.1; and 1.0-1.3, respectively (Table 2; FIG. 5).

The HeLa cell time course experiment demonstrated that mRNAs incorporating phosphorothioate linkages had increased GFP/mCherry signal over time (FIG. 5). These chemical modifications may act directly by increasing the translation efficiency per mRNA, or indirectly by reducing the rate of RNA degradation relative to the mCherry-encoding internal control mRNA, thereby increasing the observed GFP/mCherry signal.

### Discussion

Previous studies of cytoplasmic mRNA decay have identified poly(A) tail shortening as a rate-limiting step in major mRNA degradation pathways (e.g., deadenylation-dependent decay). In line with this model, shortening of the poly(A) tail was investigated as the rate-limiting step in the deactivation of mRNA vectors.

Ligation of oligonucleotides containing nuclease-resistant chemical linkages onto the 3′ end of mRNA is sufficient to increase mRNA translational activity over the course of several days (24-72 hr), resulting in up to 170%-220% more protein expression in cell culture, in the case of the 6xSr(AG) construct. This strategy can expand the chemical space of modified nucleotide derivatives in mRNA vectors for diverse purposes.

These results suggest that poly(A) shortening is a major determinant of therapeutic mRNA translational efficacy, consistent with previous models of cytoplasmic mRNA degradation. These results inform the replacement of mRNA tails with nuclease-resistant, poly(A) binding protein (PABP)-binding aptamers/oligonucleotides for enhanced mRNA stabilization. The strategy detailed herein is also compatible with other types of modifications, such as hydrolysis-resistant 7-methylguanosine 5′ caps,^{28,29} modified 5′ UTR regions,³⁰ or endonuclease/hydrolysis-resistant modified nucleotides in the mRNA body. This ligation strategy is generally suitable to combine mRNA therapeutics with easily synthesized, chemically-modified aptamers, such as peptide nucleic acids,³¹ locked nucleic acids,³² or other chemical groups.

### Methods

### Plasmid Cloning, Characterization, and Purification

hMGFP and mCherry-encoding plasmids in pCS2 vector (WX28 and WX26, respectively) were obtained. These plasmids contained (in 5′-3′ order): an SP6 promoter sequence, a 5′ UTR, a fluorescent protein coding sequence (CDS), 3′ UTR, and NotI restriction site.

The Q5® Site-Directed Mutagenesis Kit (NEB) was used to perform PCR on the plasmid using primers encoding poly(A) on the forward primer & poly(T) on the reverse primer. This was followed by KLD enzyme treatment, then transformation into NEB Stabl cells for isolation using the ZymoPURE plasmid miniprep kit, and Sanger sequencing through Genewiz.

mRNA Synthesis and Characterization

GFP mRNA was synthesized from WX28xEsp3i plasmid, which contained an SP6 promoter, followed by hMGFP CDS and template-encoded poly(A) tail. Plasmids were linearized by a single Esp3i site located immediately 3′ of the poly(A) region. Linearized plasmids were then purified using the DNA Clean & Concentrator-25 kit from Zymo Research.

5′ capped, modified mRNA was prepared using SP6 enzyme and reaction buffer from mMESSAGE mMACHINE™ SP6 Transcription Kit. The 2×NTP/Cap solution provided by the kit was replaced with a 2×NTP/Cap preparation, containing: 10 mM ATP, 10 mM CTP, 2 mM GTP, 8 mM 3′-O-Me-m⁷G(5′)ppp(5′)G RNA Cap Structure Analog, and 10 mM N1-methylpseudouridine-5′-triphosphate. Superase-In RNase Inhibitors were added to a final concentration of 1:20 (v/v). Following IVT reaction assembly and incubation at 37° C. for 2-4 hours, reactions were treated with 1-2 μl of TURBO DNase for 1 hr at 37° C. prior to reaction purification using MEGAclear™ Transcription Clean-Up Kit.

Superase-In RNase Inhibitor was added to purified mRNA samples to a final concentration of 1:50 (v/v), and stored samples at −80° C. for long term storage. Purified mRNA was measured by Nanodrop to estimate concentration prior to ligations, but mRNAs were measured using the Qubit RNA HS Assay for normalization immediately prior to transfection for cell-based testing.

For the preparation of unmodified poly(A) polymerase-tailed mRNA, dsDNA templates were generated by linearization of WX28 and WX26 plasmids using NotI-HF, and column purified digested products using Zymo DNA Clean & Concentrator-25. In vitro transcription was performed using the protocol described above, except after TURBO DNase digestion, the extra step of poly(A) tailing using the E-PAP Poly(A) Tailing Kit was included. Purification and storage of mRNA was as described above (e.g., using MEGAclear transcription cleanup kit).

Modified E. coli Poly(A) Polymerase Tailing

For modified E-PAP tailing experiments, the substrate was an untailed GFP mRNA generated from IVT's on a linearized WX28 template. The protocol utilized the enzyme and buffer from E-PAP Poly(A) Tailing Kit. “10 mM total” ATP stock solutions were prepared for each modified ATP spike-in, such that a specific percentage of ATP was replaced by a modified ATP derivative (XATP). For example, 25% dATP samples would require assembly of a 2.5 mM dATP, 7.5 mM ATP stock solution. Tailing reactions were assembled as follows:

| | | |
|---|---|---|
| 1.5 | μg | Untailed GFP mRNA |
| 5 | μl | 5X E-PAP buffer |
| 2.5 | μl | 10 mM XATP:ATP stock solution |
| 2.5 | μl | 25 mM MnC12 |
| 1 | μl | Superase-In RNase Inhibitor |
| 1 | μl | E-PAP enzyme |

Up to 25 μl total volume with nuclease free water

Reactions were incubated at 37° C. for 1 hour, then quenched with the addition of 0.5 ul of 500 mM EDTA. These tailed mRNAs were then column purified using Monarch RNA cleanup kit (50 μg). Superase-In RNase Inhibitor was added to purified mRNA to a final dilution of 1:50 (v/v), and mRNA was stored at −80° C. prior to transfection.

The following modified ATP derivatives (XATPs) were used in polyadenylation experiments: Adenosine 5′-Triphosphate (ATP); N⁶-Methyladenosine-5′-Triphosphate (m⁶A); 2′-O-Methyladenosine-5′-Triphosphate; Adenosine-5′-O-(1-Thiotriphosphate); deoxyadenosine triphosphate (dATP); 2′-Amino-2′-deoxyadenosine-5′-Triphosphate.

### Modified Oligonucleotide 3′ End Ligations

Ligation reactions were performed using T4 RNA Ligase I. Reactions were assembled as follows:

| | | |
|---|---|---|
| 2 | μg | capped mRNA |
| 200 | pmol | chemically modified oligo |
| 2 | μl | Superase-In RNase Inhibitor |
| 20 | μl | 50% PEG-8000 |
| 5 | μl | 100% DMSO |
| 5 | μl | 10X T4 RNA ligase buffer |
| 5 | μl | T4 RNA ligase (Promega) |

Up to 50 μl total volume (with nuclease-free water)

Reactions were incubated at 37° C. for 30 minutes, followed by inactivation of the reaction via the addition of 1 ul of 500 mM EDTA, pH 8.0. Reactions were diluted by the addition of 1 volume of nuclease free water (e.g. 50 μl), followed by the addition of 0.5 volumes of AMPure XP containing 1 μl Superase-In (e.g. 25 μl). Reactions were purified according to the manufacturer's protocol, and mRNA was eluted from AMPure beads using nuclease free water containing Superase-In at a 1:50 (v/v) ratio.

For ligations that were incomplete according to the RNase H gel-based assay, ligations were performed using a modified condition, in which DMSO was omitted from the reaction. This generally resulted in more efficient ligation, when necessary.

### RNase H Assays

Potassium chloride (KCl) stock solution was prepared and used for annealing an ssDNA oligo to mRNA prior to RNase H assays. KCl tock solution contained: 50 mM KCl, 2.5 mM EDTA, 1:200 (v/v) Superase-In RNase inhibitor, brought to its final volume using nuclease free water. The ssDNA probe was ordered from IDT and had the sequence GCATCACAAATTTCACAAATAAAGCATTTTTTTCAC (SEQ ID NO: 18).

The following reaction was prepared to anneal mRNA to the aforementioned ssDNA probe:

| | | |
|---|---|---|
| 200 | ng | mRNA sample (purified) |
| 2 | pmol | ssDNA probe |
| 2 | μl | Stock solution: 50 mM KCl, 2.5 mM EDTA, 1:200 |
| | | Superase-In |

Up to 10 ul volume using nuclease-free water

Reactions were denatured at 70° C. for 5 minutes, followed by cooling to room temperature (25° C.) at a rate of 0.2° C./sec in a benchtop thermocycler. Following probe annealing, 1 μl of Thermostable RNase H and 1 μl of the 10× buffer were added to each reaction, which was incubated at 50° C. for 30 min. Following reaction incubation, samples were digested by the addition of 1 μl Proteinase K and incubated at room temperature for 5 minutes. Subsequently, samples were mixed with 1 volume of Gel Loading Buffer II, which had been supplemented with EDTA to a final concentration of 50 mM.

Samples in 1× loading buffer were denatured at 70° C. for 3-5 minutes prior to loading and resolution on 6% Novex™ TBE-Urea Gels.

### RNase R Digestion of Oligonucleotides

200 ng of oligo was incubated in a 10 μl total reaction volume containing 1×RNase R reaction buffer and 10 units of RNase R. Reactions were incubated at 37° C. for 1 hr, then digested with 1 μl Proteinase K and denatured in 1× Gel Loading Buffer II. They were run on 15% Novex TBE-Urea gels.

Mammalian Cell Culture and mRNA Transfection

HeLa cells (CCL-2, ATCC) were maintained in Dulbecco's Modified Eagle's Medium (DMEM) culture media containing 10% FBS in a 37° C. incubator with 5% CO₂ and passaged at the ratio of 1.8 every three days. The cell culture was confirmed free of mycoplasma contamination regularly with Hoechst staining and microscopy imaging.

On the day before mRNA transfection, the cells were seeded at 75% confluence in individual wells on a 12-well plate. The day after, 500 ng mCherry (internal control) mRNA and 500 ng GFP mRNA with synthetic tails (concentrations determined by Qubit) were transfected into each well using 3 uL Lipofectamine MessengerMAX Transfection Reagent. Additional controls that contain only mCherry mRNA, or only transfection reagents, or non-transfected cells were included. After a 6-hour incubation, the lipofectamine/mRNA transfection mixture was removed, and cells were rinsed once with DPBS and trypsinized to reseed into three glass bottom 24-well plates (poly-D-lysine coated) at a ratio of 6:4:3, respectively, for fluorescent protein quantification at 24 hours, 48 hours, and 72 hours after transfection.

### Confocal Imaging and Quantification of Fluorescent Proteins

Before fluorescent protein imaging, the culture media was removed and the cells were rinsed with DPBS once before being incubated in the nuclei staining media (FluoroBrite DMEM with 1:2000 dilution of Hoechst 33342) at 37° C. for 10 mins.

Confocal images of the nuclei (Hoechst), GFP, and mCherry were taken by Leica Stellaris 8 with a 10× air objective at the pixel size of 900 nm*900 nm. Four representative fields of view were taken for each well, one from each quadrant. The same imaging setting was used for all the samples to be compared. Excitation/detection wavelengths were, in “Excitation wavelength/˜[Detection wavelength range]” format: Hoechst: Diode 405 nm/˜[430-480] nm; GFP: WLL 489 nm/˜[500-576] nm; mCherry: WLL 587 nm/˜[602-676] nm.

mRNA Quantification in Transfected Cell Culture Using STARmap

mCherry and GFP mRNA quantities were measured in transfected cells using STARmap,³³ an imaging-based method that detects individual mRNA molecules as a barcoded DNA colony. The STARmap procedure for cell cultures described by Wang et al. was followed.³³

Briefly, following fluorescent protein imaging, the cells were fixed with 1.6% PFA/1XPBS at room temperature for 10 min before further fixation and permeabilization with pre-chilled Methanol at −20° C. (up to one week) before the next step Subsequently, the methanol was removed and the cells were rehydrated with PBSTR/Glycine/tRNA (PBS with 0.1% Tween-20, 0.5% SUPERaseIn, 100 mM Glycine, 1% Yeast tRNA) at room temperature for 15 min followed by washing once with PBSTR. The samples were then hybridized with SNAIL probes targeting mCherry and GFP mRNA sequences in the hybridization buffer (2×SSC, 10% Formamide, 1% Tween-20, 20 mM RVC, 0.5% SUPERaseIn, 1% Yeast tRNA, 100 nM each probe) at 40° C. overnight. The cells were then washed with PBSTR twice at 37° C. (20 min each wash) and high-salt wash buffer (PBSTR with 4×SSC) once at 37° C. before rinsing once with PBSTR at room temperature. The ligation reaction was performed for 2 hours at room temperature to circularize padlocks probes that are adjacent to a primer. After two washes with PBSTR, rolling circle amplification was initiated from the primer using Phi29 at 30° C. for 2 hours with amino-dUTP spiked in. After two more washes with PBSTR, the DNA amplicons were modified to be polymerizable by 20 mM MA-NHS in PBST buffer at room temperature for 2 hours. The samples were then converted into a hydrogel-cell hybrid before Proteinase K digestion of fluorescent proteins at room temperature overnight. The samples were washed three times with PBST before being stained with fluorescent detection oligonucleotide in the wash and imaging buffer (2×SSC, 10% Formamide) at 37° C. for 1 hour. Finally, the samples were washed three times with the wash and imaging buffer at room temperature and stained with DAPI before imaging in the wash and imaging buffer.

Confocal imaging stacks were taken by Leica Stellaris 8 with a 40× oil objective at the pixel size of 283 nm*283 nm. A 14-um stack was imaged with 1 um/step for 15 steps. Four representative fields of view were taken for each well, one from each quadrant.

TABLE 3
SNAIL probe sequences

| | |
|---|---|
| mCherry-01 | /5Phos/ACATTATTGGTGCCGCGCAGCTTCACCTAATTATTACTGAGG |
| | CATACACTAAAGATA (SEQ ID NO: 19) |
| mCherry-02 | /SPhos/ACATTACTTCTTGGCCTTGTAGGTGGTAATTATTACTGAGGC |
| | ATACACTAAAGATA (SEQ ID NO: 20) |
| mCherry-03 | /5Phos/ACATTACACGGTCACCACGCCGCCGTAATTATTACTGAGGCA |
| | TACACTAAAGATA (SEQ ID NO: 21) |
| mCherry-11 | ACGGGGCCGTCGGAGGGGAATAATGTTATCTT (SEQ ID NO: 22) |
| mCherry-12 | GGCGCCGGGCAGCTGCACGGTAATGTTATCTT (SEQ ID NO: 23) |
| mCherry-13 | GTCCTGCAGGGAGGAGTCCTGGTAATGTTATCTT (SEQ ID NO: 24) |
| hMGFP-01 | /5Phos/ACATTAAGTCGCAGCGGTAGTGGCCAATTATTACTGAAATCG |
| | TAGACTAAGATA (SEQ ID NO: 25) |
| bMGFP-02 | /5Phos/ACATTACATTAGCAGGGAAGTTGACCCCGTAATTATTACTGA |
| | AATCGTAGACTAAGATA (SEQ ID NO: 26) |
| hMGFP-03 | /5Phos/ACATTAGCTTCGGCGTGCTCGTACAGCTAATTATTACTGAAA |
| | TCGTAGACTAAGATA (SEQ ID NO: 27) |
| hMGFP-11 | CCTCCCTCCAAGAGCAGTGCCATTAATGTTATCTT (SEQ ID NO: 28) |
| hMGFP-12 | TGCGCTGCATCACCGGGCTAATGTTATCTT (SEQ ID NO: 29) |
| hMGFP-13 | CCTGGCGGGGTAGTCCGCTGTGTAATGTTATCTT (SEQ ID NO: 30) |

### Fluorescent Detection Probe Sequences

mCherry amplicon detection probe:
(SEQ ID NO: 31)
/5Alexa647N/CATACACTAAAGATAACAT
hMGFP amplicon detection probe:
(SEQ ID NO: 32)
/5Alex546N/TCGTAGACTAAGATAACAT

### REFERENCES

1. Polack, F. P. et al. Safety and Efficacy of the BNT162b2 mRNA Covid-19 Vaccine. N. Engl. J. Med. 383, 2603-2615 (2020).
2. Lombardi, A. et al. Mini Review Immunological Consequences of Immunization With COVID-19 mRNA Vaccines: Preliminary Results. Front. Immunol. 12, 657711 (2021).
3. Sahin, U., Karikó, K. & Türeci, O. mRNA-based therapeutics—developing a new class of drugs. Nat. Rev. Drug Discov. 13, 759-780 (2014).
4. Dammes, N. & Peer, D. Paving the Road for RNA Therapeutics. Trends Pharmacol. Sci. 41, 755-775 (2020)
5 Weng, Y. et al. The challenge and prospect of mRNA therapeutics landscape. Biotechnol. Adv. 40, 107534 (2020).
6 Chen, C.-Y. A., Ezzeddine, N. & Shyu, A.-B. Messenger RNA half-life measurements in mammalian cells. Methods Enzymol. 448, 335-357 (2008).
7 Petsch, B. et al. Protective efficacy of in vitro synthesized, specific mRNA vaccines against influenza A virus infection. Nat. Biotechnol. 30, 1210-1216 (2012).
8. Richner, J. M. et al. Vaccine Mediated Protection Against Zika Virus-Induced Congenital Disease. Cell 170, 273-283.e12 (2017).
9. Tai, W. et al. A novel receptor-binding domain (RBD)-based mRNA vaccine against SARS-COV-2. Cell Res. 30, 932-935 (2020).
10. Karikó, K. et al. Incorporation of pseudouridine into mRNA yields superior nonimmunogenic vector with increased translational capacity and biological stability. Mol. Ther. 16, 1833-1840 (2008).
11. Karikó, K., Buckstein, M., Ni, H. & Weissman, D. Suppression of RNA recognition by Toll-like receptors: the impact of nucleoside modification and the evolutionary origin of RNA. Immunity 23, 165-175 (2005).
12. Andries, O. et al. N(1)-methylpseudouridine-incorporated mRNA outperforms pseudouridine-incorporated mRNA by providing enhanced protein expression and reduced immunogenicity in mammalian cell lines and mice. J. Control. Release 217, 337-344 (2015).
13. Wesselhoeft, R. A., Kowalski, P. 5. & Anderson, D. G. Engineering circular RNA for potent and stable translation in eukaryotic cells. Nat. Commun. 9, 2629 (2018).
14. Wesselhoeft, R. A. et al. RNA Circularization Diminishes Immunogenicity and Can Extend Translation Duration In Vivo. Mol. Cell 74, 508-520.e4 (2019).
15 Carmona, E. M. Circular RNA: Design Criteria for Optimal Therapeutical Utility. (Harvard University, 2019).
16. Anhäuser, L., Hüwel, S., Zobel, T. & Rentmeister, A. Multiple covalent fluorescence labeling of eukaryotic mRNA at the poly(A) tail enhances translation and can be performed in living cells. Nucleic Acids Res. 47, e42 (2019).
17. Strzelecka, D. et al. Phosphodiester modifications in mRNA poly(A) tail prevent deadenylation without compromising protein expression. RNA 26, 1815-1837 (2020).
18. Li, B., Luo, X. & Dong, Y. Effects of Chemically Modified Messenger RNA on Protein Expression. Bioconjug. Chem. 27, 849-853 (2016).
19. Aurup, H., Siebert, A., Benseler, F., Williams, D. & Eckstein, F. Translation of 2′-modified mRNA in vitro and in vivo. Nucleic Acids Res. 22, 4963-4968 (1994)
20. Choi, J. et al. 2′-O-methylation in mRNA disrupts tRNA decoding during translation elongation. Nat. Struct. Mol. Biol. 25, 208-216 (2018)
21. Łabno, A., Tomecki, R. & Dziembowski, A. Cytoplasmic RNA decay pathways—Enzymes and mechanisms. Biochim. Biophys. Acta 1863, 3125-3147 (2016).
22. Eckstein, F. Phosphorothioates, essential components of therapeutic oligonucleotides. Nucleic Acid Ther. 24, 374-387 (2014).
23. Stowell, J. A. W. et al. Reconstitution of Targeted Deadenylation by the Ccr4-Not Complex and the YTH Domain Protein Mmil. Cell Rep. 17, 1978-1989 (2016).
24. Chen, J., Chiang, Y.-C. & Denis, C. L. CCR4, a 3′-5′ poly(A) RNA and ssDNA exonuclease, is the catalytic component of the cytoplasmic deadenylase. EMBO J. 21, 1414-1426 (2002).
25. Cerritelli, 5. M. & Crouch, R. J. Ribonuclease H: the enzymes in eukaryotes. FEBS J. 276, 1494-1505 (2009).
26. Yang, Y.-G., Lindahl, T. & Barnes, D. E. Trex1 exonuclease degrades ssDNA to prevent chronic checkpoint activation and autoimmune disease. Cell 131, 873-886 (2007).
27. Takahashi, A. et al. Downregulation of cytoplasmic DNases is implicated in cytoplasmic DNA accumulation and SASP in senescent cells. Nat. Commun. 9, 1249 (2018).
28. Rydzik, A. M. et al. mRNA cap analogues substituted in the tetraphosphate chain with CX2: identification of O-to-CC12 as the first bridging modification that confers resistance to decapping without impairing translation. Nucleic Acids Res. 45, 8661-8675 (2017).
29 Strenkowska, M et al. Towards mRNA with superior translational activity: synthesis and properties of ARCA tetraphosphates with single phosphorothioate modifications. New J. Chem. 34, 993-1007 (2010).
30. Kawaguchi, D. et al. Phosphorothioate Modification of mRNA Accelerates the Rate of Translation Initiation to Provide More Efficient Protein Synthesis. Angew. Chem. Int. Ed Engl. 59, 17403-17407 (2020).
31. Wu, J.-C. et al. Recent advances in peptide nucleic acid for cancer bionanotechnology. Acta Pharmacol. Sin. 38, 798-805 (2017).
32. Doessing, H. & Vester, B. Locked and unlocked nucleosides in functional nucleic acids. Molecules 16, 4511-4526 (2011).
33. Wang, X. et al. Three-dimensional intact-tissue sequencing of single-cell transcriptional states. Science 361, (2018).
34. Zangi, Lior, et al. “Modified mRNA directs the fate of heart progenitor cells and induces vascular regeneration after myocardial infarction.” Nature Biotechnology. 31.10 (2013): 898.
35 Bahl, Kapil, et al. “Preclinical and clinical demonstration of immunogenicity by mRNA vaccines against H10N8 and H7N9 influenza viruses.” Molecular Therapy. 25.6 (2017): 1316-1327.
36. Richner, Justin M., et al. “Modified mRNA vaccines protect against Zika virus infection.” Cell. 168.6 (2017): 1114-1125.

### Example 5: Chemically Modified mocRNAs for Highly Efficient Protein Expression in Mammalian Cells

As evidenced by recent clinical trials and approvals of messenger RNA (mRNA) vaccines for SARS-COV-2^{1,2}, mRNA is an emerging and promising alternative to conventional protein-based drugs. This is mainly due to its programmability, rapid production of proteins in vivo, relatively low-cost manufacturing, and potential scalability to produce multiple proteins simultaneously³⁻⁵. However, while mRNAs have been shown to robustly generate therapeutic proteins in vivo^{3,6-8}, their relatively short lifetimes may limit their clinical applications where high quantities of protein production are required^{3,9}. Depending on the intended functions of therapeutic proteins, the dosage and treatment duration of mRNA drugs could vary by orders of magnitude. For vaccines, the expression of nanogram to microgram ranges of an antigen could be sufficient for eliciting an immune response³. However, for growth factors, hormones, or antibodies, the therapeutic dose could range from microgram to milligram, or potentially up to gram quantities of protein³. Simply scaling up mRNA quantity to achieve high protein production may lead to dose-dependent toxicity, due to the innate immune stimulation inherent to transfection of mRNA³. This combination of factors drives the need for engineering mRNA vectors to boost transgenic protein production without increasing dosage, particularly through enhancements to mRNA lifetime and/or translational efficiency.

Chemical modification is an effective way to enhance the performance of mRNA vectors. Exogenous mRNAs prepared by in vitro transcription (IVT) consisting of “unmodified” adenosine (A), guanosine (G), cytidine (C), and uridine (U) strongly trigger innate immune toxicity that suppresses protein expression¹⁰⁻¹². Incorporation of modified U derivatives, such as pseudouridine and N1-methylpseudouridine, has been widely used to increase translation, specifically by decreasing innate immune toxicity through blocking Toll-like receptor recognition¹⁰⁻¹⁴. However, this strategy currently limits the chemical space of mRNA modifications available for incorporation, as many modified nucleoside triphosphates (NTPs) are not tolerated by RNA polymerases or ribosomal machinery. Moreover, certain chemical modifications in the protein-coding region of mRNAs could potentially cause impaired translation¹⁴⁻¹⁶. An alternative strategy to increase mRNA stability without modifying the coding region is to selectively incorporate modified NTPs during enzymatic extension of the mRNA poly(A) tail, which is particularly sensitive to exonucleases in the cell^{17,18} While promising, this strategy relies on poly(A) polymerases, which again face limited chemical repertoires, variable efficiencies of enzymatic incorporation, and generation of a variable distribution of poly(A) tail lengths¹⁸.

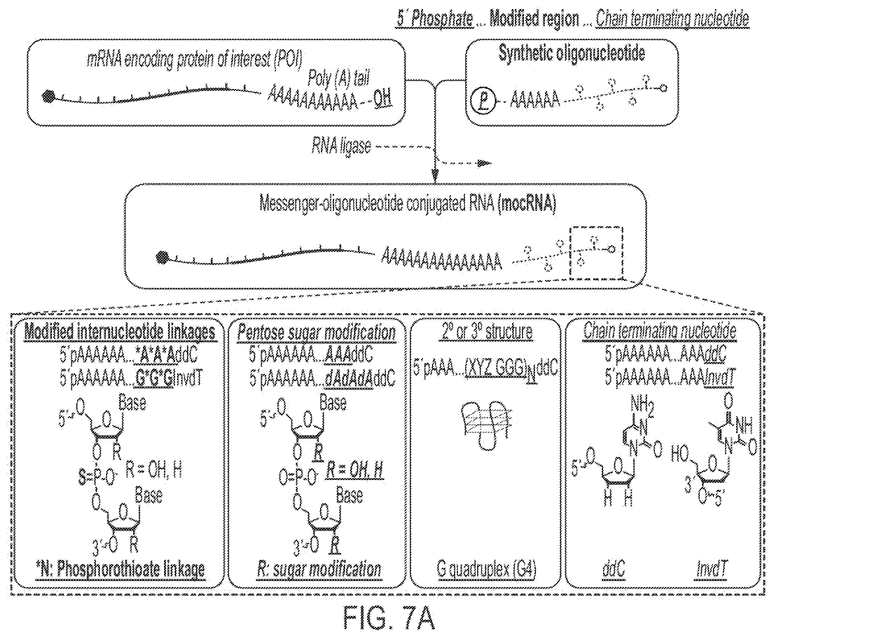

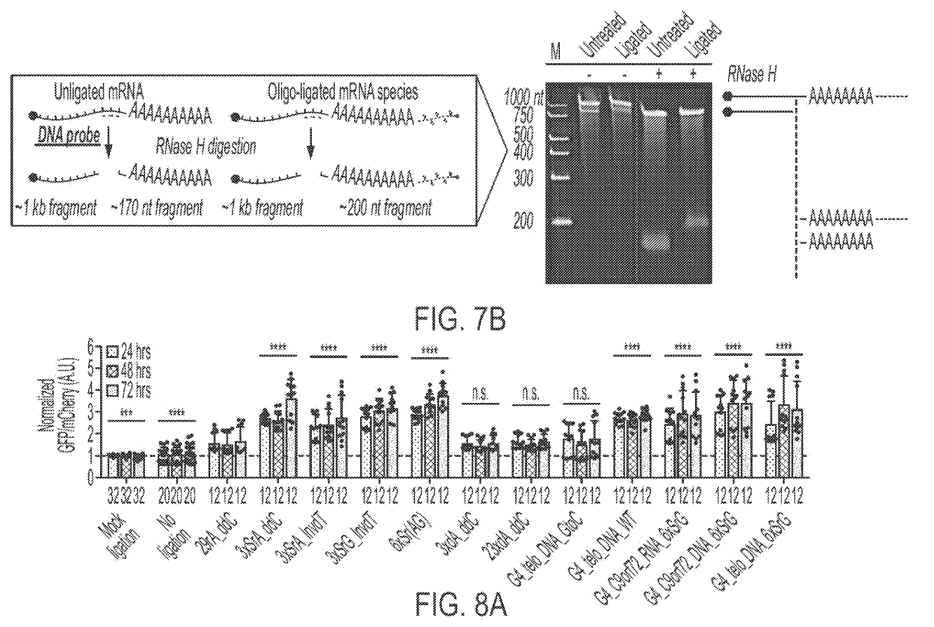

To overcome the aforementioned limitations, a ligation-based strategy was developed to efficiently construct messenger-oligonucleotide conjugated RNAs (mocRNAs), an mRNA-based expression system with augmented protein production capacity. In this approach, synthetic oligonucleotides (oligos) are ligated with the 3′ ends of mRNAs containing template-encoded poly(A) tails (FIGS. 7A and 7B). This enables precise and modularized encoding of chemical modifications into RNA vectors, which is not possible using RNA polymerase-mediated incorporation. Shortening of the poly(A) tail is identified as a critical step in cellular mRNA decay, and the poly(A) tail is indispensable for cap-dependent translation^{19,20}. Thus, as a proof-of-concept of the mocRNA system, various nuclease-resistant motifs²¹ were designed and tested in synthetic oligonucleotides to protect poly(A) tails, which demonstrated superior protein expression in comparison with alternative variants of mRNA vectors.

### Results and Discussion

Highly Efficient Synthesis of mocRNA by Ligation

To enable the conjugation between in vitro transcribed (IVT) mRNA and a synthetic oligo, each oligo was designed with the following elements (FIG. 7A, Table 4): (1) a 5′ phosphate and at least six unstructured RNA nucleotides at the 5′ end of the oligos to ligate with the 3′ terminus of IVT mRNAs by T4 RNA Ligase I; (2) a 3′ blocking group (2′-3′-dideoxycytidine [ddC] or inverted-2′-deoxythymidine [InvdT]) to prevent oligo self-ligation; (3) comparable lengths of poly(A) regions to enable reliable comparison of translation enhancement. The 3′ blocking group of the oligo enables a large molar excess of oligo in the reaction to ensure nearly 100% conversion of the IVT mRNA to a mocRNA product (FIGS. 7A and 7B, Table 4).

TABLE 4
Sequences of oligonucleotides used for mocRNA syntheses.

| Modified | |
|---|---|
| oligonucleotide | |
| sequence name | Sequence (IDT format) |
| 29rA_ddC | /5Phos/rArArA rArArA rArArA rArArA rArArA rArArA rArArA |
| | rArArA rArArA rArA/3ddC/ (SEQ ID NO: 6) |
| 3xSrA_ddC | /5Phos/rArArA rArArA rArArA rArArA rArArA rArArA rArArA |
| | rArArA rArA*rA*rA* rA*rA/3ddC/ (SEQ ID NO: 7) |
| 3xSrA_InvdT | /5Phos/rArArA rArArA rArArA rArArA rArArA rArArA rArArA |
| | rArArA rArArA* rA*rA*/3InvdT/ (SEQ ID NO: 8) |
| 3xSrG_InvdT | /5Phos/rArArA rArArA rArArA rArArA rArArA rArArA rArArA |
| | rArArA rArArG* rG*rG*/3InvdT/ (SEQ ID NO: 9) |
| 6xSr(AG) | /5Phos/rArArA rArArA rArArA rArArA rArArA rArArA rArArA |
| | ArArA* rA*rA*rG* rG*rG*/3InvdT/ (SEQ ID NO: 14) |
| 3xdA_ddC | /5Phos/rArArA rArArA rArArA rArArA rArArA rArArA rArArA |
| | rArArA rArAA AA/3ddC/ (SEQ ID NO: 10) |
| 23xdA_ddC | /5Phos/rArArA rArArA AAA AAA AAA AAA AAA AAA AAA |
| | AA/3ddC/ (SEQ ID NO: 11) |
| G4_telo_DNA_GtoC | /5Phos/rArArA rArArA TAC CCT TAC CCT T?C CCT TAC |
| | CC/3ddC/ (SEQ ID NO: 12) |
| G4_telo_DNA_WT | /5Phos/rArArA rArArA TAG GGT TAG GGT TAG GGT TAG |
| | GG/3ddC/ (SEQ ID NO: 13) |
| G4_C9orf72_RNA_ | /5Phos/rArArA rArArA rArGrG rGrGrC rCrGrG rGrGrC rCrGrG |
| 6xSrG | rGrGrC* rC*rG*rG* rG*rG*/3InvdT/ (SEQ ID NO: 15) |
| G4_C9orf72_DNA_ | /5Phos/rArArA rArArA rAGG GGC CGG GGC CGG GGC* C*G*G* |
| 6xSrG | G*G*/3InvdT/ (SEQ ID NO: 16) |
| G4_telo_DNA_6xSrG | /5Phos/fArArA rArArA TAG GGT TAG GGT TAG GGT* T*A*G* |
| | G*G*/3InvdT/ (SEQ ID NO: 17) |
| 26rA_G4_C9orf72_ | /5Phos/rArArA rArArA rArArA rArArA rArArA rArArA rArArA |
| RNA_6xSrG | rArArA rArArGrGrGrGrCrCrGrGrGrG rCrCrGrGrGrG rC*rC*G* |
| | rG*rG*rG* /3InvdT/ (SEQ ID NO: 33) |
| 26rA_G4_C9orf72_ | /5Phos/rArArA rArArA rArArA rArArA rArArA rArArA rArArA |
| DNA_6xSrG | rArArA rArAG GGG CCG GGG CCG GGG C*C*G* G*G*G* |
| | /3InvdT/ (SEQ ID NO: 34) |
| 26rA_G4_telo_DNA_ | /5Phos/rArArA rArArA rArArA rArArA rArArA rArArA rArArA |
| 6xSrG | rArArA rArAT AGG GTT AGG GTT AGG GT*T* A*G*G* |
| | G*/3InvdT/ (SEQ ID NO: 35) |

RNA bases: r_; RNA phosphorothioate bases: r_*; DNA phosphorothioate bases: _*; 5′ Phosphate modification: /5Phos/; 2′-3′-dideoxycytidine [ddC] modification: /3ddC/; Inverted-2′-deoxythymidine [InvdT] modification: /3InvdT/.

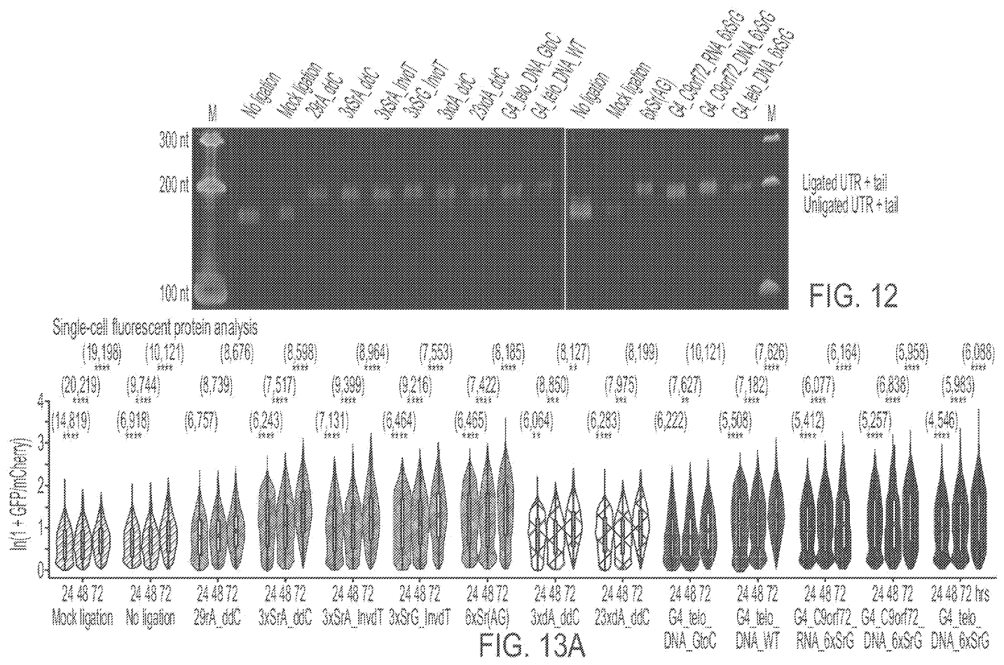

To demonstrate the mocRNA expression system, a plasmid template was cloned containing a humanized Monster Green Fluorescent Protein (GFP) followed by a template-encoded poly(A) tail (plasmid: pCS2_GFP-60A), which ensures translatable mRNAs with homogeneous poly(A) lengths. The GFP-encoding mRNAs (GFP-60A) were synthesized using IVT by SP6 polymerase, with a 5′ anti-reverse cap analog (ARCA) and 100% replacement of uridine with N1-methylpseudouridine. The IVT mRNAs were further modified into mocRNAs by 3′ oligo ligation using T4 RNA ligase I. The conjugation efficiency was determined via sequence-specific RNA cleavage, using RNase H and a DNA oligo targeting the 3′ untranslated region (UTR), followed by gel electrophoresis to resolve conjugated and unconjugated mRNA 3′ ends. The RNase H assay showed nearly 100% conjugation efficiency for all the mocRNA constructs using the aforementioned GFP-60A mRNA (FIGS. 7B, 12A), suggesting the general applicability of this conjugation strategy.

Nuclease-Resistant mocRNA Increases Protein Production and RNA Stability in Human Cells

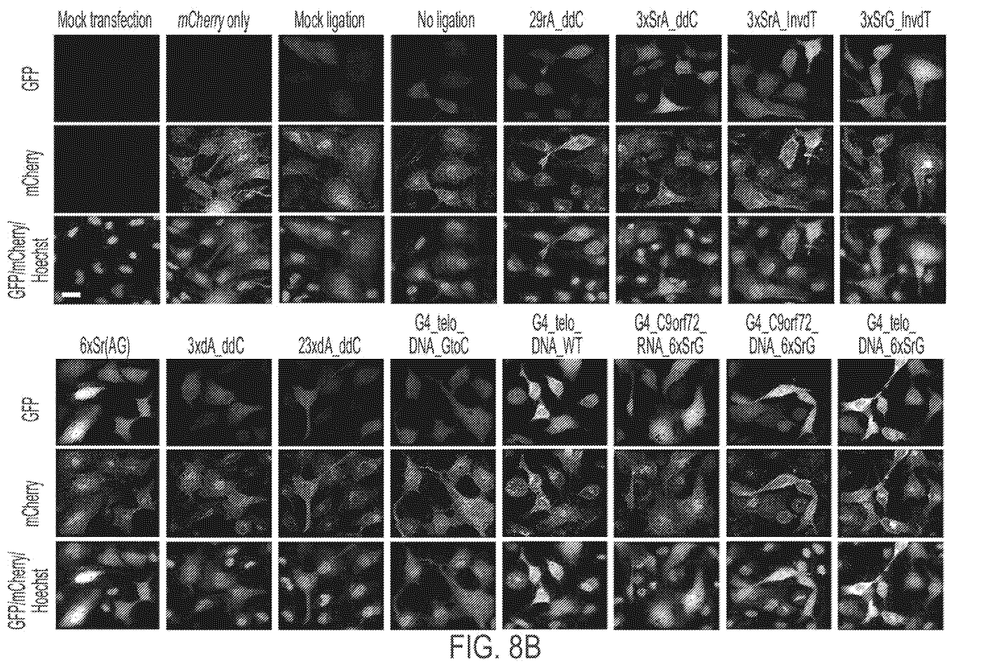

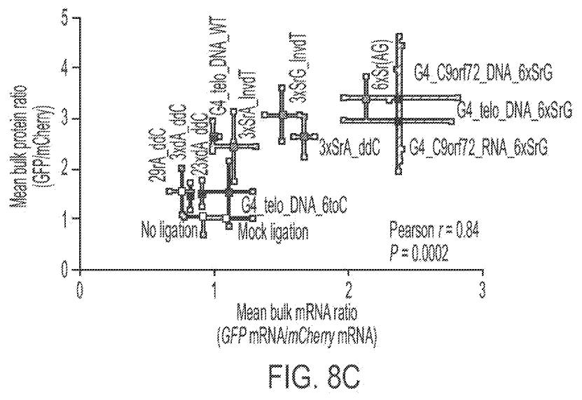

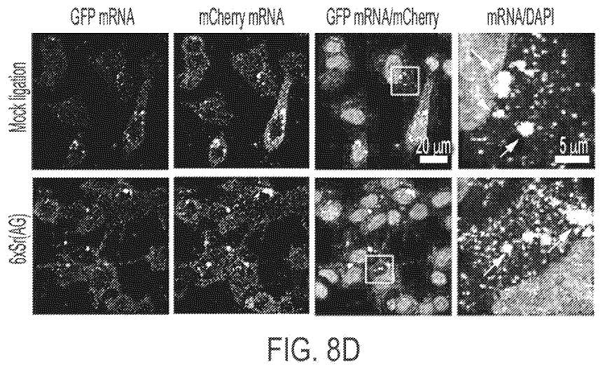

Given that endogenous deadenylation machinery is a 3′ to 5′ exonuclease complex and deadenylation is the rate-limiting step of canonical RNA decay inside cells, it was hypothesized that introducing nuclease-resistant elements at the 3′ terminus after the poly(A) tail would be an effective way to increase RNA translation capacity by keeping the poly(A) tail intact. To this end, mocRNA constructs were synthesized using synthetic oligos (3xSrA_ddC, 3xSrA_InvdT, and 3xSrG_InvdT, and 6xSr(AG), Table 4) containing 3′ terminal deadenylase-resistant modifications, such as phosphorothioate PS linkages¹⁸ and A-to-G substitutions²². GFP-encoding mocRNA constructs were transfected into HeLa cells along with E-PAP poly(A) tailed mCherry mRNA, which served as an internal transfection control. GFP/mCherry fluorescence intensity ratios were quantified at 24 hr, 48 hr, and 72 hr time points after transfection with confocal microscopy. Fluorescence quantification showed that the control mocRNA construct, which contained a 29 nt-long poly(A) tract followed by a 3′ ddC (29rA_ddC), increased GFP fluorescence by up to 69% in comparison with a mock ligation control (GFP-60A mRNA treated with ligase but no modified oligo). This increase was likely due to the extension of the poly(A) tail and possibly the presence of the chain-terminating nucleotide. Among all the oligos containing terminal PS linkages, the unstructured single-stranded (ss) RNA oligo with six sequential phosphorothioates (6xSr(AG), sequence in Table 4) consistently provided the highest expression of GFP (290%-377% at 24-72 hrs, normalized to “mock ligation”) compared to the other modified oligos tested (FIGS. 8A-8B; Table 6).

TABLE 6
Statistics for GFP/mCherry fluorescence ratio data from FIG.
8A (normalized to “mock ligation” samples).

| | 24 hr statistics | | | 48 hr statistics | | | 72 hr statistics | | |
|---|---|---|---|---|---|---|---|---|---|
| | Mean | s.d. | s.e.m | Mean | s.d. | s.e.m. | Mean | s.d. | s.e.m. |
| Mock ligation | 1 | 0.032 | 0.006 | 1 | 0.052 | 0.0092 | 1 | 0.089 | 0.016 |
| No ligation | 1.05 | 0.33 | 0.07 | 1.05 | 0.36 | 0.081 | 1.15 | 0.42 | 0.093 |
| 29rA_ddC | 1.59 | 0.47 | 0.13 | 1.54 | 0.48 | 0.14 | 1.69 | 0.64 | 0.19 |
| 3 × SrA_ddC | 2.73 | 0.23 | 0.07 | 2.63 | 0.41 | 0.12 | 3.64 | 0.84 | 0.24 |
| 3 × SrA_InvdT | 2.31 | 0.57 | 0.16 | 2.43 | 0.71 | 0.21 | 2.76 | 1.01 | 0.29 |
| 3 × SrG_InvdT | 2.78 | 0.44 | 0.127 | 3.07 | 0.55 | 0.16 | 3.2 | 0.7 | 0.2 |
| 6 × Sr(AG) | 2.9 | 0.26 | 0.074 | 3.39 | 0.45 | 0.13 | 3.77 | 0.55 | 0.16 |
| 3 × dA_ddC | 1.6 | 0.3 | 0.091 | 1.45 | 0.28 | 0.081 | 1.58 | 0.36 | 0.11 |
| 23 × dA_ddC | 1.66 | 0.3 | 0.088 | 1.49 | 0.28 | 0.08 | 1.64 | 0.35 | 0.1 |
| G4_telo_{—} | 1.76 | 0.69 | 0.2 | 1.51 | 0.66 | 0.19 | 1.78 | 0.83 | 0.24 |
| DNA_GtoC | | | | | | | | | |
| G4_telo_{—} | 2.71 | 0.27 | 0.078 | 2.63 | 0.31 | 0.089 | 2.86 | 0.3 | 0.086 |
| DNA_WT | | | | | | | | | |
| G4_C9orf72_{—} | 2.46 | 0.7 | 0.201 | 2.95 | 1.02 | 0.29 | 2.87 | 1.04 | 0.3 |
| RNA_6 × SrG | | | | | | | | | |
| G4_C9orf72_{—} | 3.02 | 0.8 | 0.23 | 3.41 | 1.03 | 0.3 | 3.43 | 1.08 | 0.31 |
| DNA_6 × SrG | | | | | | | | | |
| G4_telo_{—} | 2.47 | 1.04 | 0.3 | 3.37 | 1.25 | 0.36 | 3.14 | 1.28 | 0.37 |
| DNA_6 × SrG | | | | | | | | | |

Given the success of PS-modified mocRNAs, it was hypothesized that 3′ terminal RNase-resistant DNA linkages could similarly increase protein translation. The telomere-derived DNA quadruplex (G4_telo_DNA_WT) sequence significantly enhanced protein translation (150%-170% at 24-72 hrs) compared to the unstructured “G to C” DNA oligo control ligation (FIGS. 8A and 8B; Table 6). These results suggest that mocRNAs containing unstructured ssDNA at their 3′ ends may remain susceptible to cellular nucleases, such as ssDNA-specific nucleases^{23,24} and CCR4 (a component of the deadenylation complex), which contains some ssDNase activity²⁵. An alternative possibility is that unstructured ssDNA may trigger mRNA degradation via RNase H if they are partially complementary to mRNA sequences²⁶. Collectively, these results indicate that mocRNAs containing a structured DNA quadruplex at the 3′ terminus may increase protein expression most effectively, while an unstructured ssDNA tail may enhance expression to a somewhat lesser degree.

It was further explored whether combining PS modifications with G4 secondary structures could synergistically stabilize mocRNAs. The ssDNA and ssRNA G4 oligos containing six sequential PS linkages (G4_C9orf72_RNA 6xSrG, G4_C9orf72_DNA_6 xSrG, and G4_telo_DNA 6xSrG) resulted in levels of enhanced translation similar to the mocRNAs containing an unstructured 6xSr(AG) oligo (FIGS. 8A and 8B; Table 6).

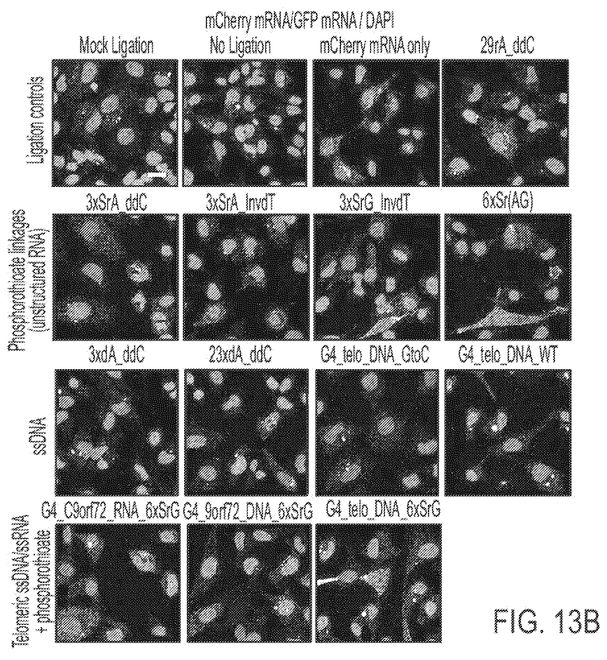

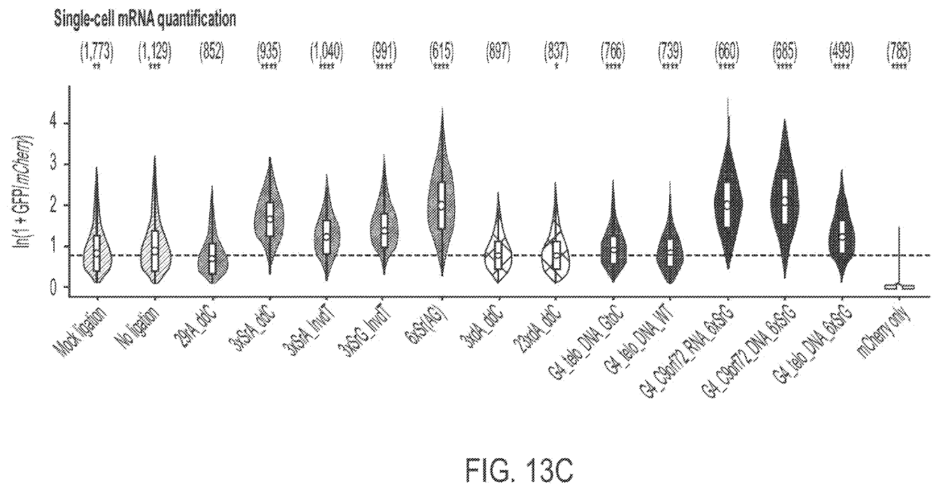

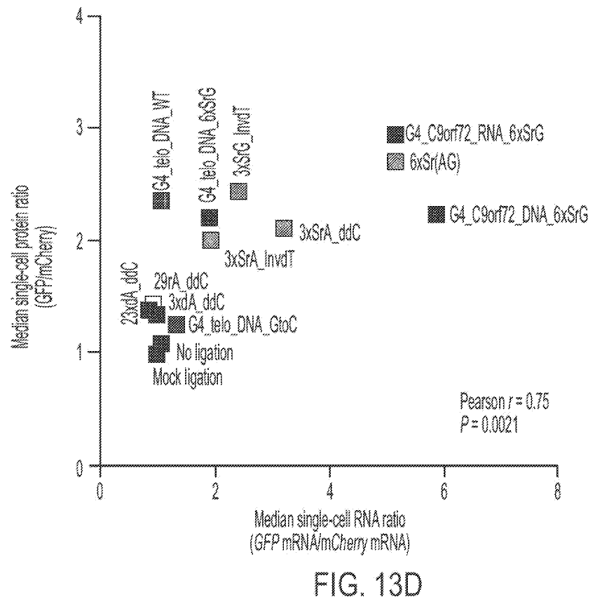

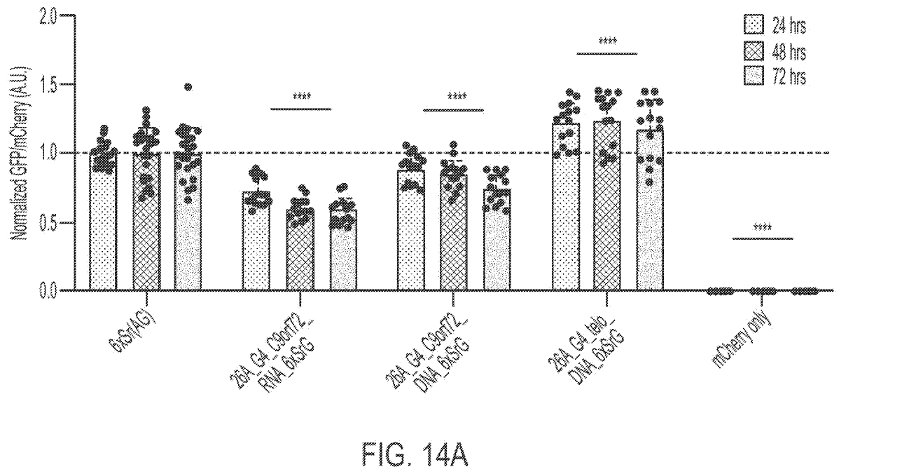

The enhanced translation of mocRNAs may have been due to either a reduced RNA degradation rate or a direct increase in the translational efficiency per mRNA, without affecting mRNA degradation kinetics. To verify the mechanism of translational enhancement, RT-qPCR quantification was performed on HeLa cells transfected with various mocRNA ligation constructs at 48 hours post-transfection (Table 7). It was found that the relative GFP/mCherry RNA ratios correlated well with the observed bulk GFP/mCherry protein fluorescence ratio for each construct (FIG. 8C, Pearson r=0.84, P=2e-4; FIGS. 13B and 13C), suggesting that modified oligos enhance protein translation primarily by stabilizing mRNA quantities in cells.

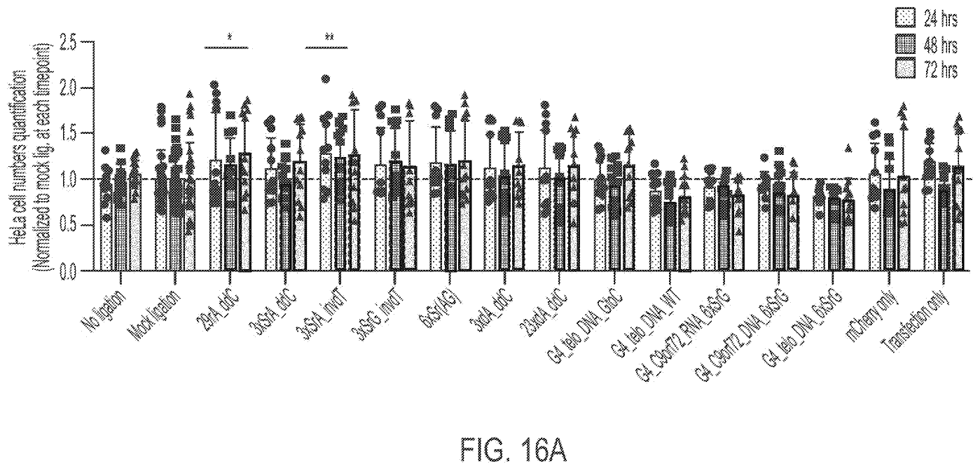

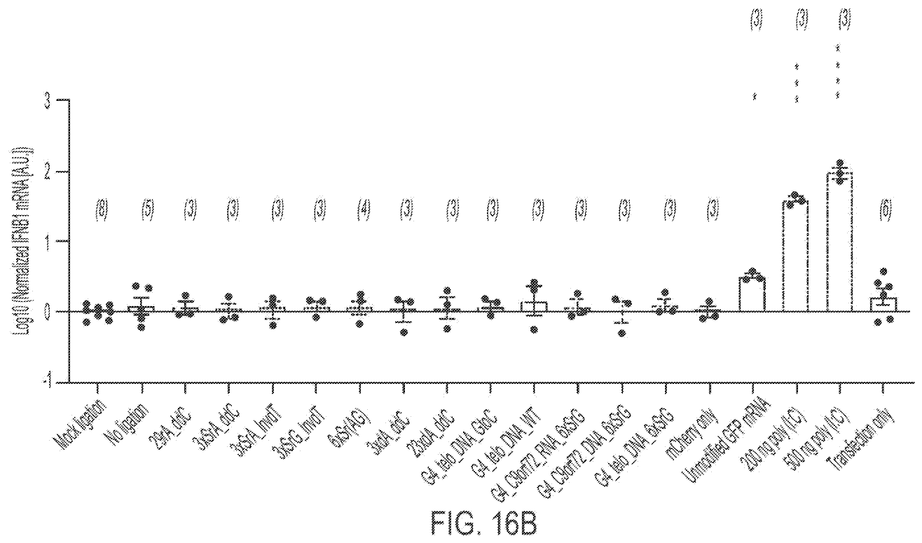

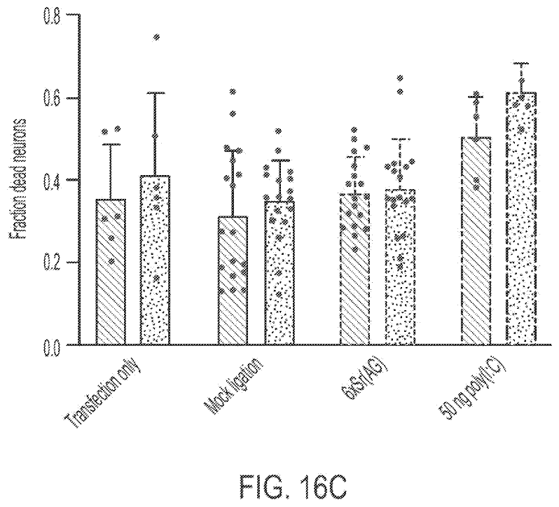

TABLE 7
Statistics for RT-qPCR data (48 hr
time point), from FIGs. 8C and 16B

| | GFP/(mCherry + | | | |
|---|---|---|---|---|
| | hActb), | | IfnB1/hActb, | |
| | normalized to | | normalized to | |
| | “Mock ligation” | | “Mock ligation” | |
| | Mean | s.d. | Mean | s.d. |
| Mock ligation | 1.09 | 0.5 | 1.02 | 0.2 |
| No ligation | 0.92 | 0.28 | 1.42 | 0.73 |
| 29rA_ddC | 0.76 | 0.13 | 1.2 | 0.37 |
| 3xSrA_ddC | 1.67 | 0.11 | 1.09 | 0.41 |
| 3xSrA_InvdT | 1.15 | 0.24 | 1.15 | 0.38 |
| 3xSrG_InvdT | 1.51 | 0.2 | 1.24 | 0.28 |
| 6xSr(AG) | 2.13 | 0.29 | 1.23 | 0.41 |
| 3xdA_ddC | 0.82 | 0.05 | 1.15 | 0.45 |
| 23xdA_ddC | 0.91 | 0.04 | 1.28 | 0.58 |
| G4_telo_DNA_GtoC | 1.11 | 0.25 | 1.25 | 0.27 |
| G4_telo_DNA_WT | 0.99 | 0.08 | 1.74 | 0.86 |
| G4_C9orf72_RNA_6xSrG | 2.37 | 0.57 | 1.28 | 0.45 |
| G4_C9orf72_DNA_6xSrG | 2.39 | 0.6 | 1.1 | 0.44 |
| G4_telo_DNA_6xSrG | 2.38 | 0.11 | 1.31 | 0.4 |
| mCherry only | 0.0005 | 0.0001 | 1.04 | 0.27 |
| Unmodified GFP mRNA | — | — | 3.24 | 0.37 |
| 200 ng poly(I:C) | — | — | 40 | 6 |
| 500 ng poly(I:C) | — | — | 100 | 20 |
| Transfection only | — | — | 2 | 1 |

Given the stochastic nature of lipid-mediated transfection and endosomal rupture, there can be a large variance in the number of transfected mRNAs across individual cells²⁷. To characterize whether the observed translational enhancement of mocRNAs represented a general increase in translation throughout the entire cell population, or if it resulted from a small set of high-expressing cells, the ratios of GFP/mCherry protein fluorescence and RNA copy numbers were quantified at the single-cell level. Single-cell fluorescence analyses of GFP/mCherry fluorescence ratios (FIG. 13A) recapitulated the trends observed in bulk measurements (FIG. 8A). mRNA abundance in transfected cells was further quantified using STARmap²⁸, an in situ transcriptomic method capable of identifying copy numbers of target mRNA sequences in fixed cell or tissue samples at subcellular resolution (FIGS. 8D, 13B). In the STARmap images, fluorescent puncta correspond to free “cytosolic” GFP-mocRNAs or mCherry mRNAs, respectively. Large intracellular granules likely correspond to lipid transfection vesicles containing many copies of GFP-mocRNAs and mCherry mRNAs (FIG. 8D). While RT-qPCR provides bulk measurements of mRNA (cytosolic and contained in the transfection reagent), STARmap enables the spatial separation of these two signals, enabling direct quantification of individual cytosolic mRNAs by filtering out signal from large aggregates. Importantly, the quantification of the cytosolic RNA fraction at the single-cell level indicates that the stabilization effects of mocRNAs also occur throughout the entire cell population (FIGS. 13C and 13D).

Protein and RNA Kinetics Show Increased Stability of mocRNAs in Cells

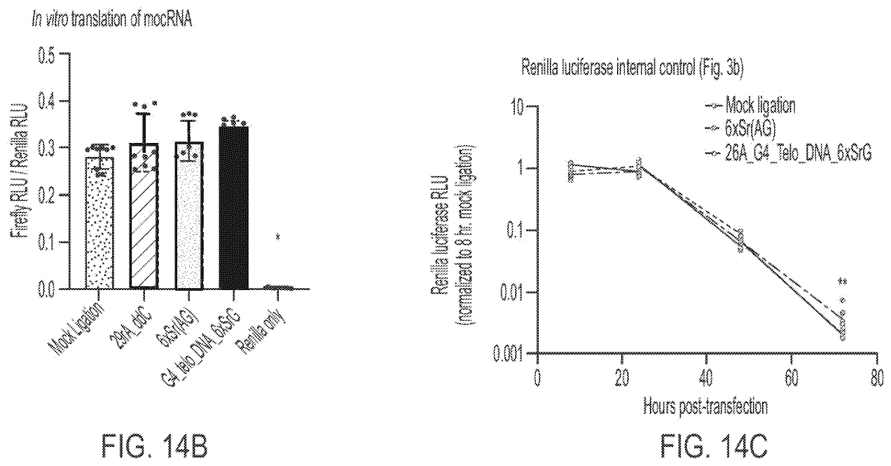

It seemed plausible that translation observed from the initial screen of PS+G4 oligos could be potentially confounded by the extension of the poly(A) tail by different lengths (26 A's in 6xSr(AG) and 6 A's in G4_C9orf72_RNA_6 xSrG, G4_C9orf72_DNA_6 xSrG, and G4_telo_DNA_6 xSrG). To address this point directly, a comparison was performed between 6xSr(AG) and redesigned longer PS+G4 oligos containing a similar number of A's: 26rA_G4_C9orf72_RNA_6xSrG, 26rA_G4_C9orf72_DNA_6 xSrG, and 26rA_G4_telo_DNA_6xSrG. The HeLa expression time course indicated that 6xSr(AG) outperformed the 26A-containing C9orf72 oligos in expression enhancement. However, 26rA_G4_telo_DNA_6xSrG demonstrated modest translation enhancements over 6xSr(AG) (17-24% between 24-72 hrs, FIG. 14A). These data suggests that specific telomere structures may add relatively low levels of additional stabilization, beyond the stabilization afforded by PS linkages. Due to the similar levels of expression between 6xSr(AG) and 26rA_G4_telo_DNA_6xSrG mocRNAs, these two oligos were examined in a downstream kinetic analysis of protein expression.

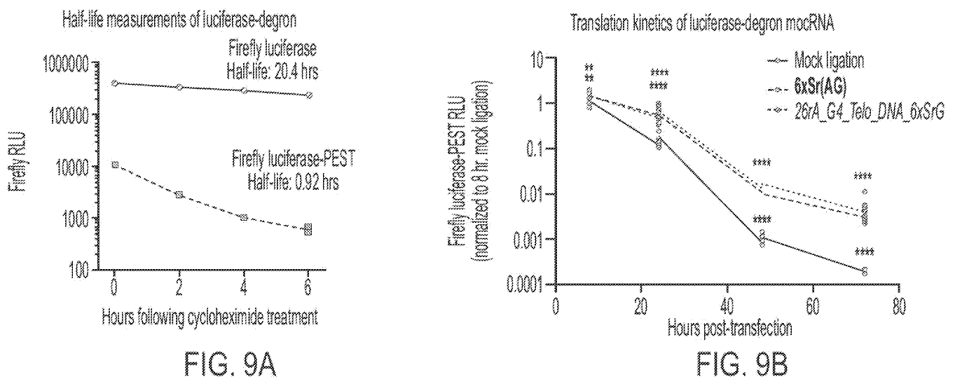

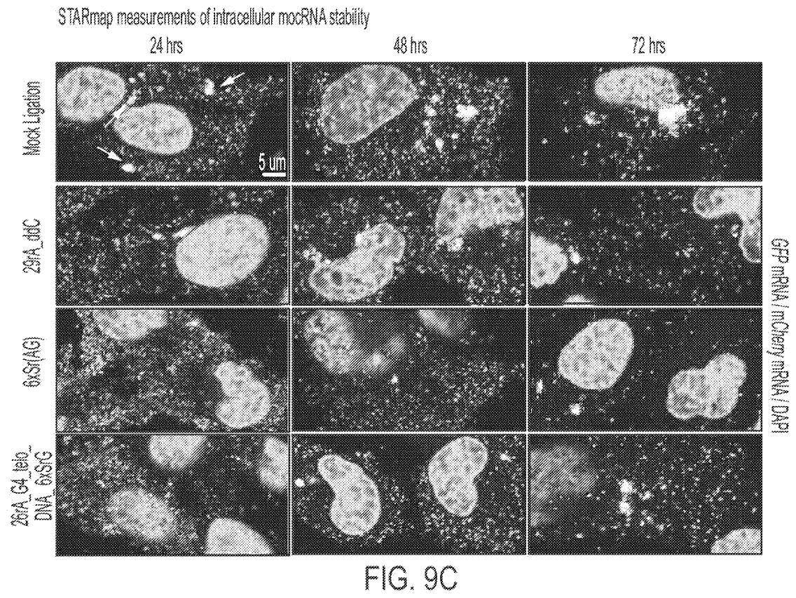

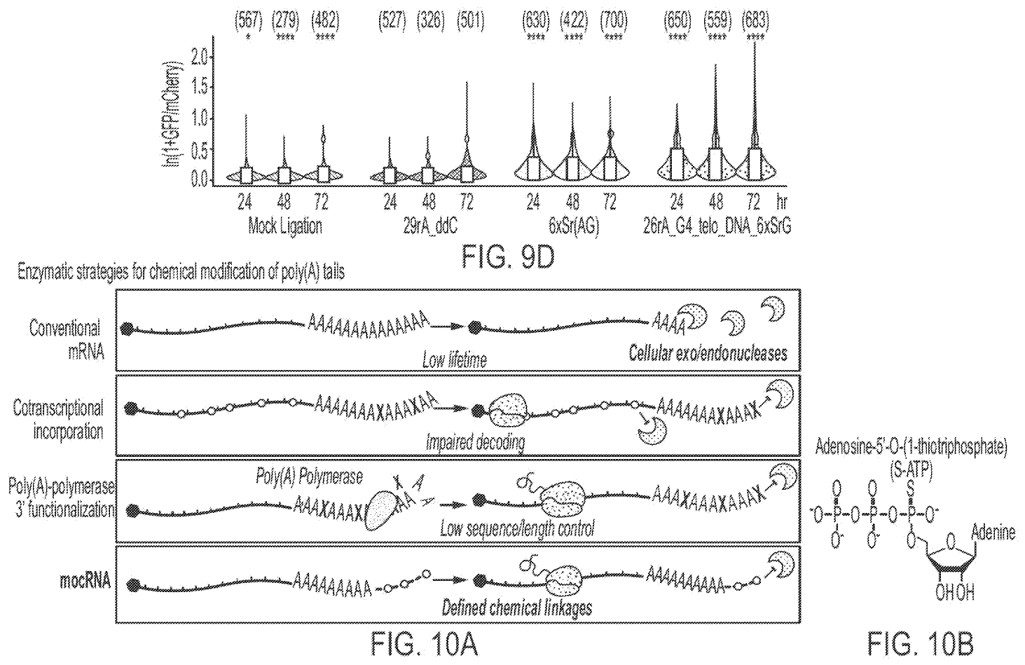

To characterize the kinetics of mocRNA translation at varying timepoints, mocRNAs encoding a degron-tagged Firefly luciferase were generated. The degron (PEST derived from mouse ornithine decarboxylase²⁹) reduced luciferase half-life in Hela cells from 20.4 hrs to an estimated 0.92 hrs (FIG. 9A) Luciferase-PEST mocRNAs were generated containing either of the two best-performing oligos, 6xSr(AG) and 26rA_G4_telo_DNA_6 xSrG, and luminescence was recorded as a function of time following mRNA transfection into Hela cells. At 8 hours post-transfection, 6xSr(AG) and 26rA_G4_telo_DNA_6xSrG mocRNAs (encoding luciferase-degron) demonstrated slightly greater levels of translation than the mock ligation (44% and 39% greater signal, respectively), However, by 48 and 72 hours, both mocRNAs substantially outperformed the mock ligation, with 6xSr(AG) demonstrating 10-fold and 15-fold more signal, respectively, and 26rA_G4_telo_DNA_6xSrG demonstrating 15-fold and 25-fold more signal (FIG. 9B). This translational enhancement was not due to differences in transfection efficiency between samples, as comparable significant differences were not observed in the translation of a co-transfected Renilla luciferase mRNA internal control (FIG. 14C). The observed kinetics of mocRNA translation is consistent with 6xSr(AG) and 26rA_G4_telo_DNA_6 xSrG possessing intact poly(A) tails at these timepoints (enabling translation), in contrast to the mock ligation. Furthermore, in vitro translation experiments performed on mocRNAs did not show substantial differences in translation efficiency between mocRNA and controls (FIG. 14B). This indicates that increased protein expression from mocRNA is primarily attributed to enhanced mRNA lifetime, rather than enhanced translation initiation efficiency.

The kinetics of mocRNA decay was further verified in cells by performing in situ mRNA visualization using STARmap at 24, 48, and 72 hours post-transfection into Hela cells (FIG. 9C). GFP-60A mocRNAs containing 29rA_ddC, 6xSr(AG), or 26rA_G4_telo_DNA_6xSrG, were transfected into HeLa cells and relative mRNA abundance was quantified over time. The 6xSr(AG) mocRNA samples displayed 1.7-2.5-fold higher GFP/mCherry mRNA count ratios (averaged from single cells) than 29rA_ddC at each time point. Additionally, 26rA_G4_telo_DNA 6xSrG had 1.7-3.1-fold higher GFP/mCherry mRNA count ratios compared to the 29rA_ddC control at each time point (FIG. 9D).

mocRNA Outperforms Alternative Strategies for mRNA Modification

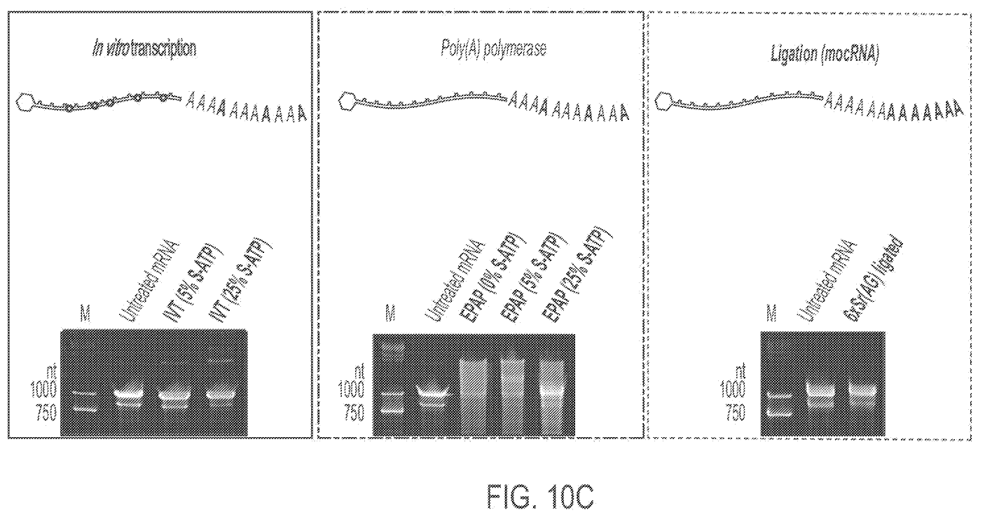

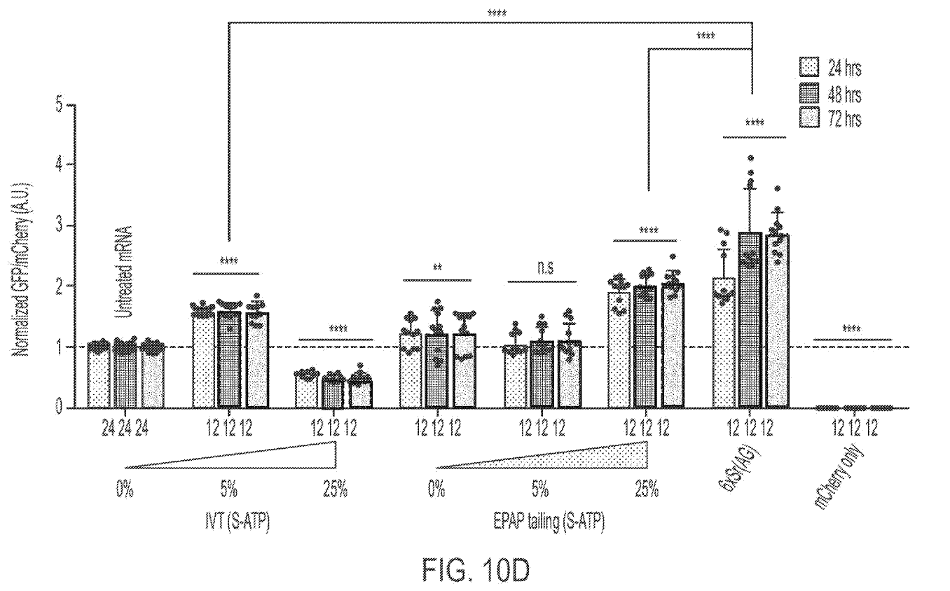

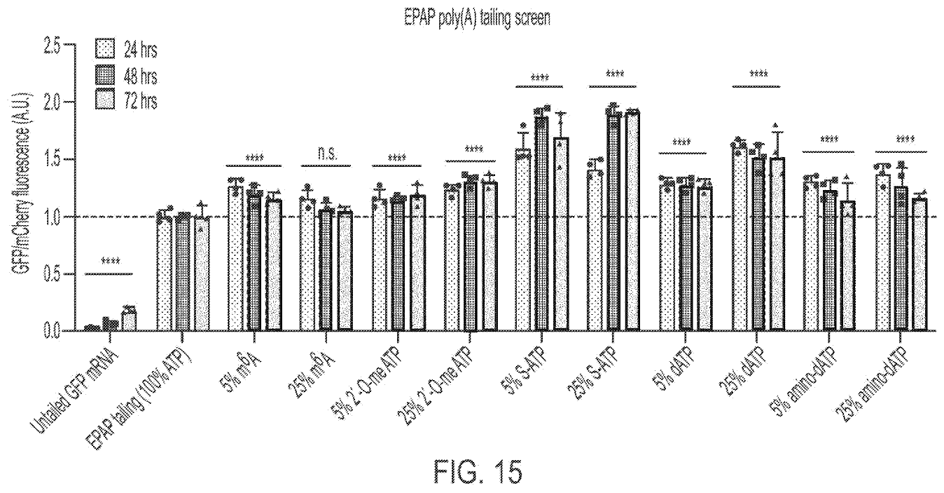

Previous work has reported that PS linkages incorporated by E. coli poly(A) polymerase (E-PAP) into the poly(A) tail can enhance mRNA stability¹⁸. Therefore, the E-PAP modification strategy of poly(A) tails was also explored. A panel of chemically modified ATP derivatives (XATP) was screened by introducing XATP spike-ins into poly(A) tailing reactions on a capped GFP mRNA containing N1-methylpseudouridine instead of uridine (FIG. 15). HeLa cells were co-transfected with various tail-modified GFP mRNAs along with an internal transfection control, tail-unmodified mCherry mRNAs (100% ATP, E-PAP tailed) and monitored the GFP/mCherry fluorescence ratio over a three-day time course. The initial screen in HeLa cell experiments revealed that poly(A) modification by XATP spike-ins increased normalized GFP production in comparison with the unmodified poly(A) construct, particularly for dATP (2′-deoxyadenosine triphosphate, 25-62% increase in normalized GFP/mCherry) and 5-ATP (adenosine-5′-O-(1-thiotriphosphate), 42-91% increase) (FIG. 15). 5-ATP spike-in resulted in the greatest enhancement of GFP expression (consistent with previously reported work¹⁸) and thus was used to compare different mRNA modification strategies (FIG. 10A).

6xSr(AG) to GFP-60A mRNAs functionalized by S-ATP were compared via IVT or E-PAP incorporation (FIGS. 10A to 10C) in RNA length homogeneity and protein production. mocRNAs and IVT-modified constructs showed uniform length distributions, whereas E-PAP-tailed mRNAs have a wide distribution of tailing lengths, with shorter lengths as the percentage of S-ATP spike-in increased (FIG. 10C). Using mCherry mRNA (E-PAP tailed with 100% A) as an internal transfection control, GFP/mCherry fluorescence ratios were quantified at 24, 48, and 72 hours post-transfection in HeLa cells. After normalizing to the untreated GFP-60A control, the 6xSr(AG) mocRNA resulted in the highest enhancement of GFP expression at various times post-transfection (24 hr: 214±45%; 48 hr: 289 #: 68%, 72 hr: 286±32%; mean±s.d.) (FIG. 10D). Among all the E-PAP tailed mRNA constructs, 25% 5-ATP spike-in had the highest enhancement of GFP expression in comparison with the untreated GFP-60A control (24 hrs, 93±21% increase). IVT-mediated incorporation of S-ATP proved beneficial for small percentages of modified ATP (24 hrs, 5% 5-ATP: 160±7%). Decreased translation of the reporter at 25% 5-ATP (54±5%) was observed compared to the untreated GFP-60A mRNA. Overall, this systematic comparison between different modification methods of mRNA tails demonstrated the superior performance of mocRNAs over E-PAP and IVT-modified mRNA (FIG. 10D).

mocRNA Constructs Enhance Protein Expression in Primary Rat Cortical Neuronal Cultures

Neurons are the main therapeutic targets in a variety of brain and nervous system-related diseases^{30,31}. While chemical/lipid-mediated transfection of DNA plasmids demonstrates limited expression efficiency in postmitotic cells, such as neurons, mRNA transfection is an alternative to introduce transgenic protein expression in neurons with a higher efficiency³². To explore whether mocRNA could increase protein production in primary cell culture, the modified constructs were tested in primary cultures of rat cortical neurons.

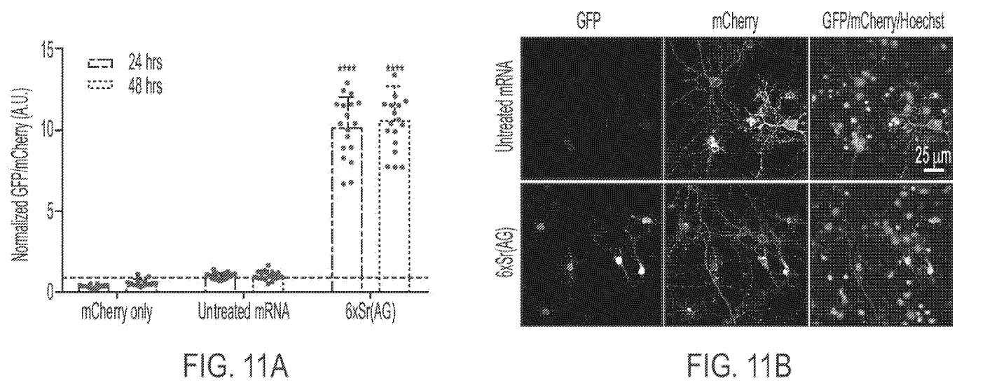

GFP mocRNA prepared by 6xSr(AG) oligos and unligated controls were co-transfected with mCherry mRNA (E-PAP tailed with 100% rA, transfection control) for comparisons at 24 hours and 48 hours post-transfection (FIG. 11A). In comparison with unligated GFP samples, the GFP expression of 6xSr(AG) mocRNA samples showed an order of magnitude higher expression at both time points (24 hours: 1015±190%; 48 hours: 1061±210) (FIGS. 11A-11B, Table 8). These results demonstrated that mocRNAs can offer robust enhancement of protein expression in neuronal cell culture, compared to conventional mRNA vectors.

TABLE 8
Statistics for GFP/mCherry fluorescence ratio data, from FIG.
11A (normalized to “mock ligation” samples).

| | mCherry only | Untreated mRNA | 6xSr(AG) |
|---|---|---|---|
| 24 hour statistics | | | |
| Mean | 0.24 | 1.00 | 10.15 |
| s.d. | 0.19 | 0.19 | 1.90 |
| s.e.m. | 0.04 | 0.04 | 0.45 |
| Sample Size | 12 | 12 | 12 |
| 48 hr statistics | | | |
| Mean | 0.47 | 1.00 | 10.61 |
| s.d. | 0.32 | 0.26 | 2.10 |
| s.e.m. | 0.07 | 0.06 | 0.49 |
| Sample Size | 12 | 12 | 12 |

s.d.: standard deviation; s.e.m: standard error of the mean.

mocRNA Retains Similar Toxicity Profiles to Therapeutic mRNA

Unmodified IVT mRNA triggers strong immune responses upon transfection, which suppress its protein production¹⁰⁻¹². While 100% replacement of uridine with N1-methylpseudouridine is used in therapeutic mRNA (and mocRNA) preparations to minimize immune toxicity¹², it was further evaluated whether chain-terminating nucleotides, PS linkages, or the covalent DNA-RNA bonds introduced by the synthetic oligos into mocRNAs would trigger additional cellular toxicity. First, cell numbers were quantified from imaging data displayed in FIG. 8, to check for substantial decreases in cell proliferation and viability. Significant decreases in HeLa cell numbers were not observed between any mocRNA condition and the unligated mRNA control (FIG. 16A). Additionally, innate immune stimulation in HeLa cells was measured through RT-qPCR measurements of IFNB1 mRNA on the 48-hour post-transfection samples shown in FIG. 8. IFNB1 upregulation is a consequence of RIG-I and MDAS activation, which are innate immune sensors that recognize foreign RNA species³³⁻³⁵. Positive controls of unmodified GFP mRNA (100% uridine) and poly(I:C) transfection (a potent RIG-I agonist³⁶) induced statistically significant IFNB1 mRNA upregulation when compared to the 29rA_ddC mocRNA control (Welch's t-test). However, no significant differences were observed between any mocRNAs, unligated mRNA, and the transfection only control (FIG. 16B). These results indicate that mocRNAs do not inherently increase innate immune responses beyond untreated mRNAs, at least for the constructs explored in this study.

Finally, mocRNA-mediated toxicity was analyzed in neurons using live-dead cell staining on transfected rat cortical neuron cultures (with Hoechst stain and NucRed Dead 647). The percentage of dead neurons was calculated in each culture condition to test for differences in cellular toxicity between mocRNA and conventional mRNA transfection. Significant differences in neuronal toxicity caused by 6xSr(AG) ligation were not observed, as compared to a transfection control (FIG. 16C). Taken together, these results suggest that the modifications identified in this study did not substantially alter the toxicity profiles of mRNAs in the cell cultures tested.

### Summary and Conclusions

Existing methods that utilize poly(A) polymerase to synthesize chemically modified poly(A) tails often result in wide distributions of tail lengths that could complicate batch-to-batch homogeneity and cannot precisely control modification sites. In contrast, mocRNA synthesis demonstrates nearly 100% yields and can fully preserve mRNA homogeneity, which makes it compatible with existing pipelines for the development of mRNA therapeutics. More importantly, the mocRNA expression system can introduce chemical modifications that cannot be incorporated by RNA polymerases and enables precise control of modification sites to maximize the effects of RNA modifications. As the first demonstration, mocRNA with clustered nuclease-resistant motifs at the 3′ terminus enhanced protein expression by protecting the poly(A) tail of mRNA vectors. Fluorescent protein measurements demonstrated that mocRNAs containing 3′ terminal PS linkages increased protein production by factors of 2-4 in human HeLa cell lines (FIG. 8A) and by 10-fold in primary rat cortical neuronal cultures (FIG. 11A). Combined bulk RT-qPCR measurement and single-cell resolved in situ STARmap measurements indicate that mocRNAs containing 3′ terminal PS modifications and specific telomere sequences improve protein expression primarily by stabilizing RNAs (FIGS. 8A, 14A)³⁷. These mocRNA constructs have higher translation capacity than existing variants of mRNA vectors relying on random incorporation of modified NTPs during IVT and polyadenylation^{14,15,18} (FIG. 10D).

In summary, a modular, programmable, and effective strategy to synthesize mocRNAs was developed, enabling diversified and precise chemical modifications of RNA vectors to enhance protein translation capacity and RNA stability. mocRNAs can potentially be combined with other types of modification strategies, such as poly(A) binding protein (PABP)-binding oligos (see, e.g., Barragán-Iglesias, et al. Nat Commun, 9(1): 10)³⁸, hydrolysis-resistant 7-methylguanosine caps^{39,40}, modified 5′ UTR regions⁴¹, and other types of modified nucleotides in the mRNA body⁴². mocRNA design could serve as a generalizable platform for integrating organic synthesis with enzymatic synthesis, to diversify chemical moieties and boost functional efficacy of RNA-based protein expression systems.

### Methods

### Plasmid Cloning, Characterization, and Purification

hMGFP and mCherry-encoding plasmids (pCS2_hMGFP and pCS2_mCherry, respectively) were obtained from Xiao Wang. These plasmids contained (in order) an SP6 promoter sequence, a 5′ UTR, a fluorescent protein coding sequence (CDS), 3′ UTR, and NotI restriction cut site. Sequences can be found in the original reference⁴³.

The Q5® Site-Directed Mutagenesis Kit (NEB: E0554S) was used to perform PCRs on template plasmids using primers (Table 4) containing site-specific modifications. This was followed by KLD enzyme treatment, then transformation into NEB Stabl cells (NEB: C3040H) for isolation using the ZymoPURE plasmid miniprep kit, and Sanger sequencing through Genewiz.

For the site-specific installation of 60xA template-encoded poly(A) tails in front of an Esp3I site, two sequential rounds of cloning were performed using Q5 site-directed mutagenesis. The first round of cloning installed an Esp3I restriction site 5′ of the previous NotI restriction site (Esp3i_insert_F and Esp3i_insert_R). The resulting Sanger sequencing-verified plasmid was used as a template for the installation of the 60xA poly(A) tail (60A_insert_F and 60A_insert_R). The clone selected from the second round of cloning was verified using Sanger sequencing. See Supplementary Table 4 for primer sequences. The name of the construct containing ˜60 nt long template-encoded tails prior to the Esp3I site was pCS2_hMGFP-60A.

Firefly luciferase constructs were generated first by deletion of the hMGFP coding region from pCS2_hMGFP-60A vector using PCR. Next, the Firefly luciferase coding sequence was PCR amplified from pmirGLO Dual-Luciferase miRNA Target Expression Vector (Promega: E1330), with PCR primers designed to contain 15-20 nucleotide complementary overhang regions to the vector of interest. Vector and insert were assembled using NEBuilder HiFi DNA Assembly Master Mix (NEB: E2621S), transformed into Stabl cells, and sequence-verified by Sanger sequencing. Renilla luciferase constructs were cloned by an analogous process to Firefly luciferase, except with a Renilla luciferase coding sequence from the pmirGLO vector.

The destabilized Firefly luciferase construct (i.e., Firefly-PEST) contains a degron derived from mouse ornithine decarboxylase²⁹. The aforementioned Firefly luciferase vector was PCR-linearized around the stop codon, into which a GeneBlock (IDT, human codon-optimized) encoding the PEST sequence was inserted using the NEBuilder HiFi method.

mRNA Synthesis and Characterization

GFP mRNA was synthesized from pCS2_hMGFP-60A plasmid, which contained an SP6 promoter, followed by hMGFP CDS and template-encoded poly(A) tail. Plasmids were linearized by a single Esp3I site located immediately 3′ of the poly(A) region, which was installed during cloning. Linearized plasmids were then purified using the DNA Clean & Concentrator-25 kit from Zymo Research (D4033) and checked for purity via agarose gel electrophoresis. Capped, modified mRNA was prepared using SP6 enzyme and reaction buffer from mMESSAGE mMACHINE™ SP6 Transcription Kit (ThermoFisher Scientific: AM1340). The 2×NTP/Cap solution provided by the kit was replaced with a 2×NTP/Cap preparation containing: 10 mM ATP (NEB: N0451AVIAL), 10 mM CTP (NEB: N0454AVIAL), 2 mM GTP (NEB: N0452A VIAL), 8 mM 3′-O-Me-m7G(5′)ppp(5′)G RNA Cap Structure Analog (NEB: S1411S), and 10 mM N1-Methylpseudouridine-5′-Triphosphate (TriLink Biotechnologies: N-1081-1). SUPERase-In RNase Inhibitor (ThermoFisher Scientific: AM2694) was added to a final concentration of 1:20 (v/v). Following IVT reaction assembly and incubation at 37° C. for 2-4 hours, reactions were treated with 1-2 μl of TURBO DNase (provided in AM1340) for 1 hr at 37° C. prior to reaction purification using MEGAclear™ Transcription Clean-Up Kit (ThermoFisher Scientific: AM1908). Superase-In RNase Inhibitor was added to purified mRNA samples to a final concentration of 1:50 (v/v), and stored samples at −80° C. for long-term storage. Purified mRNA was measured by Nanodrop to estimate concentration prior to ligations, and mRNAs and mocRNAs were measured using the Qubit RNA HS Assay (ThermoFisher Scientific: Q32852) for normalization immediately prior to transfection for cell-based testing.

For the preparation of poly(A) polymerase-tailed mRNA (FIG. 15), dsDNA templates generated by linearization of pCS2_hMGFP and pCS2_mCherry plasmids using NotI-HF (NEB: R3189S) were used, and column purified digested products using Zymo DNA Clean & Concentrator-25. In vitro transcription was performed using the protocol described above, except after TURBO DNase digestion, the extra step of poly(A) tailing was included using the E-PAP Poly(A) Tailing Kit (ThermoFisher Scientific: AM1350). Purification and storage of mRNA were as described above (e.g., using MEGAclear transcription cleanup kit).

For FIG. 10, adenosine-5′-O-(1-thiotriphosphate) spike-in mRNAs were synthesized using a modified protocol to the one listed above. Adenosine-5′-O-(1-thiotriphosphate) (S-ATP) was used for co-transcriptional incorporation experiments. Qualitative differences in S-ATP incorporation were observed when stock tubes that had been opened previously were used, possibly due to oxidation. For this reason, new tubes were used prior to every tailing experiment, to limit the effects of possible oxidation as a confounding factor in these experiments. 5-ATP in vitro transcription reactions were performed with the same setup as listed above, but the final 5 mM ATP in the reaction was replaced with either 4.75 mM ATP+0.25 mM S-ATP (5% 5-ATP incorporation) or with 3.75 mM ATP+1.25 mM S-ATP (25% 5-ATP). IVT templates containing the GFP coding sequence with a 60xA template-encoded poly(A) tail were used.

Modified E. coli Poly(A) Polymerase Tailing

For modified E-PAP tailing experiments in FIG. 15, the substrate was an untailed GFP mRNA generated from IVT's on a NotI-HF linearized pCS2_hMGFP template (see IVT protocol above). This protocol utilized the enzyme and buffer from E-PAP Poly(A) Tailing Kit (ThermoFisher Scientific: AM1350). “10 mM total” ATP stock solutions were prepared for each modified ATP spike-in, such that a specific percentage of ATP was replaced by a modified ATP derivative (XATP). For example, 25% dATP samples would require the assembly of a 2.5 mM dATP, 7.5 mM ATP stock solution. Tailing reactions were assembled as follows: 1.5 μg of untailed GFP mRNA: 5 ul of 5×E-PAP buffer; 2.5 ul of 10 mM XATP:ATP stock solution (different for each sample); 2.5 ul of 25 mM MnCl₂; 1 μl of Superase-In RNase Inhibitor; 1 μl of E-PAP enzyme; and nuclease-free water up to a total volume of 25 μl. Reactions were incubated at 37° C. for 1 hour, then quenched with the addition of 0.5 ul of 500 mM EDTA. These tailed mRNAs were then column purified using Monarch RNA cleanup kit (50 μg) (NEB: T2040S). Superase-In RNase Inhibitor was added to purified mRNA to a final dilution of 1:50 (v/v), and mRNA was stored at −80° C. prior to transfection.

The following modified ATP derivatives (XATPs) were used in these experiments: Adenosine 5′-Triphosphate (ATP) (NEB: P0756S); N6-Methyladenosine-S′-Triphosphate (TriLink Biotechnologies: N-1013-1); 2′-O-Methyladenosine-5′-Triphosphate (TriLink Biotechnologies: N-1015-1); Adenosine-5′-O-(1-Thiotriphosphate) (TriLink Biotechnologies: N-8005-1); dATP solution (NEB: N0440S); 2′-Amino-2′-deoxyadenosine-5′-Triphosphate (TriLink Biotechnologies: N-1046-1).

For modified E-PAP-tailing seen in FIG. 10 (methods comparison), E-PAP tailing was performed using the hMGFP-encoding mRNA containing a template-encoded 60A tail (in contrast to FIG. 15). Adenosine-5′-O-(1-thiotriphosphate) (S-ATP) was used for co-transcriptional or modified poly(A) tailing experiments. Qualitative differences were observed in S-ATP incorporation when stock tubes that had been opened previously were used, potentially due to oxidation. For this reason, new tubes were used prior to every tailing experiment, to limit the effects of possible oxidation as a confounding factor in these experiments. EPAP tailing reactions (with S-ATP spike-ins) were otherwise set up consistently with the protocol described above.

### Modified Oligo 3′ End Ligations

Ligation reactions were performed using T4 RNA Ligase I (Promega: M1051). Reactions were assembled as follows: 2 μg of GFP mRNA; 200 pmol of the synthetic oligo; 2 μl of Superase-In RNase Inhibitor, 20 μl of 50% PEG-8000; 5 μl of 100% DMSO; 5 ul of 10×T4 RNA ligase buffer; 5-7.5 μl of T4 RNA ligase (Promega); and nuclease-free water to a total reaction volume of 50 μl. Reactions were incubated at 37° C.′ for 30 minutes, followed by inactivation of the reaction via the addition of 1 ul of 500 mM EDTA, pH 8.0. Reactions were diluted by the addition of 1 volume of nuclease-free water (e.g., 50 μl), followed by the addition of 0.5 volumes of AMPure XP (Beckman Coulter: A63880) containing 1 μl Superase-In (e.g., 25 μl). Reactions were purified according to the manufacturer's protocol, and mRNA was eluted from AMPure beads using nuclease-free water containing Superase-In at a 1:50 (v/v) ratio. mRNA samples that appeared to contain residual oligo on a gel were purified a second time using AMPure XP beads.

For ligations that were incomplete according to the RNase H gel-based assay, ligations were performed using a modified condition, in which DMSO was omitted from the reaction. This generally resulted in more efficient ligation. For ligation-prepared samples shown in FIGS. 9 to 11, the modified protocol was used for ligations, as this was generally more efficient. For Firefly luciferase and Firefly-PEST mRNA ligations, these were purified using 2× serial Ampure XP bead clean-ups, using a 1:1 bead volume to mRNA volume. For example, a 50 μl ligation reaction was cleaned up using 50 μl of Ampure XP beads. Following elution of the product mRNA, a second clean-up was performed using an equal volume of beads to the eluted mRNA product.

### RNase H Assays

A potassium chloride (KCl) stock solution was used for annealing an ssDNA oligo to mRNA prior to RNase H assays. The annealing stock solution contained: 50 mM KCl, 2.5 mM EDTA, 1:200 (v/v) Superase-In RNase inhibitor, brought to its final volume using nuclease free water. The ssDNA probe (RNaseH_probe_GFP) was ordered from Integrated DNA Technologies (IDT). Sequences are listed in Table 5.

TABLE 5
Oligonucleotides used for cloning, RT-qPCR, RNase H assays, and STARmap
characterization.

| | |
|---|---|
| Primer name | Sequence (5′ to 3′) |

Cloning

| | |
|---|---|
| Esp3i_insert_F | AGAGACGTTCGCGGCCGCGGOCGCC (SEQ ID NO: 36) |
| Esp3i insert_R | TTAAAAAACCTCCCACACCTCCCCCTGAACCTGAAAC |
| | (SEQ ID NO: 37) |
| 60A_insert_F | AAAAAAAAAAAAAAAAAAAAAAAAAAAAAAAAAAAA |
| | AGAGACGTTCGCGGCCGCGGCGCC (SEQ ID NO: 38) |
| 60A_insert_R | TTTTTTTTTTTTTTTTTTTTTTTTTTTTTTTCCTCCCACAC |
| | CTCCCCCTGAACCTGAAAC (SEQ ID NO: 39) |

RNase H assay

| | |
|---|---|
| RNaseH_probe_GFP | GCATCACAAATTTCACAAATAAAGCATTTTTTTCAC |
| | (SEQ ID NO: 18) |

RT-qPCR quantification

| | |
|---|---|
| hMGFP_qPCR_F | TGACATTCTCACCACCGTGT (SEQ ID NO: 40) |
| hMGFP_qPCR_R | AGTCGTCCACACCCTTCATC (SEQ ID NO: 41) |
| mCherry_qPCR_F | TTCTTGGCCATGTAGGTGGTC (SEQ ID NO: 42) |
| mCherry_qPCR_R | AGGACGGCGAGTTCATCTAC (SEQ ID NO: 43) |
| hActb_qPCR_F | CACCATTGGCAATGAGCGGTTC (SEQ ID NO: 44) |
| hActb_qPCR_R | AGGTCTTTGCGGATGTCCACGT (SEQ ID NO: 45) |
| Origene_IFNB1_qPCR_F | CTTGGATTCCTACAAAGAAGCAGC (SEQ ID NO: 46) |
| Origene_IFNB1_qPCR_R | TCCTCCTTCTGGAACTGCTGCA (SEQ ID NO: 47) |

STARmap: SNAIL probes

| | |
|---|---|
| mCherry-01 | /5Phos/ACATTATTGGTGCCGCGCAGCTTCACCTAATTAT |
| | TACTGAGGCATACACTAAAGATA (SEQ ID NO: 19) |
| mCherry-02 | /5Phos/ACATTACTTCTTGGCCTTGTAGGTGGTAATTATT |
| | ACTGAGGCATACACTAAAGATA (SEQ ID NO: 20) |
| mCherry-03 | /5Phos/ACATTACACGGTCACCACGCCGCCGTAATTATTA |
| | CTGAGGCATACACTAAAGATA (SEQ ID NO: 21) |
| mCherry-11 | ACGGGGCCGTCGGAGGGGAATAATGTTATCTT (SEQ ID |
| | NO: 22) |
| mCherry-12 | GGCGCCGGGCAGCTGCACGGTAATGTTATCTT (SEQ ID |
| | NO: 23) |
| mCherry-13 | GTCCTGCAGGGAGGAGTCCTGGTAATGTTATCTT (SEQ |
| | ID NO: 24) |
| hMGFP-01 | /5Phos/ACATTAAGTCGCAGCGGTAGTGGCCAATTATTAC |
| | TGAAATCGTAGACTAAGATA (SEQ ID NO: 25) |
| hMGFP-02 | /5Phos/ACATTACATTAGCAGGGAAGTTGACCCCGTAATT |
| | ATTACTGAAATCGTAGACTAAGATA (SEQ ID NO: 26) |
| hMGFP-03 | /5Phos/ACATTAGCTTCGGCGTGCTCGTACAGCTAATTAT |
| | TACTGAAATCGTAGACTAAGATA (SEQ ID NO: 27) |
| hMGFP-11 | CCTCCCTCCAAGAGCAGTGCCATTAATGTTATCTT (SEQ |
| | ID NO: 28) |
| hMGFP-12 | TGCGCTGCATCACCGGGCTAATGTTATCTT (SEQ ID NO: |
| | 29) |
| hMGFP-13 | CCTGGCGGGGTAGTCCGCTGTGTAATGTTATCTT (SEQ |
| | ID NO: 30) |

STARmap: fluorescent detection probes

| | |
|---|---|
| mCherry_detect_Alexa647 | /5Alexa647N/CATACACTAAAGATAACAT (SEQ ID NO: 31) |
| hMGFP_detect_Alexa546 | /SAlex546N/TCGTAGACTAAGATAACAT (SEQ ID NO: 32) |

The following reaction was prepared to anneal mRNA to the aforementioned ssDNA probe: 200 ng of purified mRNA sample (ligated or unligated); 2 pmol of RNaseH_probe_GFP; 2 μl of annealing stock solution (50 mM KCl, 2.5 mM EDTA, 1:200 Superase-In); and nuclease-free water up to a total volume of 10 μl. Reactions were denatured at 70° C. for 5 minutes, followed by cooling to room temperature at a rate of 0.2° C./see in a benchtop thermocycler. Following probe annealing, 1 μl of Thermostable RNase H (NEB: M0523S) and 1 μl of the 10× buffer were added to each reaction, which was incubated at 50° C. for 30 min. Following reaction incubation, samples were digested by the addition of 1 μl Proteinase K (ThermoFisher Scientific: 25530049) and incubated at room temperature for 5 minutes. Subsequently, samples were mixed with 1 volume of Gel Loading Buffer II (ThermoFisher Scientific: AM8546G), which had been supplemented with EDTA to a final concentration of 50 mM. Samples in 1× loading buffer were denatured at 70° C. for 3-5 minutes prior to loading and resolution on 6% Novex™ TBE-Urea Gels (ThermoFisher Scientific: EC68655BOX), run in 1× Tris-borate-EDTA (TBE) buffer. Ladder used for gels was Century-Plus RNA Markers (ThermoFisher Scientific: AM7145). All gels were stained in 1×SYBR Gold (ThermoFisher Scientific: S11494) in 1×TBE buffer for 5-15 minutes prior to visualization using the BioRad ChemiDoc MP Imaging System (12003154) or the MP Imager (Universal Hood III), and images were exported using the corresponding Image Lab software.

Mammalian Cell Culture and mRNA Transfection

HeLa cells (CCL-2, ATCC) are maintained in DMEM culture media (ThermoFisher 11995) containing 10% FBS in a 37° C. incubator with 5% CO2 and passaged at the ratio of 1:8 every three days. The cell culture was confirmed to be free of mycoplasma contamination regularly with Hoechst staining and microscopy imaging.

On the day before mRNA transfection, the cells were seeded at 75% confluence in individual wells on a 12-well plate. The day after, 500 ng mCherry (internal control) mRNA and 500 ng GFP mRNA with synthetic tails or other modifications (concentrations determined by Qubit) were transfected into each well using 3 μL Lipofectamine™ MessengerMAX™ Transfection Reagent (ThermoFisher, LMRNA003) Additional controls that contain only mCherry mRNA, or only transfection reagents, or non-transfected cells are included. After a 6 hr incubation, the lipofectamine/mRNA transfection mixture was removed, and cells were rinsed once with DPBS and trypsinized to reseed into three glass-bottom 24-well plates (MatTek, P24G-1.5-13-F, poly-D-lysine coated) at a ratio of 6:4:3, respectively, for fluorescent protein quantification at 24 hours, 48 hours, and 72 hours after transfection.

Freshly dissociated rat primary cortical neurons were kindly provided by Sheng Lab at the Broad Institute. Briefly, rat cortical neuronal cultures were prepared from embryonic day 18 (E18) embryos from CO₂-euthanized pregnant Sprague Dawley rats (Charles River Laboratories). Embryo cortices were dissected in ice-cold Hank's Balanced Salt Solution (HBSS, Gibco, 14175-095) supplemented with 100 U/mL Penicillin/Streptomycin (Gibco, 15140-122). Cortical tissues were washed 3× with 4° C. PBS (Sigma, D8537), digested in 0.25% Trypsin-EDTA (Gibco, 25200-056) for 20 min at 37° C., and then washed again 3× with room temperature PBS. Cortical tissue was gently dissociated in 37° C. NBActiv4 media (Brainbits, NB4-500) and centrifuged at 300×g for 5 min. The pellet was resuspended in fresh NBActiv4 and passed through a 70 μm filter (Corning, 352350).

Neurons were seeded at a density of 1×10⁵/cm² on poly-D-lysine coated (Sigma, A-003-E, 50 g/mL for at least one hour at room temperature followed by three rinses with sterile distilled H₂O and air dried) 24-well glass-bottom plates (MatTek, P24G-1.5-13-F) in 0.5 mL NbActiv4 media with half of the media changed every four days. On SDIV, neurons in 24-well plates were transfected with 250 ng mCherry (internal control) mRNA and 250 ng GFP mRNA with synthetic tails (concentrations determined by Qubit) mixed with 1.5 μL Lipofectamine™ MessengerMAX™ Transfection Reagent (ThermoFisher, LMRNA003). The neurons were incubated with the transfection mixture for 2 hours before changing back to the normal culture media (half old, half fresh). Procedures for rat neuronal culture were reviewed and approved for use by the Broad Institutional Animal Care and Use Committee. All procedures involving animals were in accordance with the US National Institutes of Health Guide for the Care and Use of Laboratory Animals.

### Confocal Imaging and Quantification of Fluorescent Proteins

Before fluorescent protein imaging, the culture media was removed and the cells were rinsed with DPBS once before being incubated in the nuclei staining media (FluoroBrite™ DMEM [ThermoFisher, A1896701] with 1:2000 dilution of Hoechst 33342 [ThermoFisher, 62249]) at 37° C. for 10 mins.

For Hela cells, confocal images of the nuclei (Hoechst), GFP, and mCherry were taken by Leica Stellaris 8 with a 10× air objective at the pixel size of 900 nm×900 nm. Four representative fields of view were taken for each well, one from each quadrant. For neurons, confocal image stacks of the nuclei (Hoechst), GFP, and mCherry are taken by Leica Stellaris 8 with a 25× water immersion objective at the pixel size of 450 nm*450 nm, and step size of 1 μm for 9 steps. Six representative fields of view are taken for each well (FIG. 11). For toxicity measurements in neurons, NucRed Dead 647 (Invitrogen: R37113) was added to the Fluorobrite staining media prior to imaging and used the corresponding channel to obtain images for the nuclei of dead cells. The same imaging setting was used for all the samples to be compared. Excitation/detection wavelengths are as the following: Hoechst: Diode 405 nm/˜[430-480] nm; GFP: WLL 489 nm/˜[500-576] nm; mCherry: WLL 587 nm/˜[602-676] nm. CellProfiler 4.0.744 was used to calculate the number of objects in the Hoechst (e.g., total number of nuclei) versus NucRed Dead channel (e.g., dead nuclei), to yield fraction dead neurons in each field of view.

For bulk analyses in cultured neurons (FIG. 8A), first, the mCherry intensity and GFP intensity in each image were measured. The average fluorescence signals in the mCherry channel and GFP channel in the “Transfection only” samples were considered as background signals. Background signals were subtracted from each figure. Finally, the ratio between GFP intensity and mCherry intensity in each image was calculated. And outliers within each sample, determined by GraphPad Prism 9, were removed. The means of the ratios between GFP intensity and mCherry intensity in all the “Untreated mRNA” samples were calculated and normalized to 1.

Analyses were performed on the maximum projection image of the raw image stacks. CellProfiler 4.0.7 is used for single-cell protein quantification (FIG. 13A). For single-cell analyses in Hela cells, first, Hoechst-stained nuclei were identified as primary objects. Then, the Hoechst channel, mCherry channel, and GFP channel were merged and subsequently converted to a grayscale image. Cells were identified as secondary objects on this grayscale image. Following cell segmentation, mCherry intensity and GFP intensity in each cell were measured. Finally, the ratio between GFP intensity and mCherry intensity in each cell was calculated. To remove batch effects, the average ratios between GFP intensity and mCherry intensity in all the “mock ligation” samples in different batches were calculated and normalized to 1. The assumption was that the average ratios between GFP intensity and mCherry intensity in all the “mock ligation” samples are the same. Cells that contained similar intensities to those of control samples (transfection reagents only or untransfected cells) were considered unsuccessfully transfected and thus excluded from this analysis.

### Firefly Luciferase Degron Characterization

HeLa cells were transfected with Firefly-60A or Firefly-degron-60A mRNAs, using the aforementioned protocol for GFP mRNA transfection. For luciferase decay measurements, cells were grown for 24 hours, then transferred to media containing 100 ug/mL cycloheximide (CHX) to halt translation. At various timepoints following CHX addition, cells were lysed and luciferase activity was measured using the Promega Dual-Glo Luciferase Assay System (Promega: E2920). For luciferase-degron mocRNA time course, mocRNAs were generated as previously described. 250 ng of Firefly-PEST mocRNAs were co-transfected into Hela cells in a 24 well-plate along with 250 ng of Renilla luciferase mRNA (E-PAP-tailed) as an internal control. Six hours after transfection, cells were reseeded into 4 separate opaque white plates for lysis at varying timepoints, as specified.

For in vitro translation experiments, 100 ng of each Firefly-PEST mocRNA was mixed with 200 ng of Renilla mRNA (E-PAP-tailed) to serve as an internal control. These were denatured at 65° C. for 5 min, placed on ice, and added to serve as templates for a 50 μl rabbit reticulocyte lysate reaction (Promega: L4960), assembled and incubated according to the manufacturer's protocol. Following a 1.5 hr incubation, 2 μl of each reaction was diluted in 20 μl 1×PBS and measured using the Promega Dual-Glo assay. Three technical replicates were taken for each of three biological replicates for each condition tested.

RNA Isolation and cDNA Preparation

Hela cells were seeded to ˜75% confluency on 12-well plastic plates and transfected with mRNA using the protocol described earlier. For the preparation of positive controls, either 200 ng poly(I:C), 500 ng poly(I:C) (InvivoGen: tlrl-picw), or 500 ng unmodified GFP mRNA (containing 100% replacement of N1-methylpseudouridine with uridine, and was E-PAP poly(A) tailed using 100% rATP) was transfected into cells using 3 μl Lipofectamine MessengerMax (Thermo Fisher Scientific). Following transfection and cell reseeding, cells were collected at 48 hours post-transfection, media was removed, and 350 μl Trizol was pipetted into each well for RNA storage at −80° C. Unmodified GFP mRNA was prepared from the pCS2_hMGFP template, which did not contain a 60A template-encoded tail. Unmodified GFP mRNA contained 100% UTP instead of N1-methylpseudouridine, and it was poly(A) tailed using E-PAP tailing.

Total RNA was extracted from Trizol-stored samples using Direct-zol RNA Miniprep Kit (Zymo Research: R2051) according to the manufacturer's protocol. The optional DNase digestion was performed, also according to the manufacturer's protocol. Isolated RNA was then concentrated using RNA Clean & Concentrator-5 (Zymo Research: R1013) and eluted in nuclease-free water containing 1.100 Superase-In. RNA was then quantified using a Nanodrop prior to storage at −80° C.

Reverse transcription of total RNA was performed using SuperScript IV Reverse Transcriptase (ThermoFisher Scientific: 18090200). 500 ng of total RNA was mixed with 1 μl of Random Primer Mix (NEB: S1330S) and brought up to a total volume of 13 μl. This mixture was heated at 65° C.′ for 5 min, then immediately placed on ice during the next step of reaction assembly. The following reagents and volumes were then added to the 13 μl annealed mixture: 4 ul of 5×SSIV reaction buffer; 1 μl (0.5 mM final) of 10 mM dNTP mix (ThermoFisher Scientific: 18427013); 1 ul of 100 mM DTT; 0.5 μl of Superase RNase-In; and 1 μl of SuperScript IV RT enzyme (200 U/μl).

Reactions were mixed, then incubated at 23° C. for 10 min., followed by incubation at 50° C. for 10 min., and terminated by incubation at 80° C. for 10 min. A portion of select cDNA reactions were saved to be used as standards for the calibration/dilution curve. However, for all samples to be quantified by RT-qPCR, 5× dilutions from these cDNA reactions were prepared by the addition of nuclease free water and stored at −80° C. prior to use.

### RT-qPCR

RT-qPCR was performed in clear LightCycler 384-well plates (Roche: 04729749001), using Power SYBR Green PCR Master Mix (ThermoFisher Scientific: 4367659). Each reaction contained 1 μl of cDNA template (previously diluted 5×); 500 nM each (final concentration) of the forward and reverse primers (see Table 5 for sequences); and 10 ul of 2× Power SYBR Green Master Mix. Reaction total volumes were brought up to 20 μl total prior to processing on a Bio-Rad CFX384 Touch Real-Time PCR Detection System. Cycling settings used for hMGFP, mCherry, and hActb were: 95° C. for 10 min. (×1); 95° C. for 10 sec., 60° C. for 30 sec., [Plate Read] (×40), followed by melt curve analysis (65.0° C. to 95.0° C., increment 0.5° C.+[Plate Read]) For IFNB1 qPCR, cycling settings used were: 95° C. for 10 min. (×1); 95° C. for 10 sec., 57° C. for 15 sec, 60° C. for 30 sec., [Plate Read] (×40), followed by melt curve analysis (65.0° C. to 95.0° C., increment 0.5° C.+[Plate Read]).

Relative mRNA quantities were calculated using the relative quantification method, which requires a standard curve. “Positive control” samples were selected as standards and a 2-fold dilution series was performed to produce standard curves from which to calculate reaction efficiencies (E) for each measured gene (using linear fitting on a log-log scale). For GFP & mCherry quantification, a cDNA stock solution was selected corresponding to one of the biological replicates of unligated GFP-60 mRNA+mCherry transfections as the standard. For IFNB1 quantification, one of the biological replicates for the 500 ng poly(I:C) transfection condition was used as the standard. For hActb quantification, cDNA from one of the “transfection conditions only” samples was used as the standard. To ensure all cDNA measurements of unknown samples would be within range of linearity determined by the standard curves, all cDNA stocks were diluted 5× (as mentioned previously) prior to being measured by RT-qPCR.

Following linear fitting of the standard curves (3× technical replicates for each dilution), PCR reaction efficiencies were calculated (GFP: 2.05; mCherry: 2.24; IFNB1: 2.11; hActb: 2.09). 3 technical replicates were performed for each cDNA sample to be tested, and technical replicate Cq values were averaged to obtain a value corresponding to each biological replicate. To perform normalization to a specific sample (e.g., “mock ligation”), the biological replicates' Cq values for normalization standard were averaged to give a “standard Cq”. Then, each test sample's Cq values were subtracted from this “standard Cq” to give a dCq value. Reaction efficiencies (E) were raised to the power of these dCq values to give individual “fold changes” for each biological test sample. To normalize GFP by both mCherry & hActb, the geometric mean was taken of mCherry & hActb “fold changes” that were calculated previously. The GFP “fold changes” were then divided by these normalization factors to produce the final values used for quantification of GFP (FIG. 8C, Table 7). For the normalization of IFNB1, hActb values for each sample were used directly (without the geometric mean calculation) (Table 7). Datapoints shown in each graph correspond to the averages of three technical replicates performed for every biologically replicate. Negative controls (e.g., N.T.C. and transfection only) for specific conditions were omitted from calculations, when they did not produce a Cq value.

mRNA Quantification in Transfected Cell Culture Using STARmap

mCherry and GFP mRNA quantities were measured in transfected cells using STARmap²⁸, an imaging-based method that reads out individual mRNA molecules as a barcoded DNA colony. The STARmap procedure for cell cultures was followed as published²⁸. Briefly, following fluorescent protein imaging, the cells were fixed with 1.6% PFA PFA (Electron Microscope Sciences, 15710-S)/1XPBS (Gibco, 10010-023) at room temperature for 10 min before further fixation and permeabilization with pre-chilled methanol at −20° C. (up to one week) before the next step. Subsequently, the methanol was removed, and the cells were rehydrated with PBSTR/Glycine/YtRNA (PBS with 0.1% Tween-20 [TEKNOVA INC, 100216-360], 0.5% SUPERaseIn [Invitrogen™, AM2696], 100 mM Glycine, 1% Yeast tRNA) at room temperature for 15 min followed by PBSTR wash once. The samples were then hybridized with SNAIL probes targeting mCherry and GFP mRNA sequences in the hybridization buffer (2×SSC [Sigma-Aldrich, S6639], 10% Formamide [Calbiochem, 655206], 1% Tween-20, 20 mM RVC [Ribonucleoside vanadyl complex, New England Biolabs, S1402S], 0.5% SUPERaseIn, 1% Yeast tRNA, 100 nM each probe) at 40° C. overnight (see Table 5 for “SNAIL probe” sequences). The cells were then washed with PBSTR twice at 37° C. (20 min each wash) and High salt wash buffer (PBSTR with 4×SSC) once at 37° C. before rinsing once with PBSTR at room temperature. Ligation reaction was performed for 2 hr at room temperature to circularize padlock probes that were adjacent to a primer. After two washes with PBSTR, rolling circle amplification was initiated from the primer using Phi29 (ThermoFisher, EP0094) at 30° C. for 2 hr with amino-dUTP (Invitrogen™, AM8439) spiked in. After two more washes with PBSTR, the DNA amplicons were modified to be polymerizable by 20 mM MA-NHS (Sigma-Aldrich, 730300-1G) in PBST buffer at room temperature for 2 hr. The samples were then converted into a hydrogel-cell hybrid before Proteinase K (Invitrogen™, 25530049) clearing of fluorescent proteins at room temperature overnight. The samples were washed three times with PBST before being stained with fluorescent detection oligo in the wash and imaging buffer (2×SSC, 10% formamide) at 37° C. for 1 hr (see Table 5 for “fluorescent detection probe” sequences). Finally, the samples were washed three times with the wash and imaging buffer at room temperature and stained with DAPI before imaging in the wash and imaging buffer. Confocal imaging stacks were taken by Leica Stellaris 8 or SP8 with a 40× oil objective at the pixel size of 283 nm*283 nm. A 14-μm stack is imaged with 1 μm/step*15 steps. Four representative fields of view are taken for each well, one from each quadrant. The same imaging setting was used for all the samples to be compared. Excitation/detection wavelengths are as the following: DAPI: Diode 405 nm/˜[420-489] nm; Alexa546: WLL 557 nm/˜[569-612] nm; Alexa647: WLL 653 nm/˜[668-738] nm.

MATLAB 2021a and CellProfiler 4.0.7 were used for the amplicon count-based STARmap fluorescence image analysis (FIG. 8C). First, the centroids of amplicons in each fluorescent channel (GFP, mCherry) were identified by finding extended maxima on images. Then a 3*3*3 voxel volume centering the centroid of each fluorescent dot was defined. Within each voxel volume, the integrated intensities in the mCherry and GFP channels were calculated, and the ratio between mCherry intensity and GFP intensity was used for amplicon classification. After these measurements had been performed on all the images in a batch, all the measurements were pooled together, and the distribution of log(mCherry/GFP) values were plotted. The corresponding ratio values at the nadirs (local minimum) on the distribution plot were identified as cutoff values. The first cutoff value less than 0 was noted as cutoff1, and the first cutoff value greater than 0 was noted as cutoff2. Any amplicon with a log(mCherry/GFP) value smaller than cutoff1 were identified as a GFP amplicon. Any amplicon with a log(mCherry/GFP) value larger than cutoff2 were identified as a mCherry amplicon. Any amplicon with a log(mCherry/GFP) value between cutoff1 and cutoff2 were identified as a granule. Amplicon classification information, as well as the location of every amplicon, was saved in a file. In bulk STARmap quantification, in each figure, the ratio between the number of GFP amplicons and the number of mCherry amplicons were calculated and used to reflect the amount of GFP mRNAs. In single-cell STARmap quantification, cell segmentation was performed using the same method as cell segmentation in single-cell protein quantification, and the segmentation masks were saved as uint16 images Amplicons were the assigned to cells according to where they were located on the masks. The ratio between the number of GFP amplicons and the number of mCherry amplicons in each cell was calculated and used to reflect the amount of GFP mRNAs in a single cell. Cells with no GFP amplicons or no mCherry amplicons were considered unsuccessfully transfected and thus excluded from these analyses.

### REFERENCES

1. Polack, F. P., Thomas, S. J., Kitchin, N., Absalon, J., Gurtman, A., Lockhart, S., Perez, J. L., Pérez, M. G., Moreira, E. D., Zerbini, C., et al. (2020) Safety and Efficacy of the BNT162b2 mRNA Covid-19 Vaccine. N. Engl. J. Med. 383, 2603-2615.
2. Lombardi, A., Bozzi, G., Ungaro, R., Villa, S., Castelli, V., Mangioni, D., Muscatello, A., Gori, A., and Bandera, A. (2021) Mini Review Immunological Consequences of Immunization With COVID-19 mRNA Vaccines: Preliminary Results. Front. Immunol. 12, 657711.
3. Sahin, U., Karikó, K., and Türeci, Ö. (2014) mRNA-based therapeutics—developing a new class of drugs. Nat. Rev. Drug Discov. 13, 759-780.
4. Dammes, N., and Peer, D. Paving the Road for RNA Therapeutics. (2020) Trends Pharmacol. Sci. 41, 755-775
5. Weng, Y., Li, C., Yang, T., Hu, B., Zhang, M., Guo, S., Xiao, H., Liang, X. J. and Huang, Y. (2020) The challenge and prospect of mRNA therapeutics landscape. Biotechnol. Adv. 40, 107534.
6. Petsch, B., Schnee, M., Vogel, A. B., Lange, E., Hoffmann, B., Voss, D., Schlake, T., Thess, A., Kallen, K. J., Stitz, L., et al. (2012) Protective efficacy of in vitro synthesized, specific mRNA vaccines against influenza A virus infection. Nat. Biotechnol. 30, 1210-1216.
7. Richner, J. M., Jagger, B. W., Shan, C., Fontes, C. R., Dowd, K. A., Cao, B., Himansu, S., Caine, E. A., Nunes, B. T. D., Medeiros, D. B. A., et al. (2017) Vaccine Mediated Protection Against Zika Virus-Induced Congenital Disease. Cell 170, 273-283.e12.
8. Tai, W., Zhang, X., Drelich, A., Shi, J., Hsu, J. C., Luchsinger, L., Hillyer, C. D., Tseng, C. T. K., Jiang, S. and Du, L. (2020) A novel receptor-binding domain (RBD)-based mRNA vaccine against SARS-COV-2. Cell Res. 30, 932-935.
9. Chen, C., Ezzeddine, N., and Shyu, A. (2008) Messenger RNA half-life measurements in mammalian cells. Methods Enzymol. 448, 335-357.
10. Karikó, K., Muramatsu, H., Welsh, F. A., Ludwig, J., Kato, H., Akira, S. and Weissman, D. (2008) Incorporation of pseudouridine into mRNA yields superior nonimmunogenic vector with increased translational capacity and biological stability. Mol. Ther. 16, 1833-1840.
11. Kariko, K., Buckstein, M., Ni, H., and Weissman, D. Suppression of RNA recognition by Toll-like receptors: the impact of nucleoside modification and the evolutionary origin of RNA. (2005) Immunity 23, 165-175.
12. Andries, O., McCafferty, S., De Smedt, S. C., Weiss, R., Sanders, N. N. and Kitada, T. (2015) N1-methylpseudouridine-incorporated mRNA outperforms pseudouridine-incorporated mRNA by providing enhanced protein expression and reduced immunogenicity in mammalian cell lines and mice. J. Control. Release 217, 337-344.
13. Kormann, M. S., Hasenpusch, G., Aneja, M. K., Nica, G., Flemmer, A W., Herber-Jonat, S., Huppmann, M., Mays, L. E., Illenyi, M, Schams, A., et al. (2011) Expression of therapeutic proteins after delivery of chemically modified mRNA in mice. Nature Biotechnology 29, 154-157.
14. Li, B., Luo, X., and Dong, Y. (2016) Effects of Chemically Modified Messenger RNA on Protein Expression. Bioconjug. Chem. 27, 849-853.
15. Aurup, H., Siebert, A., Benseler, F., Williams, D., and Eckstein, F. (1994) Translation of 2′-modified mRNA in vitro and in vivo. Nucleic Acids Res. 22, 4963-4968.
16. Choi, J., Indrisiunaite, G., DeMirci, H., Ieong, K. W., Wang, J., Petrov, A., Prabhakar, A., Rechavi, G., Dominissini, D., He, C., et al. (2018) 2′-O-methylation in mRNA disrupts tRNA decoding during translation elongation. Nat. Struct. Mol. Biol. 25, 208-216.
17. Anhäuser, L., Hüwel, S., Zobel, T., and Rentmeister, A. (2019) Multiple covalent fluorescence labeling of eukaryotic mRNA at the poly(A) tail enhances translation and can be performed in living cells. Nucleic Acids Res. 47, e42.
18. Strzelecka, D., Smietanski, M., Sikorski, P. J., Warminski, M., Kowalska, J. and Jemielity, J. (2020) Phosphodiester modifications in mRNA poly(A) tail prevent deadenylation without compromising protein expression. RNA 26, 1815-1837.
19. Łabno, A., Tomecki, R., and Dziembowski, A. (2016) Cytoplasmic RNA decay pathways—Enzymes and mechanisms. Biochim. Biophys. Acta 1863, 3125-3147.
20. Garneau, N. L., Wilusz, J., and Wilusz, C. J. (2007) The highways and byways of mRNA decay. Nature Reviews Molecular Cell Biology 8, 113-126.
21. Eckstein, F. (2014) Phosphorothioates, essential components of therapeutic oligonucleotides Nucleic Acid Ther. 24, 374-387.
22. Stowell, J. A., Webster, M. W., Kögel, A., Wolf, J., Shelley, K. L., and Passmore, L. A. (2016) Reconstitution of Targeted Deadenylation by the Cer4-Not Complex and the YTH Domain Protein Mmil. Cell Rep. 17, 1978-1989.
23. Yang, Y, Lindahl, T., and Barnes, D. (2007) Trex1 exonuclease degrades ssDNA to prevent chronic checkpoint activation and autoimmune disease. Cell 131, 873-886.
24. Takahashi, A., Loo, T. M., Okada, R., Kamachi, F., Watanabe, Y., Wakita, M., Watanabe, S., Kawamoto, S., Miyata, K., Barber, G. N., et al. (2018) Downregulation of cytoplasmic DNases is implicated in cytoplasmic DNA accumulation and SASP in senescent cells. Nat. Commun. 9, 1249.
25. Chen, J., Chiang, Y., and Denis, C. (2002) CCR4, a 3′-5′ poly(A) RNA and ssDNA exonuclease, is the catalytic component of the cytoplasmic deadenylase. EMBO J. 21, 1414-1426.
26. Cerritelli, S and Crouch, R. Ribonuclease H: the enzymes in eukaryotes. (2009) FEBS J. 276, 1494-1505.
27. Leonhardt, C., Schwake, G., Stögbauer, T. R., Rappl, S., Kuhr, J. T., Ligon, T. S, and Rädler, J. O. (2014) Single-cell mRNA transfection studies: delivery, kinetics and statistics by numbers. Nanomedicine: Nanotechnology, Biology and Medicine 10, 679-688.
28. Wang, X., Allen, W., Wright, M., Sylwestrak, E., Samusik, N., Vesuna, S., Evans, K., Liu, C., Ramakrishnan, C., Liu, J., et al. (2018) Three-dimensional intact-tissue sequencing of single-cell transcriptional states. Science 361, eaat5691.
29. Li, X., Zhao, X., Fang, Y., Jiang, X., Duong, T., Fan, C., Huang, C. C., and Kain, S. R. (1998) Generation of destabilized green fluorescent protein as a transcription reporter. Journal of Biological Chemistry 273.52, 34970-34975.
30. Lin, C. Y., Perche, F., Ikegami, M., Uchida, S., Kataoka, K., and Itaka, K. (2016) Messenger RNA-based therapeutics for brain diseases: An animal study for augmenting clearance of beta-amyloid by intracerebral administration of neprilysin mRNA loaded in polyplex nanomicelles. Journal of Controlled Release 235, 268-275.
31. Williams, D. J., Puhl, H. L., and Ikeda, S. R. (2010) A simple, highly efficient method for heterologous expression in mammalian primary neurons using cationic lipid-mediated mRNA transfection. Frontiers in Neuroscience 4, 181.
32. Ingusci, S., Verlengia, G., Soukupova, M., Zucchini, S., and Simonato, M. (2019) Gene therapy tools for brain diseases. Frontiers in Pharmacology 10, 724.
33 Honda, K., Yanai, H., Takaoka, A., and Taniguchi, T. (2005) Regulation of the type I IFN induction: a current view. International Immunology 17, 1367-1378.
34. Wienert, B., Shin, J., Zelin, E., Pestal, K., and Corn, J. E. (2018) In vitro-transcribed guide RNAs trigger an innate immune response via the RIG-I pathway. PLoS biology 16, e2005840.
35. Linehan, M. M., Dickey, T. H., Molinari, E. S., Fitzgerald, M. E., Potapova, O., Iwasaki, A., and Pyle, A. M. (2018) A minimal RNA ligand for potent RIG-I activation in living mice. Science Advances 4, e1701854.
36. Hausmann, S., Marq, J. B., Tapparel, C., Kolakofsky, D., and Garcin, D. (2008) RIG-I and dsRNA-induced IFNß activation PLoS One 3, e3965.
37. Cao Z., Huang, C. C., and Tan, W. (2006) Nuclease resistance of telomere-like oligonucleotides monitored in live cells by fluorescence anisotropy imaging. Analytical Chemistry 78, 1478-1484.
38. Barragán-Iglesias, P., Lou, T. F., Bhat, V. D., Megat, S., Burton, M. D., Price, T. J., and Campbell, Z. T. (2018) Inhibition of Poly (A)-binding protein with a synthetic RNA mimic reduces pain sensitization in mice. Nature Communications 9, 1-17.
39. Rydzik, A. M., Warminski, M., Sikorski, P. J., Baranowski, M. R., Walczak, S., Kowalska, J., Zuberek, J., Lukaszewicz, M., Nowak, E., Claridge, T. D., et al. (2017) mRNA cap analogues substituted in the tetraphosphate chain with CX2: identification of O-to-CC12 as the first bridging modification that confers resistance to decapping without impairing translation. Nucleic Acids Res. 45, 8661-8675.
40. Strenkowska, M., Kowalska, J., Lukaszewicz, M., Zuberek, J., Su, W., Rhoads, R. E., Darzynkiewicz, E., and Jemielity, J. (2010) Towards mRNA with superior translational activity: synthesis and properties of ARCA tetraphosphates with single phosphorothioate modifications. New J. Chem. 34, 993-1007.
41. Kawaguchi, D., Kodama, A., Abe, N., Takebuchi, K., Hashiya, F., Tomoike, F., Nakamoto, K., Kimura, Y, Shimizu, Y., and Abe, H. (2020) Phosphorothioate Modification of mRNA Accelerates the Rate of Translation Initiation to Provide More Efficient Protein Synthesis. Angew. Chem. Int. Ed Engl. 59, 17403-17407.
42. Boo, S. and Kim, Y. (2020) The emerging role of RNA modifications in the regulation of mRNA stability. Experimental & molecular medicine 52, 400-408.
43 Zhao, B. S., Wang, X., Beadell, A. V., Lu, Z., Shi, H., Kuuspalu, A., Ho, R. K., and He, C. (2017) m6A-dependent maternal mRNA clearance facilitates zebrafish maternal-to-zygotic transition. Nature 542, 475-478.
44. Carpenter, A. E., Jones, T. R., Lamprecht, M. R., Clarke, C., Kang, I. H., Friman, O., Guertin, D. A., Chang, J. H., Lindquist, R. A., Moffat, J., et al. (2006) CellProfiler: image analysis software for identifying and quantifying cell phenotypes. Genome biology 7, 1-11.

### Example 6: Chemically Modified mocRNAs Provide Efficient Protein Expression In Vivo

Messenger-oligonucleotide conjugated RNA (mocRNA), which are therapeutic mRNA ligated to chemically modified oligonucleotides, are described. The therapeutic mRNA contains (from 5′ to 3′): (1) an mRNA cap analog (NEB: S1411); (2) a 5′ untranslated region (UTR); (3) protein-coding region (luciferase reporter); (4) 3′ UTR; and (5) poly(A) tail (20 to 200 nt). The mRNA contains a 100% substitution of uridine with N1-methylpseudouridine (Trilink Biotechnologies: N1081) to increase expression.

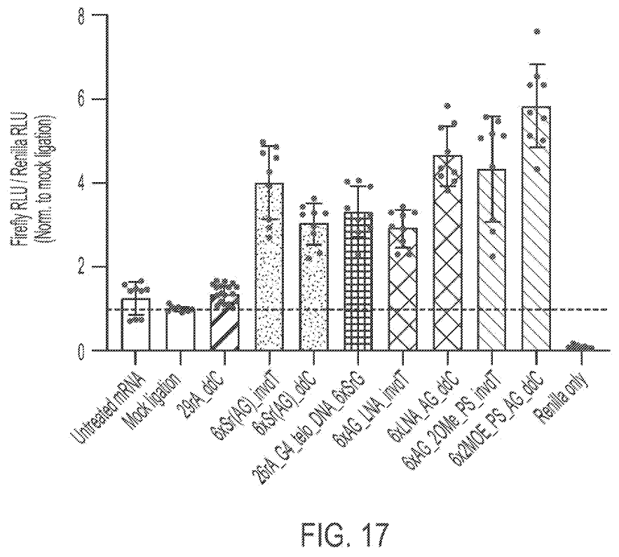

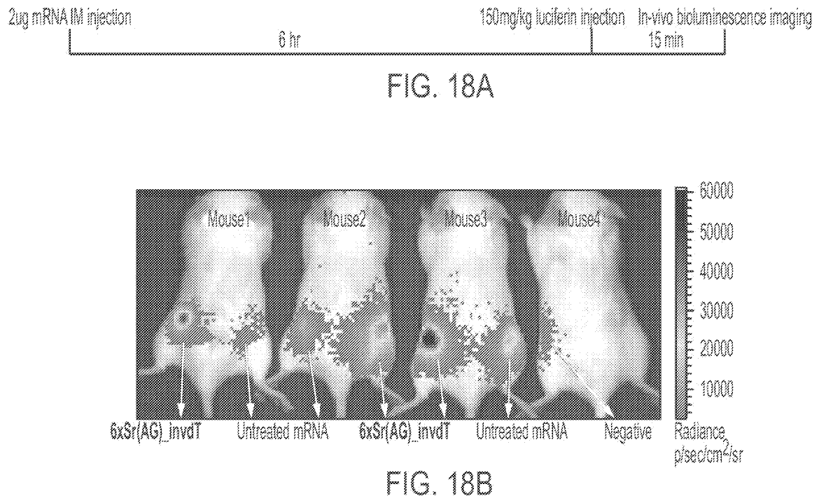

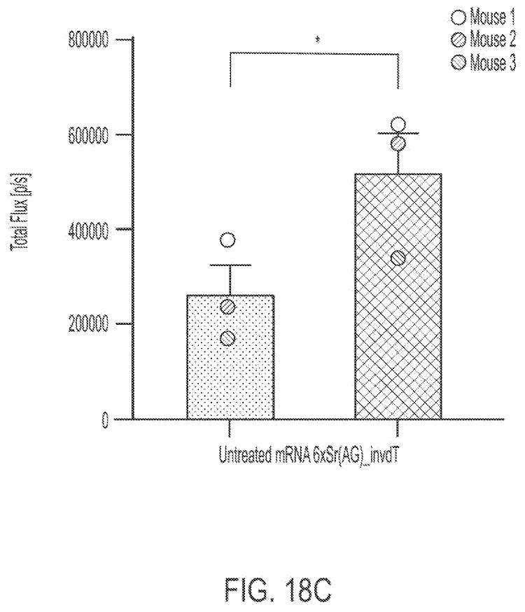

mocRNA are synthesized by ligating chemically-synthesized oligonucleotides (Table 9) to the 3′ end of therapeutic mRNA. Oligonucleotides containing nuclease-resistant groups protect the poly(A) tail from deadenylation and increase expression at longer timepoints in HeLa cell culture (FIG. 17). Furthermore, mocRNA injection into mice increases expression of a luciferase reporter compared to an untreated mRNA (FIGS. 18A-18C).

These oligonucleotides may similarly be ligated to the 3′ end of a non-protein coding RNA, in order to enhance the stability of such RNAs in cells.

TABLE 4
Additional sequences of oligonucleotides used for mocRNA syntheses.

| Modified | |
|---|---|
| oligonucleotide | |
| sequence name | Sequence (IDT format) |
| 29rA_ddC | /5Phos/rArArA rArArA rArArA rArArA rArArA rArArA rArArA |
| | rArArA rArArA rArA/3ddC/ (SEQ ID NO: 6) |
| 6xSr(AG)_invdT | /5Phos/rArArA rArArA rArArA rArArA rArArA rArArA rArArA |
| | rArArA* rA ** A*rG* rG*rG*/3InvdT/ (SEQ ID NO: 14) |
| 6xSr(AG)_ddC | /SPhos/rArArA rArArA rArArA rArArA rArArA rArArA rArArA |
| | rArArA* rA*A*rG* G*G*/3ddC/ (SEQ ID NO: 48) |
| 26rA_G4_telo_DNA_ | /5Phos/rArArA rArArA rArArA rArArA rArArA rArArA rArArA |
| 6xSrG | rArArA rArAT AGG GTT AGG GTT AGG GT*T* A*G*G* |
| | G*/3InvdT/ (SEQ ID NO: 35) |
| 6xAG_LNA_invdT | /5Phos/rArArA rArArA rArArA rArArA rArArA rArArA rArArA |
| | rArA+A +A+A+G +G+G/3InvdT/ (SEQ ID NO: 49) |
| 6xLNA_AG_ddC | /5Phos/rArArA rArArA rArArA rArArA rArArA rArArA rArArA |
| | rArA+A +A+A+G +G+G/3ddC/ (SEQ ID NO: 50) |
| 6xAG_20Me_PS_ | /5Phos/rArArA rArArA rArArA rArArA rArArA rArAmA mAmAmA |
| invdT | mAmAmA* mA*mA*mG* mG*mG*/3InvdT/ (SEQ ID NO: 51) |
| 6x2MOE_PS_AG_ddC | /SPhos/rArArA rArArA rArArA rArArA rArArA rArArA rArArA |
| | rArA/i2MOErA/*/i2MOErA/*/i2MOErA/*/i2MOErG/*/ |
| | i2MOErG/*/i2MOErG/*/3ddC/ (SEQ ID NO: 52) |

RNA bases: rN ; RNA phosphorothioate bases: rN* ; DNA phosphorothioate bases: N*; 2′-O-methyl phosphorothioate bases: mN*; locked nucleic acid [LNA] bases: +N; Internal 2′-O-methoxy-ethyl RNA bases: /i2MOErN/; 5′ Phosphate modification: /5Phos/; 2′-3′-dideoxycytidine [ddC] modification: /3ddC/; Inverted-2′-deoxythymidine [InvdT] modification: /3InvdT/.

### Equivalents and Scope

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. A modified mRNA comprising:
(i) an open reading frame (ORF) encoding a protein; and
(ii) a poly-A region,
wherein the poly-A region is 3′ to the open reading frame and comprises 10 or more nucleotides, wherein 1% to 90% of the nucleotides of the poly-A region are modified nucleotides, and wherein 3 or more of the 10 last nucleotides of the poly-A region are modified nucleotides.

2. The modified mRNA of claim 1, wherein the poly-A region is 3′ to the open reading frame and comprises 25 or more adenosine nucleotides, wherein 1% to 90% of the nucleotides of the poly-A region are modified nucleotides, and wherein 3 or more of the 25 last nucleotides of the poly-A region are modified nucleotides.

3. The modified mRNA of claim 1 or claim 2, wherein 4 or more of the 25 last nucleotides of the poly-A region are modified nucleotides.

4. The modified mRNA of any one of claims 1-3, wherein 2 or more consecutive nucleotides of the 25 last nucleotides of the poly-A region are linked by a modified internucleotide linkage.

5. The modified mRNA of any one of claims 1-4, wherein 3 or more consecutive nucleotides of the 25 last nucleotides of the poly-A region are modified nucleotides independently selected from a deoxyribonucleotide, a 2′-modified nucleotide, and a phosphorothioate-linked nucleotide.

6. The modified mRNA of any one of claims 1-5, wherein the 3 or more modified nucleotides are consecutive nucleotides located at the 3′ terminus of the poly-A region.

7. The modified mRNA of any one of claims 1-6, wherein 6 or more consecutive nucleotides of the 25 last nucleotides of the poly-A region comprise the same type of nucleotide or internucleoside modification.

8. The modified mRNA of any one of claims 1-7, wherein at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the poly-A region are modified nucleotides.

9. The modified mRNA of any one of claims 1-8, wherein at least 4, 5, 6, 7, 8, 9, 10, 15, 20, or 25 of the 25 last nucleotides of the poly-A region are modified nucleotides.

10. The modified mRNA of any one of claims 1-9, wherein the modified mRNA comprises a 5′ untranslated region (5′ UTR) and a 3′ untranslated region (3′ UTR), wherein the ORF is between the 5′ UTR and the 3′ UTR, wherein the 3′ UTR is between the ORF and the poly-A region.

11. The modified mRNA of any one of claims 1-10, wherein the modified mRNA is a circular mRNA, wherein the poly-A region is between the 3′ UTR and the 5′ UTR.

12. A modified mRNA comprising:
(i) an open reading frame (ORF) encoding a protein;
(ii) a poly-A region;
(iii) one or more copies of a structural sequence comprising at least two nucleotides that are capable of forming a secondary structure,
wherein the poly-A region is 3′ to the open reading frame and comprises 10 or more nucleotides,
wherein the one or more copies of the structural sequence are 3′ to the poly-A region, and wherein the modified mRNA comprises a secondary structure, wherein the secondary structure comprises one or more copies of the structural sequence.

13. The modified mRNA of claim 12, wherein the poly-A region comprises 25 or more adenosine nucleotides.

14. The modified mRNA of claim 12 or claim 13, wherein the modified mRNA comprises a 5′ untranslated region (5′ UTR) and a 3′ untranslated region (3′ UTR), wherein the ORF is between the 5′ UTR and the 3′ UTR, wherein the 3′ UTR is between the ORF and the poly-A region.

15. The modified mRNA of claim 14, wherein the modified mRNA is a circular mRNA, wherein the one or more copies of the structural sequence are between the poly-A region and the 5′ UTR.

16. The modified mRNA of any one of claims 12-15, wherein the structural sequence is a G-quadruplex sequence.

17. The modified mRNA of claim 16, wherein the G-quadruplex is an RNA G-quadruplex sequence.

18. The modified mRNA of claim 17, wherein the RNA G-quadruplex sequence comprises the nucleic acid sequence of SEQ ID NO: 2.

19. The modified mRNA of claim 18, wherein the modified mRNA comprises at least 3 copies of the nucleic acid sequence of SEQ ID NO: 2.

20. The modified mRNA of claim 16, wherein the G-quadruplex is a DNA G-quadruplex sequence.

21. The modified mRNA of claim 20, wherein the DNA G-quadruplex sequence comprises the nucleic acid sequence of SEQ ID NO: 3.

22. The modified mRNA of claim 21, wherein the modified mRNA comprises at least 3 copies of the nucleic acid sequence of SEQ ID NO: 3.

23. The modified mRNA of any one of claims 12-15, wherein the structural sequence is a telomeric repeat sequence.

24. The modified mRNA of claim 23, wherein the telomeric repeat sequence comprises the nucleic acid sequence of SEQ ID NO: 4.

25. The modified mRNA of claim 24, wherein the modified mRNA comprises at least 3 copies of the nucleic acid sequence of SEQ ID NO: 4.

26. The modified mRNA of any one of claims 12-15, wherein the secondary structure of the mRNA is an aptamer that is capable of binding to a target molecule.

27. The modified mRNA of any one of claims 12-26, wherein the poly-A region of the modified mRNA comprises at least one modified nucleotide.

28. The modified mRNA of any one of claim 1-11 or 27, wherein at least one modified nucleotide comprises a modified nucleobase.

29. The modified mRNA of claim 28, wherein the modified nucleobase is selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

30. The modified mRNA of claim 28 or 29, wherein at least one modified nucleotide comprises a modified sugar.

31. The modified mRNA of claim 30, wherein the modified sugar is selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, S′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose.

32. The modified mRNA of any one of claims 27-31, wherein at least one modified nucleotide comprises a 2′ modification.

33. The modified mRNA of claim 32, wherein the 2′ modification is selected from the group consisting of a locked-nucleic acid (LNA) modification, 2′-fluoro (2′-F), 2′-O-methoxy-ethyl (2′-MOE), and 2′-O-methylation (2′-OMe).

34. The modified mRNA of any one of claims 27-33, wherein at least one modified nucleotide comprises a modified phosphate.

35. The modified mRNA of claim 34, wherein the modified phosphate is selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

36. The modified mRNA of any one of claims 27-35, wherein the poly-A region comprises at least 3, at least 4, at least 5, or at least 6 phosphorothioates.

37. The modified mRNA of claim 36, wherein the poly-A region comprises at least 6 phosphorothioates.

38. The modified mRNA of any one of claims 27-35, wherein the poly-A region comprises at least 3 guanine nucleotides and least 3 phosphorothioates.

39. The modified mRNA of any one of claims 27-38, wherein the poly-A region comprises at least 6 nucleotides comprising a 2′ modification.

40. The modified mRNA of any one of claims 27-39, wherein the poly-A region comprises at least 3 deoxyribose sugars.

41. The modified mRNA of claim 40, wherein the poly-A region comprises at least 5, at least 10, at least 15, at least 20, or at least 23 deoxyribose sugars.

42. The modified mRNA of claim 41, wherein the poly-A region comprises at least 23 deoxyribose sugars.

43. The modified mRNA of any one of claims 1-42, wherein the 3′ terminal nucleotide of the mRNA does not comprise hydroxy at the 3′ position of the 3′ terminal nucleotide.

44. The modified mRNA of any one of claims 1-43, wherein the 3′ terminal nucleotide of the mRNA comprises an inverted nucleotide.

45. The modified mRNA of any one of claims 1-44, wherein the 3′ terminal nucleotide of the mRNA comprises a dideoxyadenosine, dideoxycytidine, dideoxyguanosine, dideoxythymidine, dideoxyuridine, or inverted-deoxythymidine.

46. The modified mRNA of claim 45, wherein the 3′ terminal nucleotide of the mRNA comprises a dideoxycytidine.

47. The modified mRNA of any one of claims 1-46, wherein the mRNA comprises a peptide-binding sequence.

48. The modified mRNA of claim 47, wherein the peptide-binding sequence is a poly-A binding protein (PABP)-binding sequence.

49. The modified mRNA of any one of claim 1-11 or 27-48, wherein the modified mRNA comprises a first modified nucleotide and a second modified nucleotide, wherein the first and second modified nucleosides comprise different structures.

50. The modified mRNA of any one of claims 1-46, wherein the poly-A region comprises at least 25-500 nucleotides.

51. The modified mRNA of claim 50, wherein the poly-A region comprises at least 50, at least 100, at least 150, or at least 200 nucleotides.

52. The modified mRNA of any one of claims 1-51, wherein at least 25%, at least 30%, at least 40%, at least 50%, at least 60%, at least 70%, at least 80%, at least 90%, at least 95%, at least 96%, at least 97%, at least 98%, or at least 99% of nucleotides of the poly-A region are adenosine nucleotides.

53. The modified mRNA of any one of claim 1-10, 12, 14, or 16-52, wherein the modified mRNA is a linear mRNA, wherein the linear mRNA comprises a 5′ cap.

54. The modified mRNA of claim 53, wherein the 5′ cap comprises a 7-methylguanosine.

55. The modified mRNA of claim 54, wherein the 5′ cap further comprises one or more phosphates connecting the 7-methylguanosine to an adjacent nucleotide of the modified mRNA.

56. The modified mRNA of claim 53, wherein the 5′ cap comprises a 3′-O-Me-m7G(5′)ppp(5′)G.

57. The modified mRNA of claim 55 or 56, wherein one or more phosphates of the 5′ cap is a modified phosphate selected from the group consisting of phosphorothioate, triazole ring, dihalogenmethylenebisphosphonate, imidodiphosphate, and methylenebis(phosphonate).

58. The modified mRNA of 1-57, wherein the modified mRNA comprises a 5′ UTR comprising 1 or more modified nucleotides.

59. The modified mRNA of 1-58, wherein the modified mRNA comprises an ORF comprising 1 or more modified nucleotides.

60. A modified non-coding RNA comprising:
(i) a non-coding RNA sequence; and
(ii) a poly-A region,
wherein the poly-A region is 3′ to the non-coding RNA sequence and comprises 10 or more nucleotides, wherein 1% to 90% of the nucleotides of the poly-A region are modified nucleotides, and wherein 3 or more of the 10 last nucleotides of the poly-A region are modified nucleotides.

61. The modified non-coding RNA of claim 60, wherein the poly-A region is 3′ to the open reading frame and comprises 25 or more adenosine nucleotides, wherein 1% to 90% of the nucleotides of the poly-A region are modified nucleotides, and wherein 3 or more of the 25 last nucleotides of the poly-A region are modified nucleotides.

62. The modified non-coding RNA of claim 60 or claim 61, wherein 4 or more of the 25 last nucleotides of the poly-A region are modified nucleotides.

63. The modified non-coding RNA of any one of claims 60-62, wherein 2 or more consecutive nucleotides of the 25 last nucleotides of the poly-A region are linked by a modified internucleotide linkage.

64. The modified non-coding RNA of any one of claims 60-63, wherein 3 or more consecutive nucleotides of the 25 last nucleotides of the poly-A region are modified nucleotides independently selected from a deoxyribonucleotide, a 2′-modified nucleotide, and a phosphorothioate-linked nucleotide.

65. The modified non-coding RNA of any one of claims 60-64, wherein the 3 or more modified nucleotides are consecutive nucleotides located at the 3′ terminus of the poly-A region.

66. The modified non-coding RNA of any one of claims 60-65, wherein 6 or more consecutive nucleotides of the 25 last nucleotides of the poly-A region comprise the same type of nucleotide or internucleoside modification.

67. The modified non-coding RNA of any one of claims 60-66, wherein at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the poly-A region are modified nucleotides.

68. The modified non-coding RNA of any one of claims 60-67, wherein at least 4, 5, 6, 7, 8, 9, 10, 15, 20, or 25 of the 25 last nucleotides of the poly-A region are modified nucleotides.

69. The modified non-coding RNA of any one of claims 60-68, wherein the modified non-coding RNA is a circular non-coding RNA, wherein the poly-A region is 5′ to the non-coding RNA sequence.

70. The modified non-coding RNA of any one of claims 60-69, wherein the modified non-coding RNA further comprises one or more copies of a structural sequence comprising at least two nucleotides that are capable of forming a secondary structure,
wherein the one or more copies of the structural sequence are 3′ to the poly-A region, and wherein the modified non-coding RNA comprises a secondary structure, and
wherein the secondary structure comprises one or more copies of the structural sequence.

71. The modified non-coding RNA of claim 70, wherein the modified non-coding RNA is a circular mRNA, wherein the one or more copies of the structural sequence are between the poly-A region and the non-coding RNA sequence.

72. The modified non-coding RNA of claim 70 or 71, wherein the structural sequence is a G-quadruplex sequence.

73. The modified non-coding RNA of claim 72, wherein the G-quadruplex is an RNA G-quadruplex sequence.

74. The modified non-coding RNA of claim 73, wherein the RNA G-quadruplex sequence comprises the nucleic acid sequence of SEQ ID NO: 2.

75. The modified non-coding RNA of claim 74, wherein the modified non-coding RNA comprises at least 3 copies of the nucleic acid sequence of SEQ ID NO: 2.

76. The modified non-coding RNA of claim 72, wherein the G-quadruplex is a DNA G-quadruplex sequence.

77. The modified non-coding RNA of claim 76, wherein the DNA G-quadruplex sequence comprises the nucleic acid sequence of SEQ ID NO: 3.

78. The modified non-coding RNA of claim 77, wherein the modified non-coding RNA comprises at least 3 copies of the nucleic acid sequence of SEQ ID NO: 3.

79. The modified non-coding RNA of claim 70 or 71, wherein the structural sequence is a telomeric repeat sequence.

80. The modified non-coding RNA of claim 79, wherein the telomeric repeat sequence comprises the nucleic acid sequence of SEQ ID NO: 4.

81. The modified non-coding RNA of claim 80, wherein the modified non-coding RNA comprises at least 3 copies of the nucleic acid sequence of SEQ ID NO: 4.

82. The modified non-coding RNA of claim 70 or 71, wherein the secondary structure of the non-coding RNA is an aptamer that is capable of binding to a target molecule.

83. The modified non-coding RNA of any one of claims 60-82, wherein at least one modified nucleotide comprises a modified nucleobase.

84. The modified non-coding RNA of claim 83, wherein the modified nucleobase is selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, S-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (i6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

85. The modified non-coding RNA of any one of 60-84, wherein at least one modified nucleotide comprises a modified sugar.

86. The modified non-coding RNA of claim 85, wherein the modified sugar is selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, S′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose.

87. The modified non-coding RNA of any one of claims 60-86, wherein at least one modified nucleotide comprises a 2′ modification.

88. The modified non-coding RNA of claim 87, wherein the 2′ modification is selected from the group consisting of a locked-nucleic acid (LNA) modification, 2′-fluoro (2′-F), 2′-O-methoxy-ethyl (2′-MOE), and 2′-O-methylation (2′-OMe).

89. The modified non-coding RNA of any one of claims 57-88, wherein at least one modified nucleotide comprises a modified phosphate.

90. The modified non-coding RNA of claim 89, wherein the modified phosphate is selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

91. The modified non-coding RNA of any one of claims 60-90, wherein the poly-A region comprises at least 3, at least 4, at least 5, or at least 6 phosphorothioates.

92. The modified non-coding RNA of claim 91, wherein the poly-A region comprises at least 6 phosphorothioates.

93. The modified non-coding RNA of any one of claims 60-92, wherein the poly-A region comprises at least 3 guanine nucleotides and least 3 phosphorothioates.

94. The modified non-coding RNA of any one of claims 60-93, wherein the poly-A region comprises at least 6 nucleotides comprising a 2′ modification.

95. The modified non-coding RNA of any one of claims 60-94, wherein the poly-A region comprises at least 3 deoxyribose sugars.

96. The modified non-coding RNA of claim 95, wherein the poly-A region comprises at least 5, at least 10, at least 15, at least 20, or at least 23 deoxyribose sugars.

97. The modified non-coding RNA of claim 96, wherein the poly-A region comprises at least 23 deoxyribose sugars.

98. The modified non-coding RNA of any one of claims 60-97, wherein the 3′ terminal nucleotide of the non-coding RNA does not comprise hydroxy at the 3′ position of the 3′ terminal nucleotide.

99. The modified non-coding RNA of any one of claims 60-98, wherein the 3′ terminal nucleotide of the non-coding RNA comprises an inverted nucleotide.

100. The modified non-coding RNA of claim 98 or 99, wherein the 3′ terminal nucleotide of the mRNA comprises a dideoxyadenosine, dideoxycytidine, dideoxyguanosine, dideoxythymidine, dideoxyuridine, or inverted-deoxythymidine.

101. The modified non-coding RNA of claim 100, wherein the 3′ terminal nucleotide of the mRNA comprises a dideoxycytidine.

102. The modified non-coding RNA of any one of claims 60-101, wherein the modified non-coding RNA comprises a first modified nucleotide and a second modified nucleotide, wherein the first and second modified nucleosides comprise different structures.

103. The modified non-coding RNA of any one of claims 60-102, wherein the poly-A region comprises at least 25-500 nucleotides.

104. The modified non-coding RNA of claim 103, wherein the poly-A region comprises at least 50, at least 100, at least 150, or at least 200 nucleotides.

105. The modified non-coding RNA of any one of claims 60-104, wherein at least 25%, at least 30%, at least 40%, at least 50%, at least 60%, at least 70%, at least 80%, at least 90%, at least 95%, at least 96%, at least 97%, at least 98%, or at least 99% of nucleotides of the poly-A region are adenosine nucleotides.

106. A method of producing a modified mRNA of any one of claim 1-11 or 27-59, the method comprising ligating a first RNA comprising an open reading frame encoding a protein to a tailing nucleic acid comprising one or more modified nucleotides, in the presence of an RNA ligase, whereby the RNA ligase forms a covalent bond between the 3′ nucleotide of the RNA and the 5′ nucleotide of the tailing nucleic acid to produce the modified mRNA.

107. The method of claim 106, wherein the modified mRNA comprises a 5′ untranslated region (5′ UTR) and a 3′ untranslated region (3′ UTR), wherein the ORF is between the 5′ UTR and the 3′ UTR, wherein the 3′ UTR is between the ORF and the poly-A region.

108. The method of claim 107, further comprising circularizing the modified mRNA in the presence of a ribozyme, wherein the modified mRNA comprises a 3′ intron and a 5′ intron, wherein the 3′ intron is 5′ to the 5′ UTR, wherein the 5′ intron is 3′ to the poly-A region, whereby the ribozyme forms a covalent bond between a nucleotide that is 3′ to the 3′ intron and a nucleotide that is 5′ to the 5′ intron to produce a circular mRNA that does not comprise the 5′ intron or the 3′ intron, wherein the poly-A region is between the 3′ UTR and the 5′ UTR of the circular mRNA.

109. The method of claim 107, further comprising the steps of:
(i) introducing a 5′ terminal phosphate group onto the first nucleotide of the modified mRNA;
(ii) cleaving one or more 3′ terminal nucleotides of the modified mRNA to produce a modified mRNA with a 3′ terminal hydroxyl group; and
(iii) circularizing the modified mRNA produced in step (ii) in the presence of a circularizing ligase;
whereby the circularizing ligase forms a covalent bond between the 3′ nucleotide of the modified mRNA and the 5′ nucleotide of the modified mRNA to produce a circular modified mRNA, wherein the poly-A region is between the 3′ UTR and the 5′ UTR.

110. A method of producing the modified mRNA of any one of claims 12-59, the method comprising ligating an RNA comprising an open reading frame encoding a protein to a tailing nucleic acid comprising one or more copies of a structural sequence in the presence of an RNA ligase, whereby the ligase forms a covalent bond between the 3′ nucleotide of the RNA and the 5′ nucleotide of the tailing nucleic acid to produce the modified mRNA.

111. The method of claim 110, wherein the modified mRNA comprises a 5′ untranslated region (5′ UTR) and a 3′ untranslated region (3′ UTR), wherein the ORF is between the 5′ UTR and the 3′ UTR, wherein the 3′ UTR is between the ORF and the poly-A region, wherein the poly-A region is between the 3′ UTR and the one or more copies of the structural sequence.

112. The method of claim 111, further comprising circularizing the modified mRNA in the presence of a ribozyme, wherein the modified mRNA comprises a 3′ intron and a 5′ intron, wherein the 3′ intron is 5′ to the 5′ UTR, wherein the 5′ intron is 3′ to the one or more copies of the structural sequence, whereby the ribozyme forms a covalent bond between a nucleotide that is 3′ to the 3′ intron and a nucleotide that is 5′ to the 5′ intron to produce a circular mRNA that does not comprise the 5′ intron or the 3′ intron, wherein the one or more copies of the structural sequence are between the poly-A region and the 5′ UTR of the circular mRNA.

113. The method of claim 111, further comprising the steps of:
(i) introducing a 5′ terminal phosphate group onto the first nucleotide of the modified mRNA;
(ii) cleaving one or more 3′ terminal nucleotides of the modified mRNA to produce a modified mRNA with a 3′ terminal hydroxyl group; and
(iii) circularizing the modified mRNA produced in step (ii) in the presence of a circularizing ligase;
whereby the circularizing ligase forms a covalent bond between the 3′ nucleotide of the modified mRNA and the 5′ nucleotide of the modified mRNA to produce a circular modified mRNA, wherein the one or more copies of the structural sequence are between the 3′ UTR and the 5′ UTR.

114. The method of claim 109 or 113, wherein the modified mRNA is circularized in the presence of a scaffold nucleic acid, wherein the scaffold nucleic acid is a nucleic acid that is capable of hybridizing with the modified mRNA, wherein the modified mRNA forms a circular secondary structure when bound to the scaffold nucleic acid.

115. The method of claim 114, wherein the scaffold nucleic acid comprises:
(a) a first hybridization sequence comprising 5 or more nucleotides, wherein the first hybridization sequence is complementary to at least the first five (5) nucleotides of the modified mRNA; and
(b) a second hybridization sequence comprising 5 or more nucleotides, wherein the second hybridization sequence is complementary to at least the last five (5) nucleotides of the modified mRNA;
wherein at least the first five (5) nucleotides of the modified mRNA hybridize with the first hybridization sequence, and at least the last five (5) nucleotides of the modified mRNA hybridize with the second hybridization sequence.

116. The method of claim 114 or 115, wherein a last nucleotide of the first hybridization sequence and a first nucleotide of the second hybridization sequence are adjacent in the scaffold nucleic acid and not separated by any other nucleotides.

117. The method of claim 109 or 113, wherein the modified mRNA comprises:
(i) a first self-hybridization sequence that is 5′ to the open reading frame;
(ii) a second self-hybridization sequence that is 3′ to the open reading frame;
(iii) a first non-hybridization sequence that is 5′ to the first self-hybridization sequence; and
(iv) a second non-hybridization sequence that is 3′ to the second self-hybridization sequence,
wherein the first and second self-hybridization sequences are capable of hybridizing with each other,
wherein the first and second self-hybridization sequences are not capable of hybridizing with each other.

118. The method of claim 117, wherein hybridization of the first and second self-hybridization sequences forms a secondary structure in which the 5′ terminal nucleotide and the 3′ terminal nucleotide of the modified mRNA are separated by a distance of less than 100 Å.

119. The method of claim 118, wherein the 5′ terminal nucleotide and the 3′ terminal nucleotide are separated by a distance of less than 90 Å, less than 80 Å, less than 70 Å, less than 60 Å, less than 50 Å, less than 40 Å, less than 30 Å, less than 20 Å, or less than 10 Å.

120. The method of any one of claim 109 or 113-119, wherein the circularizing ligase is T4 RNA ligase.

121. The method of any one of claims 110-120, wherein the structural sequence is a G-quadruplex sequence.

122. The method of claim 121, wherein the G-quadruplex is an RNA G-quadruplex sequence.

123. The method of claim 122, wherein the RNA G-quadruplex sequence comprises the nucleic acid sequence of SEQ ID NO: 2.

124. The method of claim 123, wherein the tailing nucleic acid comprises at least 3 copies of the nucleic acid sequence of SEQ ID NO: 2.

125. The method of claim 121, wherein the G-quadruplex is a DNA G-quadruplex sequence.

126. The method of claim 125, wherein the DNA G-quadruplex sequence comprises the nucleic acid sequence of SEQ ID NO: 3.

127. The method of claim 126, wherein the tailing nucleic acid comprises at least 3 copies of the nucleic acid sequence of SEQ ID NO: 3.

128. The method of any one of claims 110-120, wherein the structural sequence is a telomeric repeat sequence.

129. The method of claim 128, wherein the telomeric repeat sequence comprises the nucleic acid sequence of SEQ ID NO: 4.

130. The method of claim 129, wherein the tailing nucleic acid comprises at least 3 copies of the nucleic acid sequence of SEQ ID NO: 4.

131. The method of any one of claims 110-120, wherein the structural sequence is an aptamer sequence comprising at least two nucleotides that are capable of interacting to form an aptamer, wherein the aptamer is a secondary structure that is capable of binding to a target molecule.

132. The method of any one of claims 110-131, wherein the tailing nucleic acid comprises at least one modified nucleotide.

133. The method of any one of claim 106, 107, or 132, wherein the 5′ nucleotide of the RNA does not comprise a 5′ terminal phosphate group;
wherein the 3′ nucleotide of the RNA comprises a 3′ terminal hydroxyl group;
wherein the 5′ nucleotide of the tailing nucleic acid comprises a 5′ terminal phosphate group; and
wherein the 3′ nucleotide of the tailing nucleic acid does not comprise a 3′ terminal hydroxyl group.

134. The method of any one of claim 106, 107, or 132, wherein the 5′ nucleotide of the RNA does not comprise a 5′ terminal hydroxyl group;
wherein the 3′ nucleotide of the RNA comprises a 3′ terminal phosphate group;
wherein the 5′ nucleotide of the tailing nucleic acid comprises a 5′ terminal hydroxyl group;
wherein the 3′ nucleotide of the tailing nucleic acid does not comprise a 3′ terminal phosphate group; and
wherein the RNA ligase is an RtcB ligase.

135. The method of any one of claim 106, 107, or 132-134, wherein at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the tailing nucleic acid are modified nucleotides.

136. The method of any one of claim 106, 107, or 132-135, wherein at least 4, 5, 6, 7, 8, 9, 10, 15, 20, or 25 of the 25 last nucleotides of the tailing nucleic acid are modified nucleotides.

137. The method of any one of claim 106, 107, or 132-136, wherein at least one modified nucleotide comprises a modified nucleobase.

138. The method of claim 137, wherein the modified nucleobase is selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (i6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

139. The method of any one of claim 106, 107, or 132-138, wherein at least one modified nucleotide comprises a modified sugar.

140. The method of claim 139, wherein the modified sugar is selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose.

141. The method of any one of claim 106, 107, or 132-140, wherein at least one modified nucleotide comprises a 2′ modification.

142. The method of claim 141, wherein the 2′ modification is selected from the group consisting of a locked-nucleic acid (LNA) modification, 2′-fluoro (2′-F), 2′-O-methoxy-ethyl (2′-MOE), and 2′-O-methylation (2′-OMe).

143. The method of any one of claim 106, 107, or 132-142, wherein at least one modified nucleotide comprises a modified phosphate.

144. The method of claim 143, wherein the modified phosphate is selected from the group consisting of phosphorothioate (PS), phosphorodithioate, thiophosphate, 5′-O-methylphosphonate, 3′-O-methylphosphonate, S′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

145. The method of any one of claim 106, 107, or 132-144, wherein the tailing nucleic acid comprises at least 3, at least 4, at least 5, or at least 6 phosphorothioates.

146. The method of claim 145, wherein the tailing nucleic acid comprises at least 6 phosphorothioates.

147. The method of any one of claim 106, 107, or 132-145, wherein the tailing nucleic acid comprises at least 3 guanine nucleotides and least 3 phosphorothioates.

148. The method of any one of claim 106, 107, or 132-147, wherein the tailing nucleic acid comprises at least 6 nucleotides comprising a 2′ modification.

149. The method of any one of claim 106, 107, or 132-148, wherein the tailing nucleic acid comprises at least 3 deoxyribose sugars.

150. The method of claim 149, wherein the tailing nucleic acid comprises at least 5, at least 10, at least 15, at least 20, or at least 23 deoxyribose sugars.

151. The method of claim 140, wherein the tailing nucleic acid comprises at least 23 deoxyribose sugars.

152. The method of any one of claim 106, 107, 110, 111 or 132-151, wherein the 3′ terminal nucleotide of the tailing nucleic acid comprises a dideoxyadenosine, dideoxycytidine, dideoxyguanosine, dideoxythymidine, dideoxyuridine, or inverted-deoxythymidine.

153. The method of any one of claims 106-152, wherein the tailing nucleic acid comprises a first modified nucleotide and a second modified nucleotide, wherein the first and second modified nucleotides comprise different structures.

154. The method of any one of claims 106-153, wherein at least 25%, at least 30%, at least 40%, at least 50%, at least 60%, at least 70%, at least 80%, at least 90%, at least 95%, at least 96%, at least 97%, at least 98%, or at least 99% of the poly-A region of the modified mRNA are adenosine nucleotides.

155. The method of any one of claims 106-154, wherein the poly-A region of the modified mRNA comprises at least 25-500 nucleotides.

156. The method of claim 155, wherein the poly-A region of the modified mRNA comprises at least 50, at least 100, at least 150, or at least 200 nucleotides.

157. The method of any one of claim 106, 107, 110, 111, or 121-156, wherein the modified mRNA is a linear mRNA, wherein the linear mRNA comprises a 5′ cap.

158. The modified mRNA of claim 157, wherein the 5′ cap comprises a 7-methylguanosine.

159. The modified mRNA of claim 158, wherein the 5′ cap further comprises one or more phosphates connecting the 7-methylguanosine to an adjacent nucleotide of the modified mRNA.

160. The modified mRNA of claim 157, wherein the 5′ cap comprises a 3′-O-Me-m7G(5′)ppp(5′)G.

161. The modified mRNA of claim 159 or 160, wherein one or more phosphates of the 5′ cap is a modified phosphate selected from the group consisting of phosphorothioate, triazole ring, dihalogenmethylenebisphosphonate, imidodiphosphate, and methylenebis(phosphonate).

162. The method of any one of claims 106-161, wherein the RNA ligase is T4 RNA ligase.

163. A method of producing a modified non-coding RNA of any one of claims 60-105, the method comprising ligating a first RNA comprising a non-coding RNA sequence to a tailing nucleic acid comprising one or more modified nucleotides, in the presence of an RNA ligase, whereby the RNA ligase forms a covalent bond between the 3′ nucleotide of the RNA and the 5′ nucleotide of the tailing nucleic acid to produce the modified non-coding RNA.

164. The method of claim 163, wherein the modified non-coding RNA comprises a poly-A region that is 3′ to the non-coding RNA sequence.

165. The method of claim 164, further comprising circularizing the modified non-coding RNA in the presence of a ribozyme, wherein the modified non-coding RNA comprises a 3′ intron and a 5′ intron, wherein the 3′ intron is 5′ to the non-coding RNA sequence, wherein the 5′ intron is 3′ to the poly-A region, whereby the ribozyme forms a covalent bond between a nucleotide that is 3′ to the 3′ intron and a nucleotide that is 5′ to the 5′ intron to produce a circular non-coding RNA that does not comprise the 5′ intron or the 3′ intron, wherein the poly-A region is between the 3′ and 5′ nucleotides of the non-coding RNA.

166. The method of claim 164, further comprising the steps of:
(i) introducing a 5′ terminal phosphate group onto the first nucleotide of the modified non-coding RNA;
(ii) cleaving one or more 3′ terminal nucleotides of the modified non-coding RNA to produce a modified non-coding RNA with a 3′ terminal hydroxyl group; and
(iii) circularizing the modified non-coding RNA produced in step (ii) in the presence of a circularizing ligase;
whereby the circularizing ligase forms a covalent bond between the 3′ nucleotide of the modified non-coding RNA and the 5′ nucleotide of the modified non-coding RNA to produce a circular modified non-coding RNA, wherein the poly-A region is between the 3′ and 5′ nucleotides of the non-coding RNA.

167. A method of any one of claims 163-166, wherein the tailing nucleic acid further comprises one or more copies of a structural sequence.

168. The method of claim 167, wherein the modified non-coding RNA comprises a poly-A region is between the non-coding RNA sequence and the one or more copies of the structural sequence.

169. The method of claim 168, further comprising circularizing the modified non-coding RNA in the presence of a ribozyme, wherein the modified non-coding RNA comprises a 3′ intron and a 5′ intron, wherein the 3′ intron is 5′ to the non-coding RNA sequence, wherein the 5′ intron is 3′ to the one or more copies of the structural sequence, whereby the ribozyme forms a covalent bond between a nucleotide that is 3′ to the 3′ intron and a nucleotide that is 5′ to the 5′ intron to produce a circular non-coding RNA that does not comprise the 5′ intron or the 3′ intron, wherein the one or more copies of the structural sequence are between the poly-A region and the non-coding RNA sequence of the circular non-coding RNA.

170. The method of claim 168, further comprising the steps of:
(i) introducing a 5′ terminal phosphate group onto the first nucleotide of the modified non-coding RNA;
(ii) cleaving one or more 3′ terminal nucleotides of the modified non-coding RNA to produce a modified non-coding RNA with a 3′ terminal hydroxyl group; and
(iii) circularizing the modified non-coding RNA produced in step (ii) in the presence of a circularizing ligase;
whereby the circularizing ligase forms a covalent bond between the 3′ nucleotide of the modified non-coding RNA and the 5′ nucleotide of the modified non-coding RNA to produce a circular modified non-coding RNA, wherein the one or more copies of the structural sequence are between the poly-A region and the non-coding RNA sequence.

171. The method of claim 166 or 170, wherein the modified non-coding RNA is circularized in the presence of a scaffold nucleic acid, wherein the scaffold nucleic acid is a nucleic acid that is capable of hybridizing with the modified non-coding RNA, wherein the modified non-coding RNA forms a circular secondary structure when bound to the scaffold nucleic acid.

172. The method of claim 171, wherein the scaffold nucleic acid comprises:
(a) a first hybridization sequence comprising 5 or more nucleotides, wherein the first hybridization sequence is complementary to at least the first five (5) nucleotides of the modified non-coding RNA; and
(b) a second hybridization sequence comprising 5 or more nucleotides, wherein the second hybridization sequence is complementary to at least the last five (5) nucleotides of the modified non-coding RNA;
wherein at least the first five (5) nucleotides of the modified non-coding RNA hybridize with the first hybridization sequence, and at least the last five (5) nucleotides of the modified non-coding RNA hybridize with the second hybridization sequence.

173. The method of claim 171 or 172, wherein a last nucleotide of the first hybridization sequence and a first nucleotide of the second hybridization sequence are adjacent in the scaffold nucleic acid and not separated by any other nucleotides.

174. The method of claim 166 or 170, wherein the modified non-coding RNA comprises:
(i) a first self-hybridization sequence that is 5′ to the open reading frame;
(ii) a second self-hybridization sequence that is 3′ to the open reading frame;
(iii) a first non-hybridization sequence that is 5′ to the first self-hybridization sequence; and
(iv) a second non-hybridization sequence that is 3′ to the second self-hybridization sequence,
wherein the first and second self-hybridization sequences are capable of hybridizing with each other,
wherein the first and second self-hybridization sequences are not capable of hybridizing with each other.

175. The method of claim 174, wherein hybridization of the first and second self-hybridization sequences forms a secondary structure in which the 5′ terminal nucleotide and the 3′ terminal nucleotide of the modified non-coding RNA are separated by a distance of less than 100 Å.

176. The method of claim 175, wherein the 5′ terminal nucleotide and the 3′ terminal nucleotide are separated by a distance of less than 90 Å, less than 80 Å, less than 70 Å, less than 60 Å, less than 50 Å, less than 40 Å, less than 30 Å, less than 20 Å, or less than 10 Å.

177. The method of any one of claim 166 or 170-176, wherein the circularizing ligase is T4 RNA ligase.

178. The method of any one of claims 168-177, wherein the structural sequence is a G-quadruplex sequence.

179. The method of claim 178, wherein the G-quadruplex is an RNA G-quadruplex sequence.

180. The method of claim 179, wherein the RNA G-quadruplex sequence comprises the nucleic acid sequence of SEQ ID NO: 2.

181. The method of claim 180, wherein the tailing nucleic acid comprises at least 3 copies of the nucleic acid sequence of SEQ ID NO: 2.

182. The method of claim 178, wherein the G-quadruplex is a DNA G-quadruplex sequence.

183. The method of claim 182, wherein the DNA G-quadruplex sequence comprises the nucleic acid sequence of SEQ ID NO: 3.

184. The method of claim 183, wherein the tailing nucleic acid comprises at least 3 copies of the nucleic acid sequence of SEQ ID NO: 3.

185. The method of any one of claims 168-177, wherein the structural sequence is a telomeric repeat sequence.

186. The method of claim 185, wherein the telomeric repeat sequence comprises the nucleic acid sequence of SEQ ID NO: 4.

187. The method of claim 186, wherein the tailing nucleic acid comprises at least 3 copies of the nucleic acid sequence of SEQ ID NO: 4.

188. The method of any one of claims 168-177, wherein the structural sequence is an aptamer sequence comprising at least two nucleotides that are capable of interacting to form an aptamer, wherein the aptamer is a secondary structure that is capable of binding to a target molecule.

189. The method of any one of claims 163-188, wherein the 5′ nucleotide of the RNA does not comprise a 5′ terminal phosphate group;
wherein the 3′ nucleotide of the RNA comprises a 3′ terminal hydroxyl group;
wherein the 5′ nucleotide of the tailing nucleic acid comprises a 5′ terminal phosphate group; and
wherein the 3′ nucleotide of the tailing nucleic acid does not comprise a 3′ terminal hydroxyl group.

190. The method of any one of claims 163-188, wherein the 5′ nucleotide of the RNA does not comprise a 5′ terminal hydroxyl group;
wherein the 3′ nucleotide of the RNA comprises a 3′ terminal phosphate group;
wherein the 5′ nucleotide of the tailing nucleic acid comprises a 5′ terminal hydroxyl group;
wherein the 3′ nucleotide of the tailing nucleic acid does not comprise a 3′ terminal phosphate group; and
wherein the RNA ligase is an RtcB ligase.

191. The method of any one of claims 163-190, wherein at least 2%, at least 3%, at least 4%, at least 5%, at least 6%, at least 7%, at least 8%, at least 9%, at least 10%, at least 12%, at least 14%, at least 16%, at least 18%, at least 20%, at least 25%, at least 30%, at least 35%, at least 40%, at least 45%, or at least 50% of the nucleotides of the tailing nucleic acid are modified nucleotides.

192. The method of any one of claims 163-191, wherein at least 4, 5, 6, 7, 8, 9, 10, 15, 20, or 25 of the 25 last nucleotides of the tailing nucleic acid are modified nucleotides.

193. The method of any one of claims 163-192, wherein at least one modified nucleotide comprises a modified nucleobase.

194. The method of claim 193, wherein the modified nucleobase is selected from the group consisting of xanthine, allyaminouracil, allyaminothymidine, hypoxanthine, digoxigeninated adenine, digoxigeninated cytosine, digoxigeninated guanine, digoxigeninated uracil, 6-chloropurineriboside, N6-methyladenine, methylpseudouracil, 2-thiocytosine, 2-thiouracil, 5-methyluracil, 4-thiothymidine, 4-thiouracil, 5,6-dihydro-5-methyluracil, 5,6-dihydrouracil, 5-[(3-Indolyl)propionamide-N-allyl]uracil, 5-aminoallylcytosine, 5-aminoallyluracil, 5-bromouracil, 5-bromocytosine, 5-carboxycytosine, 5-carboxymethylesteruracil, 5-carboxyuracil, 5-fluorouracil, 5-formylcytosine, 5-formyluracil, 5-hydroxycytosine, 5-hydroxymethylcytosine, 5-hydroxymethyluracil, 5-hydroxyuracil, 5-iodocytosine, 5-iodouracil, 5-methoxycytosine, 5-methoxyuracil, 5-methylcytosine, 5-methyluracil, 5-propargylaminocytosine, 5-propargylaminouracil, 5-propynylcytosine, 5-propynyluracil, 6-azacytosine, 6-azauracil, 6-chloropurine, 6-thioguanine, 7-deazaadenine, 7-deazaguanine, 7-deaza-7-propargylaminoadenine, 7-deaza-7-propargylaminoguanine, 8-azaadenine, 8-azidoadenine, 8-chloroadenine, 8-oxoadenine, 8-oxoguanine, araadenine, aracytosine, araguanine, arauracil, biotin-16-7-deaza-7-propargylaminoguanine, biotin-16-aminoallylcytosine, biotin-16-aminoallyluracil, cyanine 3-5-propargylaminocytosine, cyanine 3-6-propargylaminouracil, cyanine 3-aminoallylcytosine, cyanine 3-aminoallyluracil, cyanine 5-6-propargylaminocytosine, cyanine 5-6-propargylaminouracil, cyanine 5-aminoallylcytosine, cyanine 5-aminoallyluracil, cyanine 7-aminoallyluracil, dabcyl-5-3-aminoallyluracil, desthiobiotin-16-aminoallyl-uracil, desthiobiotin-6-aminoallylcytosine, isoguanine, N1-ethylpseudouracil, N1-methoxymethylpseudouracil, N1-methyladenine, N1-methylpseudouracil, N1-propylpseudouracil, N2-methylguanine, N4-biotin-OBEA-cytosine, N4-methylcytosine, N6-methyladenine, O6-methylguanine, pseudoisocytosine, pseudouracil, thienocytosine, thienoguanine, thienouracil, xanthosine, 3-deazaadenine, 2,6-diaminoadenine, 2,6-daminoguanine, 5-carboxamide-uracil, 5-ethynyluracil, N6-isopentenyladenine (i6A), 2-methyl-thio-N6-isopentenyladenine (ms2i6A), 2-methylthio-N6-methyladenine (ms2m6A), N6-(cis-hydroxyisopentenyl)adenine (io6A), 2-methylthio-N6-(cis-hydroxyisopentenyl)adenine (ms2io6A), N6-glycinylcarbamoyladenine (g6A), N6-threonylcarbamoyladenine (i6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

195. The method of any one of claims 163-194, wherein at least one modified nucleotide comprises a modified sugar.

196. The method of claim 195, wherein the modified sugar is selected from the group consisting of 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose.

197. The method of any one of claims 163-196, wherein at least one modified nucleotide comprises a 2′ modification.

198. The method of claim 197, wherein the 2′ modification is selected from the group consisting of a locked-nucleic acid (LNA) modification, 2′-fluoro (2′-F), 2′-O-methoxy-ethyl (2′-MOE), and 2′-O-methylation (2′-OMe).

199. The method of any one of claims 163-198, wherein at least one modified nucleotide comprises a modified phosphate.

200. The method of claim 199, wherein the modified phosphate is selected from the group consisting of phosphorothioate (PS), phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

201. The method of any one of claims 163-200, wherein the tailing nucleic acid comprises at least 3, at least 4, at least 5, or at least 6 phosphorothioates.

202. The method of claim 201, wherein the tailing nucleic acid comprises at least 6 phosphorothioates.

203. The method of any one of claims 163-201, wherein the tailing nucleic acid comprises at least 3 guanine nucleotides and least 3 phosphorothioates.

204. The method of any one of claims 163-203, wherein the tailing nucleic acid comprises at least 6 nucleotides comprising a 2′ modification.

205. The method of any one of claims 163-204, wherein the tailing nucleic acid comprises at least 3 deoxyribose sugars.

206. The method of claim 205, wherein the tailing nucleic acid comprises at least 5, at least 10, at least 15, at least 20, or at least 23 deoxyribose sugars.

207. The method of claim 206, wherein the tailing nucleic acid comprises at least 23 deoxyribose sugars.

208. The method of any one of claim 163-168 or 178-207, wherein the 3′ terminal nucleotide of the tailing nucleic acid comprises a dideoxyadenosine, dideoxycytidine, dideoxyguanosine, dideoxythymidine, dideoxyuridine, or inverted-deoxythymidine.

209. The method of any one of claims 163-208, wherein the tailing nucleic acid comprises a first modified nucleotide and a second modified nucleotide, wherein the first and second modified nucleotides comprise different structures.

210. The method of any one of claims 163-209, wherein at least 25%, at least 30%, at least 40%, at least 50%, at least 60%, at least 70%, at least 80%, at least 90%, at least 95%, at least 96%, at least 97%, at least 98%, or at least 99% of the poly-A region of the modified non-coding RNA are adenosine nucleotides.

211. The method of any one of claims 163-210, wherein the poly-A region of the modified non-coding RNA comprises at least 25-500 nucleotides.

212. The method of claim 211, wherein the poly-A region of the modified non-coding RNA comprises at least 50, at least 100, at least 150, or at least 200 nucleotides.

213. The method of any one of claims 163-212, wherein the RNA ligase is T4 RNA ligase.

214. A modified mRNA produced by the method of any one of claims 106-162.

215. The modified mRNA of any one of claim 1-59 or 214, wherein the mRNA encodes an antigen or a therapeutic protein.

216. The modified mRNA of claim 215, wherein the antigen is a viral antigen, bacterial antigen, protozoal antigen, or fungal antigen.

217. The modified mRNA of claim 215, wherein the therapeutic protein is an enzyme, transcription factor, cell surface receptor, growth factor, or clotting factor.

218. The modified mRNA of any one of claim 1-59 or 215-217, wherein the open reading frame is codon-optimized for expression in a cell.

219. The modified mRNA of claim 218, wherein the modified mRNA is codon-optimized for expression in a mammalian cell.

220. The modified mRNA of claim 219, wherein the modified mRNA is codon-optimized for expression in a human cell.

221. A modified non-coding RNA produced by the method of any one of claims 163-213.

222. The modified non-coding RNA of any one of claim 60-105 or 221, wherein the modified non-coding RNA is a guide RNA (gRNA), a prime editing guide RNA (pegRNA), or a long non-coding RNA (lncRNA).

223. A lipid nanoparticle comprising the modified mRNA of any one of claim 1-59 or 214-220 or the modified non-coding RNA of any one of claim 60-105, 221, or 222.

224. A cell comprising the modified mRNA of any one of claim 1-59 or 214-220 or the modified non-coding RNA of any one of claim 60-105, 221, or 222.

225. The cell of claim 224, wherein the cell is a mammalian cell.

226. The cell of claim 225, wherein the cell is a human cell.

227. A composition comprising the modified mRNA of any one of claim 1-59 or 214-220, the modified non-coding RNA of any one of claim 60-105, 221, or 222, the lipid nanoparticle of claim 223, or the cell of any one of claims 224-226.

228. A pharmaceutical composition comprising the composition of claim 227, and a pharmaceutically acceptable excipient.

229. A method comprising introducing the mRNA of any one of claim 1-59 or 214-220, the modified non-coding RNA of any one of claim 60-105, 221, or 222, or the lipid nanoparticle of claim 223, into a cell.

230. A method comprising introducing the mRNA of any one of claim 1-59 or 214-220, the modified non-coding RNA of any one of claim 60-105, 221, or 222, the lipid nanoparticle of claim 223, the cell of any one of claims 224-226, or the composition of claim 227 or 228, into a subject.

231. A method of vaccinating a subject, comprising introducing the modified mRNA of any one of claim 1-59 or 214-220, the lipid nanoparticle of claim 223, the cell of any one of claims 224-226, or the composition of claim 227 or 228 into the subject, wherein the open reading frame of the modified mRNA encodes an antigen.

232. A method of replacing an enzyme in a subject, comprising introducing the modified mRNA of any one of claim 1-59 or 214-220, the lipid nanoparticle of claim 223, the cell of any one of claims 224-226, or the composition of claim 227 or 228, into the subject, wherein the open reading frame of the modified mRNA encodes an enzyme.

233. A method of modifying the genome of a subject, comprising introducing the modified non-coding RNA of any one of claim 60-105, 221, or 222, the lipid nanoparticle of claim 223, or the composition of claim 227 or 228 into a subject.

234. The method of any one of claims 230-233, wherein the subject is a mammal.

235. The method of claim 234, wherein the subject is a human.

236. The modified mRNA of any one of claim 1-59 or 214-220, the modified non-coding RNA of any one of claim 60-105, 221, or 222, the lipid nanoparticle of claim 223, the cell of any one of claims 224-226, or the composition of claim 227 or 228, for use as a medicament.

237. A kit comprising the RNA and the tailing nucleic acid of any one of claims 106-213.

238. The kit of claim 237, further comprising an RNA ligase.

239. A kit comprising the pharmaceutical composition of claim 228 and a delivery device.

240. A method for purifying a modified mRNA or a modified non-coding RNA, comprising:
contacting a mixture comprising the modified mRNA of any one of claims 1-59 or the modified non-coding RNA of any one of claims 60-105 with a purification medium, wherein the modified mRNA or modified non-coding RNA interacts with the purification medium to form a modified RNA-purification medium conjugate;
separating the modified RNA-purification medium conjugate from the mixture; and
eluting the modified mRNA or the modified non-coding RNA from the modified RNA-purification medium conjugate with a solvent.

241. The method of claim 240, wherein the purification medium comprises a paramagnetic bead.


SEQUENCE LISTING (52 sequences)

SEQ ID NO: 1 - RNA, 14 nt, Artificial Sequence
  Synthetic
aaaaaaaaaaaaaa

SEQ ID NO: 2 - RNA, 6 nt, Artificial Sequence
  Synthetic
ggggcc

SEQ ID NO: 3 - DNA, 6 bp, Artificial Sequence
  Synthetic
ggggcc

SEQ ID NO: 4 - DNA, 6 bp, Artificial Sequence
  Synthetic
tagggt

SEQ ID NO: 5 - DNA, 6 bp, Artificial Sequence
  Synthetic
taccct

SEQ ID NO: 6 - DNA, 30 bp, Artificial Sequence
  Synthetic
aaaaaaaaaaaaaaaaaaaaaaaaaaaaac

SEQ ID NO: 7 - DNA, 30 bp, Artificial Sequence; identical to SEQ ID NO: 6
  Synthetic

SEQ ID NO: 8 - DNA, 30 bp, Artificial Sequence
  Synthetic
aaaaaaaaaaaaaaaaaaaaaaaaaaaaat

SEQ ID NO: 9 - DNA, 30 bp, Artificial Sequence
  Synthetic
aaaaaaaaaaaaaaaaaaaaaaaaaagggt

SEQ ID NO: 10 - DNA, 30 bp, Artificial Sequence; identical to SEQ ID NO: 6
  Synthetic

SEQ ID NO: 11 - DNA, 30 bp, Artificial Sequence; identical to SEQ ID NO: 6
  Synthetic

SEQ ID NO: 12 - DNA, 30 bp, Artificial Sequence
  Synthetic
aaaaaatacccttacccttacccttacccc

SEQ ID NO: 13 - DNA, 30 bp, Artificial Sequence
  Synthetic
aaaaaatagggttagggttagggttagggc

SEQ ID NO: 14 - DNA, 30 bp, Artificial Sequence; identical to SEQ ID NO: 9
  Synthetic

SEQ ID NO: 15 - DNA, 30 bp, Artificial Sequence
  Synthetic
aaaaaaaggggccggggccggggccggggt

SEQ ID NO: 16 - DNA, 30 bp, Artificial Sequence; identical to SEQ ID NO: 15
  Synthetic

SEQ ID NO: 17 - DNA, 30 bp, Artificial Sequence
  Synthetic
aaaaaatagggttagggttagggttagggt

SEQ ID NO: 18 - DNA, 36 bp, Artificial Sequence
  Synthetic
gcatcacaaatttcacaaataaagcatttttttcac

SEQ ID NO: 19 - DNA, 57 bp, Artificial Sequence
  Synthetic
acattattggtgccgcgcagcttcacctaattattactgaggcatacactaaagata

SEQ ID NO: 20 - DNA, 56 bp, Artificial Sequence
  Synthetic
acattacttcttggccttgtaggtggtaattattactgaggcatacactaaagata

SEQ ID NO: 21 - DNA, 55 bp, Artificial Sequence
  Synthetic
acattacacggtcaccacgccgccgtaattattactgaggcatacactaaagata

SEQ ID NO: 22 - DNA, 32 bp, Artificial Sequence
  Synthetic
acggggccgtcggaggggaataatgttatctt

SEQ ID NO: 23 - DNA, 32 bp, Artificial Sequence
  Synthetic
ggcgccgggcagctgcacggtaatgttatctt

SEQ ID NO: 24 - DNA, 34 bp, Artificial Sequence
  Synthetic
gtcctgcagggaggagtcctggtaatgttatctt

SEQ ID NO: 25 - DNA, 54 bp, Artificial Sequence
  Synthetic
acattaagtcgcagcggtagtggccaattattactgaaatcgtagactaagata

SEQ ID NO: 26 - DNA, 59 bp, Artificial Sequence
  Synthetic
acattacattagcagggaagttgaccccgtaattattactgaaatcgtagactaagata

SEQ ID NO: 27 - DNA, 57 bp, Artificial Sequence
  Synthetic
acattagcttcggcgtgctcgtacagctaattattactgaaatcgtagactaagata

SEQ ID NO: 28 - DNA, 35 bp, Artificial Sequence
  Synthetic
cctccctccaagagcagtgccattaatgttatctt

SEQ ID NO: 29 - DNA, 30 bp, Artificial Sequence
  Synthetic
tgcgctgcatcaccgggctaatgttatctt

SEQ ID NO: 30 - DNA, 34 bp, Artificial Sequence
  Synthetic
cctggcggggtagtccgctgtgtaatgttatctt

SEQ ID NO: 31 - DNA, 19 bp, Artificial Sequence
  Synthetic
catacactaaagataacat

SEQ ID NO: 32 - DNA, 19 bp, Artificial Sequence
  Synthetic
tcgtagactaagataacat

SEQ ID NO: 33 - DNA, 49 bp, Artificial Sequence
  Synthetic
aaaaaaaaaaaaaaaaaaaaaaaaaaggggccggggccggggccggggt

SEQ ID NO: 34 - DNA, 49 bp, Artificial Sequence; identical to SEQ ID NO: 33
  Synthetic

SEQ ID NO: 35 - DNA, 50 bp, Artificial Sequence
  Synthetic
aaaaaaaaaaaaaaaaaaaaaaaaaatagggttagggttagggttagggt

SEQ ID NO: 36 - DNA, 24 bp, Artificial Sequence
  Synthetic
agagacgttcgcggccgcggcgcc

SEQ ID NO: 37 - DNA, 37 bp, Artificial Sequence
  Synthetic
ttaaaaaacctcccacacctccccctgaacctgaaac

SEQ ID NO: 38 - DNA, 60 bp, Artificial Sequence
  Synthetic
aaaaaaaaaaaaaaaaaaaaaaaaaaaaaaaaaaaaagagacgttcgcggccgcggcgcc

SEQ ID NO: 39 - DNA, 60 bp, Artificial Sequence
  Synthetic
tttttttttttttttttttttttttttttttcctcccacacctccccctgaacctgaaac

SEQ ID NO: 40 - DNA, 20 bp, Artificial Sequence
  Synthetic
tgacattctcaccaccgtgt

SEQ ID NO: 41 - DNA, 20 bp, Artificial Sequence
  Synthetic
agtcgtccacacccttcatc

SEQ ID NO: 42 - DNA, 21 bp, Artificial Sequence
  Synthetic
ttcttggccatgtaggtggtc

SEQ ID NO: 43 - DNA, 20 bp, Artificial Sequence
  Synthetic
aggacggcgagttcatctac

SEQ ID NO: 44 - DNA, 22 bp, Artificial Sequence
  Synthetic
caccattggcaatgagcggttc

SEQ ID NO: 45 - DNA, 22 bp, Artificial Sequence
  Synthetic
aggtctttgcggatgtccacgt

SEQ ID NO: 46 - DNA, 24 bp, Artificial Sequence
  Synthetic
cttggattcctacaaagaagcagc

SEQ ID NO: 47 - DNA, 22 bp, Artificial Sequence
  Synthetic
tcctccttctggaactgctgca

SEQ ID NO: 48 - DNA, 30 bp, Artificial Sequence
  Synthetic
aaaaaaaaaaaaaaaaaaaaaaaaaagggc

SEQ ID NO: 49 - DNA, 30 bp, Artificial Sequence; identical to SEQ ID NO: 9
  Synthetic

SEQ ID NO: 50 - DNA, 30 bp, Artificial Sequence; identical to SEQ ID NO: 48
  Synthetic

SEQ ID NO: 51 - DNA, 30 bp, Artificial Sequence; identical to SEQ ID NO: 9
  Synthetic

SEQ ID NO: 52 - DNA, 30 bp, Artificial Sequence; identical to SEQ ID NO: 48
  Synthetic